SEQUENCE LISTING (343 sequences)

SEQ ID NO: 1 - protein, 9 aa, synthetic construct
  REGION 1..9: note = antibody sequence
ATVSGFSIG

SEQ ID NO: 2 - protein, 9 aa, synthetic construct
  REGION 1..9: note = antibody sequence
ASGFTFSSS

SEQ ID NO: 3 - protein, 9 aa, synthetic construct
  REGION 1..9: note = antibody sequence
ASGRTFSSF

SEQ ID NO: 4 - protein, 9 aa, synthetic construct
  REGION 1..9: note = antibody sequence
ASGLIFSIN

SEQ ID NO: 5 - protein, 9 aa, synthetic construct
  REGION 1..9: note = antibody sequence
ASGNIFRIN

SEQ ID NO: 6 - protein, 9 aa, synthetic construct
  REGION 1..9: note = antibody sequence
ASTSVFGNT

SEQ ID NO: 7 - protein, 9 aa, synthetic construct
  REGION 1..9: note = antibody sequence
ASGSIYHVN

SEQ ID NO: 8 - protein, 9 aa, synthetic construct
  REGION 1..9: note = antibody sequence
ASGNIFRIN

SEQ ID NO: 9 - protein, 9 aa, synthetic construct
  REGION 1..9: note = antibody sequence
ASGNIFRIN

SEQ ID NO: 10 - protein, 9 aa, synthetic construct
  REGION 1..9: note = antibody sequence
ASRSFFSIN

SEQ ID NO: 11 - protein, 9 aa, synthetic construct
  REGION 1..9: note = antibody sequence
HSTITFRIN

SEQ ID NO: 12 - protein, 9 aa, synthetic construct
  REGION 1..9: note = antibody sequence
ASMTTLGFK

SEQ ID NO: 13 - protein, 9 aa, synthetic construct
  REGION 1..9: note = antibody sequence
ASTSVFGNT

SEQ ID NO: 14 - protein, 9 aa, synthetic construct
  REGION 1..9: note = antibody sequence
ASGMRSSLA

SEQ ID NO: 15 - protein, 10 aa, synthetic construct
  REGION 1..10: note = antibody sequence
ASGSTFISIK

SEQ ID NO: 16 - protein, 9 aa, synthetic construct
  REGION 1..9: note = antibody sequence
ASGSTFRFN

SEQ ID NO: 17 - protein, 9 aa, synthetic construct
  REGION 1..9: note = antibody sequence
ASGSRFSIN

SEQ ID NO: 18 - protein, 9 aa, synthetic construct
  REGION 1..9: note = antibody sequence
ASGSIFSIN

SEQ ID NO: 19 - protein, 9 aa, synthetic construct
  REGION 1..9: note = antibody sequence
ASGSIFRIN

SEQ ID NO: 20 - protein, 9 aa, synthetic construct
  REGION 1..9: note = antibody sequence
ASTSVFGNT

SEQ ID NO: 21 - protein, 9 aa, synthetic construct
  REGION 1..9: note = antibody sequence
ASGSIFSIN

SEQ ID NO: 22 - protein, 9 aa, synthetic construct
  REGION 1..9: note = antibody sequence
ASRNFFTFR

SEQ ID NO: 23 - protein, 9 aa, synthetic construct
  REGION 1..9: note = antibody sequence
ASGITFRFN

SEQ ID NO: 24 - protein, 9 aa, synthetic construct
  REGION 1..9: note = antibody sequence
ASGRSILIK

SEQ ID NO: 25 - protein, 9 aa, synthetic construct
  REGION 1..9: note = antibody sequence
ASGRIFGRN

SEQ ID NO: 26 - protein, 9 aa, synthetic construct
  REGION 1..9: note = antibody sequence
ASRSTFRFN

SEQ ID NO: 27 - protein, 9 aa, synthetic construct
  REGION 1..9: note = antibody sequence
TSGSIFSIN

SEQ ID NO: 28 - protein, 9 aa, synthetic construct
  REGION 1..9: note = antibody sequence
LSTTMFGFW

SEQ ID NO: 29 - protein, 9 aa, synthetic construct
  REGION 1..9: note = antibody sequence
ASGNIFRIN

SEQ ID NO: 30 - protein, 9 aa, synthetic construct
  REGION 1..9: note = antibody sequence
ASGSIFSTN

SEQ ID NO: 31 - protein, 9 aa, synthetic construct
  REGION 1..9: note = antibody sequence
ASRSSFRIT

SEQ ID NO: 32 - protein, 9 aa, synthetic construct
  REGION 1..9: note = antibody sequence
ASGNIFRIN

SEQ ID NO: 33 - protein, 9 aa, synthetic construct
  REGION 1..9: note = antibody sequence
ASVVPFRYF

SEQ ID NO: 34 - protein, 9 aa, synthetic construct
  REGION 1..9: note = antibody sequence
ASGSIFSIK

SEQ ID NO: 35 - protein, 9 aa, synthetic construct
  REGION 1..9: note = antibody sequence
ASGSAFRMN

SEQ ID NO: 36 - protein, 9 aa, synthetic construct
  REGION 1..9: note = antibody sequence
ASGSIFSIN

SEQ ID NO: 37 - protein, 10 aa, synthetic construct
  REGION 1..10: note = antibody sequence
ANVSPSGAKY

SEQ ID NO: 38 - protein, 10 aa, synthetic construct
  REGION 1..10: note = antibody sequence
ASIMRYGTTT

SEQ ID NO: 39 - protein, 11 aa, synthetic construct
  REGION 1..11: note = antibody sequence
TAINWSGSSTY

SEQ ID NO: 40 - protein, 10 aa, synthetic construct
  REGION 1..10: note = antibody sequence
ARITTGGSTN

SEQ ID NO: 41 - protein, 10 aa, synthetic construct
  REGION 1..10: note = antibody sequence
AHIISGGSTN

SEQ ID NO: 42 - protein, 10 aa, synthetic construct
  REGION 1..10: note = antibody sequence
ARITTLGFTY

SEQ ID NO: 43 - protein, 10 aa, synthetic construct
  REGION 1..10: note = antibody sequence
ATLTHNNRVT

SEQ ID NO: 44 - protein, 10 aa, synthetic construct
  REGION 1..10: note = antibody sequence
AVVKSGGSTN

SEQ ID NO: 45 - protein, 10 aa, synthetic construct
  REGION 1..10: note = antibody sequence
AHIISGGSTN

SEQ ID NO: 46 - protein, 10 aa, synthetic construct
  REGION 1..10: note = antibody sequence
ATITSRDSTN

SEQ ID NO: 47 - protein, 10 aa, synthetic construct
  REGION 1..10: note = antibody sequence
ARINPAGRTY

SEQ ID NO: 48 - protein, 10 aa, synthetic construct
  REGION 1..10: note = antibody sequence
ATISSIGIST

SEQ ID NO: 49 - protein, 10 aa, synthetic construct
  REGION 1..10: note = antibody sequence
ARITTLGFTY

SEQ ID NO: 50 - protein, 10 aa, synthetic construct
  REGION 1..10: note = antibody sequence
ATITIGGNTN

SEQ ID NO: 51 - protein, 10 aa, synthetic construct
  REGION 1..10: note = antibody sequence
AGITKNNYIN

SEQ ID NO: 52 - protein, 10 aa, synthetic construct
  REGION 1..10: note = antibody sequence
ANINSSGRTM

SEQ ID NO: 53 - protein, 10 aa, synthetic construct
  REGION 1..10: note = antibody sequence
AGITSLGFTN

SEQ ID NO: 54 - protein, 10 aa, synthetic construct
  REGION 1..10: note = antibody sequence
AGISRGGRTK

SEQ ID NO: 55 - protein, 10 aa, synthetic construct
  REGION 1..10: note = antibody sequence
ATITNEGNTY

SEQ ID NO: 56 - protein, 10 aa, synthetic construct
  REGION 1..10: note = antibody sequence
ARITTLGFTY

SEQ ID NO: 57 - protein, 10 aa, synthetic construct
  REGION 1..10: note = antibody sequence
AGISRGGRTK

SEQ ID NO: 58 - protein, 10 aa, synthetic construct
  REGION 1..10: note = antibody sequence
ASITTGGRTV

SEQ ID NO: 59 - protein, 10 aa, synthetic construct
  REGION 1..10: note = antibody sequence
ARVSSGGSTT

SEQ ID NO: 60 - protein, 10 aa, synthetic construct
  REGION 1..10: note = antibody sequence
ATISMAGVTN

SEQ ID NO: 61 - protein, 10 aa, synthetic construct
  REGION 1..10: note = antibody sequence
ARITRDGRTM

SEQ ID NO: 62 - protein, 11 aa, synthetic construct
  REGION 1..11: note = antibody sequence
AAISSRGGSTN

SEQ ID NO: 63 - protein, 10 aa, synthetic construct
  REGION 1..10: note = antibody sequence
AAISGRGSTH

SEQ ID NO: 64 - protein, 10 aa, synthetic construct
  REGION 1..10: note = antibody sequence
ATIDSRGRTN

SEQ ID NO: 65 - protein, 10 aa, synthetic construct
  REGION 1..10: note = antibody sequence
ARISSGGSTN

SEQ ID NO: 66 - protein, 10 aa, synthetic construct
  REGION 1..10: note = antibody sequence
ASITKFGNTD

SEQ ID NO: 67 - protein, 10 aa, synthetic construct
  REGION 1..10: note = antibody sequence
ASITTGGRTV

SEQ ID NO: 68 - protein, 10 aa, synthetic construct
  REGION 1..10: note = antibody sequence
AHIISGGSTN

SEQ ID NO: 69 - protein, 10 aa, synthetic construct
  REGION 1..10: note = antibody sequence
ASITSGGGVN

SEQ ID NO: 70 - protein, 10 aa, synthetic construct
  REGION 1..10: note = antibody sequence
AAIASGGFTN

SEQ ID NO: 71 - protein, 10 aa, synthetic construct
  REGION 1..10: note = antibody sequence
AAISFRGSAN

SEQ ID NO: 72 - protein, 10 aa, synthetic construct
  REGION 1..10: note = antibody sequence
AGISRGGRTK

SEQ ID NO: 73 - protein, 11 aa, synthetic construct
  REGION 1..11: note = antibody sequence
NIRRFSYLSGD

SEQ ID NO: 74 - protein, 9 aa, synthetic construct
  REGION 1..9: note = antibody sequence
NVRSFVRTY

SEQ ID NO: 75 - protein, 11 aa, synthetic construct
  REGION 1..11: note = antibody sequence
NAQRRWPLRDY

SEQ ID NO: 76 - protein, 12 aa, synthetic construct
  REGION 1..12: note = antibody sequence
AADVRFGERTPY

SEQ ID NO: 77 - protein, 9 aa, synthetic construct
  REGION 1..9: note = antibody sequence
NARTFVRTY

SEQ ID NO: 78 - protein, 8 aa, synthetic construct
  REGION 1..8: note = antibody sequence
NRRGFRSY

SEQ ID NO: 79 - protein, 18 aa, synthetic construct
  REGION 1..18: note = antibody sequence
YYFVPRNPLFGRRIDFDA

SEQ ID NO: 80 - protein, 9 aa, synthetic construct
  REGION 1..9: note = antibody sequence
NAQTRLWSY

SEQ ID NO: 81 - protein, 9 aa, synthetic construct
  REGION 1..9: note = antibody sequence
NAQTRLWSY

SEQ ID NO: 82 - protein, 10 aa, synthetic construct
  REGION 1..10: note = antibody sequence
YADQPWRGRA

SEQ ID NO: 83 - protein, 9 aa, synthetic construct
  REGION 1..9: note = antibody sequence
STWRLGRNY

SEQ ID NO: 84 - protein, 11 aa, synthetic construct
  REGION 1..11: note = antibody sequence
HVIRPSWMPQY

SEQ ID NO: 85 - protein, 8 aa, synthetic construct
  REGION 1..8: note = antibody sequence
NRLWRPLA

SEQ ID NO: 86 - protein, 9 aa, synthetic construct
  REGION 1..9: note = antibody sequence
NVRSFVRTY

SEQ ID NO: 87 - protein, 10 aa, synthetic construct
  REGION 1..10: note = antibody sequence
TVQRRLGRVY

SEQ ID NO: 88 - protein, 9 aa, synthetic construct
  REGION 1..9: note = antibody sequence
NVRSFVRTY

SEQ ID NO: 89 - protein, 8 aa, synthetic construct
  REGION 1..8: note = antibody sequence
NRRGFRSY

SEQ ID NO: 90 - protein, 9 aa, synthetic construct
  REGION 1..9: note = antibody sequence
NVRSFVRTY

SEQ ID NO: 91 - protein, 8 aa, synthetic construct
  REGION 1..8: note = antibody sequence
AGKVIRWY

SEQ ID NO: 92 - protein, 10 aa, synthetic construct
  REGION 1..10: note = antibody sequence
RARRALRESH

SEQ ID NO: 93 - protein, 9 aa, synthetic construct
  REGION 1..9: note = antibody sequence
NARSFVRTY

SEQ ID NO: 94 - protein, 14 aa, synthetic construct
  REGION 1..14: note = antibody sequence
NARRRFPVPGPTDY

SEQ ID NO: 95 - protein, 5 aa, synthetic construct
  REGION 1..5: note = antibody sequence
NVGNF

SEQ ID NO: 96 - protein, 9 aa, synthetic construct
  REGION 1..9: note = antibody sequence
NAQTRLWSY

SEQ ID NO: 97 - protein, 9 aa, synthetic construct
  REGION 1..9: note = antibody sequence
NAQTRLWSY

SEQ ID NO: 98 - protein, 9 aa, synthetic construct
  REGION 1..9: note = antibody sequence
NVRSFVRTY

SEQ ID NO: 99 - protein, 17 aa, synthetic construct
  REGION 1..17: note = antibody sequence
ALDQHMEVIVSPGRIGS

SEQ ID NO: 100 - protein, 11 aa, synthetic construct
  REGION 1..11: note = antibody sequence
NAQRRWPLRDY

SEQ ID NO: 101 - protein, 11 aa, synthetic construct
  REGION 1..11: note = antibody sequence
NARRPLRWYEY

SEQ ID NO: 102 - protein, 15 aa, synthetic construct
  REGION 1..15: note = antibody sequence
YQNSRGRWYDIFRDY

SEQ ID NO: 103 - protein, 11 aa, synthetic construct
  REGION 1..11: note = antibody sequence
NAQRRWPLRDY

SEQ ID NO: 104 - protein, 11 aa, synthetic construct
  REGION 1..11: note = antibody sequence
NAERRFGMRQV

SEQ ID NO: 105 - protein, 16 aa, synthetic construct
  REGION 1..16: note = antibody sequence
ARLLSLGSRWGYGMFT

SEQ ID NO: 106 - protein, 11 aa, synthetic construct
  REGION 1..11: note = antibody sequence
NAQRRWPLRDY

SEQ ID NO: 107 - protein, 11 aa, synthetic construct
  REGION 1..11: note = antibody sequence
AAGRPWQRTFY

SEQ ID NO: 108 - protein, 12 aa, synthetic construct
  REGION 1..12: note = antibody sequence
AATRWSWGTKSY

SEQ ID NO: 109 - protein, 117 aa, synthetic construct
  REGION 1..117: note = antibody sequence
QVQLQQSGGGLVQAGGSLILRCRATVSGFSIGTMGWYRQAPGKEREFVANVSPSGAKYFADSVKGRFTISRDNANNTVYLQMNSLKPEDTGVYYCNIRRFSYLSGDWGQGTQVTVSS

SEQ ID NO: 110 - protein, 115 aa, synthetic construct
  REGION 1..115: note = antibody sequence
QVQLQESGGGLVQAGGSLRLSCAASGFTFSSSSMGWYRQAPGKQRELVASIMRYGTTTYTDSVKGRFTISRDNGQRTVYLQMNSLKPEDTAVYYCNVRSFVRTYWGQGTLVTVSS

SEQ ID NO: 111 - protein, 118 aa, synthetic construct
  REGION 1..118: note = antibody sequence
QVQLQESGGGLVQAGGSLRLSCAASGRTFSSFAMGWFRQAPGKEREFVTAINWSGSSTYYADSVKGRFTISRDNAKNTVYLQMNSLKPEDTAVYYCNAQRRWPLRDYWGQGTQVTVSS

SEQ ID NO: 112 - protein, 118 aa, synthetic construct
  REGION 1..118: note = antibody sequence
QVQLQESGGGLVQAGGSLRLSCAASGLIFSINAMAWYRQAPGNQRELVARITTGGSTNYADSVKGRFTISRDNAKNTVYLQMNSLKPEDTAVYFCAADVRFGERTPYWGQGTQVTVSS

SEQ ID NO: 113 - protein, 115 aa, synthetic construct
  REGION 1..115: note = antibody sequence
QVQLQASGGGVVQSGGSLRLSCVASGNIFRINAMGWYRQAPGKQRELVAHIISGGSTNYADSVKGRFTISREYAKNMVYLQMNSLKPEDTAVYYCNARTFVRTYWGQGTQVTVSS

SEQ ID NO: 114 - protein, 114 aa, synthetic construct
  REGION 1..114: note = antibody sequence
QVQLQQFGGGLVQPGGSLRLSCQASTSVFGNTAMAWYRQAPGKQRELVARITTLGFTYYADSAKGRFTISRDSAMNTVYLQMNSLKPEDTAVYYCNRRGFRSYWGQGTLVTVSS

SEQ ID NO: 115 - protein, 124 aa, synthetic construct
  REGION 1..124: note = antibody sequence
QVQLQESGGGLVQSGDSLRLSCAASGSIYHVNTMGWYRQSPGKQRELVATLTHNNRVTYADSVKGRFTISRDNAKMTVYLQMDSLKPDDTAVYYCYYFVPRNPLFGRRIDFDAWGQGTQVTVSS

SEQ ID NO: 116 - protein, 115 aa, synthetic construct
  REGION 1..115: note = antibody sequence
QVQLQESGGGVVQSGGSLRLSCVASGNIFRINVMGWYRQAPGKQRELVAVVKSGGSTNYVDSAKGRFTISRDNAKNTAYLHMDSLKPEDTAVYYCNAQTRLWSYWGQGTQVTVSS

SEQ ID NO: 117 - protein, 115 aa, synthetic construct
  REGION 1..115: note = antibody sequence
QVQLQEFGGGVVQSGGSLRLSCVASGNIFRINAMGWYRQAPGKQRELVAHIISGGSTNYADSVKGRFTISREYAKNMVYLQMNSLKPEDTAVYYCNAQTRLWSYWGQGTQVTVSS

SEQ ID NO: 118 - protein, 116 aa, synthetic construct
  REGION 1..116: note = antibody sequence
QVQLQESGGGLVQPGGSLRLSCAASRSFFSINAMGWYRQAPGKQRELVATITSRDSTNVADSVKGRFTISRDYAKNIVYLQMDSLRPEDTATYYCYADQPWRGRAWGQGTQVTVSS

SEQ ID NO: 119 - protein, 115 aa, synthetic construct
  REGION 1..115: note = antibody sequence
QVQLQESGGGVVQAGGSLNLSCTHSTITFRINTMAYYRQAPGSQRALVARINPAGRTYYPDSVKGRFTISRDNAKNQVYLQMNDLKPEDTAVYYCSTWRLGRNYWGQGTLVTVSS

SEQ ID NO: 120 - protein, 117 aa, synthetic construct
  REGION 1..117: note = antibody sequence
QVQLQEFGGGLVQAGGVLRLSCVASMTTLGFKTMGWYRQAPGKQRELVATISSIGISTYANSVKGRFTISRDNAKNTVYLQMNSLKPEDTAVYFCHVIRPSWMPQYWGQGTLVTVSS

SEQ ID NO: 121 - protein, 114 aa, synthetic construct
  REGION 1..114: note = antibody sequence
QVQLQEFGGGLVQPGGSLRLSCQASTSVFGNTAMAWYRQAPGKQRELVARITTLGFTYYADSAKGRFTISRDSAMNTVYLQMNSLKPEDTAVYYCNRLWRPLAWGQGTQVTVSS

SEQ ID NO: 122 - protein, 115 aa, synthetic construct
  REGION 1..115: note = antibody sequence
QVQLQESGGGLVQPGGSLRLSCAASGMRSSLAIMGWYRQAPGKQRELVATITIGGNTNYADSVKGRFAISRDNTKRTVYLQMNSLTPEDTAVYYCNVRSFVRTYWGQGTQVTVSS

SEQ ID NO: 123 - protein, 117 aa, synthetic construct
  REGION 1..117: note = antibody sequence
QVQLQQSGGGLVQAGGSLRLSCAASGSTFISIKTMGWYRQAPGKQRELVAGITKNNYINYADSVKGRFTISRDNGKNTVYLQMNGLKPEDTAVYYCTVQRRLGRVYWGQGTLVTVSS

SEQ ID NO: 124 - protein, 115 aa, synthetic construct
  REGION 1..115: note = antibody sequence
QVQLQESGGGLVQAGGSLRLSCTASGSTFRFNDMGWYRQAPGKQRELVANINSSGRTMYPDSVKGRFTISKDNVKNTVYLQMNSLTPEDTAVYYCNVRSFVRTYWGQGTQVTVSS

SEQ ID NO: 125 - protein, 114 aa, synthetic construct
  REGION 1..114: note = antibody sequence
QVQLQESGGGLVQAGGSLTLSCVASGSRFSINTMGWYRQAPGKQRELVAGITSLGFTNYADSVKGRFTISRDNAKNTVYLQMNNLKVEDTAVYYCNRRGFRSYWGQGTLVTVSS

SEQ ID NO: 126 - protein, 114 aa, synthetic construct
  REGION 1..114: note = antibody sequence
QVQLVESGGGFVQAGGSLRLSCAASGSIFSINYGNWYRQAPGKQRELVAGISRGGRTKYADSVKGRFTISRDSAKTLTLQMTSLKPEDTAVYYCNVRSFVRTYWGQGTQVTVSS

SEQ ID NO: 127 - protein, 114 aa, synthetic construct
  REGION 1..114: note = antibody sequence
QVQLQESGGGLVQSGGSLRLSCSASGSIFRINLMGWYRQAPGKQRELVATITNEGNTYYADSVKGRFTISRDNANNTWYLQMNSLKPEDTAVYECAGKVIRWYWGQGTQVTVSS

SEQ ID NO: 128 - protein, 116 aa, synthetic construct
  REGION 1..116: note = antibody sequence
QVQLQEFGGGLVQPGGSLRLSCQASTSVFGNTAMAWYRQAPGKQRELVARITTLGFTYYADSAKGRFTISRDSAMNTVYLQMNSLKPEDTAVYYCRARRALRESHWGQGTQVTVSS

SEQ ID NO: 129 - protein, 114 aa, synthetic construct
  REGION 1..114: note = antibody sequence
QVQLQESGGGFVQAGGSLRLSCAASGSIFSINYGNWYRQAPGKQRELVAGISRGGRTKYADSVKGRFTISRDSAKTLTLQMTSLKPEDTAIYSCNARSFVRTYWGQGTLVTVSS

SEQ ID NO: 130 - protein, 120 aa, synthetic construct
  REGION 1..120: note = antibody sequence
QVQLQESGGGLVTAGGSLRLSCAASRNFFTFRAMGWYRQAPGKQREMVASITTGGRTVYADSVKGRFTISKSNANNTVYLQMNSLEAEDTAVYYCNARRRFPVPGPTDYWGRGTLVTVSS

SEQ ID NO: 131 - protein, 111 aa, synthetic construct
  REGION 1..111: note = antibody sequence
QVQLQESGGGLVQAGGSLRLSCAASGITFRFNAMGWYRQAPGKERELVARVSSGGSTTYADSVKARFTTFRDNVKNIGYLQMTSLKPEDTAVYYCNVGNFWGQGTQVTVSS

SEQ ID NO: 132 - protein, 115 aa, synthetic construct
  REGION 1..115: note = antibody sequence
QVQLQESGGGLVRTGESLGLSCAASGRSILIKGMGWYRQAPGKEREMVATISMAGVTNYSDSVKGRFTISRDNYKKTVSLQMNNLRPEDTAVYVCNAQTRLWSYWGQGTQVTVSS

SEQ ID NO: 133 - protein, 115 aa, synthetic construct
  REGION 1..115: note = antibody sequence
QVQLVESGGGLVQAGGSLRLSCAASGRIFGRNAMAWYRQVPGKERELVARITRDGRTMYVDSAKGRFTISRDNAKNTAYLHMDSLKPEDTAVYYCNAQTRLWSYWGQGTQVTVSS

SEQ ID NO: 134 - protein, 116 aa, synthetic construct
  REGION 1..116: note = antibody sequence
QVQLQESGGGLVQAGGSLRLSCAASRSTFRFNVMGWYRQAPGKQRELVAAISSRGGSTNYADSVQGRFTISRDNAKNTVSLQMNSLKPEDTAVYYCNVRSFVRTYWGQGTQVTVSS

SEQ ID NO: 135 - protein, 123 aa, synthetic construct
  REGION 1..123: note = antibody sequence
QVQLQEFGGGLVQAGGSLRLSCATSGSIFSINAVGWYRQAPGNQRELVAAISGRGSTHYADSVKGRFTISRDTAKNTVYLQMNSLKPEDTAVYYCALDQHMEVIVSPGRIGSWGQGTLVTVSS

SEQ ID NO: 136 - protein, 117 aa, synthetic construct
  REGION 1..117: note = antibody sequence
QVQLQESGGGLVQAGESLTLSCALSTTMFGFWPMAWFRQTPGQRRELIATIDSRGRTNIADSVKGRFTISRDNTKNTLYLRMNSLKPEDTAVYYCNAQRRWPLRDYWGQGTQVTVSS

SEQ ID NO: 137 - protein, 117 aa, synthetic construct
  REGION 1..117: note = antibody sequence
QVQLQEFGGGVVQSGGSLRLSCVASGNIFRINAMGWYRQAPGKQRELVARISSGGSTNYADSVKGRFTISRDNVKNTVTLQMNSLKPEDTAVYYCNARRPLRWYEYWGQGTLVTVSS

SEQ ID NO: 138 - protein, 121 aa, synthetic construct
  REGION 1..121: note = antibody sequence
QVQLQASGGGLVQPGGSLRLSCAASGSIFSTNAMGWYRQAPGKQREVIASITKFGNTDYADSVKGRFTISRDNAKNIVYLQMNSLKPEDTAVYYCYQNSRGRWYDIFRDYWGQGTLVTVSS

SEQ ID NO: 139 - protein, 117 aa, synthetic construct
  REGION 1..117: note = antibody sequence
QVQLVESGGGFVQAGGSLRLSCVASRSSFRITTMNWYRQAPGKQREMVASITTGGRTVYADSVKGRFTISKSNANNTVYLQMNSLEAEDTAVYYCNAQRRWPLRDYWGQGTLVTVSS

SEQ ID NO: 140 - protein, 115 aa, synthetic construct
  REGION 1..115: note = antibody sequence
QVQLQESGGGVVQSGGSLRLSCVASGNIFRINVMGWYRQAPGKQRELVAVVKSGGSTNYVDSAKGRFTISRDNAKNTAYLHMDSLKPEDTAVYYCNAQTRLWSYWGQGTLVTVSS

SEQ ID NO: 141 - protein, 117 aa, synthetic construct
  REGION 1..117: note = antibody sequence
QVQLQEFGGGVVQSGGSLRLSCVASGNIFRINAMGWYRQAPGKQRELVAHIISGGSTNYADSVKGRFTISREYAKNMVYLQMNSLKPEDTAVYYCNAERRFGMRQVWGQGTQVTVSS

SEQ ID NO: 142 - protein, 122 aa, synthetic construct
  REGION 1..122: note = antibody sequence
QVQLQESGGGLVQAGGSLRLSCAASVVPFRYFPMGWYRQAPGRQRELVASITSGGGVNYADFVEGRFTISRDNAKNTFYLQMSSLKPEDTAVYYCARLLSLGSRWGYGMFTWGKGTLVTVSS

SEQ ID NO: 143 - protein, 117 aa, synthetic construct
  REGION 1..117: note = antibody sequence
QVQLQESGGGLVQPGGSLRLSCAASGSIFSIKTMGWYRQAPGKQRELVAAIASGGFTNYADSVKGRFTISRDNARNTVYLQMNSLKPEDTAVYYCNAQRRWPLRDYWGQGTLVTVSS

SEQ ID NO: 144 - protein, 117 aa, synthetic construct
  REGION 1..117: note = antibody sequence
QVQLQESGGGLVRDGGSLTLSCAASGSAFRMNSMAWYRQVPGKQRELVAAISFRGSANYANSVKGRFTISRDNGKNTVYLQMNSLKPEDTAVYYCAAGRPWQRTFYWGQGTQVTVSS

SEQ ID NO: 145 - protein, 117 aa, synthetic construct
  REGION 1..117: note = antibody sequence
QVQLQESGGGFVQAGGSLRLSCAASGSIFSINYGNWYRQAPGKQRELVAGISRGGRTKYADSVKGRFTISRDSAKTLTLQMTSLKPEDSGVYYCAATRWSWGTKSYWGQGTQVTVSS

SEQ ID NO: 146 - protein, 10 aa, synthetic construct
  REGION 1..10: note = antibody sequence
ASGGTFGAGV

SEQ ID NO: 147 - protein, 10 aa, synthetic construct
  REGION 1..10: note = antibody sequence
ASGSNISTNV

SEQ ID NO: 148 - protein, 10 aa, synthetic construct
  REGION 1..10: note = antibody sequence
ASGSTFSITY

SEQ ID NO: 149 - protein, 10 aa, synthetic construct
  REGION 1..10: note = antibody sequence
DSRTIFIFNA

SEQ ID NO: 150 - protein, 10 aa, synthetic construct
  REGION 1..10: note = antibody sequence
ASGIPFSIIY

SEQ ID NO: 151 - protein, 10 aa, synthetic construct
  REGION 1..10: note = antibody sequence
ASGNIFGINS

SEQ ID NO: 152 - protein, 10 aa, synthetic construct
  REGION 1..10: note = antibody sequence
ASGFTFSSYW

SEQ ID NO: 153 - protein, 10 aa, synthetic construct
  REGION 1..10: note = antibody sequence
VSGSIISHNV

SEQ ID NO: 154 - protein, 10 aa, synthetic construct
  REGION 1..10: note = antibody sequence
ASGRIFNIED

SEQ ID NO: 155 - protein, 10 aa, synthetic construct
  REGION 1..10: note = antibody sequence
ASGSVSAIET

SEQ ID NO: 156 - protein, 10 aa, synthetic construct
  REGION 1..10: note = antibody sequence
AFGSTSSITY

SEQ ID NO: 157 - protein, 10 aa, synthetic construct
  REGION 1..10: note = antibody sequence
ASGGPVSDNV

SEQ ID NO: 158 - protein, 10 aa, synthetic construct
  REGION 1..10: note = antibody sequence
ASGSTFSITY

SEQ ID NO: 159 - protein, 10 aa, synthetic construct
  REGION 1..10: note = antibody sequence
GAGSAFGWNA

SEQ ID NO: 160 - protein, 10 aa, synthetic construct
  REGION 1..10: note = antibody sequence
PSGSIFSFDV

SEQ ID NO: 161 - protein, 12 aa, synthetic construct
  REGION 1..12: note = antibody sequence
SMGSDGFTQIEN

SEQ ID NO: 162 - protein, 12 aa, synthetic construct
  REGION 1..12: note = antibody sequence
SISTSGTTNYLA

SEQ ID NO: 163 - protein, 12 aa, synthetic construct
  REGION 1..12: note = antibody sequence
EMSRRGSTFYAD

SEQ ID NO: 164 - protein, 12 aa, synthetic construct
  REGION 1..12: note = antibody sequence
AISSGGSTKYAD

SEQ ID NO: 165 - protein, 12 aa, synthetic construct
  REGION 1..12: note = antibody sequence
EMSSRGSKFYAD

SEQ ID NO: 166 - protein, 11 aa, synthetic construct
  REGION 1..11: note = antibody sequence
DITRGNRKYAD

SEQ ID NO: 167 - protein, 12 aa, synthetic construct
  REGION 1..12: note = antibody sequence
TINTGGYTTYYS

SEQ ID NO: 168 - protein, 12 aa, synthetic construct
  REGION 1..12: note = antibody sequence
CISGSGFTNYIA

SEQ ID NO: 169 - protein, 12 aa, synthetic construct
  REGION 1..12: note = antibody sequence
TITRTGAPTYAN

SEQ ID NO: 170 - protein, 12 aa, synthetic construct
  REGION 1..12: note = antibody sequence
VISTGGTTKYAP

SEQ ID NO: 171 - protein, 12 aa, synthetic construct
  REGION 1..12: note = antibody sequence
EMSRRGSTFYAD

SEQ ID NO: 172 - protein, 12 aa, synthetic construct
  REGION 1..12: note = antibody sequence
QITSGGATSYAD

SEQ ID NO: 173 - protein, 12 aa, synthetic construct
  REGION 1..12: note = antibody sequence
EISSRGSVFYAD

SEQ ID NO: 174 - protein, 12 aa, synthetic construct
  REGION 1..12: note = antibody sequence
TIESGGWADYSV

SEQ ID NO: 175 - protein, 12 aa, synthetic construct
  REGION 1..12: note = antibody sequence
QHRTPGAIDYAD

SEQ ID NO: 176 - protein, 9 aa, synthetic construct
  REGION 1..9: note = antibody sequence
HYADGRFGS

SEQ ID NO: 177 - protein, 8 aa, synthetic construct
  REGION 1..8: note = antibody sequence
YAAWPLNT

SEQ ID NO: 178 - protein, 13 aa, synthetic construct
  REGION 1..13: note = antibody sequence
SVGARRDEDDYVY

SEQ ID NO: 179 - protein, 13 aa, synthetic construct
  REGION 1..13: note = antibody sequence
AASRSGRWLDDAR

SEQ ID NO: 180 - protein, 13 aa, synthetic construct
  REGION 1..13: note = antibody sequence
SVGARRDDNDYVY

SEQ ID NO: 181 - protein, 13 aa, synthetic construct
  REGION 1..13: note = antibody sequence
NAEIVTQIPFPPR

SEQ ID NO: 182 - protein, 13 aa, synthetic construct
  REGION 1..13: note = antibody sequence
CAKAYGSMWSGIW

SEQ ID NO: 183 - protein, 7 aa, synthetic construct
  REGION 1..7: note = antibody sequence
YTAWPNT

SEQ ID NO: 184 - protein, 13 aa, synthetic construct
  REGION 1..13: note = antibody sequence
NAKDVTVIPFPPK

SEQ ID NO: 185 - protein, 13 aa, synthetic construct
  REGION 1..13: note = antibody sequence
AADWRTILGWKTR

SEQ ID NO: 186 - protein, 13 aa, synthetic construct
  REGION 1..13: note = antibody sequence
SVGARRDEDDYVY

SEQ ID NO: 187 - protein, 6 aa, synthetic construct
  REGION 1..6: note = antibody sequence
NVALRY

SEQ ID NO: 188 - protein, 13 aa, synthetic construct
  REGION 1..13: note = antibody sequence
SVGARRDEDDYVY

SEQ ID NO: 189 - protein, 5 aa, synthetic construct
  REGION 1..5: note = antibody sequence
NQLTY

SEQ ID NO: 190 - protein, 9 aa, synthetic construct
  REGION 1..9: note = antibody sequence
NLRRWSYDY

SEQ ID NO: 191 - protein, 115 aa, synthetic construct
  REGION 1..115: note = antibody sequence
QVQLQESGGGLVQPGDSLKLSCAASGGTFGAGVVAWYRQSPGKQREMVGSMGSDGFTQIENGMKGRFTISGAGDKKTVFLQMNNLKPEDTAVYFCHYADGRFGSWGQGTQVTVSS

SEQ ID NO: 192 - protein, 114 aa, synthetic construct
  REGION 1..114: note = antibody sequence
QVQLQESGGGLVQAGGSLRLSCAASGSNISTNVMAWYRRAPGNQRDMVASISTSGTTNYLASVKGRFTISRDNAKNTVSLQMNSLKPEDTAVYTCYAAWPLNTWGQGTQVTVSS

SEQ ID NO: 193 - protein, 119 aa, synthetic construct
  REGION 1..119: note = antibody sequence
QVQLQESGGGLVQAGGSLRLSCAASGSTFSITYMAWFRQAPEKQRELVAEMSRRGSTFYADSVKGRFTISRDNTKNTVYLQMNSLEPEDTAVYYCSVGARRDEDDYVYWGQGTQVTVSS

SEQ ID NO: 194 - protein, 119 aa, synthetic construct
  REGION 1..119: note = antibody sequence
QVQLQQSGGGLVQAGGSLRLSCAASGSTFSITYMAWFRQAPEKQRELVAEMSRRGSTFYADSVKGRFTISRDNTKNTVYLQMNSLEPEDTAVYYCSVGARRDEDDYVYWGQGTQVTVSS

SEQ ID NO: 195 - protein, 123 aa, synthetic construct
  REGION 1..123: note = antibody sequence
QVQLQESGGGSVQAGGSLRLSCADSRTIFIFNAMAWYRQAPGKQRELVAAISSGGSTKYADSVKGRFTISSSNAKNTKYLQMNRLKPEDTAVYYCAASRSGRWLDDARDYEYWGPGTQVTVSS

SEQ ID NO: 196 - protein, 114 aa, synthetic construct
  REGION 1..114: note = antibody sequence
QVQLQESGGGLVQAGGSLRLSCAASGSNISTNVMAWYRRAPGNQRDMVASISTSGTTNYLASVKGRFTISRDNAKNTVSLQMNSLKPEDTAVYTCYAAWPLNTWGQGTLVTVSS

SEQ ID NO: 197 - protein, 119 aa, synthetic construct
  REGION 1..119: note = antibody sequence
QVQLQQSGGGLVQAGGSLRLSCAASGIPFSIIYMAWFRQAPEKQRELVAEMSSRGSKFYADSVKGRFTISRDNAKNTLYLQMNSLEPEDTAVYYCSVGARRDDNDYVYWGQGTQVTVSS

SEQ ID NO: 198 - protein, 114 aa, synthetic construct
  REGION 1..114: note = antibody sequence
QVQLQASGGGLVQAGGSLRLSCAASGSNISTNVMAWYRRAPGNQRDMVASISTSGTTNYLASVKGRFTISRDNAKNTVSLQMNSLKPEDTAVYTCYAAWPLNTWGQGTQVTVSS

SEQ ID NO: 199 - protein, 119 aa, synthetic construct
  REGION 1..119: note = antibody sequence
QVQLQESGGGLVQAGGSLRLSCAASGIPFSIIYMAWFRQAPEKQRELVAEMSSRGSKFYADSVKGRFTISRDNAKNTLYLQMNSLEPEDTAVYYCSVGARRDDNDYVYWGQGTQVTVSS

SEQ ID NO: 200 - protein, 120 aa, synthetic construct
  REGION 1..120: note = antibody sequence
QVQLQASGGGVVQSGGSLRLSCVASGNIFGINSMAWYRQAPGKQRELVADITRGNRKYADSVKGRFTISQDNAKNTVYLQMNRLKPEDTAVYFCNAEIVTQIPFPPREFWGRGTLVTVSS

SEQ ID NO: 201 - protein, 124 aa, synthetic construct
  REGION 1..124: note = antibody sequence
QVQLQASGGGLVQPGGSLRLSCAASGFTFSSYWMYWVRQAPGKGLEWVSTINTGGYTTYYSDSVKGRFTISRDNGKNTLYLEMNSLKSEDTAVYYCAKAYGSMWSGIWGGMDYWGKGTQVTVSS

SEQ ID NO: 202 - protein, 114 aa, synthetic construct
  REGION 1..114: note = antibody sequence
QVQLQQSGGGLVQAGGSLRLSCAASGSNISTNVMAWYRRAPGNQRDMVASISTSGTTNYLASVKGRFTISRDNAKNTVSLQMNSLKPEDTAVYTCYAAWPLNTWGQGTQVTVSS

SEQ ID NO: 203 - protein, 113 aa, synthetic construct
  REGION 1..113: note = antibody sequence
QVQLQESGGGLVQAGGSLRLSCAVSGSIISHNVMAWYRRAPGKQRDKVACISGSGFTNYIASVKGRFTISRDNAKNTVSLQMNNLKPEDTAVYSCYTAWPNTWGQGTQVTVSS

SEQ ID NO: 204 - protein, 121 aa, synthetic construct
  REGION 1..121: note = antibody sequence
QVQLQESGGGFVHPGGSLTLSCAASGRIFNIEDMGWYRQGPGEQRDLVATITRTGAPTYANSVKGRFTISRDNAKNTVYLQMTRLKPEDTAVYYCNAKDVTVIPFPPKDYWGRGTQVTVSS

SEQ ID NO: 205 - protein, 123 aa, synthetic construct
  REGION 1..123: note = antibody sequence
QVQLQESGGGLVQAGGSLRLSCEASGSVSAIETMGWYRQAPDEQRTFVAVISTGGTTKYAPSVKGRFTISIDNAKSTVTLQMNSLKPEDTAVYYCAADWRTILGWKTREPNYFGQGTLVTVSS

SEQ ID NO: 206 - protein, 119 aa, synthetic construct
  REGION 1..119: note = antibody sequence
QVQLQESGGGLVQAGGSLRLSCAAFGSTSSITYMAWFRQAPEKQRELVAEMSRRGSTFYADSVKGRFTIYRDNTKNTVYLQMNSLEPEDTAVYYCSVGARRDEDDYVYWGQGTQVTVSS

SEQ ID NO: 207 - protein, 112 aa, synthetic construct
  REGION 1..112: note = antibody sequence
QVQLQESGGGLVQAGGSLRLSCAASGGPVSDNVMAWFRQAPGSQRELVAQITSGGATSYADSVKGRFTISRDNARSTVDLQMNSLKPEDTAVYYCNVALRYWGRGTQVTVSS

SEQ ID NO: 208 - protein, 119 aa, synthetic construct
  REGION 1..119: note = antibody sequence
QVQLQQSGGGLVQAGGSLRLSCAASGSTFSITYMAWFRQAPGKQRELVAEISSRGSVFYADSVKGRFTISRDNAKKTVYLQMNSLETEDTAAYYCSVGARRDEDDYVYWGQGTQVTVSS

SEQ ID NO: 209 - protein, 119 aa, synthetic construct
  REGION 1..119: note = antibody sequence
QVQLQESGGGLVQAGGSLRLSCAASGSTFSITYMAWFRQAPEKQRELVAEMSRRGSTFYADSVKGRFTISRDNTKNTVYLQMNSLEPEDTAVYYCSVGARRDEDDYVYWGQGTLVTVSS

SEQ ID NO: 210 - protein, 119 aa, synthetic construct
  REGION 1..119: note = antibody sequence
QVQLQESGGGLVQAGGSLRLSCAASGIPFSIIYMAWFRQAPEKQRELVAEMSSRGSKFYADSVKGRFTISRDNAKNTLYLQMNSLEPEDTAVYYCSVGARRDDNDYVYWGQGTLVTVSS

SEQ ID NO: 211 - protein, 119 aa, synthetic construct
  REGION 1..119: note = antibody sequence
QVQLQEFGGGLVQAGGSLRLSCAASGSTFSITYMAWFRQAPEKQRELVAEMSRRGSTFYADSVKGRFTISRDNTKNTVYLQMNSLEPEDTAVYYCSVGARRDEDDYVYWGQGTQVTVSS

SEQ ID NO: 212 - protein, 114 aa, synthetic construct
  REGION 1..114: note = antibody sequence
QVQLQEFGGGLVQAGESLRLSCVASGSNISTNVMAWYRRAPGNQRDMVASISTSGTTNYLASVKGRFTISRDNAKNTVSLQMNSLKPEDTAVYTCYAAWPLNTWGQGTLVTVSS

SEQ ID NO: 213 - protein, 111 aa, synthetic construct
  REGION 1..111: note = antibody sequence
QVQLQESGGGLVQAGGSLRLSCVGAGSAFGWNAVHWYRQAPGQQREWLATIESGGWADYSVSVKGRFIVSRDNARNTAYLQMNNLKLEDTAVYYCNQLTYWGQGTQVTVSS

SEQ ID NO: 214 - protein, 119 aa, synthetic construct
  REGION 1..119: note = antibody sequence
QVQLQESGGGLVQAGGSLRLSCAASGSTFSITYMTWFRQAPEKQRELVAEMSRRGSTFYADSVKARFTISRDNTKNTVYLQMNSLEPEDTAVYYCSVGARRDEDDYVYWGQGTQVTVSS

SEQ ID NO: 215 - protein, 119 aa, synthetic construct
  REGION 1..119: note = antibody sequence
QVQLQEFGGGLVQAGGSLRLSCAASGSTFSITYMAWFRQAPEKQRELVAEMSRRGSTFYADSVKGRFTISRDNTKNTVYLQMNSLEPEDTAVYYCSVGARRDEDDYVYWGQGTLVTVSS

SEQ ID NO: 216 - protein, 119 aa, synthetic construct
  REGION 1..119: note = antibody sequence
QVQLQQSGGGLVQAGGSLRLSCAASGSTFSITYMAWFRQAPGKQRELVAEISSRGSVFYADSVKGRFTISRDNAKKTVYLQMNSLETEDTAAYYCSVGARRDEDDYVYWGQGTLVTVSS

SEQ ID NO: 217 - protein, 114 aa, synthetic construct
  REGION 1..114: note = antibody sequence
QVQLQESGGGLVQPGGSLRLSCTPSGSIFSFDVMAWYRQAPGKRRELVAQHRTPGAIDYADPVRGRFTISRDAGDVLFLQMDSLKPEDTAVYFCNLRRWSYDYWGQGTLVTVSS

SEQ ID NO: 218 - protein, 10 aa, synthetic construct
  REGION 1..10: note = antibody sequence
ASGNIFRINA

SEQ ID NO: 219 - protein, 12 aa, synthetic construct
  REGION 1..12: note = antibody sequence
ASIDSAGRTNYG

SEQ ID NO: 220 - protein, 10 aa, synthetic construct
  REGION 1..10: note = antibody sequence
CSTWRLGRNY

SEQ ID NO: 221 - protein, 115 aa, synthetic construct
  REGION 1..115: note = antibody sequence
QVQLQEFGGGVVQSGGSLRLSCVASGNIFRINAMGWYRQAPGKSRVLVASIDSAGRTNYGDAVEDRFTISRDIANNIVNLQMNSLKPEDTAVYYCSTWRLGRNYWGQGTQVTVSS

SEQ ID NO: 222 - protein, 10 aa, synthetic construct
  REGION 1..10: note = antibody sequence
ASGSTFSNNA

SEQ ID NO: 223 - protein, 10 aa, synthetic construct
  REGION 1..10: note = antibody sequence
AYISSGGFTN

SEQ ID NO: 224 - protein, 17 aa, synthetic construct
  REGION 1..17: note = antibody sequence
SAGGTYRSGNVYFFPRS

SEQ ID NO: 225 - protein, 123 aa, synthetic construct
  REGION 1..123: note = antibody sequence
QVQLQESGGGLVQAGGSLRLSCAASGSTFSNNAMAWYRQAPGKQRELVAYISSGGFTNYGDSVKGRFTISEDNAKSTVYLQMTSLKPEDTAVYYCSAGGTYRSGNVYFFPRSWGQGTQVTVSS

SEQ ID NO: 226 - protein, 10 aa, synthetic construct
  REGION 1..10: note = antibody sequence
ASGSIFSINS

SEQ ID NO: 227 - protein, 10 aa, synthetic construct
  REGION 1..10: note = antibody sequence
ATISSRSTTY

SEQ ID NO: 228 - protein, 8 aa, synthetic construct
  REGION 1..8: note = antibody sequence
KAGSVGRV

SEQ ID NO: 229 - protein, 115 aa, synthetic construct
  REGION 1..115: note = antibody sequence
QVQLQQSGGGLVQPGGSLRLSCAASGSIFSINSMAWYRQAPGNQRELVATISSRSTTYYAPSVKGRFTISRDNAKNIVYLQMNSLKPEDTAVYYCKAGSVGRVYWGQGTLVTVSS

SEQ ID NO: 230 - protein, 10 aa, synthetic construct
  REGION 1..10: note = antibody sequence
ASMTTLGFKT

SEQ ID NO: 231 - protein, 10 aa, synthetic construct
  REGION 1..10: note = antibody sequence
AAITSGGTAN

SEQ ID NO: 232 - protein, 14 aa, synthetic construct
  REGION 1..14: note = antibody sequence
ASTTGWTEVGGRND

SEQ ID NO: 233 - protein, 121 aa, synthetic construct
  REGION 1..121: note = antibody sequence
QVQLQESGGGLVQAGGSLRLSCLASMTTLGFKTMGWYRQAPGKQRELVAAITSGGTANYADSVKGRFAISRENAKNTLYLQMNSLKPEDTALYYCASTTGWTEVGGRNDYWGQGTLVTVSS

SEQ ID NO: 234 - protein, 10 aa, synthetic construct
  REGION 1..10: note = antibody sequence
ASGRTFRVNA

SEQ ID NO: 235 - protein, 10 aa, synthetic construct
  REGION 1..10: note = antibody sequence
AAVTNGGSTT

SEQ ID NO: 236 - protein, 11 aa, synthetic construct
  REGION 1..11: note = antibody sequence
NAERRFGMRQV

SEQ ID NO: 237 - protein, 117 aa, synthetic construct
  REGION 1..117: note = antibody sequence
QVQLQESGGGLVQTGGSLRLSCAASGRTFRVNAMGWYRQAPGKQREFVAAVTNGGSTTYADSVKGRFTISRDNAKNTIYLQMNRLEPEDTALYYCNAERRFGMRQVWGQGTLVTVSS

SEQ ID NO: 238 - protein, 10 aa, synthetic construct
  REGION 1..10: note = antibody sequence
ASGRVFSINT

SEQ ID NO: 239 - protein, 10 aa, synthetic construct
  REGION 1..10: note = antibody sequence
ASMTRGGSAN

SEQ ID NO: 240 - protein, 8 aa, synthetic construct
  REGION 1..8: note = antibody sequence
NAARGWRI

SEQ ID NO: 241 - protein, 115 aa, synthetic construct
  REGION 1..115: note = antibody sequence
QVQLQESGGGLVQPGGSLRLSCAASGRVFSINTMGWYRQAPGKQRELVASMTRGGSANYADSVKGRFTTSRDNAKNMVYLQMNRLKAEDTAVYYCNAARGWRIYWGKGTLVTVSS

SEQ ID NO: 242 - DNA, 498 bp, synthetic construct
  misc_feature 1..498: note = antibody sequence
  source 1..498: mol_type = other DNA
atgaaatacctattgcctacggcggccgctggattgttattactcgcggcccagccggccatggcgcaggtgcagttgcagcagtctgggggaggcttggtgcaggctgggggatctctgattctccgttgtagagcaactgtaagtggcttcagtatcgggaccatgggctggtaccgccaggctcccgggaaggagcgcgagttcgtcgcgaacgttagtcctagcggtgcaaaatacttcgctgactccgtgaagggccgattcaccatctccagagacaacgccaataatacagtgtatctgcaaatgaacagtctgaaacctgaagacacgggcgtctattattgtaatatacgaaggttttcgtacctcagtggcgactggggccaggggacccaggtcaccgtctcctcaactagtggcccgggaggccaacaccatcaccaccatcatggcgcagaacaaaaactcatctcagaagaggatctgtcttag

SEQ ID NO: 243 - DNA, 492 bp, synthetic construct
  misc_feature 1..492: note = antibody sequence
  source 1..492: mol_type = other DNA
atgaaatacctattgcctacggcggccgctggattgttattactcgcggcccagccggccatggcgcaggtgcagttgcaggagtctgggggaggcttggtgcaggctggggggtctctgagactctcctgtgcagcctctggattcaccttcagtagcagttccatgggctggtaccgccaggctccagggaagcagcgcgagttggtcgcttctattatgcgttatggtactacaacctatacagactccgtgaagggccgattcaccatctccagagacaacggccagagaacagtctatctgcaaatgaacagcctgaagcctgaggacacggccgtctattattgtaatgttcgaagtttcgttcgaacctactggggccaggggaccctggtcaccgtctcctcaactagtggcccgggaggccaacaccatcaccaccatcatggcgcagaacaaaaactcatctcagaagaggatctgtcttag

SEQ ID NO: 244 - DNA, 501 bp, synthetic construct
  misc_feature 1..501: note = antibody sequence
  misc_feature 76: note = n is a, c, g, or t
  source 1..501: mol_type = other DNA
atgaaatacctattgcctacggcggccgctggattgttattactcgcggcccagccggccatggcgcaggtgcagntgcaggagtctgggggaggattggtgcaggctgggggctctctgagactctcctgtgcagcctctggacgcaccttcagtagttttgccatgggctggttccgccaggctccagggaaggagcgtgagtttgttacagctattaactggagtggtagtagcacatactatgcagactccgtgaagggccgattcaccatctccagagacaacgccaagaacacggtgtatctgcaaatgaacagcctgaaacctgaggacacggccgtctattactgtaatgcccagcggaggtggcctcttcgtgactattggggccaggggacccaggtcaccgtctcctcaactagtggcccgggaggccaacaccatcaccaccatcatggcgcagaacaaaaactcatctcagaagaggatctgtcttag

SEQ ID NO: 245 - DNA, 501 bp, synthetic construct
  misc_feature 1..501: note = antibody sequence
  source 1..501: mol_type = other DNA
atgaaatacctattgcctacggcggccgctggattgttattactcgcggcccagccggccatggcgcaggtgcaaaaaaaagagtctgggggaggcttggtgcaggctggggggtctctgagactctcctgtgcagcctctggactcatcttcagtatcaatgccatggcctggtaccgccaggctccagggaaccagcgcgagttggtcgcacgtattactactggtggtagcacaaactatgcagactccgtgaagggccgattcaccatctccagagacaacgccaagaacacggtgtatctgcaaatgaacagcctgaaacctgaggacacagccgtctatttctgtgcagcagatgtaaggtttggggaacggactccctactggggccaggggacccaggtcaccgtctcctcaactagtggcccgggaggccaacaccatcaccaccatcatggcgcagaacaaaaactcatctcagaagaggatctgtcttag

SEQ ID NO: 246 - DNA, 492 bp, synthetic construct
  misc_feature 1..492: note = antibody sequence
  source 1..492: mol_type = other DNA
atgaaatacctattgcctacggcggccgctggattgttattactcgcggcccagccggccatggcgcaggtgcagttgcaggcgtctgggggaggcgtggtgcagtctggggggtctctgagactctcctgtgtagcctctggaaacatcttcaggatcaatgccatgggctggtaccgccaggctccagggaagcagcgcgagttggtcgcacatattattagtggtggtagcacaaactatgcagactccgtgaagggccgattcaccatctccagagaatacgccaagaatatggtgtatctgcaaatgaacagcctgaaacctgaggacacggccgtctattactgtaatgcccgaactttcgtgagaacctactggggccaggggacccaggtcaccgtctcctcaactagtggcccgggaggccaacaccatcaccaccatcatggcgcagaacaaaaactcatctcagaagaggatctgtcttag

SEQ ID NO: 247 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatggcgcaggtgcagttgcagcagtttgggggaggcttggtgcagcctggggggtctctgagactctcctgtcaagcctctacaagtgtcttcggtaacactgccatggcctggtaccgccaggctcctgggaagcagcgcgagttggtcgcacgaattactacccttggtttcacatactatgcagactccgcgaagggccgattcaccatctctagagacagcgccatgaacacggtgtatctgcaaatgaacagcctgaaacctgaggacacggccgtctattactgtaaccgcagaggatttcggagctactggggccaggggaccctggtcaccgtctcctcaactagtggcccgggaggccaacaccatcaccaccatcatggcgcagaacaaaaactcatctcagaagaggatctgtcttag

SEQ ID NO: 248 - DNA, 520 bp, synthetic construct
  misc_feature 1..520: note = antibody sequence
  misc_feature 97: note = n is a, c, g, or t
  misc_feature 99: note = n is a, c, g, or t
  source 1..520: mol_type = other DNA
atgaaatacctattgcctacggcggccgctggattgttattactcgcggcccagccggccatggcgcaggtgcagttgcaggagtccgggggaggcntnggtgcagtctggcgattctctgagactctcctgtgcagcctctggaagcatctatcatgtcaataccatgggttggtaccgccagtctccaggaaagcagcgcgagttggtcgcaactcttacacataacaaccgcgtaacctatgcagactccgtgaagggtcgattcaccatctccagagacaacgccaagatgacggtgtatctgcaaatggacagcctgaaacccgatgacacggccgtatattactgttactacttcgtcccgcgtaatccattattcgggagaaggattgactttgatgcctggggccaggggacccaggtcaccgtctcctcaactagtggcccgggaggccaacaccatcaccaccatcatggcgcagaacaaaaactcatctcagaagaggatctgtcttag

SEQ ID NO: 249 - DNA, 492 bp, synthetic construct
  misc_feature 1..492: note = antibody sequence
  source 1..492: mol_type = other DNA
atgaaatacctattgcctacggcggccgctggattgttattactcgcggcccagccggccatggcgcaggtgcagttgcaggagtctgggggaggcgtggtgcagtctggggggtctctgagactctcctgtgtagcctctggaaacatcttcaggatcaatgtcatgggctggtaccgccaggctccagggaagcagcgcgagttggtcgcggttgtaaagagtggtggtagcacaaactatgtagactccgcgaagggacgattcaccatctccagggacaacgccaagaacacagcgtatctgcacatggacagcctgaaacctgaggacacggccgtctattactgtaatgcacaaacccgactctggagctactggggccaggggacccaggtcaccgtttcctcaactagtggcccgggaggccaacaccatcaccaccatcatggcgcagaacaaaaactcatctcagaagaggatctgtcttag

SEQ ID NO: 250 - DNA, 492 bp, synthetic construct
  misc_feature 1..492: note = antibody sequence
  source 1..492: mol_type = other DNA
atgaaatacctattgcctacggcggccgctggattgttattactcgcggcccagccggccatggcgcaggtgcagctgcaggagtttgggggaggcgtggtgcagtctggggggtctctgagactctcctgtgtagcctctggaaacatcttcaggatcaatgccatgggctggtaccgccaggctccagggaagcagcgcgagttggtcgcacatattattagtggtggtagcacaaactatgcagactccgtgaagggccgattcaccatctccagagaatacgccaagaatatggtgtatctgcaaatgaacagcctgaaacctgaggacacggccgtctattactgtaatgcacaaacccgactctggagctactggggccaggggacccaggtcaccgtctcctcaactagtggcccgggaggccaacaccatcaccaccatcatggcgcagaacaaaaactcatctcagaagaggatctgtcttag

SEQ ID NO: 251 - DNA, 495 bp, synthetic construct
  misc_feature 1..495: note = antibody sequence
  source 1..495: mol_type = other DNA
atgaaatacctattgcctacggcggccgctggattgttattactcgcggcccagccggccatggcgcaggtgcagctgcaggagtctgggggaggcttggtgcagcctggggggtctctgagactctcctgtgcagcctctcgaagcttcttcagtatcaatgccatgggctggtaccgccaggctccagggaagcagcgcgagttggtcgcaactattactagtcgtgatagcacaaacgttgcagactccgtgaagggccgattcaccatctccagagactacgccaagaacatagtgtatctgcaaatggacagcctgagacctgaggacacggccacatattactgctacgctgatcaaccgtggaggggtcgtgcctggggccaggggacccaggtcaccgtctcctcaactagtggcccgggaggccaacaccatcaccaccatcatggcgcagaacaaaaactcatctcagaagaggatctgtcttag

SEQ ID NO: 252 - DNA, 492 bp, synthetic construct
  misc_feature 1..492: note = antibody sequence
  source 1..492: mol_type = other DNA
atgaaatacctattgcctacggcggccgctggattgttattactcgcggcccagccggccatggcgcaggtgcagttgcaggagtctgggggaggcgtggtgcaggccggggggtctctgaacctctcctgtacacactcaacaatcaccttcaggatcaacaccatggcgtactatcgccaggctccagggtctcagcgcgccctggtcgcgcggattaatccagcagggaggacgtattatccagattccgtgaagggccgattcaccatctccagagacaacgccaagaaccaagtgtatctacaaatgaacgacctcaaacctgaggacacggccgtctattactgttctacatggcgactaggacgcaactactggggccaggggaccctggtcaccgtctcctcaactagtggcccgggaggccaacaccatcaccaccatcatggcgcagaacaaaaactcatctcagaagaggatctgtcttag

SEQ ID NO: 253 - DNA, 498 bp, synthetic construct
  misc_feature 1..498: note = antibody sequence
  source 1..498: mol_type = other DNA
atgaaatacctattgcctacggcggccgctggattgttattactcgcggcccagccggccatggcgcaggtgcagttgcaggagtttgggggaggcttggtgcaggctgggggggttctgagactctcctgtgtagcctctatgactaccctcggtttcaagaccatgggctggtaccgccaggctccagggaagcagcgcgagttggtcgcaactattagtagtattggtatctcaacctatgcaaactccgtgaagggccgattcaccatctccagagacaatgccaagaacacagtgtatctacaaatgaacagcctgaaacctgaggacacggccgtctatttctgtcatgtaattcggcctagttggatgccgcagtactggggccaggggaccctggtcaccgtctcctcaactagtggcccgggaggccaacaccatcaccaccatcatggcgcagaacaaaaactcatctcagaagaggatctgtcttag

SEQ ID NO: 254 - DNA, 490 bp, synthetic construct
  misc_feature 1..490: note = antibody sequence
  misc_feature 62..63: note = n is a, c, g, or t
  misc_feature 67: note = n is a, c, g, or t
  source 1..490: mol_type = other DNA
atgaaatacctattgcctacggcggccgctggattgttattactcgcggcccagccggccannaaancaggtgcagctgcaggagtttgggggaggcttggtgcagcctggggggtctctgagactctcctgtcaagcctctacaagtgtcttcggtaacactgccatggcctggtaccgccaggctcctgggaagcagcgcgagttggtcgcacgaattactacccttggtttcacatactatgcagactccgcgaagggccgattcaccatctctagagacagcgccatgaacacggtgtatctgcaaatgaacagcctgaaacctgaggacacggccgtgtattactgtaatagattatggcggcctctagcgtggggtcaggggacccaggtcaccgtctcctcaactagtggcccgggaggccaacaccatcaccaccatcatggcgcagaacaaaaactcatctcagaagaggatctgtcttag

SEQ ID NO: 255 - DNA, 494 bp, synthetic construct
  misc_feature 1..494: note = antibody sequence
  misc_feature 64: note = n is a, c, g, or t
  misc_feature 70: note = n is a, c, g, or t
  source 1..494: mol_type = other DNA
atgaaatacctattgcctacggcggccgctggattgttattactcgcggcccagccggccatgnaaacanaaaacagctgcaggagtctgggggaggcttggtgcagcctggggggtctctgagactctcctgtgcagcctctggaatgcgcagcagtctcgctatcatgggctggtaccgccaggctccagggaagcagcgcgagttggtcgcaactattactattggtggtaacacaaactatgcagactccgtgaagggccggttcgccatctccagagacaacaccaagcgcacggtgtatctgcagatgaacagcctgacacctgaggacacggccgtctattactgtaatgttcgaagtttcgttagaacctactggggccaggggacccaggtcaccgtctcctcaactagtggcccgggaggccaacaccatcaccaccatcatggcgcagaacaaaaactcatctcagaagaggatctgtcttag

SEQ ID NO: 256 - DNA, 492 bp, synthetic construct; identical to SEQ ID NO: 243
  misc_feature 1..492: note = antibody sequence
  source 1..492: mol_type = other DNA

SEQ ID NO: 257 - DNA, 498 bp, synthetic construct
  misc_feature 1..498: note = antibody sequence
  source 1..498: mol_type = other DNA
atgaaatacctattgcctacggcggccgctggattgttattactcgcggcccagccggccatggcgcaggtgcagttgcagcagtctgggggaggcttggtgcaggctggggggtctctgagactctcctgtgcagcctctggaagcaccttcatcagtatcaaaaccatgggctggtaccgccaggctccagggaagcagcgcgagttggtcgctggtattactaagaataattacataaactatgcagactccgtgaagggccgattcaccatctccagagacaacggcaagaatacagtgtatctgcaaatgaacggcctgaaacctgaggacacggccgtctattactgtactgtacaacgtcgcttagggcgtgtctactggggccaggggaccctggtcaccgtctcctcaactagtggcccgggaggccaacaccatcaccaccatcatggcgcagaacaaaaactcatctcagaagaggatctgtcttag

SEQ ID NO: 258 - DNA, 492 bp, synthetic construct
  misc_feature 1..492: note = antibody sequence
  source 1..492: mol_type = other DNA
atgaaatacctattgcctacggcggccgctggattgttattactcgcggcccagccggccatggcgcaggtgcagctgcaggagtctgggggaggcttggtgcaggctggggggtctctgagactctcctgtacggcctctggaagcaccttcaggttcaatgacatgggctggtaccgccaggctccagggaagcagcgcgaattggtcgcaaatattaatagtagtggtagaaccatgtatccagactccgtcaagggccgattcacaatctccaaagacaacgtcaaaaatacagtgtatctgcagatgaacagcctgacacctgaggacacggccgtctattactgtaatgttcgaagtttcgttagaacctactggggccaggggacccaggtcaccgtctcctcaactagtggcccgggaggccaacaccatcaccaccatcatggcgcagaacaaaaactcatctcagaagaggatctgtcttag

SEQ ID NO: 259 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatggcgcaggtgcagctgcaggagtctgggggaggcttggtccaggctggggggtctctgacgctctcctgtgtagcctctggaagccgcttcagtatcaataccatgggctggtaccgccaggctccagggaagcagcgcgagttggtcgcaggtattactagccttggtttcacaaactatgcagactccgtgaagggccgattcaccatctccagagacaacgccaagaacacagtgtatctgcaaatgaacaacctgaaagttgaggacacggccgtctattactgtaaccgcagaggatttcggagctactggggccaggggaccctggtcaccgtctcctcaactagtggcccgggaggccaacaccatcaccaccatcatggcgcagaacaaaaactcatctcagaagaggatctgtcttag

SEQ ID NO: 260 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatggcgcaggtgcagctcgtggagtctgggggaggcttcgtgcaggctggggggtctctgagactctcctgtgcagcctctggaagcatcttcagtatcaattatggtaactggtaccgccaggctccagggaagcagcgcgaattggtcgcaggtattagtcgtggaggccgcacaaagtatgcagactccgtgaagggccgattcaccatctccagagatagcgccaagacactgacgctgcagatgactagcttgaaacctgaggacacggccgtctattactgtaatgttcgaagtttcgttcgaacctactggggccaggggacccaggtcaccgtttcctcaactagtggcccgggaggccaacaccatcaccaccatcatggcgcagaacaaaaactcatctcagaagaggatctgtcttag

SEQ ID NO: 261 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatggcgcaggtgcagctgcaggagtctgggggaggcttggtgcagtcgggggggtctctgagactctcctgttcggcctccggaagcatcttcaggatcaatctcatgggctggtaccgccaggctccagggaagcagcgcgagttggtcgcaactattactaatgaaggtaacacatactacgcagactccgtgaagggccgtttcaccatctccagagacaacgccaacaacacgtggtatctgcaaatgaacagcctgaaacctgaggacacagccgtctatgaatgtgcaggaaaggtcattagatggtactggggccaggggacccaggtcaccgtttcctcaactagtggcccgggaggccaacaccatcaccaccatcatggcgcagaacaaaaactcatctcagaagaggatctgtcttag

SEQ ID NO: 262 - DNA, 495 bp, synthetic construct
  misc_feature 1..495: note = antibody sequence
  source 1..495: mol_type = other DNA
atgaaatacctattgcctacggcggccgctggattgttattactcgcggcccagccggccatggcgcaggtgcagctgcaggagtttgggggaggcttggtgcagcctggggggtctctgagactctcctgtcaagcctctacaagtgtcttcggtaacactgccatggcctggtaccgccaggctcctgggaagcagcgcgagttggtcgcacgaattactacccttggtttcacatactatgcagactccgcgaagggccgattcaccatctctagagacagcgccatgaacacggtgtatctgcaaatgaacagcctgaaacctgaggacacggccgtttattactgtcgtgcacgtcgcgctctgcgagaatcgcactggggccaggggacccaggtcaccgtctcctcaactagtggcccgggaggccaacaccatcaccaccatcatggcgcagaacaaaaactcatctcagaagaggatctgtcttag

SEQ ID NO: 263 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatggcgcaggtgcagctgcaggagtctgggggaggcttcgtgcaggctggggggtctctgagactctcctgtgcagcctctggaagcatcttcagtatcaattatggtaactggtaccgccaggctccagggaagcagcgcgaattggtcgcaggtattagtcgtggaggccgcacaaagtatgcagactccgtgaagggccgattcaccatctccagagatagcgccaagacactgacgctgcagatgactagcttgaaacctgaggacacggccatctattcttgtaatgctcgaagtttcgttagaacttactggggccaggggaccctggtcaccgtctcctcaactagtggcccgggaggccaacaccatcaccaccatcatggcgcagaacaaaaactcatctcagaagaggatctgtcttag

SEQ ID NO: 264 - DNA, 507 bp, synthetic construct
  misc_feature 1..507: note = antibody sequence
  source 1..507: mol_type = other DNA
atgaaatacctattgcctacggcggccgctggattgttattactcgcggcccagccggccatggcgcaggtgcagctgcaggagtctgggggaggcttggtgacggctggagggtctctgagactctcctgtgcagcctctagaaacttcttcactttcagagccatgggctggtaccgccaggctccagggaagcagcgcgaaatggtcgcatctattactaccggtggtcgcaccgtctatgcagactccgtgaagggccgattcaccatctccaaatccaacgccaataacacagtgtatctccaaatgaacagcctggaagctgaggacacggccgtctattactgtaatgcacgacgcagatttccggtgccgggcccgaccgactactggggccgggggaccctggtcaccgtctcctcaactagtggcccgggaggccaacaccatcaccaccatcatggcgcagaacaaaaactcatctcagaagaggatctgtcttag

SEQ ID NO: 265 - DNA, 480 bp, synthetic construct
  misc_feature 1..480: note = antibody sequence
  source 1..480: mol_type = other DNA
atgaaatacctattgcctacggcggccgctggattgttattactcgcggcccagccggccatggcgcaggtgcagctgcaggagtctgggggaggcttggtgcaggctggggggtctctgagactctcctgtgcagcctctggaatcaccttcaggttcaatgccatgggctggtaccgccaggctccagggaaagagcgcgagttggtcgcaagggttagtagtggtggtagcacaacctatgcagactccgtgaaggcccgattcaccaccttcagagacaacgtcaagaacatagggtatctgcaaatgaccagcctgaaacctgaggacacggccgtctattactgtaatgtggggaatttctggggccaggggacccaggtcaccgtctcctcaactagtggcccgggaggccaacaccatcaccaccatcatggcgcagaacaaaaactcatctcagaagaggatctgtcttag

SEQ ID NO: 266 - DNA, 492 bp, synthetic construct
  misc_feature 1..492: note = antibody sequence
  source 1..492: mol_type = other DNA
atgaaatacctattgcctacggcggccgctggattgttattactcgcggcccagccggccatggcgcaggtgcagctgcaggagtctgggggaggcttggtgcggactggggagtctctgggactctcctgtgcagcctctggacgcagcatcctgatcaaaggcatgggctggtaccgccaggctccagggaaggagcgcgaaatggtcgcgactattagtatggccggtgtcactaactattcagactccgtgaagggccggttcaccatctccagagataactacaagaagacagtgtccctgcagatgaacaatttgagaccggaggacacggccgtctatgtgtgtaatgcacaaacccgactctggagctactggggccaggggacccaggtcaccgtctcctcaactagtggcccgggaggccaacaccatcaccaccatcatggcgcagaacaaaaactcatctcagaagaggatctgtcttag

SEQ ID NO: 267 - DNA, 492 bp, synthetic construct
  misc_feature 1..492: note = antibody sequence
  source 1..492: mol_type = other DNA
atgaaatacctattgcctacggcggccgctggattgttattactcgcggcccagccggccatggcgcaggtgcagctcgtggagtctgggggaggcctggtgcaggctggagggtctctgagactctcctgtgcagcctctggaaggattttcgggcgcaatgccatggcctggtaccgccaggttccagggaaggagcgcgagctggttgcacgtattactagggatggacggacaatgtatgtagactccgcgaagggacgattcaccatctccagggacaacgccaagaacacagcgtatctgcacatggacagcctgaaacctgaggacacggccgtctattactgtaatgcacaaacccgactctggagctactggggccaggggacccaggtcaccgtctcctcaactagtggcccgggaggccaacaccatcaccaccatcatggcgcagaacaaaaactcatctcagaagaggatctgtcttag

SEQ ID NO: 268 - DNA, 496 bp, synthetic construct
  misc_feature 1..496: note = antibody sequence
  misc_feature 70..73: note = n is a, c, g, or t
  source 1..496: mol_type = other DNA
atgaaatacctattgcctacggcggccgctggattgttattactcgcggcccagccggccatggcgcagnnnncagctgcaggagtctgggggaggcttggtgcaggctggggggtctctgagactctcctgtgcagcctctagaagcaccttcagattcaatgtcatgggctggtaccgccaggctccagggaagcagcgcgagttggtcgcagctattagtagtcgtggtggtagtacaaactatgcagactccgtgcagggccgattcaccatctccagagacaacgccaagaacacagtgtctctgcaaatgaacagcctgaaacctgaggacacggccgtctattactgtaatgttcgaagtttcgttagaacctactggggccaggggacccaggtcaccgtctcctcaactagtggcccgggaggccaacaccatcaccaccatcatggcgcagaacaaaaactcatctcagaagaggatctgtcttag

SEQ ID NO: 269 - DNA, 516 bp, synthetic construct
  misc_feature 1..516: note = antibody sequence
  source 1..516: mol_type = other DNA
atgaaatacctattgcctacggcggccgctggattgttattactcgcggcccagccggccatggcgcaggtgcagctgcaggagtttgggggaggcttggtgcaggctggggggtctctgagactctcctgtgcaacctctggaagcatcttcagtatcaacgccgtgggctggtaccgccaggctccagggaatcagcgcgagttggtcgcagctattagtggacgtggtagtacacactatgcagactccgtgaagggccgattcaccatctccagagacaccgccaagaacacggtgtatctgcaaatgaacagcctgaaacctgaggacacagccgtctattactgtgcattagatcaacatatggaggttattgtatcgccgggacgtattggttcctggggccaggggaccctggtcaccgtctcctcaactagtggcccgggaggccaacaccatcaccaccatcatggcgcagaacaaaaactcatctcagaagaggatctgtcttag

SEQ ID NO: 270 - DNA, 498 bp, synthetic construct
  misc_feature 1..498: note = antibody sequence
  source 1..498: mol_type = other DNA
atgaaatacctattgcctacggcggccgctggattgttattactcgcggcccagccggccatggcgcaggtgcagttgcaggagtctgggggaggcttggtgcaggctggggagtctctgacactctcctgtgcactctcaacaaccatgttcggtttctggcccatggcctggttccgccagactccaggacagcggcgcgaattgattgcgactattgatagtcgtggtcgcacaaacatcgcagactccgtgaagggccgatttaccatctccagagacaacaccaagaacacactgtatctgcggatgaacagcctgaaacctgaggacacggccgtctattactgtaatgcccagcggaggtggcctcttcgtgactattggggccaggggacccaggtcaccgtctcctcaactagtggcccgggaggccaacaccatcaccaccatcatggcgcagaacaaaaactcatctcagaagaggatctgtcttag

SEQ ID NO: 271 - DNA, 498 bp, synthetic construct
  misc_feature 1..498: note = antibody sequence
  source 1..498: mol_type = other DNA
atgaaatacctattgcctacggcggccgctggattgttattactcgcggcccagccggccatggcgcaggtgcagctgcaggagtttgggggaggcgtggtgcagtctggggggtctctgagactctcctgtgtagcctctggaaacatcttcaggatcaatgccatgggctggtaccgccaggctccagggaagcagcgcgagttggtcgcccgtattagtagtggtggtagcacaaactatgcagactccgtgaagggccgattcaccatctccagagacaacgtcaagaacacagtgactctgcaaatgaacagcctgaaacctgaggacacggccgtctattactgtaatgcgcggaggccattgcgttggtatgagtactggggccaggggaccctggtcaccgtctcctcaactagtggcccgggaggccaacaccatcaccaccatcatggcgcagaacaaaaactcatctcagaagaggatctgtcttag

SEQ ID NO: 272 - DNA, 510 bp, synthetic construct
  misc_feature 1..510: note = antibody sequence
  source 1..510: mol_type = other DNA
atgaaatacctattgcctacggcggccgctggattgttattactcgcggcccagccggccatggcgcaggtgcagctgcaggcgtctgggggaggcttggtgcagcctgggggatctctgagactctcctgtgcagcctcgggaagcatcttcagtaccaatgccatgggctggtaccgccaggctccagggaagcagcgcgaggtgatcgcatctattacaaaatttgggaacacagactatgcagactctgtgaagggccgattcaccatctccagagacaacgccaagaacatagtgtatctgcaaatgaacagcctgaaacctgaggacacggccgtttattactgttatcaaaacagtcgggggcgctggtatgatattttcagggactactggggccaggggaccctggtcaccgtctcctcaactagtggcccgggaggccaacaccatcaccaccatcatggcgcagaacaaaaactcatctcagaagaggatctgtcttag

SEQ ID NO: 273 - DNA, 498 bp, synthetic construct
  misc_feature 1..498: note = antibody sequence
  source 1..498: mol_type = other DNA
atgaaatacctattgcctacggcggccgctggattgttattactcgcggcccagccggccatggcgcaggtgcagctcgtggagtctgggggtggcttcgtgcaggcagggggatctctaagactctcctgtgtagcctcgcgaagcagcttcaggatcactaccatgaactggtaccgccaggctccagggaagcagcgcgaaatggtcgcatctattactaccggtggtcgcaccgtctatgcagactccgtgaagggccgattcaccatctccaaatccaacgccaataacacagtgtatctccaaatgaacagcctggaagctgaggacacggccgtctattactgtaatgcccagcggaggtggcctcttcgtgactattggggccaggggaccctggtcaccgtctcctcaactagtggcccgggaggccaacaccatcaccaccatcatggcgcagaacaaaaactcatctcagaagaggatctgtcttag

SEQ ID NO: 274 - DNA, 492 bp, synthetic construct
  misc_feature 1..492: note = antibody sequence
  source 1..492: mol_type = other DNA
atgaaatacctattgcctacggcggccgctggattgttattactcgcggcccagccggccatggcgcaggtgcagttgcaggagtctgggggaggcgtggtgcagtctggggggtctctgagactctcctgtgtagcctctggaaacatcttcaggatcaatgtcatgggctggtaccgccaggctccagggaagcagcgcgagttggtcgcggttgtaaagagtggtggtagcacaaactatgtagactccgcgaagggacgattcaccatctccagggacaacgccaagaacacagcgtatctgcacatggacagcctgaaacctgaggacacggccgtctattactgtaatgcacaaacccgactctggagctactggggccaggggaccctggtcaccgtctcctcaactagtggcccgggaggccaacaccatcaccaccatcatggcgcagaacaaaaactcatctcagaagaggatctgtcttag

SEQ ID NO: 275 - DNA, 498 bp, synthetic construct
  misc_feature 1..498: note = antibody sequence
  source 1..498: mol_type = other DNA
atgaaatacctattgcctacggcggccgctggattgttattactcgcggcccagccggccatggcgcaggtgcagctgcaggagtttgggggaggcgtggtgcagtctggggggtctctgagactctcctgtgtagcctctggaaacatcttcaggatcaatgccatgggctggtaccgccaggctccagggaagcagcgcgagttggtcgcacatattattagtggtggtagcacaaactatgcagactccgtgaagggccgattcaccatctccagagaatacgccaagaatatggtgtatctgcaaatgaacagcctgaaacctgaggacacggccgtctattactgtaatgccgaaaggagattcgggatgagacaggtctggggccaggggacccaggtcaccgtctcctcaactagtggcccgggaggccaacaccatcaccaccatcatggcgcagaacaaaaactcatctcagaagaggatctgtcttag

SEQ ID NO: 276 - DNA, 513 bp, synthetic construct
  misc_feature 1..513: note = antibody sequence
  source 1..513: mol_type = other DNA
atgaaatacctattgcctacggcggccgctggattgttattactcgcggcccagccggccatggcgcaggtgcagttgcaggagtctgggggaggcttggtgcaggctggggggtctctgagactctcctgtgcagcctctgtggttcccttcagatactttcccatgggctggtaccgccaggctccagggagacagcgcgagttggtcgcgtctattaccagcggtggtggcgtaaactatgcagatttcgtagagggccgattcaccatctccagagacaatgccaagaacacattttatctacaaatgagcagcctgaaacctgaggacacggccgtctattactgtgcacgacttctcagtctgggtagtaggtggggatacggcatgttcacctggggcaaagggaccctggtcaccgtctcctcaactagtggcccgggaggccaacaccatcaccaccatcatggcgcagaacaaaaactcatctcagaagaggatctgtcttag

SEQ ID NO: 277 - DNA, 498 bp, synthetic construct
  misc_feature 1..498: note = antibody sequence
  misc_feature 76: note = n is a, c, g, or t
  misc_feature 78: note = n is a, c, g, or t
  source 1..498: mol_type = other DNA
atgaaatacctattgcctacggcggccgctggattgttattactcgcggcccagccggccatggcgcaggtgcagntncaggagtctgggggaggcttggtgcagcctggggggtctctgagactctcctgtgcagcctctggaagcatcttcagtatcaagaccatgggctggtaccgccaggctccagggaagcagcgcgagttggtcgctgctatagctagtggtggtttcacaaactatgcagactccgtgaagggccgattcaccatctccagagacaacgccaggaacacggtgtatctgcaaatgaacagcctgaaacctgaggacacggccgtctattactgtaatgcccagcggaggtggcctcttcgtgactattggggccaggggaccctggtcaccgtttcctcaactagtggcccgggaggccaacaccatcaccaccatcatggcgcagaacaaaaactcatctcagaagaggatctgtcttag

SEQ ID NO: 278 - DNA, 498 bp, synthetic construct
  misc_feature 1..498: note = antibody sequence
  misc_feature 76: note = n is a, c, g, or t
  misc_feature 82: note = n is a, c, g, or t
  source 1..498: mol_type = other DNA
atgaaatacctattgcctacggcggccgctggattgttattactcgcggcccagccggccatggcgcaggtgcagntgcagnagtcagggggaggcttggtacgggacggggggtctctgacactctcctgtgcagcctctggaagtgccttcaggatgaattccatggcctggtaccgccaggttcctgggaaacagcgcgagttagtcgcagctattagcttccgtgggagcgcaaattatgctaactccgtgaagggccgattcaccatctccagagacaacggcaagaacacggtatatctacaaatgaacagcctgaaacctgaggacacagccgtctattactgtgcagcaggccgtccatggcaaaggactttctactggggccaggggacccaggtcaccgtctcctcaactagtggcccgggaggccaacaccatcaccaccatcatggcgcagaacaaaaactcatctcagaagaggatctgtcttag

SEQ ID NO: 279 - DNA, 498 bp, synthetic construct
  misc_feature 1..498: note = antibody sequence
  source 1..498: mol_type = other DNA
atgaaatacctattgcctacggcggccgctggattgttattactcgcggcccagccggccatggcgcaggtgcagttgcaggagtctgggggaggcttcgtgcaggctggggggtctctgagactctcctgtgcagcctctggaagcatcttcagtatcaattatggtaactggtaccgccaggctccagggaagcagcgcgaattggtcgcaggtattagtcgtggaggccgcacaaagtatgcagactccgtgaagggccgattcaccatctccagagatagcgccaagacactgacgctgcagatgactagcttgaaacctgaggattcgggcgtctactactgtgctgcgacccgctggagttggggtactaagagttactggggccagggaacccaggtcaccgtctcctcaactagtggcccgggaggccaacaccatcaccaccatcatggcgcagaacaaaaactcatctcagaagaggatctgtcttag

SEQ ID NO: 280 - DNA, 492 bp, synthetic construct
  misc_feature 1..492: note = antibody sequence
  source 1..492: mol_type = other DNA
atgaaatacctattgcctacggcggccgctggattgttattactcgcggcccagccggccatggcgcaggtgcagctgcaggagtttgggggaggcgtggtgcagtctggggggtctctgagactctcctgtgtagcctctggaaacatcttcaggatcaatgccatgggctggtaccgccaggctccaggcaagtcacgcgtactggtcgcaagcattgatagtgccggtaggacaaactatggtgacgccgtagaggatcgattcaccatctccagagacatcgccaacaacatagtgaatctacagatgaatagcctaaaacctgaggacacggccgtctattactgttctacatggcgactaggacgcaactactggggccaggggacccaggtcaccgtctcctcaactagtggcccgggaggccaacaccatcaccaccatcatggcgcagaacaaaaactcatctcagaagaggatctgtcttag

SEQ ID NO: 281 - DNA, 528 bp, synthetic construct
  misc_feature 1..528: note = antibody sequence
  source 1..528: mol_type = other DNA
atgaaatacctattgcctacggcggccgctggattgttattactcgcggcccagccggccatggcgcaggtgcagttgcaggagtctgggggaggcttggtgcaggctgggggctctctgagactctcctgtgcagcctctggaagcatcttccgtatcaatggcatgggctggatccgccaggctccagggaaggagcgtgaggttgtagcagccgttaactggagtggtgaacgcacatactatgttgactccgtgaagggccgattcaccatctccagagaaaaaggcaacaggatatatctacaaatgaacgatttggaacctgacgacacggccgtttattactgtgcagcagatacggattaccgtttagacggtagtacgtggattaccaacctctactctgggtccttgggccaggggacccaggtcaccgtctcctcaactagtggcccgggaggccaacaccatcaccaccatcatggcgcagaacaaaaactcatctcagaagaggatctgtcttag

SEQ ID NO: 282 - DNA, 390 bp, synthetic construct
  misc_feature 1..390: note = antibody sequence
  source 1..390: mol_type = other DNA
caggtgcagctgcaggagtctgggggaggcttggtgcaggctggggggtctctgagactctcctgtgcagcctctggaagcaccttcagtaacaatgccatggcctggtaccgccaggctccagggaagcagcgcgagttggtcgcctatattagtagtggtggtttcacaaattatggcgactccgtgaagggccgattcaccatctccgaagacaacgccaagagtacagtgtatctacaaatgaccagcctgaaacctgaggacacggccgtctattattgtagcgccgggggtacataccgtagtggtaatgtctacttctttccgcgttcctggggccaggggacccaggtcaccgtctcctcaactagtggcccgggaggccaa

SEQ ID NO: 283 - DNA, 366 bp, synthetic construct
  misc_feature 1..366: note = antibody sequence
  source 1..366: mol_type = other DNA
caggtgcagttgcagcagtctgggggagggttggtgcagcctggggggtctctgagactctcctgtgcagcctctggaagcatcttcagtatcaattccatggcctggtaccgccaggctccagggaaccagcgcgagttggtcgcgactattagtagtcgtagtaccacgtactatgcgccttccgtgaagggccggttcaccatctccagagacaacgccaagaacatagtgtacctgcaaatgaacagcctcaaacctgaggacacggccgtgtattactgtaaggcgggttcagtgggtcgcgtgtactggggccaggggaccctggtcaccgtctcctcaactagtggcccgggaggccaa

SEQ ID NO: 284 - DNA, 384 bp, synthetic construct
  misc_feature 1..384: note = antibody sequence
  source 1..384: mol_type = other DNA
caggtgcagttgcaggagtctgggggaggcttggtgcaggctggggggtctctgagactctcctgtttagcctctatgactaccctcgggttcaagaccatgggctggtaccgccaggctccagggaagcagcgcgagttggtcgccgctattacgagtggtggtaccgcaaactatgcagactccgtgaagggccgattcgccatctccagagagaacgccaagaacacgctgtatctgcaaatgaacagcctgaaacctgaggacacggccctgtattactgtgcatcgactacgggttggacagaggtcggcggacgaaatgactactggggccaggggaccctggtcaccgtttcctcaactagtggcccgggaggccaa

SEQ ID NO: 285 - DNA, 372 bp, synthetic construct
  misc_feature 1..372: note = antibody sequence
  source 1..372: mol_type = other DNA
caggtgcagctgcaggagtctgggggaggattggtgcagactggggggtctcttagactctcctgtgcggcctctgggcgcaccttcagagtcaatgccatgggctggtaccgccaggctccagggaagcagcgcgagttcgtcgcagctgttacaaatggtggtagtacaacctatgcagattccgtgaagggccgattcaccatctccagggacaacgccaagaatacaatatatctgcaaatgaacagactggaacctgaagacacggccctctattattgtaatgccgaaaggagattcgggatgagacaggtctggggccaggggaccctggtcaccgtttcctcaactagtggcccgggaggccaa

SEQ ID NO: 286 - DNA, 366 bp, synthetic construct
  misc_feature 1..366: note = antibody sequence
  source 1..366: mol_type = other DNA
caggtgcagctgcaggagtctgggggaggcttggtgcagcctggggggtctctgagactctcctgtgcagcctctggaagagtcttcagtatcaataccatgggctggtaccgccaggctccagggaagcagcgcgagttggtcgcatctatgactagaggtggtagcgcaaattatgcagactccgtgaagggccgattcaccacatccagagacaacgccaagaacatggtgtatctgcaaatgaatagactgaaagctgaggacacggccgtctattactgtaatgcagctcggggttggaggatctactggggcaaagggaccctggtcaccgtttcctcaactagtggcccgggaggccaa

SEQ ID NO: 287 - DNA, 492 bp, synthetic construct
  misc_feature 1..492: note = antibody sequence
  source 1..492: mol_type = other DNA
atgaaatacctattgcctacggcggccgctggattgttattactcgcggcccagccggccatggcgcaggtgcagctgcaggagtctgggggaggcttggtgcagcccggggactctctgaagctctcttgtgcagcctctggcggcaccttcggtgccggtgtcgtggcctggtaccgccagtctccagggaaacagcgtgagatggtcggaagtatgggtagtgatggtttcacgcaaatcgaaaacggcatgaagggccgattcactatctccggggccggcgacaagaaaacagtgtttttacagatgaacaatttgaagcctgaggacacggccgtctatttctgtcattacgccgatggccggtttggctcttggggtcaggggacccaggtcaccgtctcctcaactagtggcccgggaggccaacaccatcaccaccatcatggcgcagaacaaaaactcatctcagaagaggatctgtcttag

SEQ ID NO: 288 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatggcgcaggtgcagctgcaggagtctgggggaggcttggtgcaggctggggggtctctgagactctcctgtgcagcctctggaagtaacatcagtactaatgtgatggcctggtaccgccgcgctccagggaaccagcgcgacatggttgcttctatcagtactagtggtactaccaattatctagcctccgtgaagggccgattcactatctccagagacaacgccaagaacacggtgtcgctgcaaatgaacagcctgaaacctgaggacacggccgtctacacttgttatgcagcctggccgttgaacacttggggccaggggacccaggtcaccgtctcctcaactagtggcccgggaggccaacaccatcaccaccatcatggcgcagaacaaaaactcatctcagaagaggatctgtcttag

SEQ ID NO: 289 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatgaaacaggtgcagctgcaggagtctgggggaggcttggtgcaggctggggggtctctgagactctcctgtgcagcctctggaagtaacatcagtactaatgtgatggcctggtaccgccgcgctccagggaaccagcgcgacatggttgcttctatcagtactagtggtactaccaattatctagcctccgtgaagggccgattcactatctccagagacaacgccaagaacacggtgtcgctgcaaatgaacagcctgaaacctgaggacacggccgtctacacttgttatgcagcctggccgttgaacacttggggccaggggacccaggtcaccgtctcctcaactagtggcccgggaggccaacaccatcaccaccatcatggcgcagaacaaaaactcatctcagaagaggatctgtcttag

SEQ ID NO: 290 - DNA, 489 bp, synthetic construct; identical to SEQ ID NO: 289
  misc_feature 1..489: note = antibody sequence
  source 1..489: mol_type = other DNA

SEQ ID NO: 291 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatggcgcaggtgcagctgcaggagtctgggggaggcttggtccaggctggggggtctctgagactctcctgtgcagcctctggaagtaacatcagtactaatgtgatggcctggtaccgccgcgctccagggaaccagcgcgacatggttgcttctatcagtactagtggtaccaccaattatctagcctccgtgaagggccgattcactatctccagagacaacgccaagaacacggtgtcgctacaaatgaacagcctgaaacctgaggacacggccgtctacacttgttatgcagcctggccgttgaacacttggggccaggggacccaggtcaccgtctcctcaactagtggcccgggaggccaacaccatcaccaccatcatggcgcagaacaaaaactcatctcagaagaggatctgtcttag

SEQ ID NO: 292 - DNA, 489 bp, synthetic construct
  misc_feature 1..489: note = antibody sequence
  misc_feature 64..66: note = n is a, c, g, or t
  source 1..489: mol_type = other DNA
atgaaatacctattgcctacggcggccgctggattgttattactcgcggcccagccggccatgnnncaggtgcagctgcaggagtctgggggaggcttggtgcaggctggggggtctctgagactctcctgtgcagcctctggaagtaacatcagtactaatgtgatggcctggtaccgccgcgctccagggaaccagcgcgacatggttgcttctatcagtactagtggtactaccaattatctagcctccgtgaagggccgattcactatctccagagacaacgccaagaacacggtgtcgctgcaaatgaacagcctgaaacctgaggacacggccgtctacacttgttatgcagcctggccgttgaacacttggggccaggggacccaggtcaccgtctcctcaactagtggcccgggaggccaacaccatcaccaccatcatggcgcagaacaaaaactcatctcagaagaggatctgtcttag

SEQ ID NO: 293 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatggcacaggtgcagctgcaggagtctgggggaggcttggtgcaggctggggggtctctgagactctcctgtgcagcctctggaagtaacatcagtactaatgtgatggcctggtaccgccgcgctccagggaaccagcgcgacatggttgcttctatcagtactagtggtactaccaattatctagcctccgtgaagggccgattcactatctccagagacaacgccaagaacacggtgtcgctgcaaatgaacagcctgaaacctgaggacacggccgtctacacttgttatgcagcctggccgttgaacacttggggccaggggacccaggtcaccgtctcctcaactagtggcccgggaggccaacaccatcaccaccatcatggcgcagaacaaaaactcatctcagaagaggatctgtcttag

SEQ ID NO: 294 - DNA, 489 bp, synthetic construct; identical to SEQ ID NO: 288
  misc_feature 1..489: note = antibody sequence
  source 1..489: mol_type = other DNA

SEQ ID NO: 295 - DNA, 489 bp, synthetic construct; identical to SEQ ID NO: 288
  misc_feature 1..489: note = antibody sequence
  source 1..489: mol_type = other DNA

SEQ ID NO: 296 - DNA, 489 bp, synthetic construct; identical to SEQ ID NO: 289
  misc_feature 1..489: note = antibody sequence
  source 1..489: mol_type = other DNA

SEQ ID NO: 297 - DNA, 489 bp, synthetic construct; identical to SEQ ID NO: 288
  misc_feature 1..489: note = antibody sequence
  source 1..489: mol_type = other DNA

SEQ ID NO: 298 - DNA, 504 bp, synthetic construct
  misc_feature 1..504: note = antibody sequence
  source 1..504: mol_type = other DNA
atgaaatacctattgcctacggcggccgctggattgttattactcgcggcccagccggccatggcgcaggtgcagctgcaggagtctgggggaggcttggtgcaggctggggggtctctgagactctcctgtgcagcctctggaagcaccttcagtatcacgtacatggcctggttccgccaggctccagaaaagcagcgcgagttggtcgcagaaatgagtaggcgtggtagtacattctatgcagactccgtgaagggccgattcaccatctccagagacaacaccaagaacacagtctatctgcaaatgaacagcctagaacctgaagacacggccgtctattattgtagtgtaggcgcacgtcgcgacgaggatgattatgtctactggggccaggggacccaggtcaccgtctcctcaactagtggcccgggaggccaacaccatcaccaccatcatggcgcagaacaaaaactcatctcagaagaggatctgtcttag

SEQ ID NO: 299 - DNA, 504 bp, synthetic construct
  misc_feature 1..504: note = antibody sequence
  misc_feature 82: note = n is a, c, g, or t
  source 1..504: mol_type = other DNA
atgaaatacctattgcctacggcggccgctggattgttattactcgcggcccagccggccatggcgcaggtgcagctgcagnagtctgggggaggcttggtgcaggctggggggtctctgagactctcctgtgcagcctctggaagcaccttcagtatcacgtacatggcctggttccgccaggctccagaaaagcagcgcgagttggtcgcagaaatgagtaggcgtggtagtacattctatgcagactccgtgaagggccgattcaccatctccagagacaacaccaagaacacagtctatctgcaaatgaacagcctagaacctgaagacacggccgtctattattgtagtgtaggcgcacgtcgcgacgaggatgattatgtctactggggccaggggacccaggtcaccgtttcctcaactagtggcccgggaggccaacaccatcaccaccatcatggcgcagaacaaaaactcatctcagaagaggatctgtcttag

SEQ ID NO: 300 - DNA, 504 bp, synthetic construct; identical to SEQ ID NO: 298
  misc_feature 1..504: note = antibody sequence
  source 1..504: mol_type = other DNA

SEQ ID NO: 301 - DNA, 504 bp, synthetic construct; identical to SEQ ID NO: 298
  misc_feature 1..504: note = antibody sequence
  source 1..504: mol_type = other DNA

SEQ ID NO: 302 - DNA, 504 bp, synthetic construct
  misc_feature 1..504: note = antibody sequence
  source 1..504: mol_type = other DNA
atgaaatacctattgcctacggcggccgctggattgttattactcgcggcccagccggccatggcgcaggtgcagttgcaggagtctgggggaggcttggtgcaggctggggggtctctgagactctcctgtgcagcctctggaagcaccttcagtatcacgtacatggcctggttccgccaggctccagaaaagcagcgcgagttggtcgcagaaatgagtaggcgtggtagtacattctatgcagactccgtgaagggccgattcaccatctccagagacaacaccaagaacacagtctatctgcaaatgaacagcctagaacctgaagacacggccgtctattattgtagtgtaggcgcacgtcgcgacgaggatgattatgtctactggggccaggggacccaggtcaccgtctcctcaactagtggcccgggaggccaacaccatcaccaccatcatggcgcagaacaaaaactcatctcagaagaggatctgtcttag

SEQ ID NO: 303 - DNA, 504 bp, synthetic construct
  misc_feature 1..504: note = antibody sequence
  source 1..504: mol_type = other DNA
atgaaatacctattgcctacggcggccgctggattgttattactcgcggcccagccggccatggcgcaggtgcagctgcaggagtctgggggaggcttggtgcaggctggggggtctctgagactctcctgtgcagcctctggaagcaccttcagtatcacgtacatggcctggttccgccaggctccagaaaagcagcgcgagttggtcgcagaaatgagtaggcgtggtagtacattctatgcagactccgtgaagggccgattcaccatctccagagacaacaccaagaacacagtctatctgcaaatgaacagcctagaacctgaagacacggccgtctattattgtagtgtaggcgcacgtcgcgacgaggatgattatgtctactggggccaggggacccaggtcaccgtttcctcaactagtggcccgggaggccaacaccatcaccaccatcatggcgcagaacaaaaactcatctcagaagaggatctgtcttag

SEQ ID NO: 304 - DNA, 504 bp, synthetic construct
  misc_feature 1..504: note = antibody sequence
  source 1..504: mol_type = other DNA
atgaaatacctattgcctacggcggccgctggattgttattactcgcggcccagccggccatggcgcaggtgcagttgcagcagtctgggggaggcttggtgcaggctggggggtctctgagactctcctgtgcagcctctggaagcaccttcagtatcacgtacatggcctggttccgccaggctccagaaaagcagcgcgagttggtcgcagaaatgagtaggcgtggtagtacattctatgcagactccgtgaagggccgattcaccatctccagagacaacaccaagaacacagtctatctgcaaatgaacagcctagaacctgaagacacggccgtctattattgtagtgtaggcgcacgtcgcgacgaggatgattatgtctactggggccaggggacccaggtcaccgtctcctcaactagtggcccgggaggccaacaccatcaccaccatcatggcgcagaacaaaaactcatctcagaagaggatctgtcttag

SEQ ID NO: 305 - DNA, 516 bp, synthetic construct
  misc_feature 1..516: note = antibody sequence
  source 1..516: mol_type = other DNA
atgaaatacctattgcctacggcggccgctggattgttattactcgcggcccagccggccatggcgcaggtgcagttgcaggagtctgggggaggctcggtgcaggctggggggtctctgagactctcctgtgcagactctagaaccatcttcattttcaacgccatggcctggtaccgccaggctccagggaagcagcgcgagttggtcgcagctatttctagtggtggcagtacaaagtatgcagactccgtgaagggccgattcaccatctccagtagcaacgccaagaacacgaagtatctgcagatgaacaggctgaaacctgaggacacagccgtctattactgtgcagcctcaaggtcgggtaggtggttagatgatgcacgagactatgagtactggggcccggggacccaggtcaccgtctcctcaactagtggcccgggaggccaacaccatcaccaccatcatggcgcagaacaaaaactcatctcagaagaggatctgtcttag

SEQ ID NO: 306 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatggcgcaggtgcagctgcaggagtctgggggaggcttggtgcaggctggggggtctctgagactctcctgtgcagcctctggaagtaacatcagtactaatgtgatggcctggtaccgccgcgctccagggaaccagcgcgacatggttgcttctatcagtactagtggtactaccaattatctagcctccgtgaagggccgattcactatctccagagacaacgccaagaacacggtgtcgctgcaaatgaacagcctgaaacctgaggacacggccgtctacacttgttatgcagcctggccgttgaacacttggggccaggggaccctggtcaccgtctcctcaactagtggcccgggaggccaacaccatcaccaccatcatggcgcagaacaaaaactcatctcagaagaggatctgtcttag

SEQ ID NO: 307 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatggcgcaggtgcagttgcaggagtctgggggaggcttggtgcaggctggggggtctctgagactctcctgtgcagcctctggaagtaacatcagtactaatgtgatggcctggtaccgccgcgctccagggaaccagcgcgacatggttgcttctatcagtactagtggtactaccaattatctagcctccgtgaagggccgattcactatctccagagacaacgccaagaacacggtgtcgctgcaaatgaacagcctgaaacctgaggacacggccgtctacacttgttatgcagcctggccgttgaacacttggggccaggggaccctggtcaccgtctcctcaactagtggcccgggaggccaacaccatcaccaccatcatggcgcagaacaaaaactcatctcagaagaggatctgtcttag

SEQ ID NO: 308 - DNA, 489 bp, synthetic construct; identical to SEQ ID NO: 306
  misc_feature 1..489: note = antibody sequence
  source 1..489: mol_type = other DNA

SEQ ID NO: 309 - DNA, 489 bp, synthetic construct; identical to SEQ ID NO: 306
  misc_feature 1..489: note = antibody sequence
  source 1..489: mol_type = other DNA

SEQ ID NO: 310 - DNA, 489 bp, synthetic construct; identical to SEQ ID NO: 306
  misc_feature 1..489: note = antibody sequence
  source 1..489: mol_type = other DNA

SEQ ID NO: 311 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatgaaacaggtgcagctgcaggagtctgggggaggcttggtgcaggctggggggtctctgagactctcctgtgcagcctctggaagtaacatcagtactaatgtgatggcctggtaccgccgcgctccagggaaccagcgcgacatggttgcttctatcagtactagtggtactaccaattatctagcctccgtgaagggccgattcactatctccagagacaacgccaagaacacggtgtcgctgcaaatgaacagcctgaaacctgaggacacggccgtctacacttgttatgcagcctggccgttgaacacttggggccaggggaccctggtcaccgtctcctcaactagtggcccgggaggccaacaccatcaccaccatcatggcgcagaacaaaaactcatctcagaagaggatctgtcttag

SEQ ID NO: 312 - DNA, 504 bp, synthetic construct
  misc_feature 1..504: note = antibody sequence
  misc_feature 76..79: note = n is a, c, g, or t
  misc_feature 81: note = n is a, c, g, or t
  source 1..504: mol_type = other DNA
atgaaatacctattgcctacggcggccgctggattgttattactcgcggcccagccggccatggcgcaggtgcagnnnnancagtctgggggaggcttggtgcaggctggggggtctctgagactctcctgtgcagcctctggaattcccttcagtatcatctacatggcctggttccgccaggctccagaaaagcagcgcgagttggtcgcagaaatgagtagtcgtggtagtaaattctatgcagactccgtgaagggccgattcaccatctctagagacaacgccaagaacacactctatctgcaaatgaacagcctagaacctgaagatacggccgtctattattgcagtgtaggcgcacgtcgcgacgacaatgattatgtgtattggggccaggggacccaggtcaccgtttcctcaactagtggcccgggaggccaacaccatcaccaccatcatggcgcagaacaaaaactcatctcagaagaggatctgtcttag

SEQ ID NO: 313 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatggcgcaggtgcagttgcaggcgtctgggggaggcttggtgcaggctggggggtctctgagactctcctgtgcagcctctggaagtaacatcagtactaatgtgatggcctggtaccgccgcgctccagggaaccagcgcgacatggttgcttctatcagtactagtggtactaccaattatctagcctccgtgaagggccgattcactatctccagagacaacgccaagaacacggtgtcgctgcaaatgaacagcctgaaacctgaggacacggccgtctacacttgttatgcagcctggccgttgaacacttggggccaggggacccaggtcaccgtctcctcaactagtggcccgggaggccaacaccatcaccaccatcatggcgcagaacaaaaactcatctcagaagaggatctgtcttag

SEQ ID NO: 314 - DNA, 489 bp, synthetic construct; identical to SEQ ID NO: 313
  misc_feature 1..489: note = antibody sequence
  source 1..489: mol_type = other DNA

SEQ ID NO: 315 - DNA, 504 bp, synthetic construct
  misc_feature 1..504: note = antibody sequence
  source 1..504: mol_type = other DNA
atgaaatacctattgcctacggcggccgctggattgttattactcgcggcccagccggccatggcgcaggtgcagttgcaggagtctgggggaggcttggtgcaggctggggggtctctgagactctcctgtgcagcctctggaattcccttcagtatcatctacatggcctggttccgccaggctccagaaaagcagcgcgagttggtcgcagaaatgagtagtcgtggtagtaaattctatgcagactccgtgaagggccgattcaccatctctagagacaacgccaagaacacactctatctgcaaatgaacagcctagaacctgaagatacggccgtctattattgcagtgtaggcgcacgtcgcgacgacaatgattatgtgtattggggccaggggacccaggtcaccgtctcctcaactagtggcccgggaggccaacaccatcaccaccatcatggcgcagaacaaaaactcatctcagaagaggatctgtcttag

SEQ ID NO: 316 - DNA, 504 bp, synthetic construct
  misc_feature 1..504: note = antibody sequence
  source 1..504: mol_type = other DNA
atgaaatacctattgcctacggcggccgctggattgttattactcgcggcccagccggccatggcgcaggtgcagctgcaggagtctgggggaggcttggtgcaggctggggggtctctgagactctcctgtgcagcctctggaattcccttcagtatcatctacatggcctggttccgccaggctccagaaaagcagcgcgagttggtcgcagaaatgagtagtcgtggtagtaaattctatgcagactccgtgaagggccgattcaccatctctagagacaacgccaagaacacactctatctgcaaatgaacagcctagaacctgaagatacggccgtctattattgcagtgtaggcgcacgtcgcgacgacaatgattatgtgtattggggccaggggacccaggtcaccgtctcctcaactagtggcccgggaggccaacaccatcaccaccatcatggcgcagaacaaaaactcatctcagaagaggatctgtcttag

SEQ ID NO: 317 - DNA, 504 bp, synthetic construct
  misc_feature 1..504: note = antibody sequence
  misc_feature 79..81: note = n is a, c, g, or t
  source 1..504: mol_type = other DNA
atgaaatacctattgcctacggcggccgctggattgttattactcgcggcccagccggccatggcgcaggtgcagttgnnngagtctgggggaggcttggtgcaggctggggggtctctgagactctcctgtgcagcctctggaattcccttcagtatcatctacatggcctggttccgccaggctccagaaaagcagcgcgagttggtcgcagaaatgagtagtcgtggtagtaaattctatgcagactccgtgaagggccgattcaccatctctagagacaacgccaagaacacactctatctgcaaatgaacagcctagaacctgaagatacggccgtctattattgcagtgtaggcgcacgtcgcgacgacaatgattatgtgtattggggccaggggacccaggtcaccgtctcctcaactagtggcccgggaggccaacaccatcaccaccatcatggcgcagaacaaaaactcatctcagaagaggatctgtcttag

SEQ ID NO: 318 - DNA, 504 bp, synthetic construct
  misc_feature 1..504: note = antibody sequence
  misc_feature 76..78: note = n is a, c, g, or t
  source 1..504: mol_type = other DNA
atgaaatacctattgcctacggcggccgctggattgttattactcgcggcccagccggccatggcgcaggtgcagnnncaggagtctgggggaggcttggtgcaggctggggggtctctgagactctcctgtgcagcctctggaattcccttcagtatcatctacatggcctggttccgccaggctccagaaaagcagcgcgagttggtcgcagaaatgagtagtcgtggtagtaaattctatgcagactccgtgaagggccgattcaccatctctagagacaacgccaagaacacactctatctgcaaatgaacagcctagaacctgaagatacggccgtctattattgcagtgtaggcgcacgtcgcgacgacaatgattatgtgtattggggccaggggacccaggtcaccgtctcctcaactagtggcccgggaggccaacaccatcaccaccatcatggcgcagaacaaaaactcatctcagaagaggatctgtcttag

SEQ ID NO: 319 - DNA, 508 bp, synthetic construct
  misc_feature 1..508: note = antibody sequence
  misc_feature 82..83: note = n is a, c, g, or t
  misc_feature 86: note = n is a, c, g, or t
  source 1..508: mol_type = other DNA
atgaaatacctattgcctacggcggccgctggattgttattactcgcggcccagccggccatggcgcaggtgcagttgcagnngtnatgggggaggcgtggtgcagtctggggggtctctgagactctcctgtgtagcctctggaaacatcttcgggatcaattccatggcctggtaccgccaggctccagggaagcagcgcgaattggtcgctgacattacacgtggtaatagaaagtatgcagattccgtgaagggccgattcaccatctcccaagacaacgccaagaacacggtgtatctgcaaatgaacaggctgaaaccagaggacacggccgtctatttctgcaatgcagaaatcgtcacgcaaatccctttcccgccgcgtgagttctggggccgggggaccctggtcaccgtctcctcaactagtggcccgggaggccaacaccatcaccaccatcatggcgcagaacaaaaactcatctcagaagaggatctgtcttag

SEQ ID NO: 320 - DNA, 520 bp, synthetic construct
  misc_feature 1..520: note = antibody sequence
  misc_feature 95..98: note = n is a, c, g, or t
  source 1..520: mol_type = other DNA
atgaaatacctattgcctacggcggccgctggattgttattactcgcggcccagccggccatggcgcaggtgcagttgcaggcgtctgggggagnnnntggtgcagcctggggggtctctgagactctcctgtgcagcctctggattcacctttagtagctactggatgtattgggtccgtcaggctccagggaaggggctcgagtgggtctcaacaattaatactggtggttataccacatactattcagactccgtgaagggccgattcaccatctccagagacaacggcaagaacacgctgtatctcgaaatgaacagtctgaaatctgaggacacggccgtgtattactgtgcaaaggcgtacggtagtatgtggtcagggatctggggcggcatggactactggggcaaagggacccaggtcaccgtctcctcaactagtggcccgggaggccaacaccatcaccaccatcatggcgcagaacaaaaactcatctcagaagaggatctgtcttag

SEQ ID NO: 321 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatggggcaggtccagttgcagcagtctgggggaggcttggtccaggctggggggtctctgagactctcctgtgcagcctctggaagtaacatcagtactaatgtgatggcctggtaccgccgcgctccagggaaccagcgcgacatggttgcttctatcagtactagtggtaccaccaattatctagcctccgtgaagggccgattcactatctccagagacaacgccaagaacacggtgtcgctacaaatgaacagcctgaaacctgaggacacggccgtctacacttgttatgcagcctggccgttgaacacttggggccaggggacccaggtcaccctctcctcaactagtggcccgggaggccaacaccatcaccaccatcatggcgcagaacaaaaactcatctcagaagaggatctgtcttag

SEQ ID NO: 322 - DNA, 489 bp, synthetic construct
  misc_feature 1..489: note = antibody sequence
  misc_feature 76: note = n is a, c, g, or t
  misc_feature 132: note = n is a, c, g, or t
  source 1..489: mol_type = other DNA
atgaaatacctattgcctacggcggccgctggattgttattactcgcggcccagccggccatggcgcaggtgcagntgcaggagtctgggggaggcttggtccaggctggggggtctctgagactctcctgngcagtctctggaagtatcatcagtcataatgtgatggcctggtaccgccgcgctccagggaagcagcgcgacaaggttgcttgtatcagtggtagtggtttcaccaattatatagcctccgtgaagggccgattcactatctccagagacaacgccaagaacacggtgtctctacaaatgaacaacctgaaacctgaggacacggccgtctactcttgttatacagcctggccgtagaacacttggggccaggggacccaggtcaccgtctcctcaactagtggcccgggaggccaacaccatcaccaccatcatggcgcagaacaaaaactcatctcagaagaggatctgtcttag

SEQ ID NO: 323 - DNA, 510 bp, synthetic construct
  misc_feature 1..510: note = antibody sequence
  source 1..510: mol_type = other DNA
atgaaatacctattgcctacggcggccgctggattgttattactcgcggcccagccggccatggcgcaggtgcagttgcaggagtctgggggaggctttgtgcaccctggagggtctctgacgctctcctgtgcagcctctggcaggatcttcaatatcgaggacatgggctggtatcgccagggtccaggggaacagcgcgacttggtcgcaacgatcacccgtactggtgcgccaacctatgcaaactccgtgaagggccggttcaccatctccagagacaacgccaagaacacggtttatctgcagatgaccaggctgaaacctgaggacacggccgtctattactgtaatgcaaaagacgtaacagtcatacctttccccccgaaagactattggggccgggggacccaggtcaccgtctcctcaactagtggcccgggaggccaacaccatcaccaccatcatggcgcagaacaaaaactcatctcagaagaggatctgtcttag

SEQ ID NO: 324 - DNA, 516 bp, synthetic construct
  misc_feature 1..516: note = antibody sequence
  source 1..516: mol_type = other DNA
atgaaatacctattgcctacggcggccgctggattgttattactcgcggcccagccggccatggcgcaggtgcagctgcaggagtctgggggaggcttggtgcaggctggggggtctctgagactctcctgtgaagcctctggaagcgtttccgcgatcgaaaccatgggctggtaccgccaggctccggatgaacagcgcacatttgtcgcggttatcagtacgggtggaaccacaaaatacgcgccctccgtgaagggccgattcaccatctccatagacaacgccaagagcacggtgacgcttcaaatgaacagcctgaaacctgaggacacagccgtctactactgtgcagcggactggcgaaccattttgggttggaagacaagggagcccaactactttggccaggggaccctggtcaccgtctcctcaactagtggcccgggaggccaacaccatcaccaccatcatggcgcagaacaaaaactcatctcagaagaggatctgtcttag

SEQ ID NO: 325 - DNA, 504 bp, synthetic construct
  misc_feature 1..504: note = antibody sequence
  source 1..504: mol_type = other DNA
atgaaatacctattgcctacggcggccgctggattgttattactcgcggcccagccggccatggcgcaggtgcagttgcaggagtctgggggaggcttggtgcaggctggggggtctctgagactctcctgtgcagcctttggaagcacctccagtatcacgtacatggcctggttccgccaggctccagaaaagcagcgcgagttggtcgcagaaatgagtaggcgtggtagcacattctatgcagactccgtgaagggccgattcaccatttatagagacaacaccaagaacacagtctatctgcaaatgaacagcctagaacctgaagacacggccgtctattattgtagtgtaggcgcacgtcgcgacgaggatgattatgtctactggggccaggggacccaggtcaccgtctcctcaactagtggcccgggaggccaacaccatcaccaccatcatggcgcagaacaaaaactcatctcagaagaggatctgtcttag

SEQ ID NO: 326 - DNA, 504 bp, synthetic construct
  misc_feature 1..504: note = antibody sequence
  source 1..504: mol_type = other DNA
atgaaatacctattgcctacggcggccgctggattgttattactcgcggcccagccggccatggcgcaggtgcagttgcaggagtctgggggaggcttggtgcaggctggggggtctctgagactctcctgtgcagcctttggaagcacctccagtatcacgtacatggcctggttccgccaggctccagaaaagcagcgcgagttggtcgcagaaatgagtaggcgtggtagcacattctatgcagactccgtgaagggccgattcaccatttatagagacaacaccaagaacacagtctatctgcaaatgaacagcctagaacctgaagacacggccgtctattattgtagtgtaggcgcacgtcgcgacgaggatgattatgtctactggggccaggggacccaggtcaccgtttcctcaactagtggcccgggaggccaacaccatcaccaccatcatggcgcagaacaaaaactcatctcagaagaggatctgtcttag

SEQ ID NO: 327 - DNA, 505 bp, synthetic construct
  misc_feature 1..505: note = antibody sequence
  misc_feature 76..80: note = n is a, c, g, or t
  misc_feature 82..83: note = n is a, c, g, or t
  misc_feature 87..88: note = n is a, c, g, or t
  source 1..505: mol_type = other DNA
atgaaatacctattgcctacggcggccgctggattgttattactcgcggcccagccggccatggcgcaggtgcagnnnnnannagtnngggggaggcttggtgcaggctggggggtctctgagactctcctgtgcagcctttggaagcacctccagtatcacgtacatggcctggttccgccaggctccagaaaagcagcgcgagttggtcgcagaaatgagtaggcgtggtagcacattctatgcagactccgtgaagggccgattcaccatttatagagacaacaccaagaacacagtctatctgcaaatgaacagcctagaacctgaagacacggccgtctattattgtagtgtaggcgcacgtcgcgacgaggatgattatgtctactggggccaggggacccaggtcaccgtctcctcaactagtggcccgggaggccaacaccatcaccaccatcatggcgcagaacaaaaactcatctcagaagaggatctgtcttag

SEQ ID NO: 328 - DNA, 483 bp, synthetic construct
  misc_feature 1..483: note = antibody sequence
  source 1..483: mol_type = other DNA
atgaaatacctattgcctacggcggccgctggattgttattactcgcggcccagccggccatggcgcaggtgcagttgcaggagtctgggggaggcttggtgcaggctggggggtctctgagactctcctgtgcagcctctggaggacctgtcagtgacaatgtcatggcctggttccgccaggctccagggagccagcgcgaattggtcgcacagattacaagtggtggggccacaagctacgcggactccgtgaagggccgattcaccatctccagagacaacgccaggagcacagtggacctgcaaatgaacagcctgaaacctgaggacacggccgtctattactgtaacgtcgccttacgttactggggccgggggacccaggtcaccgtctcctcaactagtggcccgggaggccaacaccatcaccaccatcatggcgcagaacaaaaactcatctcagaagaggatctgtcttag

SEQ ID NO: 329 - DNA, 505 bp, synthetic construct
  misc_feature 1..505: note = antibody sequence
  misc_feature 76..79: note = n is a, c, g, or t
  misc_feature 82: note = n is a, c, g, or t
  misc_feature 87: note = n is a, c, g, or t
  misc_feature 457: note = n is a, c, g, or t
  misc_feature 459..460: note = n is a, c, g, or t
  source 1..505: mol_type = other DNA
atgaaatacctattgcctacggcggccgctggattgttattactcgcggcccagccggccatggcgcaggtgcagnnnnaancagtntgggggaggcttggtgcaggctggggggtctctgagactctcctgtgcagcctctggaagcaccttcagtatcacctacatggcctggttccgccaggctccagggaaacagcgcgaattggtcgcagaaataagtagccgtggtagtgtgttctatgcagactccgtgaagggccgattcaccatctccagagacaacgccaagaagacagtgtatctgcaaatgaacagcctggaaactgaagacacggccgcctattattgtagtgtaggcgcacgtcgcgacgaagatgactatgtctactggggccaggggacccaggtcaccgtttcctcaactagtggcccgggaggccaacaccatcaccancnncatggcgcagaacaaaaactcatctcagaagaggatctgtcttag

SEQ ID NO: 330 - DNA, 504 bp, synthetic construct
  misc_feature 1..504: note = antibody sequence
  source 1..504: mol_type = other DNA
atgaaatacctattgcctacggcggccgctggattgttattactcgcggcccagccggccatggcgcaggtgcagttgcaggagtctgggggaggcttggtgcaggctggggggtctctgagactctcctgtgcagcctctggaagcaccttcagtatcacgtacatggcctggttccgccaggctccagaaaagcagcgcgagttggtcgcagaaatgagtaggcgtggtagtacattctatgcagactccgtgaagggccgattcaccatctccagagacaacaccaagaacacagtctatctgcaaatgaacagcctagaacctgaagacacggccgtctattattgtagtgtaggcgcacgtcgcgacgaggatgattatgtctactggggccaggggaccctggtcaccgtttcctcaactagtggcccgggaggccaacaccatcaccaccatcatggcgcagaacaaaaactcatctcagaagaggatctgtcttag

SEQ ID NO: 331 - DNA, 504 bp, synthetic construct
  misc_feature 1..504: note = antibody sequence
  source 1..504: mol_type = other DNA
atgaaatacctattgcctacggcggccgctggattgttattactcgcggcccagccggccatggcgcaggtgcagttgcaggagtctgggggaggcttggtgcaggctggggggtctctgagactctcctgtgcagcctctggaagcaccttcagtatcacgtacatggcctggttccgccaggctccagaaaagcagcgcgagttggtcgcagaaatgagtaggcgtggtagtacattctatgcagactccgtgaagggccgattcaccatctccagagacaacaccaagaacacagtctatctgcaaatgaacagcctagaacctgaagacacggccgtctattattgtagtgtaggcgcacgtcgcgacgaggatgattatgtctactggggccaggggaccctggtcaccgtctcctcaactagtggcccgggaggccaacaccatcaccaccatcatggcgcagaacaaaaactcatctcagaagaggatctgtcttag

SEQ ID NO: 332 - DNA, 504 bp, synthetic construct
  misc_feature 1..504: note = antibody sequence
  source 1..504: mol_type = other DNA
atgaaatacctattgcctacggcggccgctggattgttattactcgcggcccagccggccatggcgcaggtgcagttgcaggagtctgggggaggcttggtgcaggctggggggtctctgagactctcctgtgcagcctctggaattcccttcagtatcatctacatggcctggttccgccaggctccagaaaagcagcgcgagttggtcgcagaaatgagtagtcgtggtagtaaattctatgcagactccgtgaagggccgattcaccatctctagagacaacgccaagaacacactctatctgcaaatgaacagcctagaacctgaagatacggccgtctattattgcagtgtaggcgcacgtcgcgacgacaatgattatgtgtattggggccaggggaccctggtcaccgtctcctcaactagtggcccgggaggccaacaccatcaccaccatcatggcgcagaacaaaaactcatctcagaagaggatctgtcttag

SEQ ID NO: 333 - DNA, 504 bp, synthetic construct
  misc_feature 1..504: note = antibody sequence
  source 1..504: mol_type = other DNA
atgaaatacctattgcctacggcggccgctggattgttattactcgcggcccagccggccatggcgcaggtgcagctgcaggagtttgggggaggcttggtgcaggctggggggtctctgagactctcctgtgcagcctctggaagcaccttcagtatcacgtacatggcctggttccgccaggctccagaaaagcagcgcgagttggtcgcagaaatgagtaggcgtggtagtacattctatgcagactccgtgaagggccgattcaccatctccagagacaacaccaagaacacagtctatctgcaaatgaacagcctagaacctgaagacacggccgtctattattgtagtgtaggcgcacgtcgcgacgaggatgattatgtctactggggccaggggacccaggtcaccgtctcctcaactagtggcccgggaggccaacaccatcaccaccatcatggcgcagaacaaaaactcatctcagaagaggatctgtcttag

SEQ ID NO: 334 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatggcgcaggtgcagctgcaggagtttgggggaggcttggtgcaggcaggggaatctctaagactctcctgtgtagcctctggaagtaacatcagtactaatgtgatggcctggtaccgccgcgctccagggaaccagcgcgacatggttgcttctatcagtactagtggtactaccaattatctagcctccgtgaagggccgattcactatctccagagacaacgccaagaacacggtgtcgctgcaaatgaacagcctgaaacctgaggacacggccgtctacacttgttatgcagcctggccgttgaacacttggggccaggggaccctggtcaccgtctcctcaactagtggcccgggaggccaacaccatcaccaccatcatggcgcagaacaaaaactcatctcagaagaggatctgtcttag

SEQ ID NO: 335 - DNA, 480 bp, synthetic construct
  misc_feature 1..480: note = antibody sequence
  source 1..480: mol_type = other DNA
atgaaatacctattgcctacggcggccgctggattgttattactcgcggcccagccggccatggcgcaggtgcagttgcaggagtctgggggaggcttggtgcaggccggggggtctctgagactctcctgtgtaggcgctggaagcgccttcggttggaatgccgtgcactggtaccgccaggctccaggtcagcagcgcgaatggctcgccactattgagagtggtggctgggcagactattcagtgtccgtgaagggccgattcatcgtctccagagacaacgccaggaacacagcgtatttgcaaatgaacaacctaaaacttgaagacacggccgtctattactgtaatcaacttacttactggggccaggggacccaggtcaccgtctcctcaactagtggcccgggaggccaacaccatcaccaccatcatggcgcagaacaaaaactcatctcagaagaggatctgtcttag

SEQ ID NO: 336 - DNA, 504 bp, synthetic construct
  misc_feature 1..504: note = antibody sequence
  misc_feature 75: note = n is a, c, g, or t
  misc_feature 86: note = n is a, c, g, or t
  source 1..504: mol_type = other DNA
atgaaatacctattgcctacggcggccgctggattgttattactcgcggcccagccggccatggcgcaggtgcanctgcaggagtntgggggaggcttggtgcaggctggggggtctctgagactctcctgtgcagcctctggaagcaccttcagtatcacgtacatgacctggttccgccaggctccagaaaagcagcgcgagttggtcgcagaaatgagtaggcgtggtagtacattctatgcagactccgtgaaggcccgattcaccatctccagagacaacaccaagaacacagtctatctgcaaatgaacagcctagaacctgaagacacggccgtctattattgtagtgtaggcgcacgtcgcgacgaggatgattatgtctactggggccaggggacccaggtcaccgtctcctcaactagtggcccgggaggccaacaccatcaccaccatcatggcgcagaacaaaaactcatctcagaagaggatctgtcttag

SEQ ID NO: 337 - DNA, 504 bp, synthetic construct
  misc_feature 1..504: note = antibody sequence
  source 1..504: mol_type = other DNA
atgaaatacctattgcctacggcggccgctggattgttattactcgcggcccagccggccatggcgcaggtgcagttgcaggagtttgggggaggcttggtgcaggctggggggtctctgagactctcctgtgcagcctctggaagcaccttcagtatcacgtacatggcctggttccgccaggctccagaaaagcagcgcgagttggtcgcagaaatgagtaggcgtggtagtacattctatgcagactccgtgaagggccgattcaccatctccagagacaacaccaagaacacagtctatctgcaaatgaacagcctagaacctgaagacacggccgtctattattgtagtgtaggcgcacgtcgcgacgaggatgattatgtctactggggccaggggaccctggtcaccgtttcctcaactagtggcccgggaggccaacaccatcaccaccatcatggcgcagaacaaaaactcatctcagaagaggatctgtcttag

SEQ ID NO: 338 - DNA, 504 bp, synthetic construct
  misc_feature 1..504: note = antibody sequence
  source 1..504: mol_type = other DNA
atgaaatacctattgcctacggcggccgctggattgttattactcgcggcccagccggccatggcgcaggtgcagttgcagcagtctgggggaggcttggtgcaggctggggggtctctgagactctcctgtgcagcctctggaagcaccttcagtatcacctacatggcctggttccgccaggctccagggaaacagcgcgaattggtcgcagaaataagtagccgtggtagtgtgttctatgcagactccgtgaagggccgattcaccatctccagagacaacgccaagaagacagtgtatctgcaaatgaacagcctggaaactgaagacacggccgcctattattgtagtgtaggcgcacgtcgcgacgaagatgactatgtctactggggccaggggaccctggtcaccgtctcctcaactagtggcccgggaggccaacaccatcaccaccatcatggcgcagaacaaaaactcatctcagaagaggatctgtcttag

SEQ ID NO: 339 - DNA, 489 bp, synthetic construct
  misc_feature 1..489: note = antibody sequence
  source 1..489: mol_type = other DNA
atgaaatacctattgcctacggcggccgctggattgttattactcgcggcccagccggccatggcgcaggtgcagctgcaggagtctgggggaggcttggtgcagcctggggggtctctgagactctcctgcacaccctctggatctatcttcagtttcgatgtcatggcctggtatcgccaggccccagggaagcggcgcgagttggtcgcacagcatcgtactccgggtgctatagattatgccgatcctgtccggggccgattcactattagcagagacgctggggacgtactgtttctgcaaatggacagcctgaaacccgaagacacggccgtctacttctgtaatctccgaaggtggtcttacgactactggggccaggggaccctggtcaccgtctcctcaactagtggcccgggaggccaacaccatcaccaccatcatggcgcagaacaaaaactcatctcagaagaggatctgtcttag

SEQ ID NO: 340 - protein, 9 aa, synthetic construct
  REGION 1..9: note = antibody sequence
SGSIFRING

SEQ ID NO: 341 - protein, 11 aa, synthetic construct
  REGION 1..11: note = antibody sequence
VAAVNWSGERT

SEQ ID NO: 342 - protein, 17 aa, synthetic construct
  REGION 1..17: note = antibody sequence
AADTDYRLDGSTWITNL

SEQ ID NO: 343 - protein, 127 aa, synthetic construct
  REGION 1..127: note = antibody sequence
QVQLQESGGGLVQAGGSLRLSCAASGSIFRINGMGWIRQAPGKEREVVAAVNWSGERTYYVDSVKGRFTISREKGNRIYLQMNDLEPDDTAVYYCAADTDYRLDGSTWITNLYSGSLGQGTQVTVSS

(19) United States
(12) Patent Application Publication
(10) Pub. No.: US 2025/0059265 A1
(43) Pub. Date: Feb. 20, 2025

(54) ALPHA-SYNUCLEIN SINGLE DOMAIN ANTIBODIES

(71) Applicant: NEW YORK UNIVERSITY, New York, NY (US)

(72) Inventor: Einar M. SIGURDSSON, Scarsdale, NY (US)

(21) Appl. No.: 18/934,067

(22) Filed: Oct. 31, 2024

Related U.S. Application Data
(62) Division of application No. 16/971,157, filed on Aug. 19, 2020, now Pat. No. 12,162,929.
(60) Provisional application No. 62/632,267, filed on Feb. 19, 2018.

(51) Int. Cl. C07K 16/18; G01N 33/68

(52) U.S. Cl. CPC C07K 16/18; G01N 33/6896; C07K 2317/21; C07K 2317/22; C07K 2317/24; C07K 2317/569; G01N 2800/28; G01N 2800/56

(57) ABSTRACT

The present disclosure is directed to single domain antibodies that bind to α-synuclein and the use of these antibodies for the treatment and diagnosis of α-synucleinopathies. The present disclosure is also directed to polynucleotides encoding the α-synuclein single domain antibodies, therapeutic vectors comprising these polynucleotides and methods of administering these therapeutic vectors for the treatment of α-synucleinopathies.

This application is a divisional of U.S. patent application Ser. No. 16/971,157, filed Aug. 19, 2020, which is a national stage application under 35 U.S.C. § 371 of International Patent Application No. PCT/US2019/018579, filed Feb. 19, 2019, which claims the priority benefit of U.S. Provisional Patent Application Ser. No. 62/632,267, filed Feb. 19, 2018, each of which are hereby incorporated by reference in their entirety.

This invention was made with government support under R21 AG059391 awarded by the National Institutes of Health. The government has certain rights in the invention.

### SEQUENCE LISTING

This application contains a computer readable Sequence Listing which has been submitted electronically in XML format and is hereby incorporated by reference in its entirety. The XML file was created on Oct. 29, 2024, is named 147462.002033.xml, and is 496,474 bytes in size.

### FIELD OF THE INVENTION

The present invention is directed to antibodies for the treatment and diagnosis of α-synucleinopathies.

### BACKGROUND OF THE INVENTION

Immunotherapies targeting various protein aggregates such as amyloid-β (Aβ), tau and α-synuclein (α-syn) are in different stages of clinical development, and collectively are the most common approach by the pharmaceutical industry to tackle diseases characterized by such depositions (Golde TE, “Open Questions for Alzheimer's Disease Immunotherapy.” Alzheimers Res Ther 6:3 (2014); Valera and Masliah, “Immunotherapy for Neurodegenerative Diseases: Focus on Alphasynucleinopathies,” Pharmacol Ther 138.311-322 (2013); and Pedersen and Sigurdsson, “Tau Immunotherapy for Alzheimer's Disease.” Trends Mol Med 21:394-402 (2015)). On the diagnostic side, a few dye-based Aß positron emission tomography (PET) tracers are already in clinical use and at least three such small molecule tau-targeting PET tracers are in clinical development (Congdon et al., “Harnessing the Immune System for Treatment and Detection of Tau Pathology,” J Alzheimers Dis 40 (Suppl 1) S113-8121 (2014)). Similar approaches targeting α-syn are not as advanced (Eberling et al . . . “Alpha-synuclein Imaging: a Critical Need for Parkinson's Disease Research,” J Parkinsons Dis 3:565-567 (2013)). There are no reports of selective or specific α-syn imaging probes. Interestingly, α-syn deposits are not only found in Parkinson's disease but also often found in Alzheimer's disease and are the main lesion in Lewy Body Dementia (Lippa et al., “DLB and PDD Boundary Issues: Diagnosis. Treatment. Molecular Pathology, and Biomarkers.” Neurology 68:812-819 (2007)) and in Multiple System Atrophy (see e.g., Goedert et al., “The Synucleinopathies. Twenty Years On,” J. Parkinsons Dis. 7 (s1): S53-S71 (2017)). Hence, it is difficult to predict who is a candidate for α-syn targeting therapy, which would be greatly facilitated if an α-syn imaging probe would exist. Furthermore, based on Braak staging of Parkinson's disease brains, α-syn deposition is widely thought to precede dopaminergic loss (Hawkes et al . . . “A Timeline for Parkinson's Disease.” Parkinsonism Relat Disord 16:79-84 (2010) and Dickson et al., “Neuropathological Assessment of Parkinson's Disease: Refining the Diagnostic Criteria.” Lancet Neurol 8:1150-1157 (2009)). Hence, its in vivo detection by imaging would allow identification of presymptomatic individuals, who could then receive prophylactic therapies under development to target α-synuclein.

The present invention is directed to overcoming these and other deficiencies in the art.

### SUMMARY OF THE INVENTION

A first aspect of the present disclosure is directed to an antibody or fragment thereof comprising a heavy chain variable region. The heavy chain variable region comprises a complementarity-determining region 1 (H-CDR1) comprising an amino acid sequence of any one of SEQ ID NOs: 1-36, 218, 222, 226, 230, 234, 238, 340 or a modified amino acid sequence of any one of SEQ ID NOs: 1-36, 218, 222, 226, 230, 234, 238, and 340 said modified sequence containing 1, 2, or 3 amino acid residue modifications as compared to any one of SEQ ID NOs: 1-36, 218, 222, 226, 230, 234, 238, and 340; a complementarity-determining region 2 (H-CDR2) comprising an amino acid sequence of any one of SEQ ID NOs: 37-72, 219, 223, 227, 231, 235, 239, 341 or a modified amino acid sequence of any one of SEQ ID NOs: 37-72, 219, 223, 227, 231, 235, 239, and 341 said modified sequence containing 1, 2, or 3 amino acid residue modifications as compared to any one of SEQ ID NOs: 37-72, 219, 223, 227, 231, 235, 239, and 341; and a complementarity-determining region 3 (H-CDR3) comprising an amino acid sequence of any one of SEQ ID NOs: 73-108, 220, 224, 228, 232, 236, 240, 342 or a modified amino acid sequence of any one of SEQ ID NOs: 73-108, 220, 224, 228, 232, 236, 240, and 342 said modified sequence containing 1, 2, or 3 amino acid residue modifications as compared to any one of SEQ ID NOs: 73-108, 220, 224, 228, 232, 236, 240, and 342.

Another aspect of the present disclosure is directed to an antibody or fragment thereof comprising a heavy chain variable region. The heavy chain variable region comprises a complementarity-determining region 1 (H-CDR1) comprising an amino acid sequence of any one of SEQ ID NOs: 146-160, or a modified amino acid sequence of any one of SEQ ID NOS: 146-160, said modified sequence containing 1, 2, or 3 amino acid residue modifications as compared to any one of SEQ ID NOs: 146-160; a complementarity-determining region 2 (H-CDR2) comprising an amino acid sequence of any one of SEQ ID NOs: 161-175, or a modified amino acid sequence of any one of SEQ ID NOs: 161-175, said modified sequence containing 1, 2, or 3 amino acid residue modifications as compared to any one of SEQ ID NOs: 161-175; and a complementarity-determining region 3 (H-CDR3) comprising an amino acid sequence of any one of SEQ ID NOs: 176-190, or a modified amino acid sequence of any one of SEQ ID NOS: 176-190, said modified sequence containing 1, 2, or 3 amino acid residue modifications as compared to any one of SEQ ID NOs: 176-190.

Another aspect of the present disclosure is directed to a method of inhibiting onset of one or more symptoms of an α-synucleinopathy in a subject. This method involves administering to the subject a pharmaceutical composition comprising one or more antibodies or binding fragments thereof as described herein or polynucleotides encoding the one or more antibodies or binding fragments thereof as described herein in an amount effective to inhibit onset of one or more symptoms of the α-synucleinopathy in the subject.

Another aspect of the present disclosure is directed to a method of treating an α-synucleinopathy in a subject. This method involves administering to the subject a pharmaceutical composition comprising one or more antibodies or binding fragments thereof as described herein or polynucleotides encoding the one or more antibodies or binding fragments thereof as described herein in an amount effective to treat the α-synucleinopathy in the subject.

Another aspect of the present disclosure is directed to a method of diagnosing an α-synucleinopathy in a subject. This method involves detecting, in the subject, the presence of accumulated α-synuclein protein or peptide using the antibody or binding fragment as described herein, and diagnosing the α-synucleinopathy based on the detection of the accumulated α-synuclein protein or peptide in the subject.

Another aspect of the present disclosure is directed to a method of monitoring the progression of an α-synucleinopathy in a subject. This method involves detecting, in the subject, the presence of α-synuclein protein or peptide using the antibody or binding fragment thereof as described herein, repeating the detecting step periodically; and monitoring the progression of the α-synucleinopathy in the subject as a result of the repeated detecting.

Another aspect of the present disclosure is directed to a diagnostic kit. The diagnostic kit contains any one or more antibodies or binding fragments thereof as described herein, and a detectable label.

### BRIEF DESCRIPTION OF THE DRAWINGS

FIG. 1A shows the α-synuclein single domain antibody (sdAb) titer generated in llama following 1-5 immunizations with recombinant α-synuclein. FIG. 1B shows immunoreactivity of the α-synuclein sdAb with brain homogenates from subjects with suspected dementia with Lewy Bodies (DLB).

FIGS. 2A-2B shows enrichment of α-synuclein sdAbs by phage display library panning against biotinylated-α-synuclein solution phase (FIG. 2A) and solid phase (FIG. 2B).

FIG. 3 shows the diverse binding profiles of the α-synuclein sdAb clones to different brain/protein fractions including (i) recombinant α-synuclein protein (rec syn), (ii) human brain having high levels of α-synuclein expression (syn brain), (iii) supernatant from DLB brain positive for tau and α-synuclein (syn+tau brain), and (iv) PHF-tau-enriched brain fraction from Alzheimer's brain (PHF).

FIGS. 4A-4I show immunohistochemical binding profiles of various sdAbs to human brain tissue with Lewy Body Dementia. FIG. 4A shows binding by a mixture of two commercial α-synuclein antibodies known to stain Lewy bodies and Lewy neurites. Immunoreactivity of polyclonal sdAbs panned against solid or solution phase α-synuclein is shown in FIGS. 4B and 4C, respectively. The binding profiles of particular clones, including sdAb 1G10 (FIG. 44), 2D10 (FIG. 4E), 2D8 (FIG. 4F), 2H7 (FIG. 4G), 2H1 (FIG. 4H), and 1D12 (FIG. 4I) are also shown.

FIGS. 5A-5B show that DLB brain has α-synuclein pathology (FIG. 5A) and tau pathology (FIG. 5B).

FIG. 6 is a sequence alignment of the anti-synuclein sdAb selected via solid-phase panning. The complementarity determining regions are shown.

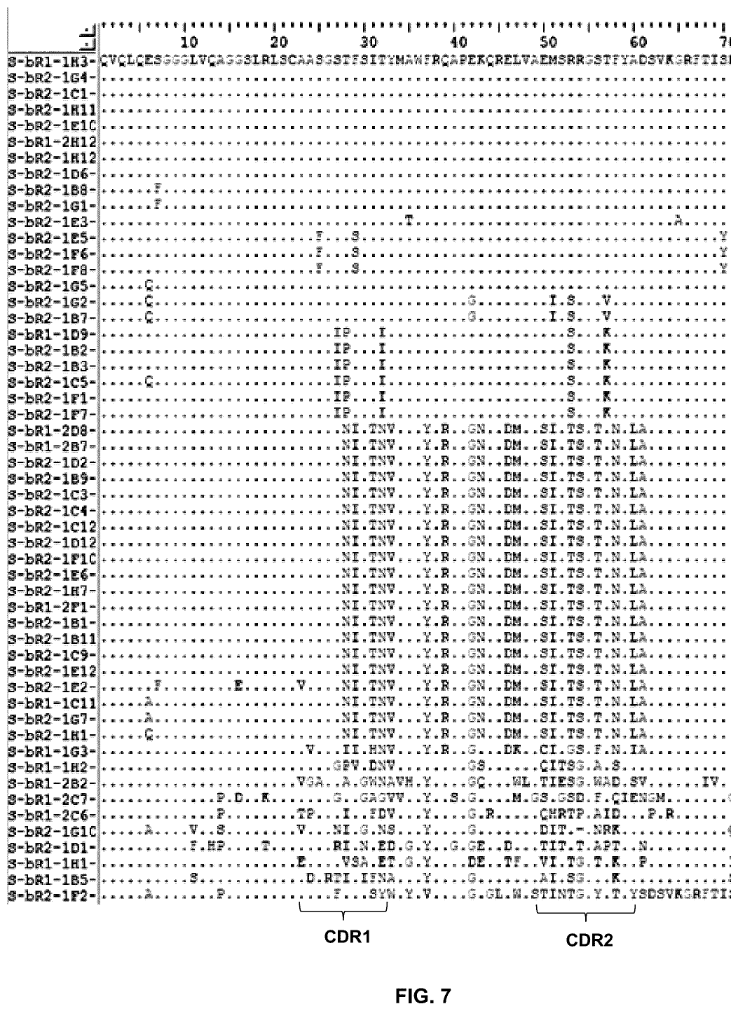

FIG. 7 is a sequence alignment of the anti-synuclein sdAb selected via solution-phase panning. The complementarity determining regions are shown.

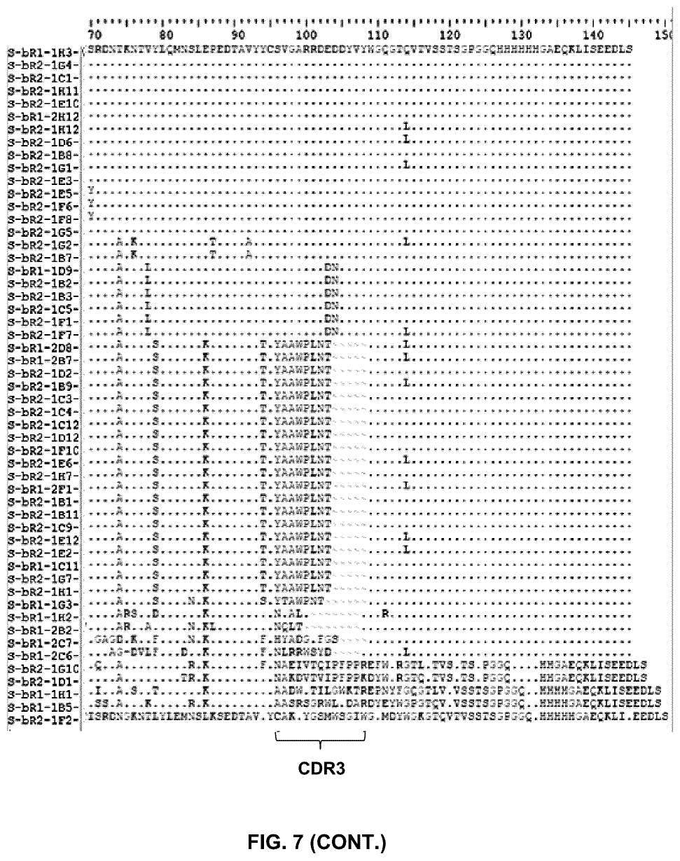

### DETAILED DESCRIPTION OF THE INVENTION

A first aspect of the present invention is directed to an antibody or binding fragment thereof that binds α-synuclein protein. In particular, the antibody or binding fragment as disclosed herein binds to human α-synuclein protein. In one embodiment, the antibody or binding fragment thereof of the present disclosure binds to aggregated forms of α-synuclein protein that are associated with the development and progression of α-synucleinopathies. In another embodiment, the antibody or binding fragment thereof of the present disclosure binds to the monomeric form of α-synuclein protein that accumulates with the development and progression of α-synucleinopathies.

In one embodiment, the antibodies described herein are single domain antibodies. The unique binding property or antigen binding specificity of a given antibody is determined by its complementarity determining regions (CDR) typically found in the light and heavy chain variable regions of an immunoglobulin. Single domain antibodies are antibodies whose CDRs are part of a single domain polypeptide. Examples include, but are not limited to, heavy chain variable domain antibodies (including three CDRs) and light chain variable domain antibodies (containing three CDRs). Single domain antibodies can be derived from heavy chains and light chains of conventional 4-chain antibodies, antibodies naturally devoid of light chains, engineered antibodies and single domain scaffolds other than those derived from antibodies. In one embodiment, the single domain antibody of the present invention is a heavy chain single domain antibody. i.e., a VHH or NANOBODYR. In one embodiment, the heavy chain single domain antibodies described herein are derived from naturally occurring heavy chain antibodies devoid of light chains.

Single domain antibodies are about 10-times smaller than conventional heavy chain and light chain containing IgG molecules. They are single polypeptides that are very stable, as they are resistant to extreme pH and temperature conditions. Moreover, unlike conventional antibodies, single domain antibodies are resistant to the action of proteases. In vitro expression of VHHs produces high yield, properly folded functional VHHs. Given their small size. VHHs are capable of recognizing unique epitopes of an antigen that cannot be bound by traditional full sized antibodies. As such, the anti-α-synuclein single domain antibodies described herein bind unique epitopes of α-synuclein and/or bind epitopes more efficiently than conventional α-30 synuclein antibodies.

The single domain antibodies as described herein can be derived from antibodies raised in Camelidae species, for example in camel, dromedary, llama, vicuña, alpaca and guanaco. Single domain antibodies produced by other species are also within the scope of the invention. For example, single domain antibodies as disclosed herein may be derived from antibodies produced in any species including, but not limited to mouse, human, camel, Ilama, goat, rabbit, bovine, and cartilaginous fish.

In another embodiment, the antibody as described herein is an antibody fragment. In one embodiment, an antibody fragment is a single-chain polypeptide comprising one CDR as described herein. In another embodiment, the antibody fragment is a single-chain polypeptide comprising two CDRs as described herein. In another embodiment, the antibody fragment is a single-chain polypeptide containing all three CDRs of the heavy chain variable regions as described herein. An antibody fragment as referred to herein may be devoid of one or more framework regions (FR1, FR2, FR3, or FR4) or any portion thereof of the heavy chain variable region.

In one embodiment, the antibody as described herein comprises a V_{H} domain coupled to one or more heavy chain constant regions (C_{H}). Mammalian heavy chain immunoglobulins typically have three or four constant region domains. Accordingly, the heavy chain variable regions described herein may be coupled to one heavy chain constant region, two heavy chain constant regions, three heavy chain constant regions, or four heavy chain constant regions.

In one embodiment, the antibody as described herein comprises a V_{H} domain coupled to an Fc region, i.e., the antibody is an Fc-fusion antibody. The Fc region can be composed of the second and third constant domain regions (as it is for IgG, IgA, and IgD antibody isotypes), or composed of the second, third, and fourth constant domain regions (as it is for IgM and IgE antibody isotypes). In one embodiment, the Fc domain is derived from a human immunoglobulin. In one embodiment, the Fc domain is derived from human IgG1 including the C_{H}2 and C_{H}3 regions.

The Fc-region or domain of the fusion polypeptides described herein may impart non-antigen binding functions to the polypeptide, termed “effector functions”, such as complement binding, antibody-dependent cell cytotoxicity (ADCC), and other functions mediated through the binding of subregions of this dimeric structure with immune cell surface receptors, Fc-receptors. Certain natural and synthetic variants of the Fc-region polypeptide sequences with altered effector functions that are suitable for use in the fusion polypeptides described herein include the subclass variants: e.g. IgGi, IgG2i, IgG3i, IgG24; and mutant polypeptides as described in e.g. U.S. Pat. No. 5,624,821 to Winter, U.S. Pat. No. 6,528,624 to Idusogie. U.S. Pat. No. 7,183,387 to Presta. and U.S. Pat. No. 7,317,091 to Lazar et al., which are hereby incorporated by reference in their entirety.

In another embodiment, the antibody or fragment thereof comprises two or more variable domain regions couple together. For example, in one embodiment, the antibody as described herein comprises two, or three, or more heavy chain variable regions linked together in tandem. In another embodiment, the heavy chain variable region is fused together with a light chain variable region to form a single-chain variable domain antibody (scFv) or a single-chain variable domain with an Fc portion (i.e., a scFv-Fc, e.g., a minibody). In another embodiment, two or more single-chain antibodies are linked together either in tandem (i.e., tandem scFvs), or such that they dimerize to form diabodies or triabodies. In another embodiment, the antibody is a tetrabody single chain variable fragment. In another embodiment, the antibody is a “linear antibody” which is an antibody comprising a pair of tandem Fd segments (V_{H}—C_{H}1—V_{H}-C_{H}1) that form a pair of antigen binding regions (see Zapata et al. Protein Eng. 8 (10): 1057-1062 (1995), which is hereby incorporated by reference in its entirety).

In another embodiment, the antibody of the disclosure is a conventional immunoglobulin (Ig) molecule comprising four polypeptide chains, i.e., two heavy chains and two light chains linked by disulfide bonds. In accordance with this embodiment, the single-domain antibodies as described herein are coupled to constant domain regions and further coupled to Ig light chains to create a four chain conventional antibody.

Antibody and antibody fragments disclosed herein can be mono-valent, bi-valent, or tri-valent with regard to binding domains, and the binding domains may be mono-specific, bi-specific, or tri-specific in binding specificity by design.

In one embodiment, the antibody or fragment thereof is isolated. As used herein, the term “isolated” refers to an antibody which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., gel filtration, ion exchange or reverse phase HPLC). Method for assessing antibody purity are known in the art (see e.g., Flatman et al., J. Chromatogr. B 848:79-87 (2007), which is hereby incorporated by reference in its entirety).

In one embodiment, the antibody or binding fragment thereof described herein is a chimeric antibody. A chimeric antibody is an antibody where one portion of the amino acid sequence of each of the heavy chains is homologous to corresponding sequences in an antibody derived from a particular species or belonging to a particular class, while the remaining segment of each chain is homologous to corresponding sequences in another species or class. Typically, the variable region mimics the variable region of an antibody derived from one species of mammals, while the constant portions are homologous to sequences of antibodies derived from another. For example, the variable region can be any one of the heavy chain variable regions disclosed herein derived from a camelid antibody coupled to one or more constant regions derived from a human immunoglobulin. Methods of making chimeric antibodies are well known in the art, see e.g., U.S. Pat. No. 4,816,567; and Morrison et al., “Chimeric human antibody molecules: mouse antigen-binding domains with human constant region domains” Proc. Natl. Acad. Sci. USA 81:6851-6855 (1984), which are hereby incorporated by reference in their entirety).

In another embodiment, the antibody or binding fragment thereof is a CDR-grafted antibody. A “CDR-grafted antibody” is an antibody which comprises variable region sequences of one species, where one or more of the CDR regions are replaced with CDR regions of another species. For example, in one embodiment the CDR grafted antibody comprises human or humanized heavy chain variable regions, where one or more of the CDRs within these regions is replaced with one or more CDRs disclosed herein that are derived from camelid heavy chain antibodies.

In another embodiment, the antibody or binding fragment thereof is a humanized antibody. A humanized antibody is an antibody or a variant, derivative, analog or portion thereof which comprises a framework region having substantially the amino acid sequence of a human antibody and a complementary determining region having substantially the amino acid sequence of a non-human antibody. As used herein, the term “substantially” in the context of a CDR refers to a CDR having an amino acid sequence that is at least 65%, at least 70%, at least 75%, at least 80%, at least 85%, at least 90%, at least 95%, at least 98% or at least 99% identical to the amino acid sequence of a non-human antibody CDR. Likewise, the term “substantially” in the context of a FR refers to a FR having an amino acid sequence that is at least 65%, at least 70%, at least 75%, at least 80%, at least 85%, at least 90%, at least 95%, at least 98% or at least 99% identical to the amino acid sequence of a human FR. A humanized antibody in accordance with the present disclosure comprises, for example, substantially all of at least one variable domains (Fv) in which all or substantially all of the CDR regions correspond to those of a non-human immunoglobulin (i.e., the donor camelid antibody) and all or substantially all of the framework regions are those of a human or humanized immunoglobulin framework sequence (i.e., the acceptor antibody).

Methods of humanizing antibodies are well known in the art, see e.g., Almagro and Fransson, “Humanization of Antibodies,” Frontiers in Bioscience 13:1619-1633 (2008), U.S. U.S. Pat. No. 6,054,297 to Carter et al., U.S. Pat. No. 8,343,489, and U.S. Patent Application Publication No. US20100261620 to Almagro et al . . . which are hereby incorporated by reference in their entirety. The human or humanized framework sequences can be chosen based on known structure, i.e., a fixed framework sequence, sequence homology to the framework sequences of the donor antibody (e.g., the antibody from which the CDRs are derived), i.e., a best-fit framework sequence, or a combination of both approaches. Regardless of the method chosen to select the human framework sequence, the sequences can be selected from mature framework sequences, germline gene sequences, or consensus framework sequences. Compatible human framework sequences are those that are similar in both length and sequence to the framework sequence of the donor antibody sequence (i.e., the antibody from which the CDRs are derived) to ensure proper folding of the antibody and binding domain formation.

In one embodiment, the humanized framework sequence of a humanized antibody of the disclosure comprises a consensus framework sequence. A consensus framework sequence is derived from a consensus immunoglobulin sequence, which is the sequence formed from the most frequently occurring amino acids (or nucleotides) in a family of related immunoglobulin sequences (see e.g., WINNAKER, “From Genes to Clones: Introduction to Gene Technology” (1987); Carter et al., Proc. Natl. Acad. Sci. USA. 89:4285 (1992); and Presta et al., J. Immunol., 151:2623 (1993), which are hereby incorporated by reference in their entirety). In a family of immunoglobulins, each position in the consensus sequence is occupied by the amino acid residue occurring most frequently at that position in the family. If two amino acids occur equally frequently, either can be included in the consensus sequence.

In another embodiment, a humanized antibody or binding fragment thereof as disclosed herein comprises a fixed framework region. Human heavy chain FR sequences known in the art can be used as heavy chain “acceptor” framework sequences (or simply. “acceptor” sequences) to humanize a non-human antibody using techniques known in the art (see e.g., Sims et al., J. Immunol . . . 151: 2296 (1993); Chothia et al., J. Mol. Biol . . . 196: 901 (1987), which are hereby incorporated by reference in their entirety). In one embodiment, human heavy chain acceptor sequences are selected from the framework sequences listed in publically available databases such as V-base or in the international ImMunoGeneTics®: (IMGT®) information system.

Humanized antibodies or binding fragments thereof as described herein may also comprise at least a portion of an immunoglobulin constant region (Fc), typically that of a human immunoglobulin. In one embodiment, the humanized antibody disclosed herein comprises the heavy chain variable domain. The humanized antibody may further comprise the CH1, hinge. CH2, CH3, and CH4 regions of a human heavy chain. In another embodiment, the humanized antibody comprises only a humanized heavy chain. Humanized antibodies and binding fragments thereof as described herein may be selected from any class of immunoglobulins, including IgM, IgG, IgD, IgA and IgE, and any isotype, including without limitation IgG1, IgG2, IgG3 and IgG4. The humanized antibody or binding fragment thereof may comprise sequences from more than one class or isotype, and particular constant domains may be selected to optimize desired effector functions using techniques well-known in the art.

The antibodies and binding fragments thereof described herein can be humanized antibodies (fully or partially humanized) as described supra. Alternatively, the antibodies and binding fragments thereof can be animal antibodies such as, but not limited to, a bird (for example, a duck or a goose), a shark, a whale, or a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.). In one embodiment, the antibodies and binding fragments thereof as described herein are derived from camelid antibodies.

Methods of antibody production, in particular, monoclonal antibody production, may be carried out using the methods described herein and those well-known in the art (MONOCLONAL ANTIBODIES—PRODUCTION, ENGINEERING AND CLINICAL APPLICATIONS (Mary A. Ritter and Heather M. Ladyman eds., 1995), which is hereby incorporated by reference in its entirety). Generally, the process involves obtaining immune cells (lymphocytes) from the spleen of an animal which has been previously immunized with the antigen of interest (e.g., full length α-synuclein as set forth in the Examples herein) either in vivo or in vitro.

The antibody-secreting lymphocytes are then fused with myeloma cells or transformed cells, which are capable of replicating indefinitely in cell culture, thereby producing an immortal, immunoglobulin-secreting cell line. Fusion with mammalian myeloma cells or other fusion partners capable of replicating indefinitely in cell culture is achieved by standard and well-known techniques, for example, by using polyethylene glycol (PEG) or other fusing agents (Milstein and Kohler. “Derivation of Specific Antibody-Producing Tissue Culture and Tumor Lines by Cell Fusion.” Eur J Immunol 6:511 (1976), which is hereby incorporated by reference in its entirety). The immortal cell line, which is preferably murine, but may also be derived from cells of other mammalian species, is selected to be deficient in enzymes necessary for the utilization of certain nutrients, to be capable of rapid growth, and have good fusion capability. The resulting fused cells, or hybridomas, are cultured, and the resulting colonies screened for the production of the desired monoclonal antibodies. Colonies producing such antibodies are cloned, and grown either in vivo or in vitro to produce large quantities of antibody.

In another embodiment, monoclonal antibodies can be isolated from antibody phage libraries generated using the techniques described herein or known in the art, see e.g., McCafferty et al., “Phage Antibodies: Filamentous Phage Displaying Antibody Variable Domains.” Nature 348:552-554 (1990), which is hereby incorporated by reference in its entirety. Clackson et al., “Making Antibody Fragments using Phage Display Libraries.” Nature 352:624-628 (1991); and Marks et al . . . “By-Passing Immunization. Human Antibodies from V-Gene Libraries Displayed on Phage.” J. Mol. Biol. 222:581-597 (1991), which are hereby incorporated by reference in their entirety, describe the isolation of murine and human antibodies, respectively, using phage libraries. Subsequent publications describe the production of high affinity (nM range) human antibodies by chain shuffling (Marks et al., BioTechnology 10:779-783 (1992), which is hereby incorporated by reference in its entirety), as well as combinatorial infection and in vivo recombination as a strategy for constructing very large phage libraries (Waterhouse et al., Nuc. Acids. Res. 21:2265-2266 (1993), which is hereby incorporated by reference in its entirety). Thus, these techniques are viable alternatives to traditional monoclonal antibody hybridoma techniques for isolation of monoclonal antibodies.

Alternatively, monoclonal antibodies can be made using recombinant DNA methods as described in U.S. Pat. No. 4,816,567 to Cabilly et al. which is hereby incorporated by reference in its entirety. The polynucleotides encoding a monoclonal antibody are isolated from mature B-cells or hybridoma cells, for example, by RT-PCR using oligonucleotide primers that specifically amplify the genes encoding the heavy and light chains of the antibody. The isolated polynucleotides encoding the heavy and light chains are then cloned into suitable expression vectors, which when transfected into host cells such as E. coli cells, simian COS cells. Chinese hamster ovary (CHO) cells, or myeloma cells that do not otherwise produce immunoglobulin protein, generate monoclonal antibodies.

The polynucleotide(s) encoding a monoclonal antibody can further be modified using recombinant DNA technology to generate alternative antibodies. For example, the heavy chain constant domains of a camelid monoclonal antibody can be substituted for those regions of a human antibody to generate a chimeric antibody. Alternatively, the heavy chain constant domains of a camelid monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

In one embodiment of the present disclosure, the antibody or binding fragment thereof as disclosed herein comprises a heavy chain variable region (HCVR) having a H-CDR1 with an amino acid sequence selected from SEQ ID NOs: 1-36, 218, 222, 226, 230, 234, 238, 340 or a modified amino acid sequence thereof containing 1, 2, 3, 4, 5, or 6 amino acid residue modifications as compared to SEQ ID NOs: 1-36, 218, 222, 226, 230, 234, 238, and 340 that maintain or enhance binding specificity of the H-CDR1. In one embodiment, the amino acid sequence of the H-CDR1 contains no more than 1, 2, or 3 amino acid modifications as compared to SEQ ID NOs: 1-36, 218, 222, 226, 230, 234, 238, and 340 respectively. The HCVR further comprises a H-CDR2 with an amino acid sequence selected from any one of SEQ ID NOs: 37-72, 219, 223, 227, 231, 235, 239, 341, or a modified amino acid sequence thereof containing 1, 2, 3, 4, 5, or 6, amino acid residue modifications as compared to SEQ ID NOs: 37-72, 219, 223, 227, 231, 235, 239, and 341 that maintain or enhance binding specificity of the H-CDR2. In one embodiment, the amino acid sequence of the H-CDR2 contains no more than 1, 2, or 3, amino acid modifications as compared to SEQ ID NOs: 37-72, 219, 223, 227, 231, 235, 239, and 341 respectively. The HCVR of the antibody or binding fragment thereof comprises a H-CDR3 with an amino acid sequence selected from any one of SEQ ID NOs: 73-108, 220, 224, 228, 232, 236, 240, 342, or a modified amino acid sequence thereof containing 1, 2, 3, 4, 5, 6 or 7 amino acid residue modifications as compared to SEQ ID NOs: 73-108, 220, 224, 228, 232, 236, 240, and 342 that maintain or enhance binding specificity of the H-CDR3. In one embodiment, the amino acid sequence of the H-CDR3 contains no more than 1, 2, or 3 amino acid modifications as compared to SEQ ID NOs: 73-108, 220, 224, 228, 232, 236, 240, and 342 respectively. The amino acid sequences of SEQ ID NOs: 1-108, 218-220, 222-224, 226-228, 230-232, 234-236, 238-240, and 340-342 are provided in Table 1 below.

TABLE 1
α-Synuclein Single Domain Antibody Complementarity Determining Regions

| | | | SEQ | | | SEQ | | | SEQ |
|---|---|---|---|---|---|---|---|---|---|
| | | | ID | | | ID | | | ID |
| Antibody ID | Descr | Sequence | NO: | Descr | Sequence | NO: | Descr | Sequence | NO: |
| 03-S-sR1-2D3 | CDR1 | ATVSGFSIG | 1 | CDR2 | ANVSPSGAKY | 37 | CDR3 | NIRRFSYLSGD | 73 |
| 07-S-sR2-1D12 | CDR1 | ASGFTFSSS | 2 | CDR2 | ASIMRYGTTT | 38 | CDR3 | NVRSFVRTY | 74 |
| 47-S-sR2-1F11 | | | | | | | | | |
| 08-S-sR2-2E10 | CDR1 | ASGRTFSSF | 3 | CDR2 | TAINWSGSSTY | 39 | CDR3 | NAQRRWPLRDY | 75 |
| 11-S-sR1-2D12 | CDR1 | ASGLIFSIN | 4 | CDR2 | ARITTGGSTN | 40 | CDR3 | AADVRFGERTPY | 76 |
| 15-S-sR2-1E7 | CDR1 | ASGNIFRIN | 5 | CDR2 | AHIISGGSTN | 41 | CDR3 | NARTFVRTY | 77 |
| 16-S-sR2-2F6 | CDR1 | ASTSVFGNT | 6 | CDR2 | ARITTLGFTY | 42 | CDR3 | NRRGFRSY | 78 |
| 19-S-sR1-2G4 | CDR1 | ASGSIYHVN | 7 | CDR2 | ATLTHNNRVT | 43 | CDR3 | YYFVPRNPLFGRRIDFDA | 79 |
| 23-S-sR2-1E9 | CDR1 | ASGNIFRIN | 8 | CDR2 | AVVKSGGSTN | 44 | CDR3 | NAQTRLWSY | 80 |
| 82-S-sR1-2C11 | | | | | | | | | |
| 24-S-sR2-2F7 | CDR1 | ASGNIFRIN | 9 | CDR2 | AHIISGGSTN | 45 | CDR3 | NAQTRLWSY | 81 |
| 31-S-sR2-1F7 | CDR1 | ASRSFFSIN | 10 | CDR2 | ATITSRDSTN | 46 | CDR3 | YADQPWRGRA | 82 |
| 32-S-sR2-2F11 | CDR1 | HSTITFRIN | 11 | CDR2 | ARINPAGRTY | 47 | CDR3 | STWRLGRNY | 83 |
| 34-5-sR1-1Al2 | CDR1 | ASMTTLGFK | 12 | CDR2 | ATISSIGIST | 48 | CDR3 | FIVIRPSWMPQY | 84 |
| 39-S-sR2-1F8 | CDR1 | ASTSVFGNT | 13 | CDR2 | ARITTLGFTY | 49 | CDR3 | NRLWRPLA | 85 |
| 40-S-sR2-2G4 | CDR1 | ASGMRSSLA | 14 | CDR2 | ATITIGGNTN | 50 | CDR3 | NVRSFVRTY | 86 |
| 48-S-sR2-2G9 | CDR1 | ASGSTFISIK | 15 | CDR2 | AGITIKNNYIN | 51 | CDR3 | TVQRRLGRVY | 87 |
| 50-S-sR1-2B2 | CDR1 | ASGSTFRFN | 16 | CDR2 | ANINSSGRTM | 52 | CDR3 | NVRSFVRTY | 88 |
| 55-S-sR2-1G3 | CDR1 | ASGSRFSIN | 17 | CDR2 | AGITSLGFTN | 53 | CDR3 | NRRGFRSY | 89 |
| 56-S-sR2-2H1 | CDR1 | ASGSIFSIN | 18 | CDR2 | AGISRGGRTK | 54 | CDR3 | NVRSFVRTY | 90 |
| 58-S-sR1-2B4 | CDR1 | ASGSIFRIN | 19 | CDR2 | ATITNEGNTY | 55 | CDR3 | AGKVIRWY | 91 |
| 62-S-sR2-1A9 | CDR1 | ASTSVFGNT | 20 | CDR2 | ARITTLGFTY | 56 | CDR3 | RARRALRESH | 92 |
| 63-S-sR2-1G9 | CDR1 | ASGSIFSIN | 21 | CDR2 | AGISRGGRTK | 57 | CDR3 | NARSFVRTY | 93 |
| 64-S-sR2-2H2 | CDR1 | ASRNFFTFR | 22 | CDR2 | ASITTGGRTV | 58 | CDR3 | NARRRFPVPGPTDY | 94 |
| 66-S-sR1-2B0 | CDR1 | ASGITFRFN | 23 | CDR2 | ARVSSGGSTT | 59 | CDR3 | NVGNF | 95 |
| 70-S-sR2-1B3 | CDR1 | ASGRSILIK | 24 | CDR2 | ATISMAGVTN | 60 | CDR3 | NAQTRLWSY | 96 |
| 71-S-sR2-2A8 | CDR1 | ASGRIFGRN | 25 | CDR2 | ARITRDGRTM | 61 | CDR3 | NAQTRLWSY | 97 |
| 72-S-sR2-2H3 | CDR1 | ASRSTFRFN | 26 | CDR2 | AAISSRGGSTN | 62 | CDR3 | NVRSFVRTY | 98 |
| 74-S-sR1-2B12 | CDR1 | TSGSIFSIN | 27 | CDR2 | AAISGRGSTH | 63 | CDR3 | ALDQHMEVIVSPGRIGS | 99 |
| 78-S-sR2-1C9 | CDR1 | LSTTMFGFW | 28 | CDR2 | ATIDSRGRTN | 64 | CDR3 | NAQRRWPLRDY | 100 |
| 79-S-sR2-2B3 | CDR1 | ASGNIFRIN | 29 | CDR2 | ARISSGGSTN | 65 | CDR3 | NARRPLRWYFY | 101 |
| 80-S-sR2-2H5 | CDR1 | ASGSIFSTN | 30 | CDR2 | ASITKFGNTD | 66 | CDR3 | YQNSRGRWYDIFRDY | 102 |
| 80-S-sR2-2H9 | CDR1 | ASRSSFRIT | 31 | CDR2 | ASITTGGRTV | 67 | CDR3 | NACIFIRWPLRDY | 103 |
| 86-S-sR2-1D5 | CDR1 | ASGNIFRIN | 32 | CDR2 | AHIISGGSTN | 68 | CDR3 | NAERRFGMRQV | 104 |
| 87-S-sR2-2D8 | CDR1 | ASVVPFRYF | 33 | CDR2 | ASITSGGGVN | 69 | CDR3 | ARLLSLGSRWGYGMFT | 105 |
| 88-S-sR2-2H8 | CDR1 | ASGSIFSIK | 34 | CDR2 | AAIASGGFTN | 70 | CDR3 | NAQRRWPLRDY | 106 |
| 94-S-sR2-1D10 | CDR1 | ASGSAFRMN | 35 | CDR2 | AAISFRGSAN | 71 | CDR3 | AAGRPWQRTFY | 107 |
| 95-S-sR2-2D10 | CDR1 | ASGSIFSIN | 36 | CDR2 | AGISRGGRTK | 72 | CDR3 | AATRWSWGTKSY | 108 |
| S-sR2-2H7 | CDR1 | ASGNIFRINA | 218 | CDR2 | ASIDSAGRTNYG | 219 | CDR3 | CSTWRLGRNY | 220 |
| S-sR2-1F6 | CDR1 | ASGSTFSNNA | 222 | CDR2 | AYISSGGFTN | 223 | CDR3 | SAGGTYRSGNVYFFPRS | 224 |
| S-sR2-1G4 | CDR1 | ASGSIFSINS | 226 | CDR2 | ATISSRSTTY | 227 | CDR3 | KAGSVGRV | 228 |
| S-sR2-2C10 | CDR1 | ASMTTLGFKT | 230 | CDR2 | AAITSGGTAN | 231 | CDR3 | ASTrGWTEVGGRND | 232 |
| S-sR2-2E9 | CDR1 | ASGRTFRVNA | 234 | CDR2 | AAVTNGGSTT | 235 | CDR3 | NAERRFGMRQV | 236 |
| S-sR2-2G11 | CDR1 | ASGRVFSINT | 238 | CDR2 | ASMTRGGSAN | 239 | CDR3 | NAARGWRI | 240 |
| 90-S-sR1-2D1 | CDR1 | SGSIFRING | 340 | CDR1 | VAAVNWSGERT | 341 | CDR3 | AADTDYRLDGSTWITNL | 342 |

Suitable amino acid modifications to the heavy chain CDR sequences of the anti-α-synuclein antibody disclosed herein include, for example, conservative substitutions or functionally equivalent amino acid residue substitutions that result in variant CDR sequences having similar or enhanced binding characteristics to those of the CDR sequences disclosed herein. Conservative substitutions are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. Alternatively, the amino acid repertoire can be grouped as (1) acidic (aspartate, glutamate); (2) basic (lysine, arginine histidine), (3) aliphatic (glycine, alanine, valine, leucine, isoleucine, serine, threonine), with serine and threonine optionally grouped separately as aliphatic-hydroxyl; (4) aromatic (phenylalanine, tyrosine, tryptophan); (5)amide (asparagine, glutamine); and (6) sulfur-containing (cysteine and methionine) (Stryer (ed.), Biochemistry, 2nd ed, WH Freeman and Co., 1981, which is hereby incorporated by reference in its entirety). Non-conservative substitutions can also be made to the heavy chain CDR sequences and the light chain CDR sequences as disclosed herein. Non-conservative substitutions involve substituting one or more amino acid residues of the CDR with one or more amino acid residues from a different class of amino acids to improve or enhance the binding properties of CDR.

The amino acid sequences of the heavy chain variable region CDRs of the anti-α-synuclein antibody described herein may further comprise one or more internal neutral amino acid insertions or deletions that do not alter synuclein protein binding. In one embodiment, the H-CDR3 having an amino acid sequence of any one of SEQ ID NOs: 73-108, 220, 224, 228, 232, 236, 240, and 342 further contains one or more internal neutral amino acid insertions or deletions that do not alter α-synuclein binding.

In one embodiment, the antibody or binding fragment thereof has a heavy chain variable region with a H-CDR1 having the amino acid sequence of SEQ ID NO: 1, or a modified amino acid sequence thereof containing 1, 2, 3, or more amino acid residue modifications as compared to SEQ ID NO: 1; a H-CDR2 having the amino acid sequence of SEQ ID NO: 37, or a modified amino acid sequence thereof containing 1, 2, 3, or more amino acid residue modifications as compared to SEQ ID NO: 37; and a H-CDR3 comprising the amino acid sequence of SEQ ID NO: 73, or a modified amino acid sequence thereof, said modified amino acid sequence containing 1, 2, 3, or more amino acid modifications as compared to SEQ ID NO: 73.

An exemplary single domain antibody having this heavy chain variable region is referred to herein as the 03-S-sR1-2D3 antibody. The 03-S-sR1-2D3 antibody comprises a V_{H} chain amino acid sequence of SEQ ID NO: 109 as shown below.

SEQ ID NO: 109
QVQLQQSGGGLVQAGGSLILRCRATVSGFSIGTMGWYRQAPGKEREFVAN
VSPSGAKYFADSVKGRFTISRDNANNTVYLQMNSLKPFDTGVYYCNIRRF
SYLSGDWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 109

In one embodiment, the antibody or binding fragment thereof of the present disclosure comprises a humanized variant of the heavy chain variable region of SEQ ID NO: 109, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 109), or a combination of these approaches. The humanized framework regions are designed to be similar in length and sequence to the parental framework sequences of SEQ ID NO: 109. In one embodiment, the humanized framework regions share 50%, 55%, 60%, 65%, 70%, 75%, 80%, 85%, 90%, 95% or more sequence identity to the framework regions of SEQ ID NO: 109. In another embodiment, the humanized framework regions are 60%, 65%, 70%, 75%, 80%, 85%, 90%, 95% or more similar in length to the framework regions of SEQ ID NO: 109. Humanized variants of the heavy chain variable region of SEQ ID NO: 109 share at least 50%, at least 55%, at least 60%, at least 65%, at least 70%, at least 75%, at least 80%, at least 85%, at least 90%, at least 95% sequence identity along the entire length of SEQ ID 109.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 109. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In another embodiment, the antibody or binding fragment thereof has a heavy chain variable region with a H-CDR1 having the amino acid sequence of SEQ ID NO: 2, or a modified amino acid sequence thereof containing 1, 2, 3, or more amino acid residue modifications as compared to SEQ ID NO: 2; a H-CDR2 having the amino acid sequence of SEQ ID NO: 38, or a modified amino acid sequence thereof containing 1, 2, 3, or more amino acid residue modifications as compared to SEQ ID NO: 38; and a H-CDR3 comprising the amino acid sequence of SEQ ID NO: 74, or a modified amino acid sequence thereof, said modified amino acid sequence containing 1, 2, 3, or more amino acid modifications as compared to SEQ ID NO: 74.

Exemplary single domain antibodies having this heavy chain variable region are referred to herein as the 07-S-sR2-1D12 antibody and the 47-S-sR2-1F11 antibody. These antibodies comprise a Vu chain amino acid sequence of SEQ ID NO: 110 as shown below.

SEQ ID NO: 110
QVQLQESGGGLVQAGGSLRLSCAASGFTFSSSSMGWYRQAPGKQRELVAS
IMRYGTTTYTDSVKGRFTISRDNGQRTVYLQMNSLKPEDTAVYYCNVRS
FVRTYWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 110.

In one embodiment, the antibody or binding fragment thereof of the present disclosure comprises a humanized variant of the heavy chain variable region of SEQ ID NO: 110, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 110), or a combination of these approaches. The humanized framework regions are designed to be similar in length and sequence to the parental framework sequences of SEQ ID NO: 110. In one embodiment, the humanized framework regions share 50%, 55%, 60%, 65%, 70%, 75%, 80%, 85%, 90%, 95% or more sequence identity to the framework regions of SEQ ID NO: 110. In another embodiment, the humanized framework regions are 60%, 65%, 70%, 75%, 80%, 85%, 90%, 95% or more similar in length to the framework regions of SEQ ID NO: 110. Humanized variants of the heavy chain variable region of SEQ ID NO: 110 share at least 50%, at least 55%, at least 60%, at least 65%, at least 70%, at least 75%, at least 80%, at least 85%, at least 90%, at least 95% sequence identity along the entire length of SEQ ID 110.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 110. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 3, or a modified amino acid sequence thereof containing 1, 2, 3, or more amino acid residue modifications as compared to SEQ ID NO: 3; a H-CDR2 having the amino acid sequence of SEQ ID NO: 39, or a modified amino acid sequence thereof containing 1, 2, 3, or more amino acid residue modifications as compared to SEQ ID NO: 39; and a H-CDR3 comprising the amino acid sequence of SEQ ID NO: 75, or a modified amino acid sequence thereof, said modified amino acid sequence containing 1, 2, 3, or more amino acid modifications as compared to SEQ ID NO: 75.

An exemplary single domain antibody having this heavy chain variable region is referred to herein as the 08-S-sR2-2E10 antibody. The 08-S-sR2-2E10 antibody comprises a VII chain amino acid sequence of SEQ ID NO: 111 as shown below.

SEQ ID NO: 111
QVQLQESGGGLVQAGGSLRLSCAASGRTFSSFAMGWFRQAPGKEREFVTA
INWSGSSTYYADSVKGRFTISRDNAKNTVYLQMNSLKPEDTAVYYCNAQR
RWPLRDYWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 111.

In one embodiment, the antibody or binding fragment thereof of the present disclosure comprises a humanized variant of the heavy chain variable region of SEQ ID NO: 111, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 111), or a combination of these approaches. The humanized framework regions are designed to be similar in length and sequence to the parental framework sequences of SEQ ID NO: 111. In one embodiment, the humanized framework regions share 50%, 55%, 60%, 65%, 70%, 75%, 80%, 85%, 90%, 95% or more sequence identity to the framework regions of SEQ ID NO: 111. In another embodiment, the humanized framework regions are 60%, 65%, 70%, 75%, 80%, 85%, 90%, 95% or more similar in length to the framework regions of SEQ ID NO: 111. Humanized variants of the heavy chain variable region of SEQ ID NO: 111 share at least 50%, at least 55%, at least 60%, at least 65%, at least 70%, at least 75%, at least 80%, at least 85%, at least 90%, at least 95% sequence identity along the entire length of SEQ ID 111.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 111. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 4, or a modified amino acid sequence thereof containing 1, 2, 3, or more amino acid residue modifications as compared to SEQ ID NO: 4; a H-CDR2 having the amino acid sequence of SEQ ID NO: 40, or a modified amino acid sequence thereof containing 1, 2, 3, or more amino acid residue modifications as compared to SEQ ID NO: 40; and a H-CDR3 comprising the amino acid sequence of SEQ ID NO: 76, or a modified amino acid sequence thereof, said modified amino acid sequence containing 1, 2, 3, or more amino acid modifications as compared to SEQ ID NO: 76.

An exemplary single domain antibody having this heavy chain variable region is referred to herein as the 11-S-sR1-2D12 antibody. The 11-S-sR1-2D12 antibody comprises a V_{H} chain amino acid sequence of SEQ ID NO: 112 as shown below.

SEQ ID NO: 112
QVQLQESGGGLVQAGGSLRLSCAASGLIFSINAMAWYRQAPGNQRELVAR
ITTGGSTNYADSVKGRFTISRDNAKNTVYLQMNSIKPEDTAVYFCAADVR
IGERTPYWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 112.

In one embodiment, the antibody or binding fragment thereof of the present disclosure comprises a humanized variant of the heavy chain variable region of SEQ ID NO: 112, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 112), or a combination of these approaches. The humanized framework regions are designed to be similar in length and sequence to the parental framework sequences of SEQ ID NO: 112. In one embodiment, the humanized framework regions share 50%, 55%, 60%, 65%, 70%, 75%, 80%, 85%, 90%, 95% or more sequence identity to the framework regions of SEQ ID NO: 112. In another embodiment, the humanized framework regions are 60%, 65%, 70%, 75%, 80%, 85%, 90%, 95% or more similar in length to the framework regions of SEQ ID NO: 112. Humanized variants of the heavy chain variable region of SEQ ID NO: 112 share at least 50%, at least 55%, at least 60%, at least 65%, at least 70%, at least 75%, at least 80%, at least 85%, at least 90%, at least 95% sequence identity along the entire length of SEQ ID 112.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 112. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 5, or a modified amino acid sequence thereof containing 1, 2, 3, or more amino acid residue modifications as compared to SEQ ID NO: 5; a H-CDR2 having the amino acid sequence of SEQ ID NO: 41, or a modified amino acid sequence thereof containing 1, 2, 3, or more amino acid residue modifications as compared to SEQ ID NO: 41; and a H-CDR3 comprising the amino acid sequence of SEQ ID NO: 77, or a modified amino acid sequence thereof, said modified amino acid sequence containing 1, 2, 3, or more amino acid modifications as compared to SEQ ID NO: 77.

An exemplary single domain antibody having this heavy chain variable region is referred to herein as the 15-S-sR2-1E7 antibody. The 15-S-sR2-1E7 antibody comprises a V_{H} chain amino acid sequence of SEQ ID NO: 113 as shown below.

SEQ ID NO: 113
QVQLQASGGGVVQSGGSLRLSCVASGNIFRINAMGWYRQAPGKQRELVAH
IISGGSTNYADSVKGRFTISREYAKNMVYLQMNSLKPEDTAVYYCNARTF
VRTYWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 113.

In one embodiment, the antibody or binding fragment thereof of the present disclosure comprises a humanized variant of the heavy chain variable region of SEQ ID NO: 113, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 113), or a combination of these approaches. The humanized framework regions are designed to be similar in length and sequence to the parental framework sequences of SEQ ID NO: 113. In one embodiment, the humanized framework regions share 50%, 55%, 60%, 65%, 70%, 75%, 80%, 85%, 90%, 95% or more sequence identity to the framework regions of SEQ ID NO: 113. In another embodiment, the humanized framework regions are 60%, 65%, 70%, 75%, 80%, 85%, 90%, 95% or more similar in length to the framework regions of SEQ ID NO: 113. Humanized variants of the heavy chain variable region of SEQ ID NO: 113 share at least 50%, at least 55%, at least 60%, at least 65%, at least 70%, at least 75%, at least 80%, at least 85%, at least 90%, at least 95% sequence identity along the entire length of SEQ ID 113.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 113. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 6, or a modified amino acid sequence thereof containing 1, 2, 3, or more amino acid residue modifications as compared to SEQ ID NO: 6; a H-CDR2 having the amino acid sequence of SEQ ID NO: 42, or a modified amino acid sequence thereof containing 1, 2, 3, or more amino acid residue modifications as compared to SEQ ID NO: 42; and a H-CDR3 comprising the amino acid sequence of SEQ ID NO: 78, or a modified amino acid sequence thereof, said modified amino acid sequence containing 1, 2, 3, or more amino acid modifications as compared to SEQ ID NO: 78.

An exemplary single domain antibody having this heavy chain variable region is referred to herein as the 16-S-sR2-2F6 antibody. The 16-S-sR2-2F6 antibody comprises a V_{H} chain amino acid sequence of SEQ ID NO: 114 as shown below.

SEQ ID NO: 114
QVQLQQFGGGLVQPGGSLRLSCQASTSVFGNTAMAWYRQAPGKQRELVAR
ITTLGFTYYADSAKGRFTISRDSAMNTVYLQMNSLKPEDTAVYYCNRRGF
RSYWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 114.

In one embodiment, the antibody or binding fragment thereof of the present disclosure comprises a humanized variant of the heavy chain variable region of SEQ ID NO: 114, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 114), or a combination of these approaches. The humanized framework regions are designed to be similar in length and sequence to the parental framework sequences of SEQ ID NO: 114. In one embodiment, the humanized framework regions share 50%, 55%, 60%, 65%, 70%, 75%, 80%, 85%, 90%, 95% or more sequence identity to the framework regions of SEQ ID NO: 114. In another embodiment, the humanized framework regions are 60%, 65%, 70%, 75%, 80%, 85%, 90%, 95% or more similar in length to the framework regions of SEQ ID NO: 114. Humanized variants of the heavy chain variable region of SEQ ID NO: 114 share at least 50%, at least 55%, at least 60%, at least 65%, at least 70%, at least 75%, at least 80%, at least 85%, at least 90%, at least 95% sequence identity along the entire length of SEQ ID 114.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 114. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 7, or a modified amino acid sequence thereof containing 1, 2, 3, or more amino acid residue modifications as compared to SEQ ID NO: 7; a H-CDR2 having the amino acid sequence of SEQ ID NO: 43, or a modified amino acid sequence thereof containing 1, 2, 3, or more amino acid residue modifications as compared to SEQ ID NO: 43; and a H-CDR3 comprising the amino acid sequence of SEQ ID NO: 79, or a modified amino acid sequence thereof, said modified amino acid sequence containing 1, 2, 3, or more amino acid modifications as compared to SEQ ID NO: 79.

An exemplary single domain antibody having this heavy chain variable region is referred to herein as the 19-S-sR1-2G4 antibody. The 19-S-sR1-2G4 antibody comprises a V_{H} chain amino acid sequence of SEQ ID NO: 115 as shown below.

SEQ ID NO: 115
QVQLQESGGGLVQSGDSLRLSCAASGSIYHVNTMGWYRQSPGKQRELVAT
LTHNNRVTYADSVKGRFTISRDNAKMTVYLQMDSLKPDDTAVYYCYYFVP
RNPLFGRRIDFDAWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 115.

In one embodiment, the antibody or binding fragment thereof of the present disclosure comprises a humanized variant of the heavy chain variable region of SEQ ID NO: 115, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 115), or a combination of these approaches. The humanized framework regions are designed to be similar in length and sequence to the parental framework sequences of SEQ ID NO: 115. In one embodiment, the humanized framework regions share 50%, 55%, 60%, 65%, 70%, 75%, 80%, 85%, 90%, 95% or more sequence identity to the framework regions of SEQ ID NO: 115. In another embodiment, the humanized framework regions are 60%, 65%, 70%, 75%, 80%, 85%, 90%, 95% or more similar in length to the framework regions of SEQ ID NO: 115. Humanized variants of the heavy chain variable region of SEQ ID NO: 115 share at least 50%, at least 55%, at least 60%, at least 65%, at least 70%, at least 75%, at least 80%, at least 85%, at least 90%, at least 95% sequence identity along the entire length of SEQ ID 115.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 115. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 8, or a modified amino acid sequence thereof containing 1, 2, 3, or more amino acid residue modifications as compared to SEQ ID NO: 8; a H-CDR2 having the amino acid sequence of SEQ ID NO: 44, or a modified amino acid sequence thereof containing 1, 2, 3, or more amino acid residue modifications as compared to SEQ ID NO: 44; and a H-CDR3 comprising the amino acid sequence of SEQ ID NO: 80, or a modified amino acid sequence thereof, said modified amino acid sequence containing 1, 2, 3, or more amino acid modifications as compared to SEQ ID NO: 80.

An exemplary single domain antibody having this heavy chain variable region is referred to herein as the 23-S-sR2-1E9 antibody. This antibody comprises a V_{H} chain amino acid sequence of SEQ ID NO: 116 as shown below.

SEQ ID NO: 116
QVQLQESGGGVVQSGGSLRLSCVASGNIFRINVMGWYRQAPGKQRELVAV
VKSGGSTNYVDSAKGRFTISRDNAKNTAYLHMDSLKPEDTAVYYCNAQTR
LWSYWGQGTQVTVSS

Another exemplary single domain antibody having this heavy chain variable region is referred to herein as the 82-S-sR1-2C11 antibody. This antibody comprises a V_{H} chain amino acid sequence of SEQ ID NO: 140 as shown below.

SEQ ID NO: 140
QVQLQESGGGVVQSGGSLRLSCVASGNIFRINVMGWYRQAPGKQRELVAV
VKSGGSTNYVDSAKGRFTISRDNAKNTAYLHMDSLKPEDTAVYYCNAQTR
LWSYWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 116 or SEQ ID NO: 140.

In one embodiment, the antibody or binding fragment thereof of the present disclosure comprises a humanized variant of the heavy chain variable region of SEQ ID NO: 116 or SEQ ID NO: 140, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 116 or SEQ ID NO: 140), or a combination of these approaches. The humanized framework regions are designed to be similar in length and sequence to the parental framework sequences of SEQ ID NO: 116 or SEQ ID NO: 140. In one embodiment, the humanized framework regions share 50%, 55%, 60%, 65%, 70%, 75%, 80%, 85%, 90%, 95% or more sequence identity to the framework regions of SEQ ID NO: 116 or SEQ ID NO: 140. In another embodiment, the humanized framework regions are 60%, 65%, 70%, 75%, 80%, 85%, 90%, 95% or more similar in length to the framework regions of SEQ ID NO: 116 or SEQ ID NO: 140. Humanized variants of the heavy chain variable region of SEQ ID NO: 116 or SEQ ID NO: 140share at least 50%, at least 55%, at least 60%, at least 65%, at least 70%, at least 75%, at least 80%, at least 85%, at least 90%, at least 95% sequence identity along the entire length of SEQ ID 116 or SEQ ID NO: 140, respectively.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 116. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 9, or a modified amino acid sequence thereof containing 1, 2, 3, or more amino acid residue modifications as compared to SEQ ID NO: 9; a H-CDR2 having the amino acid sequence of SEQ ID NO: 45, or a modified amino acid sequence thereof containing 1, 2, 3, or more amino acid residue modifications as compared to SEQ ID NO: 45; and a H-CDR3 comprising the amino acid sequence of SEQ ID NO: 81, or a modified amino acid sequence thereof, said modified amino acid sequence containing 1, 2, 3, or more amino acid modifications as compared to SEQ ID NO: 81.

An exemplary single domain antibody having this heavy chain variable region is referred to herein as the 24-S-sR2-2F7 antibody. The 24-S-sR2-2F7 antibody comprises a V_{H} chain amino acid sequence of SEQ ID NO: 117 as shown below.

SEQ ID NO: 117
QVQLQEFGGGVVQSGGSLRLSCVASGNIFRINAMGWYRQAPGKQRELVAH
IISGGSTNYADSVKGRFTISREYAKNMVYLQMNSLKPEDTAVYYCNAQTR
LWSYWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 117.

In one embodiment, the antibody or binding fragment thereof of the present disclosure comprises a humanized variant of the heavy chain variable region of SEQ ID NO: 117, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 117), or a combination of these approaches. The humanized framework regions are designed to be similar in length and sequence to the parental framework sequences of SEQ ID NO: 117. In one embodiment, the humanized framework regions share 50%, 55%, 60%, 65%, 70%, 75%, 80%, 85%, 90%, 95% or more sequence identity to the framework regions of SEQ ID NO: 117. In another embodiment, the humanized framework regions are 60%, 65%, 70%, 75%, 80%, 85%, 90%, 95% or more similar in length to the framework regions of SEQ ID NO: 117. Humanized variants of the heavy chain variable region of SEQ ID NO: 117 share at least 50%, at least 55%, at least 60%, at least 65%, at least 70%, at least 75%, at least 80%, at least 85%, at least 90%, at least 95% sequence identity along the entire length of SEQ ID 117.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 117. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 10, or a modified amino acid sequence thereof containing 1, 2, 3, or more amino acid residue modifications as compared to SEQ ID NO: 10; a H-CDR2 having the amino acid sequence of SEQ ID NO: 46, or a modified amino acid sequence thereof containing 1, 2, 3, or more amino acid residue modifications as compared to SEQ ID NO: 46; and a H-CDR3 comprising the amino acid sequence of SEQ ID NO: 82, or a modified amino acid sequence thereof, said modified amino acid sequence containing 1, 2, 3, or more amino acid modifications as compared to SEQ ID NO: 82.

An exemplary single domain antibody having this heavy chain variable region is referred to herein as the 31-S-sR2-1F7 antibody. The 31-S-sR2-1F7 antibody comprises a V_{H} I chain amino acid sequence of SEQ ID NO: 118 as shown below.

SEQ ID NO: 118
QVQLQESGGGLVQPGGSLRLSCAASRSFFSINAMGWYRQAPGKQRELVAT
ITSRDSTNVADSVKGRFTISRDYAKNIVYLQMDSLRPEDTATYYCYADQP
WRGRAWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 118.

In one embodiment, the antibody or binding fragment thereof of the present disclosure comprises a humanized variant of the heavy chain variable region of SEQ ID NO: 118, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 118), or a combination of these approaches. The humanized framework regions are designed to be similar in length and sequence to the parental framework sequences of SEQ ID NO: 118. In one embodiment, the humanized framework regions share 50%, 55%, 60%, 65%, 70%, 75%, 80%, 85%, 90%, 95% or more sequence identity to the framework regions of SEQ ID NO: 118. In another embodiment, the humanized framework regions are 60%, 65%, 70%, 75%, 80%, 85%, 90%, 95% or more similar in length to the framework regions of SEQ ID NO: 118. Humanized variants of the heavy chain variable region of SEQ ID NO: 118 share at least 50%, at least 55%, at least 60%, at least 65%, at least 70%, at least 75%, at least 80%, at least 85%, at least 90%, at least 95% sequence identity along the entire length of SEQ ID 118.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 118. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 11, or a modified amino acid sequence thereof containing 1, 2, 3, or more amino acid residue modifications as compared to SEQ ID NO: 11; a H-CDR2 having the amino acid sequence of SEQ ID NO: 47, or a modified amino acid sequence thereof containing 1, 2, 3, or more amino acid residue modifications as compared to SEQ ID NO: 47; and a H-CDR3 comprising the amino acid sequence of SEQ ID NO: 83, or a modified amino acid sequence thereof, said modified amino acid sequence containing 1, 2, 3, or more amino acid modifications as compared to SEQ ID NO: 83.

An exemplary single domain antibody having this heavy chain variable region is referred to herein as the 32-S-sR2-2F11 antibody. The 32-S-sR2-2F11 antibody comprises a V_{H} chain amino acid sequence of SEQ ID NO: 119 as shown below.

SEQ ID NO: 119
QVQLQESGGGVVQAGGSLNLSCTHSTITFRINTMAYYRQAPGSQRALVA
RINPAGRTYYPDSVKGRFTISRDNAKNQVYLQMNDLKPEDTAVYYCSTWR
LGRNYWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 119.

In one embodiment, the antibody or binding fragment thereof of the present disclosure comprises a humanized variant of the heavy chain variable region of SEQ ID NO: 119, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 119), or a combination of these approaches. The humanized framework regions are designed to be similar in length and sequence to the parental framework sequences of SEQ ID NO: 119. In one embodiment, the humanized framework regions share 50%, 55%, 60%, 65%, 70%, 75%, 80%, 85%, 90%, 95% or more sequence identity to the framework regions of SEQ ID NO: 119. In another embodiment, the humanized framework regions are 60%, 65%, 70%, 75%, 80%, 85%, 90%, 95% or more similar in length to the framework regions of SEQ ID NO: 119. Humanized variants of the heavy chain variable region of SEQ ID NO: 119 share at least 50%, at least 55%, at least 60%, at least 65%, at least 70%, at least 75%, at least 80%, at least 85%, at least 90%, at least 95% sequence identity along the entire length of SEQ ID 119.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 119. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 12, or a modified amino acid sequence thereof containing 1, 2, 3, or more amino acid residue modifications as compared to SEQ ID NO: 12; a H-CDR2 having the amino acid sequence of SEQ ID NO: 48, or a modified amino acid sequence thereof containing 1, 2, 3, or more amino acid residue modifications as compared to SEQ ID NO: 48; and a H-CDR3 comprising the amino acid sequence of SEQ ID NO: 84, or a modified amino acid sequence thereof, said modified amino acid sequence containing 1, 2, 3, or more amino acid modifications as compared to SEQ ID NO: 84.

An exemplary single domain antibody having this heavy chain variable region is referred to herein as the 34-S-sR1-1A12 antibody. The 34-S-sR1-1A12 antibody comprises a V_{H} chain amino acid sequence of SEQ ID NO: 120 as shown below.

SEQ ID NO: 120
QVQLQEFGGGLVQAGGVLRLSCVASMTTLGFKTMGWYRQAPGKQRELVAT
ISSIGISTYANSVKGRFTISRDNAKNTVYLQMNSLKPEDTAVYFCHVIRP
SWMPQYWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 120.

In one embodiment, the antibody or binding fragment thereof of the present disclosure comprises a humanized variant of the heavy chain variable region of SEQ ID NO: 120, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 120), or a combination of these approaches. The humanized framework regions are designed to be similar in length and sequence to the parental framework sequences of SEQ ID NO: 120. In one embodiment, the humanized framework regions share 50%, 55%, 60%, 65%, 70%, 75%, 80%, 85%, 90%, 95% or more sequence identity to the framework regions of SEQ ID NO: 120. In another embodiment, the humanized framework regions are 60%, 65%, 70%, 75%, 80%, 85%, 90%, 95% or more similar in length to the framework regions of SEQ ID NO: 120. Humanized variants of the heavy chain variable region of SEQ ID NO: 120 share at least 50%, at least 55%, at least 60%, at least 65%, at least 70%, at least 75%, at least 80%, at least 85%, at least 90%, at least 95% sequence identity along the entire length of SEQ ID 120.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 120. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 13, or a modified amino acid sequence thereof containing 1, 2, 3, or more amino acid residue modifications as compared to SEQ ID NO: 13; a H-CDR2 having the amino acid sequence of SEQ ID NO: 49, or a modified amino acid sequence thereof containing 1, 2, 3, or more amino acid residue modifications as compared to SEQ ID NO: 49; and a H-CDR3 comprising the amino acid sequence of SEQ ID NO: 85, or a modified amino acid sequence thereof, said modified amino acid sequence containing 1, 2, 3, or more amino acid modifications as compared to SEQ ID NO: 85.

An exemplary single domain antibody having this heavy chain variable region is referred to herein as the 39-S-sR2-1F8 antibody. The 39-S-sR2-1F8 antibody comprises a V_{H} chain amino acid sequence of SEQ ID NO: 121 as shown below.

SEQ ID NO: 121
QVQLQEFGGGLVQPGGSLRLSCQASTSVFGNTAMAWYRQAPGKQRELVAR
ITTLGFTYYADSAKGRFTISRDSAMNTVYLQMNSLKPEDTAVYYCNRLWR
PLAWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 121.

In one embodiment, the antibody or binding fragment thereof of the present disclosure comprises a humanized variant of the heavy chain variable region of SEQ ID NO: 121, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 121), or a combination of these approaches. The humanized framework regions are designed to be similar in length and sequence to the parental framework sequences of SEQ ID NO: 121. In one embodiment, the humanized framework regions share 50%, 55%, 60%, 65%, 70%, 75%, 80%, 85%, 90%, 95% or more sequence identity to the framework regions of SEQ ID NO: 121. In another embodiment, the humanized framework regions are 60%, 65%, 70%, 75%, 80%, 85%, 90%, 95% or more similar in length to the framework regions of SEQ ID NO: 121. Humanized variants of the heavy chain variable region of SEQ ID NO: 121 share at least 50%, at least 55%, at least 60%, at least 65%, at least 70%, at least 75%, at least 80%, at least 85%, at least 90%, at least 95% sequence identity along the entire length of SEQ ID 121.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 121. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 14, or a modified amino acid sequence thereof containing 1, 2, 3, or more amino acid residue modifications as compared to SEQ ID NO: 14; a H-CDR2 having the amino acid sequence of SEQ ID NO: 50, or a modified amino acid sequence thereof containing 1, 2, 3, or more amino acid residue modifications as compared to SEQ ID NO: 50; and a H-CDR3 comprising the amino acid sequence of SEQ ID NO: 86 or a modified amino acid sequence thereof, said modified amino acid sequence containing 1, 2, 3, or more amino acid modifications as compared to SEQ ID NO: 86.

An exemplary single domain antibody having this heavy chain variable region is referred to herein as the 40-S-sR2-2G4 antibody. The 40-S-sR2-2G4 antibody comprises a V_{H} I chain amino acid sequence of SEQ ID NO: 122 as shown below.

SEQ ID NO: 122
QVQLQESGGGLVQPGGSLRLSCAASGMRSSLAIMGWYRQAPGKQRELVAT
ITIGGNTNYADSVKGRFAISRDNTKRTVYLQMNSLTPEDTAVYYCNVRSF
VRTYWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 122.

In one embodiment, the antibody or binding fragment thereof of the present disclosure comprises a humanized variant of the heavy chain variable region of SEQ ID NO: 122, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 122), or a combination of these approaches. The humanized framework regions are designed to be similar in length and sequence to the parental framework sequences of SEQ ID NO: 122. In one embodiment, the humanized framework regions share 50%, 55%, 60%, 65%, 70%, 75%, 80%, 85%, 90%, 95% or more sequence identity to the framework regions of SEQ ID NO: 122. In another embodiment, the humanized framework regions are 60%, 65%, 70%, 75%, 80%, 85%, 90%, 95% or more similar in length to the framework regions of SEQ ID NO: 122. Humanized variants of the heavy chain variable region of SEQ ID NO: 122 share at least 50%, at least 55%, at least 60%, at least 65%, at least 70%, at least 75%, at least 80%, at least 85%, at least 90%, at least 95% sequence identity along the entire length of SEQ ID 122.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 122. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 15, or a modified amino acid sequence thereof containing 1, 2, 3, or more amino acid residue modifications as compared to SEQ ID NO: 15; a H-CDR2 having the amino acid sequence of SEQ ID NO: 51, or a modified amino acid sequence thereof containing 1, 2, 3, or more amino acid residue modifications as compared to SEQ ID NO: 51; and a H-CDR3 comprising the amino acid sequence of SEQ ID NO: 87, or a modified amino acid sequence thereof, said modified amino acid sequence containing 1, 2, 3, or more amino acid modifications as compared to SEQ ID NO: 87.

An exemplary single domain antibody having this heavy chain variable region is referred to herein as the 48-S-sR2-2G9 antibody. The 48-S-sR2-2G9 antibody comprises a V_{H} chain amino acid sequence of SEQ ID NO: 123 as shown below.

SEQ ID NO: 123
QVQLQQSGGGLVQAGGSLRLSCAASGSTFISIKTMGWYRQAPGKQRELVA
GITKNNYINYADSVKGRFTISRDNGKNTVYLQMNGLKPEDTAVYYCTVQR
RLGRVYWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 123.

In one embodiment, the antibody or binding fragment thereof of the present disclosure comprises a humanized variant of the heavy chain variable region of SEQ ID NO: 123, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 123), or a combination of these approaches. The humanized framework regions are designed to be similar in length and sequence to the parental framework sequences of SEQ ID NO: 123. In one embodiment, the humanized framework regions share 50%, 55%, 60%, 65%, 70%, 75%, 80%, 85%, 90%, 95% or more sequence identity to the framework regions of SEQ ID NO: 123. In another embodiment, the humanized framework regions are 60%, 65%, 70%, 75%, 80%, 85%, 90%, 95% or more similar in length to the framework regions of SEQ ID NO: 123. Humanized variants of the heavy chain variable region of SEQ ID NO: 123 share at least 50%, at least 55%, at least 60%, at least 65%, at least 70%, at least 75%, at least 80%, at least 85%, at least 90%, at least 95% sequence identity along the entire length of SEQ ID 123.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 123. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 16, or a modified amino acid sequence thereof containing 1, 2, 3, or more amino acid residue modifications as compared to SEQ ID NO: 16; a H-CDR2 having the amino acid sequence of SEQ ID NO: 52, or a modified amino acid sequence thereof containing 1, 2, 3, or more amino acid residue modifications as compared to SEQ ID NO: 52; and a H-CDR3 comprising the amino acid sequence of SEQ ID NO: 88, or a modified amino acid sequence thereof, said modified amino acid sequence containing 1, 2, 3, or more amino acid modifications as compared to SEQ ID NO: 88.

An exemplary single domain antibody having this heavy chain variable region is referred to herein as the 50-S-sR1-2B2 antibody. The 50-S-sR1-2B2 antibody comprises a V_{H} chain amino acid sequence of SEQ ID NO: 124 as shown below.

SEQ ID NO: 124
QVQLQESGGGLVQAGGSLRLSCTASGSTFRFNDMGWYRQAPGKQRELVAN
INSSGRTMYPDSVKGRFTISKDNVKNTVYLQMNSLTPEDTAVYYCNVRSF
VRTYWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 124.

In one embodiment, the antibody or binding fragment thereof of the present disclosure comprises a humanized variant of the heavy chain variable region of SEQ ID NO: 124, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 124), or a combination of these approaches. The humanized framework regions are designed to be similar in length and sequence to the parental framework sequences of SEQ ID NO: 124. In one embodiment, the humanized framework regions share 50%, 55%, 60%, 65%, 70%, 75%, 80%, 85%, 90%, 95% or more sequence identity to the framework regions of SEQ ID NO: 124. In another embodiment, the humanized framework regions are 60%, 65%, 70%, 75%, 80%, 85%, 90%, 95% or more similar in length to the framework regions of SEQ ID NO: 124. Humanized variants of the heavy chain variable region of SEQ ID NO: 124 share at least 50%, at least 55%, at least 60%, at least 65%, at least 70%, at least 75%, at least 80%, at least 85%, at least 90%, at least 95% sequence identity along the entire length of SEQ ID 124.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 124. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 17, or a modified amino acid sequence thereof containing 1, 2, 3, or more amino acid residue modifications as compared to SEQ ID NO: 17; a H-CDR2 having the amino acid sequence of SEQ ID NO: 53, or a modified amino acid sequence thereof containing 1, 2, 3, or more amino acid residue modifications as compared to SEQ ID NO: 53; and a H-CDR3 comprising the amino acid sequence of SEQ ID NO: 89, or a modified amino acid sequence thereof, said modified amino acid sequence containing 1, 2, 3, or more amino acid modifications as compared to SEQ ID NO: 89.

An exemplary single domain antibody having this heavy chain variable region is referred to herein as the 55-S-sR2-1G3 antibody. The 55-S-sR2-1G3 antibody comprises a V_{H} chain amino acid sequence of SEQ ID NO: 125 as shown below.

SEQ ID NO: 125
QVQLQESGGGLVQAGGSLTLSCVASGSRFSINTMGWYRQAPGKQRELVAG
ITSLGFTNYADSVKGRFTISRDNAKNTVYLQMNNLKVEDTAVYYCNRRGF
RSYWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 125.

In one embodiment, the antibody or binding fragment thereof of the present disclosure comprises a humanized variant of the heavy chain variable region of SEQ ID NO: 125, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 125), or a combination of these approaches. The humanized framework regions are designed to be similar in length and sequence to the parental framework sequences of SEQ ID NO: 125. In one embodiment, the humanized framework regions share 50%, 55%, 60%, 65%, 70%, 75%, 80%, 85%, 90%, 95% or more sequence identity to the framework regions of SEQ ID NO: 125. In another embodiment, the humanized framework regions are 60%, 65%, 70%, 75%, 80%, 85%, 90%, 95% or more similar in length to the framework regions of SEQ ID NO: 125. Humanized variants of the heavy chain variable region of SEQ ID NO: 125 share at least 50%, at least 55%, at least 60%, at least 65%, at least 70%, at least 75%, at least 80%, at least 85%, at least 90%, at least 95% sequence identity along the entire length of SEQ ID 125.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 125. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 18, or a modified amino acid sequence thereof containing 1, 2, 3, or more amino acid residue modifications as compared to SEQ ID NO: 18; a H-CDR2 having the amino acid sequence of SEQ ID NO: 54, or a modified amino acid sequence thereof containing 1, 2, 3, or more amino acid residue modifications as compared to SEQ ID NO: 54; and a H-CDR3 comprising the amino acid sequence of SEQ ID NO: 90, or a modified amino acid sequence thereof, said modified amino acid sequence containing 1, 2, 3, or more amino acid modifications as compared to SEQ ID NO: 90.

An exemplary single domain antibody having this heavy chain variable region is referred to herein as the 56-S-sR2-2H1 antibody. The 56-S-sR2-2H1 antibody comprises a V_{H} chain amino acid sequence of SEQ ID NO: 126 as shown below.

SEQ ID NO: 126
QVQLVESGGGFVQAGGSLRLSCAASGSIFSINYGNWYRQAPGKQRELVAG
ISRGGRTKYADSVKGRFTISRDSAKTLTLQMTSLKPEDTAVYYCNVRSFV
RTYWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 126.

In one embodiment, the antibody or binding fragment thereof of the present disclosure comprises a humanized variant of the heavy chain variable region of SEQ ID NO: 126, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 126), or a combination of these approaches. The humanized framework regions are designed to be similar in length and sequence to the parental framework sequences of SEQ ID NO: 126. In one embodiment, the humanized framework regions share 50%, 55%, 60%, 65%, 70%, 75%, 80%, 85%, 90%, 95% or more sequence identity to the framework regions of SEQ ID NO: 126. In another embodiment, the humanized framework regions are 60%, 65%, 70%, 75%, 80%, 85%, 90%, 95% or more similar in length to the framework regions of SEQ ID NO: 126. Humanized variants of the heavy chain variable region of SEQ ID NO: 126 share at least 50%, at least 55%, at least 60%, at least 65%, at least 70%, at least 75%, at least 80%, at least 85%, at least 90%, at least 95% sequence identity along the entire length of SEQ ID 126.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 126. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 19, or a modified amino acid sequence thereof containing 1, 2, 3, or more amino acid residue modifications as compared to SEQ ID NO: 19; a H-CDR2 having the amino acid sequence of SEQ ID NO: 55, or a modified amino acid sequence thereof containing 1, 2, 3, or more amino acid residue modifications as compared to SEQ ID NO: 55; and a H-CDR3 comprising the amino acid sequence of SEQ ID NO: 91, or a modified amino acid sequence thereof, said modified amino acid sequence containing 1, 2, 3, or more amino acid modifications as compared to SEQ ID NO: 91.

An exemplary single domain antibody having this heavy chain variable region is referred to herein as the 58-S-sR1-2B4 antibody. The 58-S-sR1-2B4 antibody comprises a V_{H} chain amino acid sequence of SEQ ID NO: 127 as shown below.

SEQ ID NO: 127
QVQLQESGGGLVQSGGSLRLSCSASGSIFRINLMGWYRQAPGKQRELVAT
ITNEGNTYYADSVKGRFTISRDNANNTWYLQMNSLKPEDTAVYECAGKVI
RWYWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 127.

In one embodiment, the antibody or binding fragment thereof of the present disclosure comprises a humanized variant of the heavy chain variable region of SEQ ID NO: 127, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 127), or a combination of these approaches. The humanized framework regions are designed to be similar in length and sequence to the parental framework sequences of SEQ ID NO: 127. In one embodiment, the humanized framework regions share 50%, 55%, 60%, 65%, 70%, 75%, 80%, 85%, 90%, 95% or more sequence identity to the framework regions of SEQ ID NO: 127. In another embodiment, the humanized framework regions are 60%, 65%, 70%, 75%, 80%, 85%, 90%, 95% or more similar in length to the framework regions of SEQ ID NO: 127. Humanized variants of the heavy chain variable region of SEQ ID NO: 127 share at least 50%, at least 55%, at least 60%, at least 65%, at least 70%, at least 75%, at least 80%, at least 85%, at least 90%, at least 95% sequence identity along the entire length of SEQ ID 127.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 127. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 20, or a modified amino acid sequence thereof containing 1, 2, 3, or more amino acid residue modifications as compared to SEQ ID NO: 20; a H-CDR2 having the amino acid sequence of SEQ ID NO: 56, or a modified amino acid sequence thereof containing 1, 2, 3, or more amino acid residue modifications as compared to SEQ ID NO: 56; and a H-CDR3 comprising the amino acid sequence of SEQ ID NO: 92, or a modified amino acid sequence thereof, said modified amino acid sequence containing 1, 2, 3, or more amino acid modifications as compared to SEQ ID NO: 92.

An exemplary single domain antibody having this heavy chain variable region is referred to herein as the 62-S-sR2-1A9 antibody. The 62-S-sR2-1A9 antibody comprises a V_{H} I chain amino acid sequence of SEQ ID NO: 128 as shown below.

SEQ ID NO: 128
QVQLQEFGGGLVQPGGSLRLSCQASTSVFGNTAMAWYRQAPCKQRELVAR
ITTLGFTYYADSAKGRFTISRDSAMNTVYLQMNSLKPEDTAVYYCRARRA
LRESHWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 128.

In one embodiment, the antibody or binding fragment thereof of the present disclosure comprises a humanized variant of the heavy chain variable region of SEQ ID NO: 128, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 128), or a combination of these approaches. The humanized framework regions are designed to be similar in length and sequence to the parental framework sequences of SEQ ID NO: 128. In one embodiment, the humanized framework regions share 50%, 55%, 60%, 65%, 70%, 75%, 80%, 85%, 90%, 95% or more sequence identity to the framework regions of SEQ ID NO: 128. In another embodiment, the humanized framework regions are 60%, 65%, 70%, 75%, 80%, 85%, 90%, 95% or more similar in length to the framework regions of SEQ ID NO: 128. Humanized variants of the heavy chain variable region of SEQ ID NO: 128 share at least 50%, at least 55%, at least 60%, at least 65%, at least 70%, at least 75%, at least 80%, at least 85%, at least 90%, at least 95% sequence identity along the entire length of SEQ ID 128.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 128. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 21, or a modified amino acid sequence thereof containing 1, 2, 3, or more amino acid residue modifications as compared to SEQ ID NO: 21; a H-CDR2 having the amino acid sequence of SEQ ID NO: 57, or a modified amino acid sequence thereof containing 1, 2, 3, or more amino acid residue modifications as compared to SEQ ID NO: 57; and a H-CDR3 comprising the amino acid sequence of SEQ ID NO: 93, or a modified amino acid sequence thereof, said modified amino acid sequence containing 1, 2, 3, or more amino acid modifications as compared to SEQ ID NO: 93.

An exemplary single domain antibody having this heavy chain variable region is referred to herein as the 63-S-sR2-1G9 antibody. The 63-S-sR2-1G9 antibody comprises a V_{H} chain amino acid sequence of SEQ ID NO: 129 as shown below.

SEQ ID NO: 129
QVQLQESGGGFVQAGGSLRLSCAASGSIFSINYGNWYRQAPGKQRELVAG
ISRGGRTKYADSVKGRFTISRDSAKTLTLQMTSLKPEDTAIYSCNARSFV
RTYWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 129.

In one embodiment, the antibody or binding fragment thereof of the present disclosure comprises a humanized variant of the heavy chain variable region of SEQ ID NO: 129, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 129), or a combination of these approaches. The humanized framework regions are designed to be similar in length and sequence to the parental framework sequences of SEQ ID NO: 129. In one embodiment, the humanized framework regions share 50%, 55%, 60%, 65%, 70%, 75%, 80%, 85%, 90%, 95% or more sequence identity to the framework regions of SEQ ID NO: 129. In another embodiment, the humanized framework regions are 60%, 65%, 70%, 75%, 80%, 85%, 90%, 95% or more similar in length to the framework regions of SEQ ID NO: 129. Humanized variants of the heavy chain variable region of SEQ ID NO: 129 share at least 50%, at least 55%, at least 60%, at least 65%, at least 70%, at least 75%, at least 80%, at least 85%, at least 90%, at least 95% sequence identity along the entire length of SEQ ID 129.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 129. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 22, or a modified amino acid sequence thereof containing 1, 2, 3, or more amino acid residue modifications as compared to SEQ ID NO: 22; a H-CDR2 having the amino acid sequence of SEQ ID NO: 58, or a modified amino acid sequence thereof containing 1, 2, 3, or more amino acid residue modifications as compared to SEQ ID NO: 58; and a H-CDR3 comprising the amino acid sequence of SEQ ID NO: 94, or a modified amino acid sequence thereof, said modified amino acid sequence containing 1, 2, 3, or more amino acid modifications as compared to SEQ ID NO: 94.

An exemplary single domain antibody having this heavy chain variable region is referred to herein as the 64-S-sR2-2H2 antibody. The 64-S-sR2-2H2 antibody comprises a V_{H} chain amino acid sequence of SEQ ID NO: 130 as shown below.

SEQ ID NO: 130
QVQLQESGGGLVTAGGSLRLSCAASRNFFTFRAMGWYRQAPGKQREMVAS
ITTGGRTVYADSVKGRFTISKSNANNTVYLQMNSLEAEDTAVYYCNARRR
FPVPGPTDYWGRGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 130.

In one embodiment, the antibody or binding fragment thereof of the present disclosure comprises a humanized variant of the heavy chain variable region of SEQ ID NO: 130, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 130), or a combination of these approaches. The humanized framework regions are designed to be similar in length and sequence to the parental framework sequences of SEQ ID NO: 130. In one embodiment, the humanized framework regions share 50%, 55%, 60%, 65%, 70%, 75%, 80%, 85%, 90%, 95% or more sequence identity to the framework regions of SEQ ID NO: 130. In another embodiment, the humanized framework regions are 60%, 65%, 70%, 75%, 80%, 85%, 90%, 95% or more similar in length to the framework regions of SEQ ID NO: 130. Humanized variants of the heavy chain variable region of SEQ ID NO: 130 share at least 50%, at least 55%, at least 60%, at least 65%, at least 70%, at least 75%, at least 80%, at least 85%, at least 90%, at least 95% sequence identity along the entire length of SEQ ID 130.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 130. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 23, or a modified amino acid sequence thereof containing 1, 2, 3, or more amino acid residue modifications as compared to SEQ ID NO: 23; a H-CDR2 having the amino acid sequence of SEQ ID NO: 59, or a modified amino acid sequence thereof containing 1, 2, 3, or more amino acid residue modifications as compared to SEQ ID NO: 59; and a H-CDR3 comprising the amino acid sequence of SEQ ID NO: 95, or a modified amino acid sequence thereof, said modified amino acid sequence containing 1, 2, 3, or more amino acid modifications as compared to SEQ ID NO: 95.

An exemplary single domain antibody having this heavy chain variable region is referred to herein as the 66-S-sR1-2B10 antibody. The 66-S-sR1-2B10 antibody comprises a V_{H} chain amino acid sequence of SEQ ID NO: 131 as shown below.

SEQ ID NO: 131
QVQLQESGGGLVQAGGSLRLSCAASGITRFNAMGWYRQAPGKERELVARV
SSGGSTTYADSVKARFTTFRDNVKNIGYLQMTSLKPEDTAVYYCNVGNFW
GQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 131.

In one embodiment, the antibody or binding fragment thereof of the present disclosure comprises a humanized variant of the heavy chain variable region of SEQ ID NO: 131, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 131), or a combination of these approaches. The humanized framework regions are designed to be similar in length and sequence to the parental framework sequences of SEQ ID NO: 131. In one embodiment, the humanized framework regions share 50%, 55%, 60%, 65%, 70%, 75%, 80%, 85%, 90%, 95% or more sequence identity to the framework regions of SEQ ID NO: 131. In another embodiment, the humanized framework regions are 60%, 65%, 70%, 75%, 80%, 85%, 90%, 95% or more similar in length to the framework regions of SEQ ID NO: 131. Humanized variants of the heavy chain variable region of SEQ ID NO: 131 share at least 50%, at least 55%, at least 60%, at least 65%, at least 70%, at least 75%, at least 80%, at least 85%, at least 90%, at least 95% sequence identity along the entire length of SEQ ID 131.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 131. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 24, or a modified amino acid sequence thereof containing 1, 2, 3, or more amino acid residue modifications as compared to SEQ ID NO: 24; a H-CDR2 having the amino acid sequence of SEQ ID NO: 60, or a modified amino acid sequence thereof containing 1, 2, 3, or more amino acid residue modifications as compared to SEQ ID NO: 60; and a H-CDR3 comprising the amino acid sequence of SEQ ID NO: 96, or a modified amino acid sequence thereof, said modified amino acid sequence containing 1, 2, 3, or more amino acid modifications as compared to SEQ ID NO: 96.

An exemplary single domain antibody having this heavy chain variable region is referred to herein as the 70-S-sR2-1B3 antibody. The 70-S-sR2-1B3 antibody comprises a V_{H} chain amino acid sequence of SEQ ID NO: 132 as shown below.

SEQ ID NO: 132
QVQLQESGGGLVRTGESLGLSCAASGRSILIKGMGWYRQAPGKEREMVAT
ISMAGVTNYSDSVKGRFTISRDNYKKTVSLQMNNLRPEDTAVYVCNAQTR
LWSYWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 132.

In one embodiment, the antibody or binding fragment thereof of the present disclosure comprises a humanized variant of the heavy chain variable region of SEQ ID NO: 132, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 132), or a combination of these approaches. The humanized framework regions are designed to be similar in length and sequence to the parental framework sequences of SEQ ID NO: 132. In one embodiment, the humanized framework regions share 50%, 55%, 60%, 65%, 70%, 75%, 80%, 85%, 90%, 95% or more sequence identity to the framework regions of SEQ ID NO: 132. In another embodiment, the humanized framework regions are 60%, 65%, 70%, 75%, 80%, 85%, 90%, 95% or more similar in length to the framework regions of SEQ ID NO: 132. Humanized variants of the heavy chain variable region of SEQ ID NO: 132 share at least 50%, at least 55%, at least 60%, at least 65%, at least 70%, at least 75%, at least 80%, at least 85%, at least 90%, at least 95% sequence identity along the entire length of SEQ ID 132.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 132. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 25, or a modified amino acid sequence thereof containing 1, 2, 3, or more amino acid residue modifications as compared to SEQ ID NO: 25; a H-CDR2 having the amino acid sequence of SEQ ID NO: 61, or a modified amino acid sequence thereof containing 1, 2, 3, or more amino acid residue modifications as compared to SEQ ID NO: 61; and a H-CDR3 comprising the amino acid sequence of SEQ ID NO: 97, or a modified amino acid sequence thereof, said modified amino acid sequence containing 1, 2, 3, or more amino acid modifications as compared to SEQ ID NO: 97.

An exemplary single domain antibody having this heavy chain variable region is referred to herein as the 71-S-sR2-2A8 antibody. The 71-S-sR2-2A8 antibody comprises a V_{H} chain amino acid sequence of SEQ ID NO: 133 as shown below.

SEQ ID NO: 133
QVQLVESGGGLVQAGGSLRLSCAASGRIFGRNAMAWYRQVPGKERELVAR
ITRDGRTMYVDSAKGRFTISRDNAKNTAYLHMDSLKPEDTAVYYCNAQTR
LWSYWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 133.

In one embodiment, the antibody or binding fragment thereof of the present disclosure comprises a humanized variant of the heavy chain variable region of SEQ ID NO: 133, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 133), or a combination of these approaches. The humanized framework regions are designed to be similar in length and sequence to the parental framework sequences of SEQ ID NO: 133. In one embodiment, the humanized framework regions share 50%, 55%, 60%, 65%, 70%, 75%, 80%, 85%, 90%, 95% or more sequence identity to the framework regions of SEQ ID NO: 133. In another embodiment, the humanized framework regions are 60%, 65%, 70%, 75%, 80%, 85%, 90%, 95% or more similar in length to the framework regions of SEQ ID NO: 133. Humanized variants of the heavy chain variable region of SEQ ID NO: 133 share at least 50%, at least 55%, at least 60%, at least 65%, at least 70%, at least 75%, at least 80%, at least 85%, at least 90%, at least 95% sequence identity along the entire length of SEQ ID 133.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 133. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 26, or a modified amino acid sequence thereof containing 1, 2, 3, or more amino acid residue modifications as compared to SEQ ID NO: 26; a H-CDR2 having the amino acid sequence of SEQ ID NO: 62, or a modified amino acid sequence thereof containing 1, 2, 3, or more amino acid residue modifications as compared to SEQ ID NO: 62; and a H-CDR3 comprising the amino acid sequence of SEQ ID NO: 98, or a modified amino acid sequence thereof, said modified amino acid sequence containing 1, 2, 3, or more amino acid modifications as compared to SEQ ID NO: 98.

An exemplary single domain antibody having this heavy chain variable region is referred to herein as the 72-S-sR2-2H3 antibody. The 72-S-sR2-2H3 antibody comprises a V_{H} chain amino acid sequence of SEQ ID NO: 134 as shown below.

SEQ ID NO: 134
QVQLQESGGGLVQAGGSLRLSCAASRSTFRFNYMGWYRQAPGKQRELVAA
ISSRGGSTNYADSVQGRFTISRDNAKNYVSLQMNSLKPEDTAVYYCNVRS
FVRTYWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 134.

In one embodiment, the antibody or binding fragment thereof of the present disclosure comprises a humanized variant of the heavy chain variable region of SEQ ID NO: 134, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 134), or a combination of these approaches. The humanized framework regions are designed to be similar in length and sequence to the parental framework sequences of SEQ ID NO: 134. In one embodiment, the humanized framework regions share 50%, 55%, 60%, 65%, 70%, 75%, 80%, 85%, 90%, 95% or more sequence identity to the framework regions of SEQ ID NO: 134. In another embodiment, the humanized framework regions are 60%, 65%, 70%, 75%, 80%, 85%, 90%, 95% or more similar in length to the framework regions of SEQ ID NO: 134. Humanized variants of the heavy chain variable region of SEQ ID NO: 134 share at least 50%, at least 55%, at least 60%, at least 65%, at least 70%, at least 75%, at least 80%, at least 85%, at least 90%, at least 95% sequence identity along the entire length of SEQ ID 134.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 134. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 27, or a modified amino acid sequence thereof containing 1, 2, 3, or more amino acid residue modifications as compared to SEQ ID NO: 27; a H-CDR2 having the amino acid sequence of SEQ ID NO: 63, or a modified amino acid sequence thereof containing 1, 2, 3, or more amino acid residue modifications as compared to SEQ ID NO: 63; and a H-CDR3 comprising the amino acid sequence of SEQ ID NO: 99, or a modified amino acid sequence thereof, said modified amino acid sequence containing 1, 2, 3, or more amino acid modifications as compared to SEQ ID NO: 99.

An exemplary single domain antibody having this heavy chain variable region is referred to herein as the 74-S-sR1-2B12 antibody. The 74-S-sR1-2B12 antibody comprises a Vu chain amino acid sequence of SEQ ID NO: 135 as shown below.

SEQ ID NO: 135
QVQLQEFGGGLVQAGGSLRLSCATSGSIFSINAVGWYRQAPGNQRELVAA
ISGRGSTHYADSVKGRFTISRDTAKNTVYLQMNSLKPEDTAVYYCALDQH
MEVIVSPGRIGSWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 135.

In one embodiment, the antibody or binding fragment thereof of the present disclosure comprises a humanized variant of the heavy chain variable region of SEQ ID NO: 135, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 135), or a combination of these approaches. The humanized framework regions are designed to be similar in length and sequence to the parental framework sequences of SEQ ID NO: 135. In one embodiment, the humanized framework regions share 50%, 55%, 60%, 65%, 70%, 75%, 80%, 85%, 90%, 95% or more sequence identity to the framework regions of SEQ ID NO: 135. In another embodiment, the humanized framework regions are 60%, 65%, 70%, 75%, 80%, 85%, 90%, 95% or more similar in length to the framework regions of SEQ ID NO: 135. Humanized variants of the heavy chain variable region of SEQ ID NO: 135 share at least 50%, at least 55%, at least 60%, at least 65%, at least 70%, at least 75%, at least 80%, at least 85%, at least 90%, at least 95% sequence identity along the entire length of SEQ ID 135.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 135. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 28, or a modified amino acid sequence thereof containing 1, 2, 3, or more amino acid residue modifications as compared to SEQ ID NO: 28; a H-CDR2 having the amino acid sequence of SEQ ID NO: 64, or a modified amino acid sequence thereof containing 1, 2, 3, or more amino acid residue modifications as compared to SEQ ID NO: 64; and a H-CDR3 comprising the amino acid sequence of SEQ ID NO: 100, or a modified amino acid sequence thereof, said modified amino acid sequence containing 1, 2, 3, or more amino acid modifications as compared to SEQ ID NO: 100.

An exemplary single domain antibody having this heavy chain variable region is referred to herein as the 78-S-sR2-1C9 antibody. The 78-S-sR2-1C9 antibody comprises a V_{H} chain amino acid sequence of SEQ ID NO: 136 as shown below.

SEQ ID NO: 136
QVQLQESGGGLVQAGESLTLSCALSTTMFGFWPMA
WFRQTPGQRRELIATIDSRGRTNIADSVKGRFTIS
RDNTKNTLYLRMNSLKPEDTAVYYCNAQRRWPLRD
YWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 136.

In one embodiment, the antibody or binding fragment thereof of the present disclosure comprises a humanized variant of the heavy chain variable region of SEQ ID NO: 136, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 136), or a combination of these approaches. The humanized framework regions are designed to be similar in length and sequence to the parental framework sequences of SEQ ID NO: 136. In one embodiment, the humanized framework regions share 50%, 55%, 60%, 65%, 70%, 75%, 80%, 85%, 90%, 95% or more sequence identity to the framework regions of SEQ ID NO: 136. In another embodiment, the humanized framework regions are 60%, 65%, 70%, 75%, 80%, 85%, 90%, 95% or more similar in length to the framework regions of SEQ ID NO: 136. Humanized variants of the heavy chain variable region of SEQ ID NO: 136 share at least 50%, at least 55%, at least 60%, at least 65%, at least 70%, at least 75%, at least 80%, at least 85%, at least 90%, at least 95% sequence identity along the entire length of SEQ ID 136.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 136. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 29, or a modified amino acid sequence thereof containing 1, 2, 3, or more amino acid residue modifications as compared to SEQ ID NO: 29; a H-CDR2 having the amino acid sequence of SEQ ID NO: 65, or a modified amino acid sequence thereof containing 1, 2, 3, or more amino acid residue modifications as compared to SEQ ID NO: 65; and a H-CDR3 comprising the amino acid sequence of SEQ ID NO: 101, or a modified amino acid sequence thereof, said modified amino acid sequence containing 1, 2, 3, or more amino acid modifications as compared to SEQ ID NO: 101.

An exemplary single domain antibody having this heavy chain variable region is referred to herein as the 79-S-sR2-2B3 antibody. The 79-S-sR2-2B3 antibody comprises a V_{H} chain amino acid sequence of SEQ ID NO: 137 as shown below.

SEQ ID NO: 137
QVQLQEFGGGVVQSGGSLRLSCVASGNIFRINAMG
WYRQAPGKQRELVARISSGGSTNYADSVKGRFTIS
RDNVKNTVTLQMNSLKPEDTAVYYCNARRPLRWYE
YWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 137.

In one embodiment, the antibody or binding fragment thereof of the present disclosure comprises a humanized variant of the heavy chain variable region of SEQ ID NO: 137, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 137), or a combination of these approaches. The humanized framework regions are designed to be similar in length and sequence to the parental framework sequences of SEQ ID NO: 137. In one embodiment, the humanized framework regions share 50%, 55%, 60%, 65%, 70%, 75%, 80%, 85%, 90%, 95% or more sequence identity to the framework regions of SEQ ID NO: 137. In another embodiment, the humanized framework regions are 60%, 65%, 70%, 75%, 80%, 85%, 90%, 95% or more similar in length to the framework regions of SEQ ID NO: 137. Humanized variants of the heavy chain variable region of SEQ ID NO: 137 share at least 50%, at least 55%, at least 60%, at least 65%, at least 70%, at least 75%, at least 80%, at least 85%, at least 90%, at least 95% sequence identity along the entire length of SEQ ID 137.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 137. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 30, or a modified amino acid sequence thereof containing 1, 2, 3, or more amino acid residue modifications as compared to SEQ ID NO: 30; a H-CDR2 having the amino acid sequence of SEQ ID NO: 66, or a modified amino acid sequence thereof containing 1, 2, 3, or more amino acid residue modifications as compared to SEQ ID NO: 66; and a H-CDR3 comprising the amino acid sequence of SEQ ID NO: 102, or a modified amino acid sequence thereof, said modified amino acid sequence containing 1, 2, 3, or more amino acid modifications as compared to SEQ ID NO: 102.

An exemplary single domain antibody having this heavy chain variable region is referred to herein as the 80-S-sR2-2H5 antibody. The 80-S-sR2-2H5 antibody comprises a V_{H} chain amino acid sequence of SEQ ID NO: 138 as shown below.

SEQ ID NO: 138
QVQLQASGGGLVQPGGSLRLSCAASGSIFSTNAMG
WYRQAPGKQREVIASITKFGNTDYADSVKGRFTIS
RDNAKNIVYLQMNSLKPEDTAVYYCYQNSRGRWYD
IFRDYWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 138.

In one embodiment, the antibody or binding fragment thereof of the present disclosure comprises a humanized variant of the heavy chain variable region of SEQ ID NO: 138, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 138), or a combination of these approaches. The humanized framework regions are designed to be similar in length and sequence to the parental framework sequences of SEQ ID NO: 138. In one embodiment, the humanized framework regions share 50%, 55%, 60%, 65%, 70%, 75%, 80%, 85%, 90%, 95% or more sequence identity to the framework regions of SEQ ID NO: 138. In another embodiment, the humanized framework regions are 60%, 65%, 70%, 75%, 80%, 85%, 90%, 95% or more similar in length to the framework regions of SEQ ID NO: 138. Humanized variants of the heavy chain variable region of SEQ ID NO: 138 share at least 50%, at least 55%, at least 60%, at least 65%, at least 70%, at least 75%, at least 80%, at least 85%, at least 90%, at least 95% sequence identity along the entire length of SEQ ID 138.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 138. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 31, or a modified amino acid sequence thereof containing 1, 2, 3, or more amino acid residue modifications as compared to SEQ ID NO: 31; a H-CDR2 having the amino acid sequence of SEQ ID NO: 67, or a modified amino acid sequence thereof containing 1, 2, 3, or more amino acid residue modifications as compared to SEQ ID NO: 67; and a H-CDR3 comprising the amino acid sequence of SEQ ID NO: 103, or a modified amino acid sequence thereof, said modified amino acid sequence containing 1, 2, 3, or more amino acid modifications as compared to SEQ ID NO: 103.

An exemplary single domain antibody having this heavy chain variable region is referred to herein as the 80-S-sR2-2H9 antibody. The 80-S-sR2-2H9antibody comprises a V_{H} chain amino acid sequence of SEQ ID NO: 139 as shown below.

SEQ ID NO: 139
QVQLVESGGGFVQAGGSLRLSCVASRSSFRITTMN
WYRQAPGKQREMVASITTGGRTVYADSVKGRFTIS
KSNANNTVYLQMNSLEAEDTAVYYGNAGRRWPLRD
YWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 139.

In one embodiment, the antibody or binding fragment thereof of the present disclosure comprises a humanized variant of the heavy chain variable region of SEQ ID NO: 139, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 139), or a combination of these approaches. The humanized framework regions are designed to be similar in length and sequence to the parental framework sequences of SEQ ID NO: 139. In one embodiment, the humanized framework regions share 50%, 55%, 60%, 65%, 70%, 75%, 80%, 85%, 90%, 95% or more sequence identity to the framework regions of SEQ ID NO: 139. In another embodiment, the humanized framework regions are 60%, 65%, 70%, 75%, 80%, 85%, 90%, 95% or more similar in length to the framework regions of SEQ ID NO: 139. Humanized variants of the heavy chain variable region of SEQ ID NO: 139 share at least 50%, at least 55%, at least 60%, at least 65%, at least 70%, at least 75%, at least 80%, at least 85%, at least 90%, at least 95% sequence identity along the entire length of SEQ ID 139.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 139. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 32, or a modified amino acid sequence thereof containing 1, 2, 3, or more amino acid residue modifications as compared to SEQ ID NO: 32; a H-CDR2 having the amino acid sequence of SEQ ID NO: 68, or a modified amino acid sequence thereof containing 1, 2, 3, or more amino acid residue modifications as compared to SEQ ID NO: 68; and a H-CDR3 comprising the amino acid sequence of SEQ ID NO: 104, or a modified amino acid sequence thereof, said modified amino acid sequence containing 1, 2, 3, or more amino acid modifications as compared to SEQ ID NO: 104.

An exemplary single domain antibody having this heavy chain variable region is referred to herein as the 86-S-sR2-1D5 antibody. The 86-S-sR2-1D5 antibody comprises a V_{H} chain amino acid sequence of SEQ ID NO: 141 as shown below.

SEQ ID NO: 141
QVQLQEFGGGVVQSGGSLRLSCVASGNIFRINAMG
WYRQAPGKQRELVAHIISGGSTNYADSVKGRFTIS
REYAKNMVYLQMNSLKPEDTAVYYCNAERRFGMRQ
VWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 141.

In one embodiment, the antibody or binding fragment thereof of the present disclosure comprises a humanized variant of the heavy chain variable region of SEQ ID NO: 141, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 141), or a combination of these approaches. The humanized framework regions are designed to be similar in length and sequence to the parental framework sequences of SEQ ID NO: 141. In one embodiment, the humanized framework regions share 50%, 55%, 60%, 65%, 70%, 75%, 80%, 85%, 90%, 95% or more sequence identity to the framework regions of SEQ ID NO: 141. In another embodiment, the humanized framework regions are 60%, 65%, 70%, 75%, 80%, 85%, 90%, 95% or more similar in length to the framework regions of SEQ ID NO: 141. Humanized variants of the heavy chain variable region of SEQ ID NO: 141 share at least 50%, at least 55%, at least 60%, at least 65%, at least 70%, at least 75%, at least 80%, at least 85%, at least 90%, at least 95% sequence identity along the entire length of SEQ ID 141.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 141. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 33, or a modified amino acid sequence thereof containing 1, 2, 3, or more amino acid residue modifications as compared to SEQ ID NO: 33; a H-CDR2 having the amino acid sequence of SEQ ID NO: 69, or a modified amino acid sequence thereof containing 1, 2, 3, or more amino acid residue modifications as compared to SEQ ID NO: 69; and a H-CDR3 comprising the amino acid sequence of SEQ ID NO: 105, or a modified amino acid sequence thereof, said modified amino acid sequence containing 1, 2, 3, or more amino acid modifications as compared to SEQ ID NO: 105.

An exemplary single domain antibody having this heavy chain variable region is referred to herein as the 87-S-sR2-2D8 antibody. The 87-S-sR2-2D8 antibody comprises a V_{H} chain amino acid sequence of SEQ ID NO: 142 as shown below.

SEQ ID NO: 142
QVQLQESGGGLVQAGGSLRLSCAASYYTFRYFPMG
WYRQAPGRQRELVASTTSGGGVNYADFVEGRFTIS
RDNAKNTFYLQMSSLKPEDTAVYYCARLLSLGSRW
GYGMFTWGKGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 142.

In one embodiment, the antibody or binding fragment thereof of the present disclosure comprises a humanized variant of the heavy chain variable region of SEQ ID NO: 142, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 142), or a combination of these approaches. The humanized framework regions are designed to be similar in length and sequence to the parental framework sequences of SEQ ID NO: 142. In one embodiment, the humanized framework regions share 50%, 55%, 60%, 65%, 70%, 75%, 80%, 85%, 90%, 95% or more sequence identity to the framework regions of SEQ ID NO: 142. In another embodiment, the humanized framework regions are 60%, 65%, 70%, 75%, 80%, 85%, 90%, 95% or more similar in length to the framework regions of SEQ ID NO: 142. Humanized variants of the heavy chain variable region of SEQ ID NO: 142 share at least 50%, at least 55%, at least 60%, at least 65%, at least 70%, at least 75%, at least 80%, at least 85%, at least 90%, at least 95% sequence identity along the entire length of SEQ ID 142.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 142. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 34, or a modified amino acid sequence thereof containing 1, 2, 3, or more amino acid residue modifications as compared to SEQ ID NO: 34; a H-CDR2 having the amino acid sequence of SEQ ID NO: 70, or a modified amino acid sequence thereof containing 1, 2, 3, or more amino acid residue modifications as compared to SEQ ID NO: 70; and a H-CDR3 comprising the amino acid sequence of SEQ ID NO: 106, or a modified amino acid sequence thereof, said modified amino acid sequence containing 1, 2, 3, or more amino acid modifications as compared to SEQ ID NO: 106.

An exemplary single domain antibody having this heavy chain variable region is referred to herein as the 88-S-sR2-2H8 antibody. The 88-S-sR2-2H8 antibody comprises a V_{H} I chain amino acid sequence of SEQ ID NO: 143 as shown below.

SEQ ID NO: 143
QVQLQESGGGLVQPGGSLRLSCAASGSIFSIKTMG
WYRQAPGKQRELVAAIASGGFTNYADSVKGRFTIS
RDNARNTVYLQMNSLKPEDTAVYYCNAQRRWPLRD
YWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 143.

In one embodiment, the antibody or binding fragment thereof of the present disclosure comprises a humanized variant of the heavy chain variable region of SEQ ID NO: 143, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 143), or a combination of these approaches. The humanized framework regions are designed to be similar in length and sequence to the parental framework sequences of SEQ ID NO: 143. In one embodiment, the humanized framework regions share 50%, 55%, 60%, 65%, 70%, 75%, 80%, 85%, 90%, 95% or more sequence identity to the framework regions of SEQ ID NO: 143. In another embodiment, the humanized framework regions are 60%, 65%, 70%, 75%, 80%, 85%, 90%, 95% or more similar in length to the framework regions of SEQ ID NO: 143. Humanized variants of the heavy chain variable region of SEQ ID NO: 143 share at least 50%, at least 55%, at least 60%, at least 65%, at least 70%, at least 75%, at least 80%, at least 85%, at least 90%, at least 95% sequence identity along the entire length of SEQ ID 143.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 143. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 35, or a modified amino acid sequence thereof containing 1, 2, 3, or more amino acid residue modifications as compared to SEQ ID NO: 35; a H-CDR2 having the amino acid sequence of SEQ ID NO: 71, or a modified amino acid sequence thereof containing 1, 2, 3, or more amino acid residue modifications as compared to SEQ ID NO: 71; and a H-CDR3 comprising the amino acid sequence of SEQ ID NO: 107, or a modified amino acid sequence thereof, said modified amino acid sequence containing 1, 2, 3, or more amino acid modifications as compared to SEQ ID NO: 107.

An exemplary single domain antibody having this heavy chain variable region is referred to herein as the 94-S-sR2-1D10 antibody. The 94-S-sR2-1D10 antibody comprises a V_{H} chain amino acid sequence of SEQ ID NO: 144 as shown below.

SEQ ID NO: 144
QVQLQESGGGLVRDGGSLTLSCAASGSAFRMNSMA
WYRQVPGKQRELVAAISFRGSANYANSVKGRFTIS
RDNGKNTVYLQMNSLKPEDTAYYYCAAGRPWQRTF
YWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 144.

In one embodiment, the antibody or binding fragment thereof of the present disclosure comprises a humanized variant of the heavy chain variable region of SEQ ID NO: 144, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 144), or a combination of these approaches. The humanized framework regions are designed to be similar in length and sequence to the parental framework sequences of SEQ ID NO: 144. In one embodiment, the humanized framework regions share 50%, 55%, 60%, 65%, 70%, 75%, 80%, 85%, 90%, 95% or more sequence identity to the framework regions of SEQ ID NO: 144. In another embodiment, the humanized framework regions are 60%, 65%, 70%, 75%, 80%, 85%, 90%, 95% or more similar in length to the framework regions of SEQ ID NO: 144. Humanized variants of the heavy chain variable region of SEQ ID NO: 144 share at least 50%, at least 55%, at least 60%, at least 65%, at least 70%, at least 75%, at least 80%, at least 85%, at least 90%, at least 95% sequence identity along the entire length of SEQ ID 144.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 144. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 36, or a modified amino acid sequence thereof containing 1, 2, 3, or more amino acid residue modifications as compared to SEQ ID NO: 36; a H-CDR2 having the amino acid sequence of SEQ ID NO: 72, or a modified amino acid sequence thereof containing 1, 2, 3, or more amino acid residue modifications as compared to SEQ ID NO: 72; and a H-CDR3 comprising the amino acid sequence of SEQ ID NO: 108, or a modified amino acid sequence thereof, said modified amino acid sequence containing 1, 2, 3, or more amino acid modifications as compared to SEQ ID NO: 108.

An exemplary single domain antibody having this heavy chain variable region is referred to herein as the 95-S-sR2-2D10 antibody. The 95-S-sR2-2D10 antibody comprises a V_{H} chain amino acid sequence of SEQ ID NO: 145 as shown below.

SEQ ID NO: 145
QVQLQESGGGFVQAGGSLRLSCAASGSIFSINYGN
WYRQAPGKQRELVAGISRGGRTKYADSVKGRFTIS
RDSAKTLTLQMTSLKPEDSGVYYCAATRWSWGTKS
YWGQGTQVWSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 145.

In one embodiment, the antibody or binding fragment thereof of the present disclosure comprises a humanized variant of the heavy chain variable region of SEQ ID NO: 145, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 145), or a combination of these approaches. The humanized framework regions are designed to be similar in length and sequence to the parental framework sequences of SEQ ID NO: 145. In one embodiment, the humanized framework regions share 50%, 55%, 60%, 65%, 70%, 75%, 80%, 85%, 90%, 95% or more sequence identity to the framework regions of SEQ ID NO: 145. In another embodiment, the humanized framework regions are 60%, 65%, 70%, 75%, 80%, 85%, 90%, 95% or more similar in length to the framework regions of SEQ ID NO: 145. Humanized variants of the heavy chain variable region of SEQ ID NO: 145 share at least 50%, at least 55%, at least 60%, at least 65%, at least 70%, at least 75%, at least 80%, at least 85%, at least 90%, at least 95% sequence identity along the entire length of SEQ ID 145.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 145. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 218, or a modified amino acid sequence thereof containing 1, 2, 3, or more amino acid residue modifications as compared to SEQ ID NO: 218; a H-CDR2 having the amino acid sequence of SEQ ID NO: 219, or a modified amino acid sequence thereof containing 1, 2, 3, or more amino acid residue modifications as compared to SEQ ID NO: 219; and a H-CDR3 comprising the amino acid sequence of SEQ ID NO: 220, or a modified amino acid sequence thereof, said modified amino acid sequence containing 1, 2, 3, or more amino acid modifications as compared to SEQ ID NO: 220.

An exemplary single domain antibody having this heavy chain variable region is referred to herein as the S-sR2-2H7 antibody. The S-sR2-2H7 antibody comprises a V_{H} chain amino acid sequence of SEQ ID NO: 221 as shown below.

SEQ ID NO: 221
QVQLQEFGGGVVQSGGSLRLSCVASGNLFRINAMG
WYRQAPGKSRVLVASIDSAGRTNYGDAVEDRFTIS
RDIANNTVNLQMNSLKPEDTAVYYCSTWRLGRNYW
GQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 221.

In one embodiment, the antibody or binding fragment thereof of the present disclosure comprises a humanized variant of the heavy chain variable region of SEQ ID NO: 221, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 221), or a combination of these approaches. The humanized framework regions are designed to be similar in length and sequence to the parental framework sequences of SEQ ID NO: 221. In one embodiment, the humanized framework regions share 50%, 55%, 60%, 65%, 70%, 75%, 80%, 85%, 90%, 95% or more sequence identity to the framework regions of SEQ ID NO: 221. In another embodiment, the humanized framework regions are 60%, 65%, 70%, 75%, 80%, 85%, 90%, 95% or more similar in length to the framework regions of SEQ ID NO: 221. Humanized variants of the heavy chain variable region of SEQ ID NO: 221 share at least 50%, at least 55%, at least 60%, at least 65%, at least 70%, at least 75%, at least 80%, at least 85%, at least 90%, at least 95% sequence identity along the entire length of SEQ ID 221.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 221. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 222, or a modified amino acid sequence thereof containing 1, 2, 3, or more amino acid residue modifications as compared to SEQ ID NO: 222; a H-CDR2 having the amino acid sequence of SEQ ID NO: 223, or a modified amino acid sequence thereof containing 1, 2, 3, or more amino acid residue modifications as compared to SEQ ID NO: 223; and a H-CDR3 comprising the amino acid sequence of SEQ ID NO: 224, or a modified amino acid sequence thereof, said modified amino acid sequence containing 1, 2, 3, or more amino acid modifications as compared to SEQ ID NO: 224.

An exemplary single domain antibody having this heavy chain variable region is referred to herein as the S-sR2-1F6 antibody. The S-sR2-1F6 antibody comprises a Vu chain amino acid sequence of SEQ ID NO: 225 as shown below.

SEQ ID NO: 225
QVQLQESGGGLVQAGGSLRLSCAASGSTFSNNAMA
WYRQAPGKQRELVAYTSSGGFTNYGDSVKGRFTIS
EDNAKSTVYLQMTSLKPEDTAVYYCSAGGTYRSGN
VYFFPRSWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 225.

In one embodiment, the antibody or binding fragment thereof of the present disclosure comprises a humanized variant of the heavy chain variable region of SEQ ID NO: 225, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 225), or a combination of these approaches. The humanized framework regions are designed to be similar in length and sequence to the parental framework sequences of SEQ ID NO: 225. In one embodiment, the humanized framework regions share 50%, 55%, 60%, 65%, 70%, 75%, 80%, 85%, 90%, 95% or more sequence identity to the framework regions of SEQ ID NO: 225. In another embodiment, the humanized framework regions are 60%, 65%, 70%, 75%, 80%, 85%, 90%, 95% or more similar in length to the framework regions of SEQ ID NO: 225. Humanized variants of the heavy chain variable region of SEQ ID NO: 225 share at least 50%, at least 55%, at least 60%, at least 65%, at least 70%, at least 75%, at least 80%, at least 85%, at least 90%, at least 95% sequence identity along the entire length of SEQ ID 225.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 225. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 226, or a modified amino acid sequence thereof containing 1, 2, 3, or more amino acid residue modifications as compared to SEQ ID NO: 226; a H-CDR2 having the amino acid sequence of SEQ ID NO: 227, or a modified amino acid sequence thereof containing 1, 2, 3, or more amino acid residue modifications as compared to SEQ ID NO: 227; and a H-CDR3 comprising the amino acid sequence of SEQ ID NO: 228, or a modified amino acid sequence thereof, said modified amino acid sequence containing 1, 2, 3, or more amino acid modifications as compared to SEQ ID NO: 228.

An exemplary single domain antibody having this heavy chain variable region is referred to herein as the S-sR2-1G4 antibody. The S-sR2-1G4 antibody comprises a V_{H} chain amino acid sequence of SEQ ID NO: 229 as shown below.

SEQ ID NO: 229
QVQLQQSGGGLVQPGCISLRLSCAASGSIFSINSM
AWYRQAPGNQRELVATISSRSTTYYAPSYKGRYNS
RDNAKINIVYLQMNSLKPEDTAVYYCKAGSVGIWY
WGQGTLYIVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 229.

In one embodiment, the antibody or binding fragment thereof of the present disclosure comprises a humanized variant of the heavy chain variable region of SEQ ID NO: 229, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 229), or a combination of these approaches. The humanized framework regions are designed to be similar in length and sequence to the parental framework sequences of SEQ ID NO: 229. In one embodiment, the humanized framework regions share 50%, 55%, 60%, 65%, 70%, 75%, 80%, 85%, 90%, 95% or more sequence identity to the framework regions of SEQ ID NO: 229. In another embodiment, the humanized framework regions are 60%, 65%, 70%, 75%, 80%, 85%, 90%, 95% or more similar in length to the framework regions of SEQ ID NO: 229. Humanized variants of the heavy chain variable region of SEQ ID NO: 229 share at least 50%, at least 55%, at least 60%, at least 65%, at least 70%, at least 75%, at least 80%, at least 85%, at least 90%, at least 95% sequence identity along the entire length of SEQ ID 229.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 229. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 230, or a modified amino acid sequence thereof containing 1, 2, 3, or more amino acid residue modifications as compared to SEQ ID NO: 230; a H-CDR2 having the amino acid sequence of SEQ ID NO: 231, or a modified amino acid sequence thereof containing 1, 2, 3, or more amino acid residue modifications as compared to SEQ ID NO: 231; and a H-CDR3 comprising the amino acid sequence of SEQ ID NO: 232, or a modified amino acid sequence thereof, said modified amino acid sequence containing 1, 2, 3, or more amino acid modifications as compared to SEQ ID NO: 232.

An exemplary single domain antibody having this heavy chain variable region is referred to herein as the S-sR2-2C10 antibody. The S-sR2-2C10 antibody comprises a V_{H} chain amino acid sequence of SEQ ID NO: 233 as shown below.

SEQ ID NO: 233
QVQLQESGGGLVQAGGSLRLSCLASMTTLGFKTMG
WYRQAPGKQRELVAMTSGGTANYADSVKGRFAISR
ENAKNTLYLQMNSLKPEDTALYYCASTTGWTEVGG
RNDYWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 233.

In one embodiment, the antibody or binding fragment thereof of the present disclosure comprises a humanized variant of the heavy chain variable region of SEQ ID NO: 233, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 233), or a combination of these approaches. The humanized framework regions are designed to be similar in length and sequence to the parental framework sequences of SEQ ID NO: 233. In one embodiment, the humanized framework regions share 50%, 55%, 60%, 65%, 70%, 75%, 80%, 85%, 90%, 95% or more sequence identity to the framework regions of SEQ ID NO: 233. In another embodiment, the humanized framework regions are 60%, 65%, 70%, 75%, 80%, 85%, 90%, 95% or more similar in length to the framework regions of SEQ ID NO: 233. Humanized variants of the heavy chain variable region of SEQ ID NO: 233 share at least 50%, at least 55%, at least 60%, at least 65%, at least 70%, at least 75%, at least 80%, at least 85%, at least 90%, at least 95% sequence identity along the entire length of SEQ ID 233.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 233. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 234, or a modified amino acid sequence thereof containing 1, 2, 3, or more amino acid residue modifications as compared to SEQ ID NO: 234; a H-CDR2 having the amino acid sequence of SEQ ID NO: 235, or a modified amino acid sequence thereof containing 1, 2, 3, or more amino acid residue modifications as compared to SEQ ID NO: 235; and a H-CDR3 comprising the amino acid sequence of SEQ ID NO: 236, or a modified amino acid sequence thereof, said modified amino acid sequence containing 1, 2, 3, or more amino acid modifications as compared to SEQ ID NO: 236.

An exemplary single domain antibody having this heavy chain variable region is referred to herein as the S-sR2-2E9 antibody. The S-sR2-2E9 antibody comprises a Vu chain amino acid sequence of SEQ ID NO: 237 as shown below.

SEQ ID NO: 237
QVQLQESGGGLVQTGGSLRLSCAASGRTFRVNAMG
VVYRQAPGKQREFVAAVTNGGSTTYADSVKGRFTI
SRDNAKNTIYLQMNRLEPEDTALYYCNAERRFGMR
QVWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 237.

In one embodiment, the antibody or binding fragment thereof of the present disclosure comprises a humanized variant of the heavy chain variable region of SEQ ID NO: 237, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 237), or a combination of these approaches. The humanized framework regions are designed to be similar in length and sequence to the parental framework sequences of SEQ ID NO: 237. In one embodiment, the humanized framework regions share 50%, 55%, 60%, 65%, 70%, 75%, 80%, 85%, 90%, 95% or more sequence identity to the framework regions of SEQ ID NO: 237. In another embodiment, the humanized framework regions are 60%, 65%, 70%, 75%, 80%, 85%, 90%, 95% or more similar in length to the framework regions of SEQ ID NO: 237. Humanized variants of the heavy chain variable region of SEQ ID NO: 237 share at least 50%, at least 55%, at least 60%, at least 65%, at least 70%, at least 75%, at least 80%, at least 85%, at least 90%, at least 95% sequence identity along the entire length of SEQ ID 237.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 237. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 238, or a modified amino acid sequence thereof containing 1, 2, 3, or more amino acid residue modifications as compared to SEQ ID NO: 238; a H-CDR2 having the amino acid sequence of SEQ ID NO: 239, or a modified amino acid sequence thereof containing 1, 2, 3, or more amino acid residue modifications as compared to SEQ ID NO: 239; and a H-CDR3 comprising the amino acid sequence of SEQ ID NO: 240, or a modified amino acid sequence thereof, said modified amino acid sequence containing 1, 2, 3, or more amino acid modifications as compared to SEQ ID NO: 240.

An exemplary single domain antibody having this heavy chain variable region is referred to herein as the S-sR2-G11 antibody. The S-sR2-G11 antibody comprises a V_{H} chain amino acid sequence of SEQ ID NO: 241 as shown below.

SEQ ID NO: 241
QVQLQESGGCLVQPGGSLRLSCAASGRVFSINTMG
WYRQAPGKQRELVASMTRGGSANYADSVKGRFTTS
RDNAKNMVYLQMNRLKAEDTAVYYCNAARGWRIYW
GKGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 241.

In one embodiment, the antibody or binding fragment thereof of the present disclosure comprises a humanized variant of the heavy chain variable region of SEQ ID NO: 241, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 241), or a combination of these approaches. The humanized framework regions are designed to be similar in length and sequence to the parental framework sequences of SEQ ID NO: 241. In one embodiment, the humanized framework regions share 50%, 55%, 60%, 65%, 70%, 75%, 80%, 85%, 90%, 95% or more sequence identity to the framework regions of SEQ ID NO: 241. In another embodiment, the humanized framework regions are 60%, 65%, 70%, 75%, 80%, 85%, 90%, 95% or more similar in length to the framework regions of SEQ ID NO: 241. Humanized variants of the heavy chain variable region of SEQ ID NO: 241 share at least 50%, at least 55%, at least 60%, at least 65%, at least 70%, at least 75%, at least 80%, at least 85%, at least 90%, at least 95% sequence identity along the entire length of SEQ ID 241.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 241. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 340, or a modified amino acid sequence thereof containing 1, 2, 3, or more amino acid residue modifications as compared to SEQ ID NO: 340; a H-CDR2 having the amino acid sequence of SEQ ID NO: 341, or a modified amino acid sequence thereof containing 1, 2, 3, or more amino acid residue modifications as compared to SEQ ID NO: 341; and a H-CDR3 comprising the amino acid sequence of SEQ ID NO: 342, or a modified amino acid sequence thereof, said modified amino acid sequence containing 1, 2, 3, or more amino acid modifications as compared to SEQ ID NO: 342

An exemplary single domain antibody having this heavy chain variable region is referred to herein as the 90-S-sR1-2D1 antibody. The 90-S-sR1-2D1 antibody comprises a V_{H} chain amino acid sequence of SEQ ID NO: 343 as shown below.

SEQ ID NO: 343
QVQLQESGGGLVQAGGSLRLSCAASGSIFRINGMGWIRQAPGKEREVVAAV
NWSGERTYYVDSVKGRFTISREKGNRIYLQMNDLEPDDTAVYYCAADTDYR
LDGSTWITNLYSGSLGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 343.

In one embodiment, the antibody or binding fragment thereof of the present disclosure comprises a humanized variant of the heavy chain variable region of SEQ ID NO: 343, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 343), or a combination of these approaches. The humanized framework regions are designed to be similar in length and sequence to the parental framework sequences of SEQ ID NO: 343. In one embodiment, the humanized framework regions share 50%, 55%, 60%, 65%, 70%, 75%, 80%, 85%, 90%, 95% or more sequence identity to the framework regions of SEQ ID NO: 343. In another embodiment, the humanized framework regions are 60%, 65%, 70%, 75%, 80%, 85%, 90%, 95% or more similar in length to the framework regions of SEQ ID NO: 343. Humanized variants of the heavy chain variable region of SEQ ID NO: 343 share at least 50%, at least 55%, at least 60%, at least 65%, at least 70%, at least 75%, at least 80%, at least 85%, at least 90%, at least 95% sequence identity along the entire length of SEQ ID 343.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 343. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In another embodiment, the antibody or binding fragment thereof as disclosed herein comprises a heavy chain variable region having a H-CDR1 with an amino acid sequence selected from SEQ ID NOs: 146-160, or a modified amino acid sequence thereof containing 1, 2, 3, 4, 5, or 6 amino acid residue modifications as compared to SEQ ID NOs: 146-160 that maintain or enhance binding specificity of the H-CDR1. In one embodiment, the amino acid sequence of the H-CDR1 contains no more than 1, 2, or 3 amino acid modifications as compared to SEQ ID NOs: 146-160, respectively. The HCVR further comprises a H-CDR2 with an amino acid sequence selected from SEQ ID NOs: 161-175, or a modified amino acid sequence thereof containing 1, 2, 3, 4, 5, or 6, amino acid residue modifications as compared to SEQ ID NOs: 161-175 that maintain or enhance binding specificity of the H-CDR2. In one embodiment, the amino acid sequence of the H-CDR2 contains no more than 1, 2, or 3, amino acid modifications as compared to SEQ ID NOs: 161-175, respectively. The HCVR of the antibody or binding fragment thereof comprises a H-CDR3 with an amino acid sequence selected from SEQ ID NOs: 176-190, or a modified amino acid sequence thereof containing 1, 2, 3, 4, 5, 6 or 7 amino acid residue modifications as compared to SEQ ID NOs: 176-190 that maintain or enhance binding specificity of the H-CDR3. In one embodiment, the amino acid sequence of the H-CDR3 contains no more than 1, 2, or 3 amino acid modifications as compared to SEQ ID NOs: 176-190, respectively. The amino acid sequences of SEQ ID NOs: 146-190 are provided in Table 2 below.

TABLE 2
α-Synuclein Single Domain Antibody Complementarity Determining Regions

| | | | SEQ | | | SEQ | | | SEQ |
|---|---|---|---|---|---|---|---|---|---|
| | | | ID | | | ID | | | ID |
| Antibody ID | descr | sequence | NO: | descr | sequence | NO: | descr | sequence | NO: |
| 02-S-bR1-2C7 | CDR1 | ASGGTFGAGV | 146 | CDR2 | SMGSDGFTQIEN | 161 | CDR3 | HYADGRFGS | 176 |
| 04-S-bR2-1C4; 10-S-bR1- | CDR1 | ASGSNISTNV | 147 | CDR2 | SISTSGTTNYLA | 162 | CDR3 | YAAWPLNT | 177 |
| 2D8; 13-S-bR2-1E12; 14-S- | | | | | | | | | |
| bR2-1G7; 17-S-bR1-1C11; | | | | | | | | | |
| 18-S-bR1-2F1-; 20-S-bR2- | | | | | | | | | |
| 1C9; 27-S-bR2-1B1; 28-S- | | | | | | | | | |
| bR2-1C12; 30-S-bR2-1H1; | | | | | | | | | |
| 38-S-bR2-1H7; 44-S-bR2- | | | | | | | | | |
| 1D2; 60-S-bR2-1D12; 67-S- | | | | | | | | | |
| bR2-1B9; 68-S-bR2-1E2; | | | | | | | | | |
| 69-S-bR2-1F10; 75-S-bR2- | | | | | | | | | |
| 1B11; 81-S-bR1-2B7; 91-S- | | | | | | | | | |
| bR2-1C3; 92-S-bR2-1E6 | | | | | | | | | |
| 05-S-bR2-1E10; 06-S-bR2- | CDR1 | ASGSTFSITY | 148 | CDR2 | EMSRRGSTFYAD | 163 | CDR3 | SVGARRDEDD | 178 |
| 1G5; 26-S-bR1-2H12; 46- | | | | | | | | YVY | |
| S-bR2-1H11; 52-S-bR2- | | | | | | | | | |
| 1D6; 54-S-bR2-1H12; 57-S- | | | | | | | | | |
| bR1-1H3; 59-S-bR2-1B8; | | | | | | | | | |
| 76-S-bR2-1E3; 77-S-bR2- | | | | | | | | | |
| 1G1; 83-S-bR2-1C1; 93-S- | | | | | | | | | |
| bR2-1G4 | | | | | | | | | |
| 09-S-bR1-1B5 | CDR1 | DSRTIFIFNA | 149 | CDR2 | AISSGGSTKYAD | 164 | CDR3 | AASRSGRWLD | 179 |
| | | | | | | | | DAR | |
| 12-S-bR2-1C5; 21-S-bR2- | CDR1 | ASGIPFSIIY | 150 | CDR2 | EMSSRGSKFYAD | 165 | CDR3 | SVGARRDDND | 180 |
| 1F1; 25-S-bR1-1D9; 35-S- | | | | | | | | YVY | |
| bR2-1B2; 43-S-bR2-1B3; | | | | | | | | | |
| 53-S-bR2-1F7 | | | | | | | | | |
| 22-S-bR2-1G10 | CDR1 | ASGNIFGINS | 151 | CDR2 | DITRGNRKYAD | 166 | CDR3 | NAEIVTQIPF | 181 |
| | | | | | | | | PPR | |
| 29-S-bR2-1F2 | CDR1 | ASGFTFSSYW | 152 | CDR2 | TINTGGYTTYYS | 167 | CDR3 | CAKAYGSMWS | 182 |
| | | | | | | | | GIW | |
| 33-S-bR1-1G3 | CDR1 | VSGSIISHNV | 153 | CDR2 | CISGSGFTNYIA | 168 | CDR3 | YTAWPNT | 183 |
| 36-S-bR2-1D1 | CDR1 | ASGRIFN1ED | 154 | CDR2 | TITRTGAPTYAN | 169 | CDR3 | NAKDVTVIPF | 184 |
| | | | | | | | | PPK | |
| 41-S-bR1-1H1 | CDR1 | ASGSVSAIET | 155 | CDR2 | VISTGGTTKYAP | 170 | CDR3 | AADWRTILGW | 185 |
| | | | | | | | | KTR | |
| 45-S-bR2-1F6; 61-S-bR2- | CDR1 | AFGSTSSITY | 156 | CDR2 | EMSRRGSTFYAD | 171 | CDR3 | SVGARRDEDD | 186 |
| 1F8; 84-S-bR2-1E5 | | | | | | | | YVY | |
| 49-S-bR1-1H2 | CDR1 | ASGGPVSDNV | 157 | CDR2 | QITSGGATSYAD | 172 | CDR3 | NVALRY | 187 |
| 51-S-bR2-1B7; 85-S-bR2- | CDR1 | ASGSTFSITY | 158 | CDR2 | EISSRGSVFYAD | 173 | CDR3 | SVGARRDEDD | 188 |
| 1G2 | | | | | | | | YVY | |
| 73-S-bR1-2B2 | CDR1 | GAGSAFGWNA | 159 | CDR2 | TIESGGWADYSV | 174 | CDR3 | NQLTY | 189 |
| 89-S-bR1-2C6 | CDR1 | PSGSIFSFDV | 160 | CDR2 | QHRTPGAIDYAD | 175 | CDR3 | NLRRWSYDY | 190 |

In one embodiment, the antibody or binding fragment thereof has a heavy chain variable region with a H-CDR1 having the amino acid sequence of SEQ ID NO: 146, or a modified amino acid sequence thereof containing 1, 2, 3, or more amino acid residue modifications as compared to SEQ ID NO: 146; a H-CDR2 having the amino acid sequence of SEQ ID NO: 161, or a modified amino acid sequence thereof containing 1, 2, 3, or more amino acid residue modifications as compared to SEQ ID NO: 161; and a H-CDR3 comprising the amino acid sequence of SEQ ID NO: 176, or a modified amino acid sequence thereof, said modified amino acid sequence containing 1, 2, 3, or more amino acid modifications as compared to SEQ ID NO: 176.

An exemplary single domain antibody having this heavy chain variable region is referred to herein as the 02-S-bR1-2C7 antibody. The 02-S-bR1-2C7 antibody comprises a V_{H} chain amino acid sequence of SEQ ID NO: 191 as shown below.

SEQ ID NO: 191
QVQLQESGGGLVQPGDSLKLSCAASGGTFGAGVVAWYRQSPGKQREMVGSM
GSDGFTQIENGMKGRFTISGAGDKKTVFLQMNNLKPEDTAVYFCHYADGRF
GSWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 191.

In one embodiment, the antibody or binding fragment thereof of the present disclosure comprises a humanized variant of the heavy chain variable region of SEQ ID NO: 191, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 191), or a combination of these approaches. The humanized framework regions are designed to be similar in length and sequence to the parental framework sequences of SEQ ID NO: 191. In one embodiment, the humanized framework regions share 50%, 55%, 60%, 65%, 70%, 75%, 80%, 85%, 90%, 95% or more sequence identity to the framework regions of SEQ ID NO: 191. In another embodiment, the humanized framework regions are 60%, 65%, 70%, 75%, 80%, 85%, 90%, 95% or more similar in length to the framework regions of SEQ ID NO: 191. Humanized variants of the heavy chain variable region of SEQ ID NO: 191 share at least 50%, at least 55%, at least 60%, at least 65%, at least 70%, at least 75%, at least 80%, at least 85%, at least 90%, at least 95% sequence identity along the entire length of SEQ ID 191.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 191. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 147, or a modified amino acid sequence thereof containing 1, 2, 3, or more amino acid residue modifications as compared to SEQ ID NO: 147; a H-CDR2 having the amino acid sequence of SEQ ID NO: 162, or a modified amino acid sequence thereof containing 1, 2, 3, or more amino acid residue modifications as compared to SEQ ID NO: 162; and a H-CDR3 comprising the amino acid sequence of SEQ ID NO: 177, or a modified amino acid sequence thereof, said modified amino acid sequence containing 1, 2, 3, or more amino acid modifications as compared to SEQ ID NO: 177.

An exemplary single domain antibody having this combination of heavy chain variable region CDRs is referred to herein as the 04-S-bR2-1C4 antibody. The 04-S-bR2-1C4 antibody comprises a V_{H} chain amino acid sequence of SEQ ID NO: 192 as shown below.

SEQ ID NO: 192
QVQLQESGGGLVQAGGSLRLSCAASGSNISTNVMAWYRRAPGNQRDMVASI
STSGTTNYLASVKGRFTISRDNAKNTVSLQMNSLKPEDTAVYTCYAAWPLN
TWGQGTQVTVSS

Other antibodies of the disclosure comprising the amino acid sequence of SEQ ID NO: 192 include 20-S-bR2-1C9; 27-S-bR2-1B1; 28-S-bR2-1C12; 38-S-bR2-1H7; 44-S-bR2-1D2; 60-S-bR2-1D12; 69-S-bR2-1F10; 75-S-bR2-1B11; and 91-S-bR2-1C3.

Another exemplary single domain antibody having this combination of heavy chain variable region CDRs is referred to herein as the 10-S-bR1-2D8 antibody. The 10-S-bR1-2D8 antibody comprises a V_{H} chain amino acid sequence of SEQ ID NO: 196 as shown below.

SEQ ID NO: 196
QVQLQESGGGLVQAGGSLRLSCAASGSNISTNVMAWYRRAPGNQRDMVASI
STSGTTNYLASVKGRFTISRDNAKNTVSLQMNSLKPEDTAVYTCYAAWPLN
TWGQGTLVTVSS

Other antibodies of the disclosure comprising the amino acid sequence of SEQ ID NO: 196 include 13-S-bR2-1E12; 18-S-bR1-2F1; 67-S-bR2-1B9; 81-S-bR1-2B7; and 92-S-bR2-1E6.

Other exemplary single domain antibodies having this combination of heavy chain variable region CDRs are referred to herein as the 14-S-bR2-1G7 antibody and the 17-S-bR1-1C11 antibody. These antibodies comprises a V_{H} chain amino acid sequence of SEQ ID NO: 198 as shown below.

SEQ ID NO: 198
QVQLQASGGGLVQAGGSLRLSCAASGSNISTNVMAWYRRAPGNQRDMVASI
STSGTTNYLASVKGRFTISRDNAKNTVSLQMNSLKPEDTAVYTCYAAWPLN
TWGQGTQVTVSS

Another exemplary single domain having this combination of heavy chain variable region CDRs is referred to herein as the 30-S-bR2-1H1 antibody. The 30-S-bR2-1H1 antibody comprises a V_{H} chain amino acid sequence of SEQ ID NO: 202 as shown below.

SEQ ID NO: 202
QVQLQQSGGGLVQAGGSLRLSCAASGSNISTNVMAWYRRAPGNQRDMVASI
STSGTTNYLASVKGRFTISRDNAKNTVSLQMNSLKPEDTAVYTCYAAWPLN
TWGQGTQVTVSS

Another exemplary single domain antibody having this combination of heavy chain variable region CDRs is referred to herein as the 68-S-bR2-1E2 antibody. The 68-S-bR2-1E2 antibody comprises a V_{H} chain amino acid sequence of SEQ ID NO: 212 as shown below.

SEQ ID NO: 212
QVQLQEFGGGLVQAGESLRLSCVASGSNISTNVMAWYRRAPGNQRDMVASI
STSGTTNYLASVKGRFTISRDNAKNTVSLQMNSLKPEDTAVYTCYAAWPLN
TWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 192, SEQ ID NO: 196, SEQ ID NO: 198, SEQ ID NO: 202, or SEQ ID NO: 212.

In one embodiment, the antibody or binding fragment thereof of the present disclosure comprises a humanized variant of the heavy chain variable region of SEQ ID NO: 192, SEQ ID NO: 196, SEQ ID NO: 198, SEQ ID NO: 202, or SEQ ID NO: 212, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 192, SEQ ID NO: 196, SEQ ID NO: 198, SEQ ID NO: 202, or SEQ ID NO: 212), or a combination of these approaches. The humanized framework regions are designed to be similar in length and sequence to the parental framework sequences of SEQ ID NO: 192, SEQ ID NO: 196, SEQ ID NO: 198, SEQ ID NO: 202, or SEQ ID NO: 212. In one embodiment, the humanized framework regions share 50%, 55%, 60%, 65%, 70%, 75%, 80%, 85%, 90%, 95% or more sequence identity to the framework regions of SEQ ID NO: 192, SEQ ID NO: 196, SEQ ID NO: 198, SEQ ID NO: 202, or SEQ ID NO: 212. In another embodiment, the humanized framework regions are 60%, 65%, 70%, 75%, 80%, 85%, 90%, 95% or more similar in length to the framework regions of SEQ ID NO: 192, SEQ ID NO: 196, SEQ ID NO: 198, SEQ ID NO: 202, or SEQ ID NO: 212. Humanized variants of the heavy chain variable region of SEQ ID NO: 192, SEQ ID NO: 196, SEQ ID NO: 198, SEQ ID NO: 202, or SEQ ID NO: 212 share at least 50%, at least 55%, at least 60%, at least 65%, at least 70%, at least 75%, at least 80%, at least 85%, at least 90%, at least 95% sequence identity along the entire length of SEQ ID NO: 192, SEQ ID NO: 196, SEQ ID NO: 198, SEQ ID NO: 202, or SEQ ID NO: 212, respectively.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 192, SEQ ID NO: 196, SEQ ID NO: 198, SEQ ID NO: 202, or SEQ ID NO: 212. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 148, or a modified amino acid sequence thereof containing 1, 2, 3, or more amino acid residue modifications as compared to SEQ ID NO: 148; a H-CDR2 having the amino acid sequence of SEQ ID NO: 163, or a modified amino acid sequence thereof containing 1, 2, 3, or more amino acid residue modifications as compared to SEQ ID NO: 163; and a H-CDR3 comprising the amino acid sequence of SEQ ID NO: 178, or a modified amino acid sequence thereof, said modified amino acid sequence containing 1, 2, 3, or more amino acid modifications as compared to SEQ ID NO: 178.

An exemplary single domain antibody having this combination of heavy chain variable region CDRs is referred to herein as the 05-S-bR2-1E10 antibody. The 05-S-bR2-1E10 antibody comprises a V_{H} chain amino acid sequence of SEQ ID NO: 193 as shown below.

SEQ ID NO: 193
QVQLQESGGGLVQAGGSLRLSCAASGSTFSITYMAWFRQAPEKQRELVAEM
SRRGSTFYADSVKGRFTISRDNTKNTVYLQMNSLEPEDTAVYYCSVGARRD
EDDYVYWGQGTQVTVSS

Other antibodies of the disclosure comprising the amino acid sequence of SEQ ID NO: 193 include 26-S-bR1-2H12; 46-S-bR2-1H11; 57-S-bR1-1H3; 83-S-bR2-1C1; 93-S-bR2-1G4.

Another exemplary single domain antibody having this combination of heavy chain variable region CDRs is referred to herein as the 06-S-bR2-1G5 antibody. The 06-S-bR2-1G5 antibody comprises a V_{H} chain amino acid sequence of SEQ ID NO: 194 as shown below.

SEQ ID NO: 194
QVQLQQSGGGLVQAGGSLRLSCAASGSTFSITYMAWFRQAPEKQRELVAEM
SRRGSTFYADSVKGRFTISRDNTKNTVYLQMNSLEPEDTAVYYCSVGARRD
EDDYVYWGQGTQVTVSS

Other exemplary single domain antibodies having this combination of heavy chain variable region CDRs are referred to herein as the 52-S-bR2-1D6 antibody and the 54-S-bR2-1H12 antibody. These antibodies comprises a V_{H} chain amino acid sequence of SEQ ID NO: 209 as shown below.

SEQ ID NO: 209
QVQLQESGGGLVQAGGSLRLSCAASGSTFSITYMAWFRQAPEKQRELVAEM
SRRGSTFYADSVKGRFTISRDNTKNTVYLQMNSLEPEDTAVYYCSVGARRD
EDDYVYWGQGTLVTVSS

Another exemplary single domain antibody having this combination of heavy chain variable region CDRs is referred to herein as the 59-S-bR2-1B8 antibody. The 59-S-bR2-1B8 antibody comprises a V_{H} chain amino acid sequence of SEQ ID NO: 211 as shown below.

SEQ ID NO: 211
QVQLQEFGGGLVQAGGSLRLSCAASGSTFSITYMAWFRQAPEKQRELVAEM
SRRGSTFYADSVKGRFTISRDNTKNTVYLQMNSLEPEDTAVYYCSVGARRD
EDDYVYWGQGTQVTVSS

Another exemplary single domain antibody having this combination of heavy chain variable region CDRs is referred to herein as the 76-S-bR2-1E3 antibody. The 76-S-bR2-1E3 antibody comprises a V_{H} chain amino acid sequence of SEQ ID NO: 214 as shown below.

SEQ ID NO: 214
QVQLQESGGGLVQAGGSLRLSCAASGSTFSITYMTWFRQAPEKQRELVAEM
SRRGSTFYADSVKARFTISRDNTKNTVYLQMNSLEPEDTAVYYCSVGARRD
EDDYVYWGQGTQVTVSS

Another exemplary single domain antibody having this combination of heavy chain variable region CDRs is referred to herein as the 77-S-bR2-1G1 antibody. The 77-S-bR2-1G1 antibody comprises a V_{H} chain amino acid sequence of SEQ ID NO: 215 as shown below.

SEQ ID NO: 215
QVQLQEFGGGLVQAGGSLRLSCAASGSTFSITYMAWFRQAPEKQRELVAEM
SRRGSTFYADSVKGRFTISRDNTKNTVYLQMNSLEPEDTAVYYCSVGARRD
EDDYVYWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 193, SEQ ID NO: 194, SEQ ID NO: 209, SEQ ID NO: 211, SEQ ID NO: 214, or SEQ ID NO: 215.

In one embodiment, the antibody or binding fragment thereof of the present disclosure comprises a humanized variant of the heavy chain variable region of SEQ ID NO: 193, SEQ ID NO: 194, SEQ ID NO: 209, SEQ ID NO: 211, SEQ ID NO: 214, or SEQ ID NO: 215, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 193, SEQ ID NO: 194, SEQ ID NO: 209, SEQ ID NO: 211, SEQ ID NO: 214, or SEQ ID NO: 215), or a combination of these approaches. The humanized framework regions are designed to be similar in length and sequence to the parental framework sequences of SEQ ID NO: 193, SEQ ID NO: 194, SEQ ID NO: 209, SEQ ID NO: 211, SEQ ID NO: 214, or SEQ ID NO: 215. In one embodiment, the humanized framework regions share 50%, 55%, 60%, 65%, 70%, 75%, 80%, 85%, 90%, 95% or more sequence identity to the framework regions of SEQ ID NO: 193, SEQ ID NO: 194, SEQ ID NO: 209, SEQ ID NO: 211, SEQ ID NO: 214, or SEQ ID NO: 215. In another embodiment, the humanized framework regions are 60%, 65%, 70%, 75%, 80%, 85%, 90%, 95% or more similar in length to the framework regions of SEQ ID NO: 193, SEQ ID NO: 194, SEQ ID NO: 209, SEQ ID NO: 211, SEQ ID NO: 214, or SEQ ID NO: 215. Humanized variants of the heavy chain variable region of SEQ ID NO: 193, SEQ ID NO: 194, SEQ ID NO: 209, SEQ ID NO: 211, SEQ ID NO: 214, or SEQ ID NO: 215 share at least 50%, at least 55%, at least 60%, at least 65%, at least 70%, at least 75%, at least 80%, at least 85%, at least 90%, at least 95% sequence identity along the entire length of SEQ ID NO: 193, SEQ ID NO: 194, SEQ ID NO: 209, SEQ ID NO: 211, SEQ ID NO: 214, or SEQ ID NO: 215, respectively.

Another aspect of the present disclosure relates to an antibody or binding portion thereof (e.g., a human antibody) that competes for binding to human α-synuclein protein with the anti-α-synuclein antibody comprising a heavy chain variable region with vthe amino acid sequence of SEQ ID NO: 193, SEQ ID NO: 194, SEQ ID NO: 209, SEQ ID NO: 211, SEQ ID NO: 214, or SEQ ID NO: 215. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 149, or a modified amino acid sequence thereof containing 1, 2, 3, or more amino acid residue modifications as compared to SEQ ID NO: 149; a H-CDR2 having the amino acid sequence of SEQ ID NO: 164, or a modified amino acid sequence thereof containing 1, 2, 3, or more amino acid residue modifications as compared to SEQ ID NO: 164; and a H-CDR3 comprising the amino acid sequence of SEQ ID NO: 179, or a modified amino acid sequence thereof, said modified amino acid sequence containing 1, 2, 3, or more amino acid modifications as compared to SEQ ID NO: 179.

An exemplary single domain antibody having this heavy chain variable region is referred to herein as the 09-S-bR1-1B5 antibody. The 09-S-bR1-1B5 antibody comprises a V_{H} chain amino acid sequence of SEQ ID NO: 195 as shown below.

SEQ ID NO: 195
QVQLQESGGGSVQAGGSLRLSCADSRTIFIFNAMAWYRQAPGKQRELVAAI
SSGGSTKYADSVKGRFTISSSNAKNTKYLQMNRLKPEDTAVYYCAASRSGR
WLDDARDYEYWGPGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 195.

In one embodiment, the antibody or binding fragment thereof of the present disclosure comprises a humanized variant of the heavy chain variable region of SEQ ID NO: 195, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 195), or a combination of these approaches. The humanized framework regions are designed to be similar in length and sequence to the parental framework sequences of SEQ ID NO: 195. In one embodiment, the humanized framework regions share 50%, 55%, 60%, 65%, 70%, 75%, 80%, 85%, 90%, 95% or more sequence identity to the framework regions of SEQ ID NO: 195. In another embodiment, the humanized framework regions are 60%, 65%, 70%, 75%, 80%, 85%, 90%, 95% or more similar in length to the framework regions of SEQ ID NO: 195. Humanized variants of the heavy chain variable region of SEQ ID NO: 195 share at least 50%, at least 55%, at least 60%, at least 65%, at least 70%, at least 75%, at least 80%, at least 85%, at least 90%, at least 95% sequence identity along the entire length of SEQ ID 195.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 195. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 150, or a modified amino acid sequence thereof containing 1, 2, 3, or more amino acid residue modifications as compared to SEQ ID NO: 150; a H-CDR2 having the amino acid sequence of SEQ ID NO: 165, or a modified amino acid sequence thereof containing 1, 2, 3, or more amino acid residue modifications as compared to SEQ ID NO: 165; and a H-CDR3 comprising the amino acid sequence of SEQ ID NO: 180, or a modified amino acid sequence thereof, said modified amino acid sequence containing 1, 2, 3, or more amino acid modifications as compared to SEQ ID NO: 180.

An exemplary single domain antibody having this heavy chain variable region is referred to herein as the 12-S-bR2-1C5 antibody. The 12-S-bR2-1C5 antibody comprises a V_{H} chain amino acid sequence of SEQ ID NO: 197 as shown below.

SEQ ID NO: 197
QVQLQQSGGGLVQAGGSLRLSCAASGIPFSIIYMAWFRQAPEKQRELVAE
MSSRGSKFYADSVKGRFTISRDNAKNTLYLQMNSLEPEDTAVYYCSVGAR
RDDNDYVYWGQGTQVTVSS

Other exemplary single domain antibodies having this combination of heavy chain CDRs described herein include, the 21-S-bR2-1F1 antibody, the 25-S-bR1-1D9 antibody, the 35-S-bR2-1B2 antibody, and the 43-S-bR2-1B3 antibody. These antibodies comprise the V_{H} chain amino acid sequence of SEQ ID NO: 199 as shown below.

SEQ ID NO: 199
QVQLQESGGGLVQAGGSLRLSCAASGIPFSIIYMAWFRQAPEKQRELVAE
MSSRGSKFYADSVKGRFTISRDNAKNTLYLQMNSLEPEDTAVYYCSVGAR
RDDNDYVYWGQGTQVTVSS

Another exemplary single domain antibody having this combination of heavy chain CDRs is referred to herein as the 53-S-bR2-1F7 antibody. The 53-S-bR2-1F7 antibody comprises a V_{H} chain amino acid sequence of SEQ ID NO: 210 as shown below.

SEQ ID NO: 210
QVQLQESGGGLVQAGGSLRLSCAASGIPFSIIYMAWFRQAPEKQRELVAE
MSSRGSKFYADSVKGRFTISRDNAKNTLYLQMNSLEPEDTAVYYCSVGAR
RDDNDYVYWGQGTLNTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 197, SEQ ID NO: 199, or SEQ ID NO: 210.

In one embodiment, the antibody or binding fragment thereof of the present disclosure comprises a humanized variant of the heavy chain variable region of SEQ ID NO: 197, SEQ ID NO: 199, or SEQ ID NO: 210, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 197, SEQ ID NO: 199, or SEQ ID NO: 210), or a combination of these approaches. The humanized framework regions are designed to be similar in length and sequence to the parental framework sequences of SEQ ID NO: 197, SEQ ID NO: 199, or SEQ ID NO: 210. In one embodiment, the humanized framework regions share 50%, 55%, 60%, 65%, 70%, 75%, 80%, 85%, 90%, 95% or more sequence identity to the framework regions of SEQ ID NO: 197, SEQ ID NO: 199, or SEQ ID NO: 210. In another embodiment, the humanized framework regions are 60%, 65%, 70%, 75%, 80%, 85%, 90%, 95% or more similar in length to the framework regions of SEQ ID NO: 197, SEQ ID NO: 199, or SEQ ID NO: 210. Humanized variants of the heavy chain variable region of SEQ ID NO: 197, SEQ ID NO: 199, or SEQ ID NO: 210 share at least 50%, at least 55%, at least 60%, at least 65%, at least 70%, at least 75%, at least 80%, at least 85%, at least 90%, at least 95% sequence identity along the entire length of SEQ ID NO: 197, SEQ ID NO: 199, or SEQ ID NO: 210, respectively.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 197, SEQ ID NO: 199, or SEQ ID NO: 210. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 151, or a modified amino acid sequence thereof containing 1, 2, 3, or more amino acid residue modifications as compared to SEQ ID NO: 151; a H-CDR2 having the amino acid sequence of SEQ ID NO: 166, or a modified amino acid sequence thereof containing 1, 2, 3, or more amino acid residue modifications as compared to SEQ ID NO: 166; and a H-CDR3 comprising the amino acid sequence of SEQ ID NO: 181, or a modified amino acid sequence thereof, said modified amino acid sequence containing 1, 2, 3, or more amino acid modifications as compared to SEQ ID NO: 181.

An exemplary single domain antibody having this heavy chain variable region is referred to herein as the 22-S-bR2-1G10 antibody. The 22-S-bR2-1G10 antibody comprises a V_{H} chain amino acid sequence of SEQ ID NO: 200 as shown below.

SEQ ID NO: 200
QVQLQASGGGVVQSGGSLRLSCVASGNIFGINSMAWYRQAPGKQRELVAD
ITRGNRKYADSVKGRFISQDNAKNTVYLQMNRLKPEDTAVYFCNAEIVTQ
IPFPPREFWGRGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 200.

In one embodiment, the antibody or binding fragment thereof of the present disclosure comprises a humanized variant of the heavy chain variable region of SEQ ID NO: 200, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 200), or a combination of these approaches. The humanized framework regions are designed to be similar in length and sequence to the parental framework sequences of SEQ ID NO: 200. In one embodiment, the humanized framework regions share 50%, 55%, 60%, 65%, 70%, 75%, 80%, 85%, 90%, 95% or more sequence identity to the framework regions of SEQ ID NO: 200. In another embodiment, the humanized framework regions are 60%, 65%, 70%, 75%, 80%, 85%, 90%, 95% or more similar in length to the framework regions of SEQ ID NO: 200. Humanized variants of the heavy chain variable region of SEQ ID NO: 200 share at least 50%, at least 55%, at least 60%, at least 65%, at least 70%, at least 75%, at least 80%, at least 85%, at least 90%, at least 95% sequence identity along the entire length of SEQ ID 200.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 200. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 152, or a modified amino acid sequence thereof containing 1, 2, 3, or more amino acid residue modifications as compared to SEQ ID NO: 152; a H-CDR2 having the amino acid sequence of SEQ ID NO: 167, or a modified amino acid sequence thereof containing 1, 2, 3, or more amino acid residue modifications as compared to SEQ ID NO: 167; and a H-CDR3 comprising the amino acid sequence of SEQ ID NO: 182, or a modified amino acid sequence thereof, said modified amino acid sequence containing 1, 2, 3, or more amino acid modifications as compared to SEQ ID NO: 182.

An exemplary single domain antibody having this heavy chain variable region is referred to herein as the 29-S-bR2-1F2 antibody. The 29-S-bR2-1F2 antibody comprises a V_{H} chain amino acid sequence of SEQ ID NO: 201 as shown below.

SEQ ID NO: 201
QVQLQASGGGLVQPGGSLRLSCAASGFTFSSYWMYWVRQAPGKGLEWVST
INTGGYTTYYSDSVKGRFTISRDNGKNTLYLEMNSLKSEDTAVYYCAKAY
GSMWSGIWGGMDYWGKGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 201.

In one embodiment, the antibody or binding fragment thereof of the present disclosure comprises a humanized variant of the heavy chain variable region of SEQ ID NO: 201, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 201), or a combination of these approaches. The humanized framework regions are designed to be similar in length and sequence to the parental framework sequences of SEQ ID NO: 201. In one embodiment, the humanized framework regions share 50%, 55%, 60%, 65%, 70%, 75%, 80%, 85%, 90%, 95% or more sequence identity to the framework regions of SEQ ID NO: 201. In another embodiment, the humanized framework regions are 60%, 65%, 70%, 75%, 80%, 85%, 90%, 95% or more similar in length to the framework regions of SEQ ID NO: 201. Humanized variants of the heavy chain variable region of SEQ ID NO: 201 share at least 50%, at least 55%, at least 60%, at least 65%, at least 70%, at least 75%, at least 80%, at least 85%, at least 90%, at least 95% sequence identity along the entire length of SEQ ID 201.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 201. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 153, or a modified amino acid sequence thereof containing 1, 2, 3, or more amino acid residue modifications as compared to SEQ ID NO: 153; a H-CDR2 having the amino acid sequence of SEQ ID NO: 168, or a modified amino acid sequence thereof containing 1, 2, 3, or more amino acid residue modifications as compared to SEQ ID NO: 168; and a H-CDR3 comprising the amino acid sequence of SEQ ID NO: 183, or a modified amino acid sequence thereof, said modified amino acid sequence containing 1, 2, 3, or more amino acid modifications as compared to SEQ ID NO: 183.

An exemplary single domain antibody having this heavy chain variable region is referred to herein as the 33-S-bR1-1G3 antibody. The 33-S-bR1-1G3 antibody comprises a V_{H} chain amino acid sequence of SEQ ID NO: 203 as shown below.

SEQ ID NO: 203
QVQLQESGGGLVQAGGSLRLSCAVSGSIISHNVMAWYRRAPGKQRDKVAC
ISGSGFTNYIASVKGRFTISRDNAKNTVSLQMNNLKPEDTAVYSCYTAWP
NTWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 203.

In one embodiment, the antibody or binding fragment thereof of the present disclosure comprises a humanized variant of the heavy chain variable region of SEQ ID NO: 203, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 203), or a combination of these approaches. The humanized framework regions are designed to be similar in length and sequence to the parental framework sequences of SEQ ID NO: 203. In one embodiment, the humanized framework regions share 50%, 55%, 60%, 65%, 70%, 75%, 80%, 85%, 90%, 95% or more sequence identity to the framework regions of SEQ ID NO: 203. In another embodiment, the humanized framework regions are 60%, 65%, 70%, 75%, 80%, 85%, 90%, 95% or more similar in length to the framework regions of SEQ ID NO: 203. Humanized variants of the heavy chain variable region of SEQ ID NO: 203 share at least 50%, at least 55%, at least 60%, at least 65%, at least 70%, at least 75%, at least 80%, at least 85%, at least 90%, at least 95% sequence identity along the entire length of SEQ ID 203.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 203. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 154, or a modified amino acid sequence thereof containing 1, 2, 3, or more amino acid residue modifications as compared to SEQ ID NO: 154; a H-CDR2 having the amino acid sequence of SEQ ID NO: 169, or a modified amino acid sequence thereof containing 1, 2, 3, or more amino acid residue modifications as compared to SEQ ID NO: 169; and a H-CDR3 comprising the amino acid sequence of SEQ ID NO: 184, or a modified amino acid sequence thereof, said modified amino acid sequence containing 1, 2, 3, or more amino acid modifications as compared to SEQ ID NO: 184.

An exemplary single domain antibody having this heavy chain variable region is referred to herein as the 36-S-bR2-1D1 antibody. The 36-S-bR2-1D1 antibody comprises a V_{H} chain amino acid sequence of SEQ ID NO: 204 as shown below.

SEQ ID NO: 204
QVQLQESGGGFVHPGGSLTLSCAASGRIFNIEDMGWYRQGPGEQRDLVAT
ITRTGAPTYANSVKGRFTISRDNAKNTVYLQMTRLKPEDTAVYYCNAKDV
TVIPFPPKDYWGRGTFQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 204.

In one embodiment, the antibody or binding fragment thereof of the present disclosure comprises a humanized variant of the heavy chain variable region of SEQ ID NO: 204, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 204), or a combination of these approaches. The humanized framework regions are designed to be similar in length and sequence to the parental framework sequences of SEQ ID NO: 204. In one embodiment, the humanized framework regions share 50%, 55%, 60%, 65%, 70%, 75%, 80%, 85%, 90%, 95% or more sequence identity to the framework regions of SEQ ID NO: 204. In another embodiment, the humanized framework regions are 60%, 65%, 70%, 75%, 80%, 85%, 90%, 95% or more similar in length to the framework regions of SEQ ID NO: 204. Humanized variants of the heavy chain variable region of SEQ ID NO: 204 share at least 50%, at least 55%, at least 60%, at least 65%, at least 70%, at least 75%, at least 80%, at least 85%, at least 90%, at least 95% sequence identity along the entire length of SEQ ID 204.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 204. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 155, or a modified amino acid sequence thereof containing 1, 2, 3, or more amino acid residue modifications as compared to SEQ ID NO: 155; a H-CDR2 having the amino acid sequence of SEQ ID NO: 170, or a modified amino acid sequence thereof containing 1, 2, 3, or more amino acid residue modifications as compared to SEQ ID NO: 170; and a H-CDR3 comprising the amino acid sequence of SEQ ID NO: 185, or a modified amino acid sequence thereof, said modified amino acid sequence containing 1, 2, 3, or more amino acid modifications as compared to SEQ ID NO: 185.

An exemplary single domain antibody having this heavy chain variable region is referred to herein as the 41-S-bR1-1H1 antibody. The 41-S-bR1-1H1 antibody comprises a V_{H} chain amino acid sequence of SEQ ID NO: 205 as shown below.

SEQ ID NO: 205
QVQLQESGGGLVQAGGSLRLSCEASGSVSAIETMGWYRQAPDEQRTFVAV
ISTGGTTKYAPSVKGRFTISIDNAKSTVTLQMNSLKPEDTAVYYCAADWR
TILGWKTREPNYFGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 205.

In one embodiment, the antibody or binding fragment thereof of the present disclosure comprises a humanized variant of the heavy chain variable region of SEQ ID NO: 205, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 205), or a combination of these approaches. The humanized framework regions are designed to be similar in length and sequence to the parental framework sequences of SEQ ID NO: 205. In one embodiment, the humanized framework regions share 50%, 55%, 60%, 65%, 70%, 75%, 80%, 85%, 90%, 95% or more sequence identity to the framework regions of SEQ ID NO: 205. In another embodiment, the humanized framework regions are 60%, 65%, 70%, 75%, 80%, 85%, 90%, 95% or more similar in length to the framework regions of SEQ ID NO: 205. Humanized variants of the heavy chain variable region of SEQ ID NO: 205 share at least 50%, at least 55%, at least 60%, at least 65%, at least 70%, at least 75%, at least 80%, at least 85%, at least 90%, at least 95% sequence identity along the entire length of SEQ ID 205.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 205. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 156, or a modified amino acid sequence thereof containing 1, 2, 3, or more amino acid residue modifications as compared to SEQ ID NO: 156; a H-CDR2 having the amino acid sequence of SEQ ID NO: 171, or a modified amino acid sequence thereof containing 1, 2, 3, or more amino acid residue modifications as compared to SEQ ID NO: 171; and a H-CDR3 comprising the amino acid sequence of SEQ ID NO: 186, or a modified amino acid sequence thereof, said modified amino acid sequence containing 1, 2, 3, or more amino acid modifications as compared to SEQ ID NO: 186.

Exemplary single domain antibodies having this heavy chain variable region as described herein include the 45-S-bR2-1F6 antibody, the 61-S-bR2-1F8 antibody, and the 84-S-bR2-1E5 antibody. These antibodies comprise a V_{H} chain amino acid sequence of SEQ ID NO: 206 as shown below.

SEQ ID NO: 206
QVQLQESGGGLVQAGGSLRLSCAAFGSTSSITYMAWFRQAPEKQRELVAE
MSRRGSTFYADSVKGRFTIYRDNTKNTVYLQMNSLEPEDTAVYYCSVGAR
RDEDDYVYWGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 206.

In one embodiment, the antibody or binding fragment thereof of the present disclosure comprises a humanized variant of the heavy chain variable region of SEQ ID NO: 206, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 206), or a combination of these approaches. The humanized framework regions are designed to be similar in length and sequence to the parental framework sequences of SEQ ID NO: 206. In one embodiment, the humanized framework regions share 50%, 55%, 60%, 65%, 70%, 75%, 80%, 85%, 90%, 95% or more sequence identity to the framework regions of SEQ ID NO: 206. In another embodiment, the humanized framework regions are 60%, 65%, 70%, 75%, 80%, 85%, 90%, 95% or more similar in length to the framework regions of SEQ ID NO: 206. Humanized variants of the heavy chain variable region of SEQ ID NO: 206 share at least 50%, at least 55%, at least 60%, at least 65%, at least 70%, at least 75%, at least 80%, at least 85%, at least 90%, at least 95% sequence identity along the entire length of SEQ ID 206.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 206. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 157 or a modified amino acid sequence thereof containing 1, 2, 3, or more amino acid residue modifications as compared to SEQ ID NO: 157; a H-CDR2 having the amino acid sequence of SEQ ID NO: 172, or a modified amino acid sequence thereof containing 1, 2, 3, or more amino acid residue modifications as compared to SEQ ID NO: 172; and a H-CDR3 comprising the amino acid sequence of SEQ ID NO: 187, or a modified amino acid sequence thereof, said modified amino acid sequence containing 1, 2, 3, or more amino acid modifications as compared to SEQ ID NO: 187.

An exemplary single domain antibody having this heavy chain variable region is referred to herein as the 49-S-bR1-1H2 antibody. This antibody comprises a V_{H} chain amino acid sequence of SEQ ID NO: 207 as shown below.

SEQ ID NO: 207
QVQLQESGGGLVQAGGSLRLSCAASGGPVSDNVMAWFRQAPGSQRELVAQ
ITSGGATSYADSVKGRFTISRDNARSTVDLQMNSLKPEDTAVYYCNVALR
YWGRGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 207.

In one embodiment, the antibody or binding fragment thereof of the present disclosure comprises a humanized variant of the heavy chain variable region of SEQ ID NO: 207, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 207), or a combination of these approaches. The humanized framework regions are designed to be similar in length and sequence to the parental framework sequences of SEQ ID NO: 207. In one embodiment, the humanized framework regions share 50%, 55%, 60%, 65%, 70%, 75%, 80%, 85%, 90%, 95% or more sequence identity to the framework regions of SEQ ID NO: 207. In another embodiment, the humanized framework regions are 60%, 65%, 70%, 75%, 80%, 85%, 90%, 95% or more similar in length to the framework regions of SEQ ID NO: 207. Humanized variants of the heavy chain variable region of SEQ ID NO: 207 share at least 50%, at least 55%, at least 60%, at least 65%, at least 70%, at least 75%, at least 80%, at least 85%, at least 90%, at least 95% sequence identity along the entire length of SEQ ID 207.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 207. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 158 or a modified amino acid sequence thereof containing 1, 2, 3, or more amino acid residue modifications as compared to SEQ ID NO: 158; a H-CDR2 having the amino acid sequence of SEQ ID NO: 173, or a modified amino acid sequence thereof containing 1, 2, 3, or more amino acid residue modifications as compared to SEQ ID NO: 173; and a H-CDR3 comprising the amino acid sequence of SEQ ID NO: 188, or a modified amino acid sequence thereof, said modified amino acid sequence containing 1, 2, 3, or more amino acid modifications as compared to SEQ ID NO: 188.

An exemplary single domain antibody having this heavy chain variable region is referred to herein as the 51-S-bR2-1B7 antibody. This antibody comprises a V_{H} chain amino acid sequence of SEQ ID NO: 208 as shown below.

SEQ ID NO: 208
QVQLQQSGGGLVQAGGSLRLSCAASGSTFSITYMAWFRQAPGKQRELVAE
ISSRGSVFYADSVKGRFTISRDNAKKTVYLQMNSLETEDTAAYYCSVGAR
RDEDDYVYWGQGTQVTVSS

Another exemplary single domain antibody having this heavy chain variable region is referred to herein as the 85-S-bR2-1G2 antibody. This antibody comprises a V_{H} chain amino acid sequence of SEQ ID NO: 216 as shown below.

SEQ ID NO: 216
QVQLQQSGGGLVQAGGSLRLSCAASGSTFSITYMAWFRQAPGKQRELVAE
ISSRGSVFYADSVKGRFTISRDNAKKTVYLQMNSLETEDTAAYYCSVGAR
RDEDDYVYWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 208 or SEQ ID NO: 216.

In one embodiment, the antibody or binding fragment thereof of the present disclosure comprises a humanized variant of the heavy chain variable region of SEQ ID NO: 208 or SEQ ID NO: 216, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 208 or SEQ ID NO: 216), or a combination of these approaches. The humanized framework regions are designed to be similar in length and sequence to the parental framework sequences of SEQ ID NO: 208 or SEQ ID NO: 216. In one embodiment, the humanized framework regions share 50%, 55%, 60%, 65%, 70%, 75%, 80%, 85%, 90%, 95% or more sequence identity to the framework regions of SEQ ID NO: 208 or SEQ ID NO: 216. In another embodiment, the humanized framework regions are 60%, 65%, 70%, 75%, 80%, 85%, 90%, 95% or more similar in length to the framework regions of SEQ ID NO: 208 or SEQ ID NO: 216. Humanized variants of the heavy chain variable region of SEQ ID NO: 208 or SEQ ID NO: 216 share at least 50%, at least 55%, at least 60%, at least 65%, at least 70%, at least 75%, at least 80%, at least 85%, at least 90%, at least 95% sequence identity along the entire length of SEQ ID 208 or SEQ ID NO: 216, respectively.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 208. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 159 or a modified amino acid sequence thereof containing 1, 2, 3, or more amino acid residue modifications as compared to SEQ ID NO: 159; a H-CDR2 having the amino acid sequence of SEQ ID NO: 174, or a modified amino acid sequence thereof containing 1, 2, 3, or more amino acid residue modifications as compared to SEQ ID NO: 174; and a H-CDR3 comprising the amino acid sequence of SEQ ID NO: 189, or a modified amino acid sequence thereof, said modified amino acid sequence containing 1, 2, 3, or more amino acid modifications as compared to SEQ ID NO: 189.

An exemplary single domain antibody having this heavy chain variable region is referred to herein as the 73-S-bR1-2B2 antibody. This antibody comprises a Vu chain amino acid sequence of SEQ ID NO: 213 as shown below.

SEQ ID NO: 213
QVQLQESGGGLVQAGGSLRLSCVGAGSAEGWNAVHWYRQAPGQQREWLAT
IESGGWADYSVSVKGRFIVSRDNARNTAYLQMNNLKLEDTAVYYCNQLTY
WGQGTQVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 213.

In one embodiment, the antibody or binding fragment thereof of the present disclosure comprises a humanized variant of the heavy chain variable region of SEQ ID NO: 213, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 213), or a combination of these approaches. The humanized framework regions are designed to be similar in length and sequence to the parental framework sequences of SEQ ID NO: 213. In one embodiment, the humanized framework regions share 50%, 55%, 60%, 65%, 70%, 75%, 80%, 85%, 90%, 95% or more sequence identity to the framework regions of SEQ ID NO: 213. In another embodiment, the humanized framework regions are 60%, 65%, 70%, 75%, 80%, 85%, 90%, 95% or more similar in length to the framework regions of SEQ ID NO: 213. Humanized variants of the heavy chain variable region of SEQ ID NO: 213 share at least 50%, at least 55%, at least 60%, at least 65%, at least 70%, at least 75%, at least 80%, at least 85%, at least 90%, at least 95% sequence identity along the entire length of SEQ ID 213.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 213. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

In one embodiment, the antibody or binding fragment thereof has a heavy chain variable region with a H-CDR1 having the amino acid sequence of SEQ ID NO: 160 or a modified amino acid sequence thereof containing 1, 2, 3, or more amino acid residue modifications as compared to SEQ ID NO: 160; a H-CDR2 having the amino acid sequence of SEQ ID NO: 175, or a modified amino acid sequence thereof containing 1, 2, 3, or more amino acid residue modifications as compared to SEQ ID NO: 175; and a H-CDR3 comprising the amino acid sequence of SEQ ID NO: 190, or a modified amino acid sequence thereof, said modified amino acid sequence containing 1, 2, 3, or more amino acid modifications as compared to SEQ ID NO: 190.

An exemplary single domain antibody having this heavy chain variable region is referred to herein as the 89-S-bR1-2C6 antibody. This antibody comprises a V_{H} chain amino acid sequence of SEQ ID NO: 217 as shown below.

SEQ ID NO: 217
QVQLQESGGGLVQPGGSLRLSCTPSGSIFSFDVMAWYRQAPGKRRELVAQ
HRTPGAIDYADPVRGRFTISRDAGDVLFLQMDSLKPEDTAVYFCNLRRWS
YDYWGQGTLVTVSS

In another embodiment, the antibody or binding fragment thereof comprises a heavy chain variable region having an amino acid sequence that shares at least 60%, at least 65%, at least 70%, at least 75%, at least 80%, at least 85%, at least 90%, at least 91%, at least 92%, at least 93%, at least 94% at least 95%, at least 96%, at least 97%, at least 98%, at least 99% sequence identity to SEQ ID NO: 217.

In one embodiment, the antibody or binding fragment thereof of the present disclosure comprises a humanized variant of the heavy chain variable region of SEQ ID NO: 217, where the framework regions are humanized or replaced with human immunoglobulin framework sequences. As noted supra, suitable human or humanized framework sequences can be chosen based on their known structure, a consensus sequence, sequence homology to the framework sequences of donor antibody (e.g., the framework sequences of SEQ ID NO: 217), or a combination of these approaches. The humanized framework regions are designed to be similar in length and sequence to the parental framework sequences of SEQ ID NO: 217. In one embodiment, the humanized framework regions share 50%, 55%, 60%, 65%, 70%, 75%, 80%, 85%, 90%, 95% or more sequence identity to the framework regions of SEQ ID NO: 217. In another embodiment, the humanized framework regions are 60%, 65%, 70%, 75%, 80%, 85%, 90%, 95% or more similar in length to the framework regions of SEQ ID NO: 217. Humanized variants of the heavy chain variable region of SEQ ID NO: 217 share at least 50%, at least 55%, at least 60%, at least 65%, at least 70%, at least 75%, at least 80%, at least 85%, at least 90%, at least 95% sequence identity along the entire length of SEQ ID 217.

Another aspect of the present disclosure relates to an antibody or binding portion thereof (e.g., a human antibody) that competes for binding to human α-synuclein protein with the anti-α-synuclein antibody comprising a heavy chain variable region with the amino acid sequence of SEQ ID NO: 217. In accordance with this aspect of the disclosure, a competitive binding assay, such as Bio-Layer Interferometry (BLI) can be utilized to identify an antibody or binding portion thereof that competes for binding to an α-synuclein protein with the enumerated monoclonal antibody. Other competitive binding assays known in the art can also be utilized to identify a competitive binding antibody in accordance with this aspect of the disclosure.

Antibody “specificity” refers to selective recognition of the antibody or binding portion thereof as described herein for a particular epitope of the α-synuclein protein. The term “epitope” includes any protein determinant capable of specific binding to an immunoglobulin or T-cell receptor or otherwise interacting with a molecule. Epitopic determinants generally consist of chemically active surface groupings of molecules such as amino acids or carbohydrate or sugar side chains and generally have specific three dimensional structural characteristics, as well as specific charge characteristics. The epitope of the antibodies described herein may be “linear” or “conformational”. In a linear epitope, all of the points of interaction between the protein and the antibody occur linearly along the primary amino acid sequence of the protein. In a conformational epitope, the points of interaction occur across amino acid residues on the protein that are separated from one another, i.e., noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, 6, 7, 8, 9, 10, 11, 12, 13, 14, 15, 16, 17, 18, 19, 20 or more amino acids in a unique spatial conformation. Antibodies that recognize the same epitope can be verified in a simple immunoassay showing the ability of one antibody to block the binding of another antibody to a target antigen.

In one embodiment, the epitope recognized by the isolated antibody of the present invention is a non-linear or conformational epitope, i.e. a tertiary or quaternary structure that is shared by pathological proteins. For example, the non-linear or conformational epitope recognized by the antibodies described herein is a conformational epitope that is common or shared with one or more, or all, amyloidogenic proteins, e.g., synuclein, tau, Aß, prion, etc. Accordingly, in one embodiment, the antibodies described herein have antigenic specificity for a shared conformational epitope common to all amyloidogenic proteins known in the art.

Another aspect of the present disclosure is directed to an antibody mimetic that binds α-synuclein protein. An “antibody mimetic” as referred to herein encompasses any organic compound, e.g., a peptide or polypeptide, that can specifically bind an antigen like an antibody, and is about 3-20 kDa. In one embodiment, the antibody mimetic comprises a scaffold which binds its antigen via amino acids in exposed loops similar to the CDR loops of an antibody. These antibody mimetics include, without limitation, adnectins, lipocalins, Kunitz domain-based binders, avimers, knottins, fynomers, atrimers, and cytotoxic T-lymphocyte associated protein-4 (CTLA4)-based binders (reviewed in Weidle et al., “The Emerging Role of New Protein Scaffold-based Agents for the Treatment of Cancer,” Cancer Genomics & Proteomics 10:155-168 (2013), which is hereby incorporated by reference in its entirety). In accordance with this aspect of the present disclosure, the loop binding regions of the antibody mimetic are adapted to comprise one or more of the heavy chain CDRs of the antibodies disclosed herein. For example, an antibody mimetic of the present disclosure may comprise a first loop region having an amino acid sequence of any one of SEQ ID NOs: 1-36, 218, 222, 226, 230, 234, 238, 340 or a modified amino acid sequence of any one of SEQ ID NOs: 1-36, 218, 222, 226, 230, 234, 238, and 340 said modified sequence containing 1, 2, or 3 amino acid residue modifications as compared to any one of SEQ ID NOs: 1-36, 218, 222, 226, 230, 234, 238, and 340. The antibody mimetic may comprise another loop region having an amino acid sequence of any one of SEQ ID NOs: 37-72, 219, 223, 227, 231, 235, 239, 341 or a modified amino acid sequence of any one of SEQ ID NOs: 37-72, 219, 223, 227, 231, 235, 239, and 341 said modified sequences containing 1, 2, or 3, amino acid residue modifications as compared to any one of SEQ ID NOs: 37-72, 219, 223, 227, 231, 235, 239, and 341. The antibody mimetic may comprise another loop region having an amino acid sequence any one of SEQ ID NOs: 73-108, 220, 224, 228, 232, 236, 240, 342 or a modified amino acid sequence of any one of SEQ ID NOs: 73-108, 220, 224, 228, 232, 236, 240, and 342 said modified sequence containing 1, 2, or 3 amino acid residue modifications as compared to any one of SEQ ID NOs: 73-108, 220, 224, 228, 232, 236, 240, and 342.

In another embodiment, an antibody mimetic of the present disclosure may comprise a first loop region having an amino acid sequence of any one of SEQ ID NOs: 146-160 or a modified amino acid sequence of any one of SEQ ID NOs: 146-160, said modified sequence containing 1, 2, or 3 amino acid residue modifications as compared to any one of SEQ ID NOs: 146-160. The antibody mimetic may comprise another loop region having an amino acid sequence of any one of SEQ ID NOs: 161-175, or a modified amino acid sequence of any one of SEQ ID NOs: 161-175, said modified sequences containing 1, 2, or 3, amino acid residue modifications as compared to any one of SEQ ID NOs: 161-175. The antibody mimetic may comprise another loop region having an amino acid sequence any one of SEQ ID NOs: 176-190, or a modified amino acid sequence of any one of SEQ ID NOs: 176-190, said modified sequence containing 1, 2, or 3 amino acid residue modifications as compared to any one of SEQ ID NOs: 176-190.

In one embodiment, the antibody mimetic comprises one or more modified fibronectin type III (FN3) domains (e.g., an adnectin or centyrin molecule), where each modified FN3 domain has one or more loop regions that comprise one or more CDR sequences or modified CDR sequences as disclosed herein.

The FN3 domain is an evolutionary conserved protein domain that is about 100 amino acids in length and possesses a beta sandwich structure. The beta sandwich structure of human FN3 comprises seven beta-strands, referred to as strands A, B, C, D, E, F, G, with six connecting loops, referred to as loops AB, BC, CD, DE, EF, and FG that exhibit structural homology to immunoglobulin binding domains. Three of the six loops, i.e., loops DE, BC, and FG, correspond topologically to the complementarity determining regions of an antibody, i.e., CDR1, CDR2, and CDR3. The remaining three loops are surface exposed in a manner similar to antibody CDR3. In accordance with the present disclosure, one or more of the loop regions of each FN3 domain of the binding molecule are modified to comprise one or more CDR sequences disclosed herein.

The modified FN3 domain can be a FN3 domain derived from any of the wide variety of animal, yeast, plant, and bacterial extracellular proteins containing these domains. In one embodiment, the FN3 domain is derived from a mammalian FN3 domain. Exemplary FN3 domains include, for example and without limitation, any one of the 15 different FN3 domains present in human tenascin C, or the 15 different FN3 domains present in human fibronectin (FN) (e.g., the 10th fibronectin type III domain). Exemplary FN3 domains also include non-natural synthetic FN3 domains, such as those described in U.S. Pat. Publ. No. 2010/0216708 to Jacobs et al., which is hereby incorporated by reference in its entirety. Individual FN3 domains are referred to by domain number and protein name, e.g., the 3^{rd} FN3 domain of tenascin (TN3), or the 10^{th} FN3 domain of fibronectin (FN10).

Another aspect of the present disclosure is directed to isolated polynucleotides encoding the α-synuclein antibody or binding fragment thereof or antibody mimetic as described herein. The nucleic acid molecules described herein include isolated polynucleotides, portions of expression vectors or portions of linear DNA sequences, including linear DNA sequences used for in vitro transcription/translation, and vectors compatible with prokaryotic, eukaryotic or filamentous phage expression, secretion, and/or display of the antibodies or binding fragments thereof described herein.

In one embodiment, an isolated polynucleotide encodes a H-CDR1 of any one or more of SEQ ID NOs: 1-36, 218, 222, 226, 230, 234, 238, and 340; a H-CDR2 of any one of SEQ ID NOs: 37-72, 219, 223, 227, 231, 235, 239, and 341; and a H-CRD3 of any one of SEQ ID NOs: 73-108, 220, 224, 228, 232, 236, 240, and 342. In another embodiment, an isolated polynucleotide as described herein encodes a H-CDR1 of any one or more of SEQ ID NOs: 146-160, a H-CDR2 of any one of SEQ ID NOs: 161-175, and a H-CDR3 of any one of SEQ ID NOs: 176-190.

In another embodiment, an isolated polynucleotide as described herein encodes a heavy chain variable region of an α-synuclein antibody having the amino acid sequence of any one of SEQ ID NOs: 109-145, 221, 225, 229, 233, 237, 241, and 343. The nucleotide sequences of these isolated polynucleotides are enumerated in Table 3 herein and include SEQ ID NOs: 242-286. In another embodiment, the isolated polynucleotide encodes a heavy chain variable region of an α-synuclein antibody having the amino acid sequence of any one of SEQ ID NOs: 191-217. The nucleotide sequences of these isolated polynucleotides are enumerated in Table 4 herein and include SEQ ID NOs: 287-339. Nucleic acid molecules having nucleotide sequences that differ from SEQ ID NOs: 242-339, which as a result of the degeneracy of the genetic code, also encode the α-synuclein antibody described herein are also encompassed by the present disclosure. Such nucleic acid molecules may share 80%, 85%, 90%, or 95% sequence identity to any one of the sequences of SEQ ID NOs: 249-339.

The polynucleotides of the invention may be produced by chemical synthesis such as solid phase polynucleotide synthesis on an automated polynucleotide synthesizer and assembled into complete single or double stranded molecules. Alternatively, the polynucleotides of the invention may be produced by other techniques such as PCR followed by routine cloning. Techniques for producing or obtaining polynucleotides of a known sequence are well known in the art.

The polynucleotides of the invention may comprise at least one non-coding sequence, such as a promoter or enhancer sequence, intron, polyadenylation signal, a cis sequence facilitating RepA binding, and the like. The polynucleotide sequences may also comprise additional sequences encoding additional amino acids that provide, for example, a marker or a tag sequence such as a histidine tag or an HA tag to facilitate purification or detection of the protein, a signal sequence, a fusion protein partner such as RepA, Fc or bacteriophage coat protein such as pIX or pIII.

Another embodiment of the disclosure is directed to a vector comprising at least one polynucleotide encoding the antibody or binding fragment thereof or antibody mimetic as described herein. Such vectors include, without limitation, plasmid vectors, viral vectors, including without limitation, vaccina vector, lentiviral vector, adenoviral vector, adeno-associated viral vector, vectors for baculovirus expression, transposon based vectors or any other vector suitable for introduction of the polynucleotides described herein into a given organism or genetic background by any means to facilitate expression of the encoded antibody polypeptide. The polynucleotide sequences encoding the heavy chain variable domains as described herein are combined with sequences of a promoter, a translation initiation segment (e.g., a ribosomal binding sequence and start codon), a 3′ untranslated region, polyadenylation signal, a termination codon, and transcription termination to form one or more expression vector constructs.

In one embodiment, the vector is an adenoviral-associated viral (AAV) vector. A number of therapeutic AAV vectors suitable for delivery of the polynucleotides encoding α-synuclein antibodies described herein to the central nervous system are known in the art. See e.g., Deverman et al., “Gene Therapy for Neurological Disorders: Progress and Prospects,” Nature Rev. 17:641-659 (2018), which in hereby incorporated by reference in its entirety. Suitable AAV vectors include serotypes AAV1, AAV2, AAV3, AAV4, AAV5, AAV6, AAV7, AAV8, AAV9, AAV10, or AAV11 in their native form or engineered for enhanced tropism. AAV vectors known to have tropism for the CNS that are particularly suited for therapeutic expression of the α-synuclein antibodies described herein include, AAV1, AAV2, AAV4, AAV5, AAV8 and AAV9 in their native form or engineered for enhanced tropism. In one embodiment, the AAV vector is an AAV2 vector. In another embodiment, the AAV vector is an AAV5 vector as described by Vitale et al., “Anti-tau Conformational scFv MC1 Antibody Efficiently Reduces Pathological Tau Species in Adult JNPL3 Mice,” Acta Neuropathol. Commun. 6:82 (2018), optionally containing the GFAP or CAG promoter and the Eoodchuck hepatitis virus (WPRE) post-translational regulatory element. In another embodiment, the AAV vector is an AAV9 vector as described by Haiyan et al., “Targeting Root Cause by Systemic scAAV9-hIDS Gene Delivery: Functional Correction and Reversal of Severe MPSII in Mice,” Mol. Ther. Methods Clin. Dev. 10:327-340 (2018), which is hereby incorporated by reference in its entirety. In another embodiment, the AAV vector is an AAVrh10 vector as described by Liu et al., “Vectored Intracerebral Immunizations with the Anti-Tau Monoclonal Antibody PHF1 Markedly Reduces Tau Pathology in Mutant Transgenic Mice,” J. Neurosci. 36 (49): 12425-35 (2016), which is hereby incorporated by reference in its entirety.

In another embodiment the AAV vector is a hybrid vector comprising the genome of one serotype, e.g., AAV2, and the capsid protein of another serotype, e.g., AAV1 or AAV3-9 to control tropism. See e.g., Broekman et al., “Adeno-associated Virus Vectors Serotyped with AAV8 Capsid are More Efficient than AAV-1 or 2 Serotypes for Widespread Gene Delivery to the Neonatal Mouse Brain,” Neuroscience 138:501-510 (2006), which is hereby incorporated by reference in its entirety. In one embodiment, the AAV vector is an AAV2/8 hybrid vector as described by Ising et al., “AAV-mediated Expression of Anti-Tau ScFv Decreases Tau Accumulation in a Mouse Model of Tauopathy,” J. Exp. Med. 214 (5): 1227 (2017), which is hereby incorporated by reference in its entirety. In another embodiment the AAV vector is an AAV2/9 hybrid vector as described by Simon et al., “A Rapid Gene Delivery-Based Mouse Model for Early-Stage Alzheimer Disease-Type Tauopathy,” J. Neuropath. Exp. Neurol. 72 (11): 1062-71 (2013), which is hereby incorporated by reference in its entirety.

In another embodiment, the AAV vector is one that has been engineered or selected for its enhanced CNS transduction after intraparenchymal administration, e.g., AAV-DJ (Grimm et al., J. Viol. 82:5887-5911 (2008), which is hereby incorporated by reference in its entirety); increased transduction of neural stem and progenitor cells, e.g., SCH9 and AAV4.18 (Murlidharan et al., J. Virol. 89:3976-3987 (2015) and Ojala et al., Mol. Ther. 26:304-319 (2018), which are hereby incorporated by reference in their entirety); enhanced retrograde transduction, e.g., rAAV2-retro (Muller et al., Nat. Biotechnol. 21:1040-1046 (2003), which is hereby incorporated by reference in its entirety); selective transduction into brain endothelial cells, e.g., AAV-BRI (Korbelin et al., EMBO Mol. Med. 8:609-625 (2016), which is hereby incorporated by reference in its entirety); or enhanced transduction of the adult CNS after IV administration, e.g., AAV-PHP.B and AAVPHP.eB (Deverman et al., Nat. Biotechnol. 34:204-209 (2016) andChan et al., Nat. Neurosci. 20:1172-1179 (2017), which are hereby incorporated by reference in their entirety.

In accordance with this embodiment, the expression vector construct encoding the anti-α-synuclein antibody or binding portion thereof can include the nucleic acid encoding the heavy chain variable region polypeptide, a fragment thereof, a variant thereof, or combinations thereof. In one embodiment, the heavy chain variable region polynucleotide encodes only a variable heavy chain (VH) region. In another embodiment, the heavy chain variable region polynucleotide is engineered to further comprise a region encoding at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

The promoter sequence of the expression vector construct is suitable for driving expression of the antibody or binding fragment thereof. The promoter can be inducible or constitutive. Suitable promoter sequences include, without limitation, the elongation factor 1-alpha promoter (EF1a) promoter, a phosphoglycerate kinase-1 promoter (PGK) promoter, a cytomegalovirus immediate early gene promoter (CMV), artificial CMV-chicken β-actin promoter with β-globin splice acceptor (CAG), chicken β-actin (CBA) promoter, a chimeric liver-specific promoter (LSP) a cytomegalovirus enhancer/chicken beta-actin promoter (CAG), a tetracycline responsive promoter (TRE), a transthyretin promoter (TTR), a simian virus 40 promoter (SV40) and a CK6 promoter. Other promoters suitable for driving gene expression in mammalian cells that are known in the art are also suitable for incorporation into the expression constructs disclosed herein. The expression construct can further encode enhance-promoter elements that control or restrict expression of the encoded antibody. Enhancer-promoter elements that restrict expression to several neuronal and glial cell types in the CNS are known in the art and suitable for inclusion in the vector expression constructs described herein. See e.g., Lee et al., “GF AP Promoter Elements Required for Region-Specific and Astrocyte-Specific Expression,” Glia 56:481-493 (2008); Dimidschstein et al., “A Viral Strategy for Targeting and Manipulating Interneurons Across Vertebrate Species,” Nat. Neuroscience” 19:1743-49 (2016); and de Leeuq et al., “rAAV-compatible MiniPromoters for Restricted Expression in the Brain and Eye,” Mol. Brain 9:52 (2016), which are hereby incorporated by reference in their entirety.

The expression construct can further encode a linker sequence. The linker sequence can encode an amino acid sequence that spatially separates and/or links the one or more components of the expression construct.

Another embodiment of the invention is a host cell comprising the vectors described herein. The antibodies and binding fragments thereof described herein can be optionally produced by a cell line, a mixed cell line, an immortalized cell or clonal population of immortalized cells, as well known in the art (see e.g., Ausubel et al., ed., Current Protocols in Molecular Biology, John Wiley & Sons, Inc., NY, N.Y. (1987-2001); Sambrook et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); Colligan et al., eds., Current Protocols in Immunology, John Wiley & Sons, Inc., NY (1994-2001); Colligan et al., Current Protocols in Protein Science, John Wiley & Sons, NY, N.Y., (1997-2001), which are hereby incorporated by reference in their entirety).

The host cell chosen for expression may be of mammalian origin or may be selected from COS-1, COS-7, HEK293, BHK21, CHO, BSC-1, He G2, SP2/0, HeLa, myeloma, lymphoma, yeast, insect, or plant cells, or any derivative, immortalized or transformed cell thereof. Alternatively, the host cell may be selected from a species or organism incapable of glycosylating polypeptides, e.g., a prokaryotic cell or organism, such as BL21, BL21 (DE3), BL21-GOLD (DE3), XL1-Blue, JM109, HMS174, HMS174 (DE3), and any of the natural or engineered E. coli spp, Klebsiella spp., or Pseudomonas spp strains.

The antibodies described herein can be prepared by any of a variety of techniques using the isolated polynucleotides, vectors, and host cells described supra. In general, antibodies can be produced by cell culture techniques, including the generation of monoclonal antibodies via conventional techniques, or via transfection of antibody genes, heavy chains and/or light chains into suitable bacterial or mammalian cell hosts, in order to allow for the production of antibodies, wherein the antibodies may be recombinant. Standard molecular biology techniques are used to prepare the recombinant expression vector, transfect the host cells, select for transformants, culture the host cells and recover the antibody from the culture medium. Transfecting the host cell can be carried out using a variety of techniques commonly used for the introduction of exogenous DNA into a prokaryotic or eukaryotic host cell, e.g., by electroporation, calcium-phosphate precipitation, DEAE-dextran transfection and the like. Although it is possible to express the antibodies described herein in either prokaryotic or eukaryotic host cells, expression of antibodies in eukaryotic cells is sometimes preferable, and sometimes preferable in mammalian host cells, because such eukaryotic cells (and in particular mammalian cells) are more likely than prokaryotic cells to assemble and secrete a properly folded and immunologically active antibody.

As noted above, exemplary mammalian host cells for expressing the recombinant antibodies of the invention include Chinese Hamster Ovary (CHO cells) (including dhfr-CHO cells, described in Urlaub and Chasin, Proc. Natl. Acad. Sci. USA, 77:4216-4220 (1980), which is hereby incorporated by reference in its entirety). Other suitable mammalian host cells include, without limitation, NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown.

Host cells can also be used to produce functional antibodies and fragments thereof. It is understood that variations on the above procedure are within the scope of the present disclosure. For example, it may be desirable to transfect a host cell with DNA encoding functional fragments the heavy chain of an antibody described herein. Recombinant DNA technology may also be used to remove some or all of the DNA encoding portions of the heavy chain that are not necessary for binding to the antigens of interest. The molecules expressed from such truncated DNA molecules are also encompassed by the antibodies described herein.

The antibodies and antibody binding fragments are recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

In another embodiment, the antibody or binding fragment thereof, or the polynucleotide encoding the antibody or binding fragment thereof is a component of a pharmaceutical composition. In one embodiment, the pharmaceutical composition comprises a monoclonal antibody composition. In another embodiment, the pharmaceutical composition comprises two or more different single-domain antibodies, e.g., a polyclonal antibody composition. In another embodiment, the pharmaceutical composition comprises polynucleotides encoding the monoclonal antibody composition. In another embodiment, the pharmaceutical composition comprises polynucleotides encoding two or more different single-domain antibodies as described herein. In another embodiment, the pharmaceutical composition comprises one or more antibodies or polynucleotides encoding the same as described herein and one or more prophylactic or therapeutic agents other than the antibodies described herein that are useful for preventing or treating a condition mediated by a toxic α-synuclein protein.

The therapeutically effective amount of antibody present in the pharmaceutical composition or formulation is determined by taking into account the desired dose volumes and mode(s) of administration. Exemplary antibody concentrations in the pharmaceutical compositions of the present disclosure include from about 0.1 mg/mL to about 50 mg/mL, from about 0.5 mg/mL to about 25 mg/mL, and from about 2 mg/mL to about 10 mg/mL.

An aqueous formulation is prepared comprising the antibody in a pH-buffered solution. The buffer has a pH in the range from about 4.5 to about 10, from about 5 to about 9, or from about 6 to 8. Examples of buffers include phosphate buffers (e.g., phosphate buffered saline), acetate (e.g. sodium acetate), succinate (such as sodium succinate), gluconate, histidine, citrate and other organic acid buffers.

A polyol, which acts as a tonicifier and may stabilize the antibody, may be included in the formulation. In one embodiment, the tonicifying polyol is a salt such as sodium chloride. In another embodiment, the polyol is a non-reducing sugar, such as sucrose or trehalose. The polyol is added to the formulation in an amount which may vary with respect to the desired isotonicity of the formulation. Preferably the aqueous formulation is isotonic, in which case suitable concentrations of the polyol in the formulation are in the range from about 1% to about 15% w/v, or in the range from about 2% to about 10% w/v, for example. However, hypertonic or hypotonic formulations may also be suitable. The amount of polyol added may also alter with respect to the molecular weight of the polyol. For example, a lower amount of a monosaccharide (e.g. mannitol) may be added, compared to a disaccharide (such as trehalose).

A surfactant may also be added to the pharmaceutical composition containing the antibody. Exemplary surfactants include nonionic surfactants such as polysorbates (e.g. polysorbates 20, 80 etc), poloxamers (e.g. poloxamer 188), Pluronic F68, and PEG (polyethylene glycol). The amount of surfactant added is such that it reduces aggregation of the formulated antibody and/or minimizes the formation of particulates in the formulation and/or reduces adsorption. For example, the surfactant may be present in the formulation in an amount from about 0.001% to about 0.5%, from about 0.005% to about 0.2%, or from about 0.01% to about 0.1%.

In one embodiment, the pharmaceutical composition contains the above-identified agents (i.e. antibody, buffer, polyol and surfactant) and is essentially free of one or more preservatives, such as benzyl alcohol, phenol, m-cresol, chlorobutanol and benzethonium Cl. In another embodiment, a preservative may be included in the pharmaceutical composition, particularly where the formulation is a multidose formulation. Suitable preservatives include, without limitation phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. The concentration of preservative may be in the range from about 0.01% to about 5%, from about 0.5% to about 2% and any range or value therein. Non-limiting examples include, no preservative, 0.1-2% m-cresol, 0.1-3% benzyl alcohol, 0.001-0.5% thimerosal, 0.001-2.0% phenol, 0.0005-1.0% alkylparaben(s), and the like. One or more other pharmaceutically acceptable carriers, excipients or stabilizers such as those described in Remington's Pharmaceutical Sciences 16th edition, Osol, A. Ed. (1980), which is hereby incorporated by reference in its entirety, may be included in the composition provided that they do not adversely affect the desired characteristics of the formulation. Acceptable carriers, excipients or stabilizers are nontoxic to recipients at the dosages and concentrations employed and include; additional buffering agents; co-solvents; antioxidants including ascorbic acid and methionine; chelating agents such as EDTA; metal complexes (e.g. Zn-protein complexes); biodegradable polymers such as polyesters; and/or salt-forming counterions such as sodium.

The pharmaceutical compositions to be used for in vivo administration must be sterile. This is readily accomplished by filtration through sterile filtration membranes, prior to, or following, preparation of the composition.

The pharmaceutical compositions comprising antibodies or binding fragments thereof, or polynucleotides encoding the antibodies or binding fragments thereof, are for use in, but not limited to, preventing, treating, managing, or ameliorating an α-synucleinopathy, or one or more symptoms thereof.

In another aspect of the present disclosure the anti-α-synuclein antibodies described herein, binding fragments thereof, or a pharmaceutical composition containing the same, are employed in a method of inhibiting onset of one or more symptoms of an α-synucleinopathy in a subject. This method involves administering to the subject the anti-α-synuclein antibodies described, polynucleotides encoding the anti-α-synuclein antibodies described herein, or a pharmaceutical composition containing the same to the subject in an amount effective to inhibit the onset of one or more symptoms of an α-synucleinopathy in the subject.

In another aspect of the present disclosure the α-synuclein antibodies described herein, polynucleotides encoding the α-synuclein antibodies described herein, or a pharmaceutical composition containing the same, are employed in a method of treating a subject having an α-synucleinopathy. This method involves selecting a subject having an α-synucleinopathy and administering to the subject the α-synuclein antibody, a polynucleotide encoding an α-synuclein antibody, or a pharmaceutical composition containing the same in an amount effective to treat the α-synucleinopathy in the subject.

In accordance with these embodiments, an α-synucleinopathy is any condition associated with or mediated by a pathological form of α-synuclein protein. α-synucleinopathies include, without limitation, Parkinson's disease, Alzheimer's disease, Lewy Body Dementia, and Multiple System Atrophy.

In accordance with these embodiments, the “subject” is typically a human. However, other non-human mammals amenable to treatment in accordance with the methods described herein include, without limitation, primates, dogs, cats, rodents (e.g., mouse, rat, guinea pig), horses, deer, cervids, cattle and cows, sheep, and pigs.

In prophylactic applications, the pharmaceutical compositions of the present invention are administered to a subject that is susceptible to, or otherwise at risk of developing a particular condition mediated by a pathological form of α-synuclein protein, i.e., an α-synucleinopathy, in an amount sufficient to eliminate or reduce the risk of the condition or to delay, inhibit, or prevent the onset of the condition. Prophylactic application also includes the administration of an antibody composition to prevent or delay the recurrence or relapse of a condition mediated by the α-synuclein protein or peptide in its pathological form. The present methods and compositions are especially suitable for prophylactic treatment of individuals who have a known genetic risk of developing an α-synucleinopathy. Genetic markers associated with a risk of developing an α-synucleinopathy, such as Parkinson's disease include mutations in the SNCA gene, the gene encoding α-synuclein. Mutations in SCNA gene have been linked to both familial and sporadic cases of PD (see Stefanis L., “α-Synuclein in Parkinson's Disease,” Cold Spring Harb. Perspect. Med. 2 (2): 1009399 (2012), which is hereby incorporated by reference in its entirety). Genetic markers of other α-synucleinopathies, such as Alzheimer's disease are also known in the art. For example, mutations in the APP gene, particularly mutations at position 717 and positions 670 and 671 referred to as the Hardy and Swedish mutations respectively. Other markers of risk are mutations in the presenilin genes, PS1 and PS2, and ApoE4, family history of AD, hypercholesterolemia or atherosclerosis.

In therapeutic applications, pharmaceutical compositions are administered to a subject suspected of, or already suffering from a condition associated with or caused by a pathological form of α-synuclein protein in an amount sufficient to cure, or at least partially arrest or alleviate, one or more symptoms of the condition and its complications. An amount adequate to accomplish this is defined as a therapeutically- or pharmaceutically-effective dose. In both prophylactic and therapeutic regimes, agents are usually administered in several dosages until a sufficient response has been achieved. An effective dose of the composition of the present invention, for the treatment of the above described conditions will vary depending upon many different factors, including means of administration, target site, physiological state of the patient, whether the patient is human or an animal, other medications administered, and whether treatment is prophylactic or therapeutic.

In accordance with the prophylactic and therapeutic methods described herein, compositions comprising the antibody or binding fragments thereof are administered in a dosage ranging from about 0.0001 to 100 mg/kg, and more usually 0.01 to 10 mg/kg of the recipient's body weight. For example, the antibody or binding fragment thereof is administered in a dosage of 1, 2, 3, 4, 5, 6, 7, 8, 9 or 10 mg/kg, or higher, for example 15, 20, 25, 30, 35, 40, 45, 50, 55, 60, 65, 70, 75, 80, 85, 90, 95, or 100 mg/kg. An exemplary treatment regime entails administration once per every two weeks or once a month or once every 3 to 6 months. Intervals between single dosages can be weekly, monthly or yearly. Intervals can also be irregular as indicated by measuring blood levels of antibody in the patient. Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

The mode of administration of the antibody, binding fragment thereof, or pharmaceutical composition described herein may be any suitable route that delivers the compositions to the host, such as parenteral administration, e.g., intradermal, intramuscular, intraperitoneal, intravenous or subcutaneous, pulmonary; transmucosal (e.g., oral, intranasal); using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by, for example, intraarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapericardiac, intraperitoneal, intrapleural, intraprostatic, intrapulmonary, intrarenal, intraretinal, intraspinal, intrasynovial, intrathoracic, intravascular, intravesical, intralesional, sublingual, intranasal, or transdermal delivery.

Administration can be systemic or local. In one embodiment, it may be desirable to administer the antibodies of the invention locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices.

In another embodiment, compositions containing the antibody or binding fragment thereof are delivered in a controlled release or sustained release system. In one embodiment, a pump is used to achieve controlled or sustained release. In another embodiment, polymeric materials can be used to achieve controlled or sustained release of the antibody compositions described herein. Examples of polymers used in sustained release formulations include, but are not limited to, poly(2-hydroxy ethyl methacry-late), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation is preferably inert, free of leachable impurities, stable on storage, sterile, and biodegradable. Also, the use of liposomes, microcapsules or microspheres, inclusion complexes, or other types of carriers known in the art are also contemplated.

In yet another embodiment, a controlled or sustained release system can be placed in proximity of the prophylactic or therapeutic target, thus requiring only a fraction of the systemic dose. Controlled and/or release systems for delivery of antibodies known in the art are suitable for use and delivery of compositions containing the antibodies and binding fragments thereof as described herein, see e.g., Song et al, “Antibody Mediated Lung Targeting of Long-Circulating Emulsions,” PDA Journal of Pharmaceutical Science & Technology 50:372-397 (1995); Cleek et al, “Biodegradable Polymeric Carriers for a bFGF Antibody for Cardiovascular Application,” Pro. Int'l. Symp. Control. Rel. Bioact. Mater. 24:853-854 (1997); and Lam et al., “Microencapsulation of Recombinant Humanized Monoclonal Antibody for Local Delivery,” Proc. Int'l. Symp. Control Rel. Bioact. Mater. 24:759-760 (1997), each of which is incorporated herein by reference in their entireties.

In embodiments where the pharmaceutical composition comprises polynucleotides encoding the antibody or binding fragment thereof as described herein, the nucleic acid can be administered in vivo to promote expression of its encoded antibody, by constructing it as part of an appropriate nucleic acid expression vector and administering it so that it becomes intracellular, e.g., the by use of a retroviral vector (see e.g., U.S. Pat. No. 4,980,286 to Morgan et al., which is hereby incorporated by reference in its entirety). In one embodiment, polynucleotides encoding the α-synuclein antibody as described herein are incorporated into an AAV vector as described supra (e.g., AAV2, AAV4, AAV5, AAV7, AABV8, AAV9, AAVrh10, AAV2/8, AAV2/9, etc.) and delivered via intraparenchymal administration, including convection enhanced delivery (CED), intrathecal administration, intracerebroventricular administration, subpial administration, intramuscular administration, or intravenous administration. Other forms of nucleic acid delivery can also be employed, e.g., direct injection, use of microparticle bombardment (see e.g., a gene gun; Biolistic, Dupont), coating with lipids or cell-surface receptors or transfecting agents, or by administering it in linkage to a homeobox-like peptide which is known to enter the nucleus (see e.g., Joliot et al, Proc. Natl. Acad. Sci. USA 88:1864-1868 (1991), which is hereby incorporated by reference in its entirety). Alternatively, a nucleic acid can be introduced intracellularly and incorporated within host cell DNA for expression by homologous recombination.

If the methods described herein involve intranasal administration of the antibody composition, the composition can be formulated in an aerosol form, spray, mist or in the form of drops. In particular, prophylactic or therapeutic agents for use according to the present invention can be conveniently delivered in the form of an aerosol spray presentation from pressurized packs or a nebulizer, with the use of a suitable propellant (e.g., dichlorodifluoromethane, trichloro-fluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

If the methods described herein involve oral administration of the antibody compositions described herein, the compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate.

In another embodiment, a pharmaceutical composition comprising a recombinant nucleic acid sequence encoding an antibody or binding portion thereof as described herein, is administered to a subject to facilitate in vivo expression and formation of the antibody for the treatment or prevention of conditions mediated by toxic oligomeric proteins or peptides in a subject. Expression vector constructs suitable for use in this embodiment of the disclosure are described supra.

The polynucleotide compositions can result in the generation of the antibody in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the synthetic antibody in the subject within at least about 1 day, 2 days, 3 days, 4 days, 5 days, 6 days, 7 days, 8 days, 9 days, or 10 days of administration of the composition to the subject. The composition can result in generation of the antibody in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the persistent generation of the antibody in the subject. The composition can result in the generation of the antibody in the subject for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days.

Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use. The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The methods of the invention encompass administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

The antibodies, binding fragments thereof, or pharmaceutical compositions containing the same can be packaged in hermetically sealed containers such as an ampoule or sachette indicating the quantity of the antibody or binding fragment thereof. In one embodiment, one or more of the antibodies, or pharmaceutical compositions of the invention is supplied as a dry sterilized lyophilized powder or water free concentrate in a hermetically sealed container and can be reconstituted (e.g., with water or saline) to the appropriate concentration for administration to a subject. In one embodiment, one or more of the antibodies or pharmaceutical compositions of the invention is supplied as a dry sterile lyophilized powder in a hermetically sealed container at a unit dosage of at least 5 mg, for example at least 10 mg, at least 15 mg, at least 25 mg, at least 35 mg, at least 45 mg, at least 50 mg, at least 75 mg, or at least 100 mg. The lyophilized antibodies or pharmaceutical compositions of the invention should be stored at between 2° C. and 8° C. in its original container and the antibodies, or pharmaceutical compositions of the invention should be administered within 1 week, for example within 5 days, within 72 hours, within 48 hours, within 24 hours, within 12 hours, within 6 hours, within 5 hours, within 3 hours, or within 1 hour after being reconstituted. In an alternative embodiment, one or more of the antibodies or pharmaceutical compositions of the invention are supplied in liquid form in a hermetically sealed container indicating the quantity and concentration of the antibody. In a further embodiment, the liquid form of the administered composition is supplied in a hermetically sealed container at least 0.25 mg/ml, for example at least 0.5 mg/ml, at least 1 mg/ml, at least 2.5 mg/ml, at least 5 mg/ml, at least 8 mg/ml, at least 10 mg/ml, at least 15 mg/ml, at least 25 mg/ml, at least 50 mg/ml, at least 75 mg/ml or at least 100 mg/ml. The liquid form should be stored at between 2° C. and 8° C. in its original container.

The antibodies and binding fragments described herein can be incorporated into a pharmaceutical composition suitable for parenteral administration. In one aspect, antibodies will be prepared as an injectable solution containing 0.1-250 mg/ml antibody. The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the tonicity of the solution at a concentration of 0-300 mM (optimally 150 mM for a liquid dosage form).

Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Additional surfactants include but are not limited to polysorbate 20 and BRIJ surfactants. The pharmaceutical composition comprising the antibodies described herein prepared as an injectable solution for parenteral administration, can further comprise an agent useful as an adjuvant, such as those used to increase the absorption, or dispersion of the antibody. A particularly useful adjuvant is hyaluronidase, such as Hylenex® (recombinant human hyaluronidase). Addition of hyaluronidase in the injectable solution improves human bioavailability following parenteral administration, particularly subcutaneous administration. It also allows for greater injection site volumes (i.e. greater than 1 ml) with less pain and discomfort, and minimum incidence of injection site reactions (see WO 04/078140 to Bookbinder et al., and U.S. Patent Appl. Publication No. US2006104968 to Bookbinder et al., which are hereby incorporated herein by reference in their entirety).

The antibodies and binding fragments described herein can also be employed in a number of diagnostic, prognostic and research applications.

Another aspect of the present disclosure is directed to a method of diagnosing an α-synucleinopathy or related disease in a subject. This method involves detecting, in the subject, the presence of α-synuclein protein or peptide using a diagnostic reagent, wherein the diagnostic reagent comprises an antibody or binding fragment described herein. The diagnosis of an α-synucleinopathy in the subject is based on the detection of α-synuclein protein or peptide in the subject.

In one embodiment, the method of diagnosing an α-synucleinopathy or related disease in the subject involves the detection of an aggregated form of α-synuclein protein or peptide. In another embodiment, the method of diagnosing an α-synucleinopathy or related disease in the subject involves the detection of accumulated monomeric forms of the α-synuclein protein or peptide. In another embodiment, the method of diagnosing an α-synucleinopathy or related disease in the subject involves the detection of aggregated and non-aggregated (i.e. monomeric forms) of α-synuclein protein or peptide.

Detecting the presence of α-synuclein protein or peptide in a subject using the antibodies or antibody fragments thereof as described herein can be achieved by obtaining a biological sample from the subject (e.g., blood, urine, cerebral spinal fluid, ocular lacrimal secretion, saliva, feces, nasal brushings and tissue or organ biopsy), contacting the biological sample with the diagnostic antibody reagent, and detecting binding of the diagnostic antibody reagent to α-synuclein protein or peptide if present in the sample from the subject. Assays for carrying out the detection of α-synuclein protein or peptide in a biological sample using a diagnostic antibody are well known in the art and include, without limitation, ELISA, immunohistochemistry, SIMOA (single molecule array), and Western blot.

In accordance with this and other embodiments described herein, the α-synuclein antibody or binding fragments described herein are coupled to a detectable label. The label can be any detectable moiety known and used in the art. Suitable labels include, without limitation, radioisotopes or radionuclides (e.g., ³H, ¹⁴C, ¹⁸F, ³⁵S, ⁹⁰Y, ⁹⁹Tc, ¹¹¹In, ¹²⁴I, ¹²⁵I, ¹³¹I, ¹⁷⁷Lu, ¹⁶⁶Ho, ⁸⁹Zr, or ¹⁵³Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

Detecting the presence of α-synuclein in a subject using the diagnostic antibody reagent of the present invention can also be achieved using in vivo imaging techniques. In vivo imaging involves administering to the subject the antibody or binding fragments thereof described herein, and detecting the binding of the antibody or binding fragment thereof to the α-synuclein in vivo.

Diagnostic antibodies or similar reagents can be administered by intravenous injection into the body of the patient, or directly into the brain by intracranial injection or by drilling a hole through the skull. The dosage of antibody should be within the same ranges as for treatment methods. In accordance with this embodiment, the antibody or binding fragment is coupled to an imaging agent to facilitate in vivo imaging. The imaging agent can be any agent known to one of skill in the art to be useful for imaging, preferably being a medical imaging agent. Examples of medical imaging agents include, but are not limited to, single photon emission computed tomography (SPECT) agents, positron emission tomography (PET) agents, magnetic resonance imaging (MRI) agents, nuclear magnetic resonance imaging (NMR) agents, x-ray agents, optical agents (e.g., fluorophores, bioluminescent probes, near infrared dyes, quantum dots), ultrasound agents and neutron capture therapy agents, computer assisted tomography agents, two photon fluorescence microscopy imaging agents, and multi-photon microscopy imaging agents. Exemplary detectable markers include radioisotopes (e.g., ¹⁸F, ¹¹C, ¹³N, ⁶⁴Cu, ¹²⁴I, ⁷⁶Br, ⁸²Rb, ⁶⁸Ga ^{99m}Tc, ¹¹¹In, ²⁰¹Tl, ⁸⁹Zr, or ¹⁵O, which are suitable for PET and/or SPECT use) and ultra-small superparamagnetic particles of iron oxide (USPIO) which are suitable for MRI.

Diagnosis of an α-synucleinopathy is performed by comparing the amount, size, and/or intensity of detected α-synuclein in a sample from the subject or in the subject, to corresponding baseline values. An appropriate baseline value can be the average level of α-synuclein found in a population of undiseased individuals. Alternatively, an appropriate baseline value may be the level of α-synuclein in the same subject determined at an earlier time.

The diagnostic methods described herein can also be used to monitor a subject's response to therapy. In this embodiment, detection of α-synuclein in the subject is determined prior to or concurrent with the commencement of treatment. The level of α-synuclein in the subject at this timepoint is used as a baseline value. At various times during the course of treatment the detection of α-synuclein is repeated, and the measured values thereafter compared with the baseline values. A decrease in values relative to baseline signals a positive response to treatment. No change or an increase in values relative to baseline signals an inadequate response to treatment. The treatment plan of an individual can be modified based on the results of monitoring the levels of α-synuclein in the subject.

A related aspect of the disclosure is directed to a method of identifying a subject's risk for developing an α-synucleinopathy or other condition mediated by or associated with a pathological form of α-synuclein. This method involves detecting, in the subject, the presence of accumulated α-synuclein protein or peptide using a diagnostic reagent comprising the antibody or binding fragment thereof described herein, and identifying the subject's risk of developing an α-synucleinopathy or condition mediated by or associated with accumulated α-synuclein based on the results of the detecting step.

Methods of detecting the presence of α-synuclein in the subject or in a sample from the subject include the in vitro and in vivo methods described supra. In one embodiment, the subject is not exhibiting any definitive signs or symptoms of an α-synucleinopathy, and employment of this method serves as an early diagnostic. In another embodiment, the subject is not exhibiting any signs or symptoms of an α-synucleinopathy, but has a genetic predisposition to a condition and employment of this method serves to predict the likelihood that the individual will develop the α-synucleinopathy in the future. In either embodiment, appropriate therapeutic and/or prophylactic intervention can be employed, e.g., administration of a therapeutic composition containing an antibody or polynucleotides encoding an antibody in an amount effective to slow or prevent the onset or progression of the α-synucleinopathy.

Another aspect of the present disclosure is directed to a diagnostic kit that comprises the antibody or binding fragment thereof as described herein and a detectable label.

A suitable detectable label is any moiety attached to an antibody or an analyte to render the reaction between the antibody and the analyte detectable. A label can produce a signal that is detectable by visual or instrumental means. Various labels include signal-producing substances, such as chromogens, fluorescent compounds, chemiluminescent compounds, radioactive compounds, and the like. Representative examples of detectable labels include moieties that produce light, e.g., acridinium compounds, and moieties that produce fluorescence, e.g., fluorescein. In this regard, the moiety itself may not be detectable, but becomes detectable upon reaction with yet another moiety.

Other suitable detectable labels include radioactive labels (e.g., H, I, S, C, P, and P), enzymatic labels (e.g., horseradish peroxidase, alkaline peroxidase, glucose 6-phosphate dehydrogenase, and the like), chemiluminescent labels (e.g., acridinium esters, thioesters, or sulfonamides; luminol, isoluminol, phenanthridinium esters, and the like), fluorescent labels (such as fluorescein (e.g., 5-fluorescein, 6-carboxyfluorescein, 3′6-carboxyfluorescein, 5 (6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label.

TABLE 3
Polynucleotide Sequences of Synuclein Antibodies
Enriched by Solid Phase Panning

| | | NA | AA |
|---|---|---|---|
| | | SEQ | SEQ |
| | | ID | ID |
| Antibody ID | Nucleotide Sequence | NO: | NO: |
| 03-S-sR1- | ATGAAATACCTATTGCCTACGGCGGCCGCTGGATTGTTATTACTCGCGG | 242 | 109 |
| 2D3 | CCCAGCCGGCCATGGCGCAGGTGCAGTTGCAGCAGTCTGGGGGAGGC | | |
| | TTGGTGCAGGCTGGGGGATCTCTGATTCTCCGTTGTAGAGCAACTGTA | | |
| | AGTGGCTTCAGTATCGGGACCATGGGCTGGTACCGCCAGGCTCCCGG | | |
| | GAAGGAGCGCGAGTTCGTCGCGAACGTTAGTCCTAGCGGTGCAAAAT | | |
| | ACTTCGCTGACTCCGTGAAGGGCCGATTCACCATCTCCAGAGACAACG | | |
| | CCAATAATACAGTGTATCTGCAAATGAACAGTCTGAAACCTGAAGACA | | |
| | CGGGCGTCTATTATTGTAATATACGAAGGTTTTCGTACCTCAGTGGCGA | | |
| | CTGGGGCCAGGGGACCCAGGTCACCGTCTCCTCAACTAGTGGCCCGG | | |
| | GAGGCCAACACCATCACCACCATCATGGCGCAGAACAAAAACTCATCT | | |
| | CAGAAGAGGATCTGTCTTAG | | |
| 07-S-sR2- | ATGAAATACCTATTGCCTACGGCGGCCGCTGGATTGTTATTACTCGCGG | 243 | 110 |
| 1D12 | CCCAGCCGGCCATGGCGCAGGTGCAGTTGCAGGAGTCTGGGGGAGGC | | |
| | TTGGTGCAGGCTGGGGGGTCTCTGAGACTCTCCTGTGCAGCCTCTGGA | | |
| | TTCACCTTCAGTAGCAGTTCCATGGGCTGGTACCGCCAGGCTCCAGGG | | |
| | AAGCAGCGCGAGTTGGTCGCTTCTATTATGCGTTATGGTACTACAACCT | | |
| | ATACAGACTCCGTGAAGGGCCGATTCACCATCTCCAGAGACAACGGCC | | |
| | AGAGAACAGTCTATCTGCAAATGAACAGCCTGAAGCCTGAGGACACG | | |
| | GCCGTCTATTATTGTAATGTTCGAAGTTTCGTTCGAACCTACTGGGGCC | | |
| | AGGGGACCCTGGTCACCGTCTCCTCAACTAGTGGCCCGGGAGGCCAAC | | |
| | ACCATCACCACCATCATGGCGCAGAACAAAAACTCATCTCAGAAGAGG | | |
| | ATCTGTCTTAG | | |
| 08-S-sR2- | ATGAAATACCTATTGCCTACGGCGGCCGCTGGATTGTTATTACTCGCGG | 244 | 111 |
| 2E10 | CCCAGCCGGCCATGGCGCAGGTGCAGNTGCAGGAGTCTGGGGGAGG | | |
| | TTGGTGCAGGCTGGGGGCTCTCTGAGACTCTCCTGTGCAGCCTCTGG | | |
| | ACGCACCTTCAGTAGTTTTGCCATGGGCTGGTTCCGCCAGGCTCCAGG | | |
| | GAAGGAGCGTGAGTTTGTTACAGCTATTAACTGGAGTGGTAGTAGCAC | | |
| | ATACTATGCAGACTCCGTGAAGGGCCGATTCACCATCTCCAGAGACAA | | |
| | CGCCAAGAACACGGTGTATCTGCAAATGAACAGCCTGAAACCTGAGGA | | |
| | CACGGCCGTCTATTACTGTAATGCCCAGCGGAGGTGGCCTCTTCGTGA | | |
| | CTATTGGGGCCAGGGGACCCAGGTCACCGTCTCCTCAACTAGTGGCCC | | |
| | GGGAGGCCAACACCATCACCACCATCATGGCGCAGAACAAAAACTCAT | | |
| | CTCAGAAGAGGATCTGTCTTAG | | |
| 11-S-sR1- | ATGAAATACCTATTGCCTACGGCGGCCGCTGGATTGTTATTACTCGCGG | 245 | 112 |
| 2D12 | CCCAGCCGGCCATGGCGCAGGTGCAAAAAAAAGAGTCTGGGGGAGGC | | |
| | TTGGTGCAGGCTGGGGGGTCTCTGAGACTCTCCTGTGCAGCCTCTGGA | | |
| | CTCATCTTCAGTATCAATGCCATGGCCTGGTACCGCCAGGCTCCAGGG | | |
| | AACCAGCGCGAGTTGGTCGCACGTATTACTACTGGTGGTAGCACAAAC | | |
| | TATGCAGACTCCGTGAAGGGCCGATTCACCATCTCCAGAGACAACGCC | | |
| | AAGAACACGGTGTATCTGCAAATGAACAGCCTGAAACCTGAGGACACA | | |
| | GCCGTCTATTTCTGTGCAGCAGATGTAAGGTTTGGGGAACGGACTCCC | | |
| | TACTGGGGCCAGGGGACCCAGGTCACCGTCTCCTCAACTAGTGGCCCG | | |
| | GGAGGCCAACACCATCACCACCATCATGGCGCAGAACAAAAACTCATC | | |
| | TCAGAAGAGGATCTGTCTTAG | | |
| 15-S-sR2-1E7 | ATGAAATACCTATTGCCTACGGCGGCCGCTGGATTGTTATTACTCGCGG | 246 | 113 |
| | CCCAGCCGGCCATGGCGCAGGTGCAGTTGCAGGCGTCTGGGGGAGGC | | |
| | GTGGTGCAGTCTGGGGGGTCTCTGAGACTCTCCTGTGTAGCCTCTGGA | | |
| | AACATCTTCAGGATCAATGCCATGGGCTGGTACCGCCAGGCTCCAGGG | | |
| | AAGCAGCGCGAGTTGGTCGCACATATTATTAGTGGTGGTAGCACAAAC | | |
| | TATGCAGACTCCGTGAAGGGCCGATTCACCATCTCCAGAGAATACGCC | | |
| | AAGAATATGGTGTATCTGCAAATGAACAGCCTGAAACCTGAGGACACG | | |
| | GCCGTCTATTACTGTAATGCCCGAACTTTCGTGAGAACCTACTGGGGCC | | |
| | AGGGGACCCAGGTCACCGTCTCCTCAACTAGTGGCCCGGGAGGCCAA | | |
| | CACCATCACCACCATCATGGCGCAGAACAAAAACTCATCTCAGAAGAG | | |
| | GATCTGTCTTAG | | |
| 16-S-sR2- | ATGAAATACCTATTGCCTACGGCGGCCGCTGGATTGTTATTACTCGCGG | 247 | 114 |
| 2F6 | CCCAGCCGGCCATGGCGCAGGTGCAGTTGCAGCAGTTTGGGGGAGGC | | |
| | TTGGTGCAGCCTGGGGGGTCTCTGAGACTCTCCTGTCAAGCCTCTACA | | |
| | AGTGTCTTCGGTAACACTGCCATGGCCTGGTACCGCCAGGCTCCTGGG | | |
| | AAGCAGCGCGAGTTGGTCGCACGAATTACTACCCTTGGTTTCACATACT | | |
| | ATGCAGACTCCGCGAAGGGCCGATTCACCATCTCTAGAGACAGCGCCA | | |
| | TGAACACGGTGTATCTGCAAATGAACAGCCTGAAACCTGAGGACACGG | | |
| | CCGTCTATTACTGTAACCGCAGAGGATTTCGGAGCTACTGGGGCCAGG | | |
| | GGACCCTGGTCACCGTCTCCTCAACTAGTGGCCCGGGAGGCCAACACC | | |
| | ATCACCACCATCATGGCGCAGAACAAAAACTCATCTCAGAAGAGGATC | | |
| | TGTCTTAG | | |
| 19-S-sR1- | ATGAAATACCTATTGCCTACGGCGGCCGCTGGATTGTTATTACTCGCGG | 248 | 115 |
| 2G4 | CCCAGCCGGCCATGGCGCAGGTGCAGTTGCAGGAGTCCGGGGGAGGC | | |
| | NTNGGTGCAGTCTGGCGATTCTCTGAGACTCTCCTGTGCAGCCTCTGG | | |
| | AAGCATCTATCATGTCAATACCATGGGTTGGTACCGCCAGTCTCCAGG | | |
| | AAAGCAGCGCGAGTTGGTCGCAACTCTTACACATAACAACCGCGTAAC | | |
| | CTATGCAGACTCCGTGAAGGGTCGATTCACCATCTCCAGAGACAACGC | | |
| | CAAGATGACGGTGTATCTGCAAATGGACAGCCTGAAACCCGATGACAC | | |
| | GGCCGTATATTACTGTTACTACTTCGTCCCGCGTAATCCATTATTCGGG | | |
| | AGAAGGATTGACTTTGATGCCTGGGGCCAGGGGACCCAGGTCACCGT | | |
| | CTCCTCAACTAGTGGCCCGGGAGGCCAACACCATCACCACCATCATGG | | |
| | CGCAGAACAAAAACTCATCTCAGAAGAGGATCTGTCTTAG | | |
| 23-S-sR2-1E9 | ATGAAATACCTATTGCCTACGGCGGCCGCTGGATTGTTATTACTCGCGG | 249 | 116 |
| | CCCAGCCGGCCATGGCGCAGGTGCAGTTGCAGGAGTCTGGGGGAGGC | | |
| | GTGGTGCAGTCTGGGGGGTCTCTGAGACTCTCCTGTGTAGCCTCTGGA | | |
| | AACATCTTCAGGATCAATGTCATGGGCTGGTACCGCCAGGCTCCAGGG | | |
| | AAGCAGCGCGAGTTGGTCGCGGTTGTAAAGAGTGGTGGTAGCACAAA | | |
| | CTATGTAGACTCCGCGAAGGGACGATTCACCATCTCCAGGGACAACGC | | |
| | CAAGAACACAGCGTATCTGCACATGGACAGCCTGAAACCTGAGGACAC | | |
| | GGCCGTCTATTACTGTAATGCACAAACCCGACTCTGGAGCTACTGGGG | | |
| | CCAGGGGACCCAGGTCACCGTTTCCTCAACTAGTGGCCCGGGAGGCCA | | |
| | ACACCATCACCACCATCATGGCGCAGAACAAAAACTCATCTCAGAAGA | | |
| | GGATCTGTCTTAG | | |
| 24-S-sR2- | ATGAAATACCTATTGCCTACGGCGGCCGCTGGATTGTTATTACTCGCGG | 250 | 117 |
| 2F7 | CCCAGCCGGCCATGGCGCAGGTGCAGCTGCAGGAGTTTGGGGGAGGC | | |
| | GTGGTGCAGTCTGGGGGGTCTCTGAGACTCTCCTGTGTAGCCTCTGGA | | |
| | AACATCTTCAGGATCAATGCCATGGGCTGGTACCGCCAGGCTCCAGGG | | |
| | AAGCAGCGCGAGTTGGTCGCACATATTATTAGTGGTGGTAGCACAAAC | | |
| | TATGCAGACTCCGTGAAGGGCCGATTCACCATCTCCAGAGAATACGCC | | |
| | AAGAATATGGTGTATCTGCAAATGAACAGCCTGAAACCTGAGGACACG | | |
| | GCCGTCTATTACTGTAATGCACAAACCCGACTCTGGAGCTACTGGGGC | | |
| | CAGGGGACCCAGGTCACCGTCTCCTCAACTAGTGGCCCGGGAGGCCA | | |
| | ACACCATCACCACCATCATGGCGCAGAACAAAAACTCATCTCAGAAGA | | |
| | GGATCTGTCTTAG | | |
| 31-S-sR2-1F7 | ATGAAATACCTATTGCCTACGGCGGCCGCTGGATTGTTATTACTCGCGG | 251 | 118 |
| | CCCAGCCGGCCATGGCGCAGGTGCAGCTGCAGGAGTCTGGGGGAGGC | | |
| | TTGGTGCAGCCTGGGGGGTCTCTGAGACTCTCCTGTGCAGCCTCTCGA | | |
| | AGCTTCTTCAGTATCAATGCCATGGGCTGGTACCGCCAGGCTCCAGGG | | |
| | AAGCAGCGCGAGTTGGTCGCAACTATTACTAGTCGTGATAGCACAAAC | | |
| | GTTGCAGACTCCGTGAAGGGCCGATTCACCATCTCCAGAGACTACGCC | | |
| | AAGAACATAGTGTATCTGCAAATGGACAGCCTGAGACCTGAGGACAC | | |
| | GGCCACATATTACTGCTACGCTGATCAACCGTGGAGGGGTCGTGCCTG | | |
| | GGGCCAGGGGACCCAGGTCACCGTCTCCTCAACTAGTGGCCCGGGAG | | |
| | GCCAACACCATCACCACCATCATGGCGCAGAACAAAAACTCATCTCAG | | |
| | AAGAGGATCTGTCTTAG | | |
| 32-S-sR2- | ATGAAATACCTATTGCCTACGGCGGCCGCTGGATTGTTATTACTCGCGG | 252 | 119 |
| 2F11 | CCCAGCCGGCCATGGCGCAGGTGCAGTTGCAGGAGTCTGGGGGAGGC | | |
| | GTGGTGCAGGCCGGGGGGTCTCTGAACCTCTCCTGTACACACTCAACA | | |
| | ATCACCTTCAGGATCAACACCATGGCGTACTATCGCCAGGCTCCAGGG | | |
| | TCTCAGCGCGCCCTGGTCGCGCGGATTAATCCAGCAGGGAGGACGTAT | | |
| | TATCCAGATTCCGTGAAGGGCCGATTCACCATCTCCAGAGACAACGCC | | |
| | AAGAACCAAGTGTATCTACAAATGAACGACCTCAAACCTGAGGACACG | | |
| | GCCGTCTATTACTGTTCTACATGGCGACTAGGACGCAACTACTGGGGC | | |
| | CAGGGGACCCTGGTCACCGTCTCCTCAACTAGTGGCCCGGGAGGCCAA | | |
| | CACCATCACCACCATCATGGCGCAGAACAAAAACTCATCTCAGAAGAG | | |
| | GATCTGTCTTAG | | |
| 34-S-sR1- | ATGAAATACCTATTGCCTACGGCGGCCGCTGGATTGTTATTACTCGCGG | 253 | 120 |
| 1A12 | CCCAGCCGGCCATGGCGCAGGTGCAGTTGCAGGAGTTTGGGGGAGGC | | |
| | TTGGTGCAGGCTGGGGGGGTTCTGAGACTCTCCTGTGTAGCCTCTATG | | |
| | ACTACCCTCGGTTTCAAGACCATGGGCTGGTACCGCCAGGCTCCAGGG | | |
| | AAGCAGCGCGAGTTGGTCGCAACTATTAGTAGTATTGGTATCTCAACC | | |
| | TATGCAAACTCCGTGAAGGGCCGATTCACCATCTCCAGAGACAATGCC | | |
| | AAGAACACAGTGTATCTACAAATGAACAGCCTGAAACCTGAGGACACG | | |
| | GCCGTCTATTTCTGTCATGTAATTCGGCCTAGTTGGATGCCGCAGTACT | | |
| | GGGGCCAGGGGACCCTGGTCACCGTCTCCTCAACTAGTGGCCCGGGA | | |
| | GGCCAACACCATCACCACCATCATGGCGCAGAACAAAAACTCATCTCA | | |
| | GAAGAGGATCTGTCTTAG | | |
| 39-S-sR2- | ATGAAATACCTATTGCCTACGGCGGCCGCTGGATTGTTATTACTCGCGG | 254 | 121 |
| 1F8 | CCCAGCCGGCCANNAAANCAGGTGCAGCTGCAGGAGTTTGGGGGAG | | |
| | GCTTGGTGCAGCCTGGGGGGTCTCTGAGACTCTCCTGTCAAGCCTCTA | | |
| | CAAGTGTCTTCGGTAACACTGCCATGGCCTGGTACCGCCAGGCTCCTG | | |
| | GGAAGCAGCGCGAGTTGGTCGCACGAATTACTACCCTTGGTTTCACAT | | |
| | ACTATGCAGACTCCGCGAAGGGCCGATTCACCATCTCTAGAGACAGCG | | |
| | CCATGAACACGGTGTATCTGCAAATGAACAGCCTGAAACCTGAGGACA | | |
| | CGGCCGTGTATTACTGTAATAGATTATGGCGGCCTCTAGCGTGGGGTC | | |
| | AGGGGACCCAGGTCACCGTCTCCTCAACTAGTGGCCCGGGAGGCCAA | | |
| | CACCATCACCACCATCATGGCGCAGAACAAAAACTCATCTCAGAAGAG | | |
| | GATCTGTCTTAG | | |
| 40-S-sR2-2G4 | ATGAAATACCTATTGCCTACGGCGGCCGCTGGATTGTTATTACTCGCGG | 255 | 122 |
| | CCCAGCCGGCCATGNAAACANAAAACAGCTGCAGGAGTCTGGGGGAG | | |
| | GCTTGGTGCAGCCTGGGGGGTCTCTGAGACTCTCCTGTGCAGCCTCTG | | |
| | GAATGCGCAGCAGTCTCGCTATCATGGGCTGGTACCGCCAGGCTCCAG | | |
| | GGAAGCAGCGCGAGTTGGTCGCAACTATTACTATTGGTGGTAACACAA | | |
| | ACTATGCAGACTCCGTGAAGGGCCGGTTCGCCATCTCCAGAGACAACA | | |
| | CCAAGCGCACGGTGTATCTGCAGATGAACAGCCTGACACCTGAGGACA | | |
| | CGGCCGTCTATTACTGTAATGTTCGAAGTTTCGTTAGAACCTACTGGGG | | |
| | CCAGGGGACCCAGGTCACCGTCTCCTCAACTAGTGGCCCGGGAGGCCA | | |
| | ACACCATCACCACCATCATGGCGCAGAACAAAAACTCATCTCAGAAGA | | |
| | GGATCTGTCTTAG | | |
| 47-S-sR2- | ATGAAATACCTATTGCCTACGGCGGCCGCTGGATTGTTATTACTCGCGG | 256 | 110 |
| 1F11 | CCCAGCCGGCCATGGCGCAGGTGCAGTTGCAGGAGTCTGGGGGAGGC | | |
| | TTGGTGCAGGCTGGGGGGTCTCTGAGACTCTCCTGTGCAGCCTCTGGA | | |
| | TTCACCTTCAGTAGCAGTTCCATGGGCTGGTACCGCCAGGCTCCAGGG | | |
| | AAGCAGCGCGAGTTGGTCGCTTCTATTATGCGTTATGGTACTACAACCT | | |
| | ATACAGACTCCGTGAAGGGCCGATTCACCATCTCCAGAGACAACGGCC | | |
| | AGAGAACAGTCTATCTGCAAATGAACAGCCTGAAGCCTGAGGACACG | | |
| | GCCGTCTATTATTGTAATGTTCGAAGTTTCGTTCGAACCTACTGGGGCC | | |
| | AGGGGACCCTGGTCACCGTCTCCTCAACTAGTGGCCCGGGAGGCCAAC | | |
| | ACCATCACCACCATCATGGCGCAGAACAAAAACTCATCTCAGAAGAGG | | |
| | ATCTGTCTTAG | | |
| 48-S-sR2- | ATGAAATACCTATTGCCTACGGCGGCCGCTGGATTGTTATTACTCGCGG | 257 | 123 |
| 2G9 | CCCAGCCGGCCATGGCGCAGGTGCAGTTGCAGCAGTCTGGGGGAGGC | | |
| | TTGGTGCAGGCTGGGGGGTCTCTGAGACTCTCCTGTGCAGCCTCTGGA | | |
| | AGCACCTTCATCAGTATCAAAACCATGGGCTGGTACCGCCAGGCTCCA | | |
| | GGGAAGCAGCGCGAGTTGGTCGCTGGTATTACTAAGAATAATTACATA | | |
| | AACTATGCAGACTCCGTGAAGGGCCGATTCACCATCTCCAGAGACAAC | | |
| | GGCAAGAATACAGTGTATCTGCAAATGAACGGCCTGAAACCTGAGGA | | |
| | CACGGCCGTCTATTACTGTACTGTACAACGTCGCTTAGGGCGTGTCTAC | | |
| | TGGGGCCAGGGGACCCTGGTCACCGTCTCCTCAACTAGTGGCCCGGGA | | |
| | GGCCAACACCATCACCACCATCATGGCGCAGAACAAAAACTCATCTCA | | |
| | GAAGAGGATCTGTCTTAG | | |
| 50-S-sR1-2B2 | ATGAAATACCTATTGCCTACGGCGGCCGCTGGATTGTTATTACTCGCGG | 258 | 124 |
| | CCCAGCCGGCCATGGCGCAGGTGCAGCTGCAGGAGTCTGGGGGAGGC | | |
| | TTGGTGCAGGCTGGGGGGTCTCTGAGACTCTCCTGTACGGCCTCTGGA | | |
| | AGCACCTTCAGGTTCAATGACATGGGCTGGTACCGCCAGGCTCCAGGG | | |
| | AAGCAGCGCGAATTGGTCGCAAATATTAATAGTAGTGGTAGAACCATG | | |
| | TATCCAGACTCCGTCAAGGGCCGATTCACAATCTCCAAAGACAACGTCA | | |
| | AAAATACAGTGTATCTGCAGATGAACAGCCTGACACCTGAGGACACGG | | |
| | CCGTCTATTACTGTAATGTTCGAAGTTTCGTTAGAACCTACTGGGGCCA | | |
| | GGGGACCCAGGTCACCGTCTCCTCAACTAGTGGCCCGGGAGGCCAAC | | |
| | ACCATCACCACCATCATGGCGCAGAACAAAAACTCATCTCAGAAGAGG | | |
| | ATCTGTCTTAG | | |
| 55-S-sR2- | ATGAAATACCTATTGCCTACGGCGGCCGCTGGATTGTTATTACTCGCGG | 259 | 125 |
| 1G3 | CCCAGCCGGCCATGGCGCAGGTGCAGCTGCAGGAGTCTGGGGGAGGC | | |
| | TTGGTCCAGGCTGGGGGGTCTCTGACGCTCTCCTGTGTAGCCTCTGGA | | |
| | AGCCGCTTCAGTATCAATACCATGGGCTGGTACCGCCAGGCTCCAGGG | | |
| | AAGCAGCGCGAGTTGGTCGCAGGTATTACTAGCCTTGGTTTCACAAAC | | |
| | TATGCAGACTCCGTGAAGGGCCGATTCACCATCTCCAGAGACAACGCC | | |
| | AAGAACACAGTGTATCTGCAAATGAACAACCTGAAAGTTGAGGACACG | | |
| | GCCGTCTATTACTGTAACCGCAGAGGATTTCGGAGCTACTGGGGCCAG | | |
| | GGGACCCTGGTCACCGTCTCCTCAACTAGTGGCCCGGGAGGCCAACAC | | |
| | CATCACCACCATCATGGCGCAGAACAAAAACTCATCTCAGAAGAGGAT | | |
| | CTGTCTTAG | | |
| 56-S-sR2- | ATGAAATACCTATTGCCTACGGCGGCCGCTGGATTGTTATTACTCGCGG | 260 | 126 |
| 2H1 | CCCAGCCGGCCATGGCGCAGGTGCAGCTCGTGGAGTCTGGGGGAGGC | | |
| | TTCGTGCAGGCTGGGGGGTCTCTGAGACTCTCCTGTGCAGCCTCTGGA | | |
| | AGCATCTTCAGTATCAATTATGGTAACTGGTACCGCCAGGCTCCAGGG | | |
| | AAGCAGCGCGAATTGGTCGCAGGTATTAGTCGTGGAGGCCGCACAAA | | |
| | GTATGCAGACTCCGTGAAGGGCCGATTCACCATCTCCAGAGATAGCGC | | |
| | CAAGACACTGACGCTGCAGATGACTAGCTTGAAACCTGAGGACACGGC | | |
| | CGTCTATTACTGTAATGTTCGAAGTTTCGTTCGAACCTACTGGGGCCAG | | |
| | GGGACCCAGGTCACCGTTTCCTCAACTAGTGGCCCGGGAGGCCAACAC | | |
| | CATCACCACCATCATGGCGCAGAACAAAAACTCATCTCAGAAGAGGAT | | |
| | CTGTCTTAG | | |
| 58-S-sR1-2B4 | ATGAAATACCTATTGCCTACGGCGGCCGCTGGATTGTTATTACTCGCGG | 261 | 127 |
| | CCCAGCCGGCCATGGCGCAGGTGCAGCTGCAGGAGTCTGGGGGAGGC | | |
| | TTGGTGCAGTCGGGGGGGTCTCTGAGACTCTCCTGTTCGGCCTCCGGA | | |
| | AGCATCTTCAGGATCAATCTCATGGGCTGGTACCGCCAGGCTCCAGGG | | |
| | AAGCAGCGCGAGTTGGTCGCAACTATTACTAATGAAGGTAACACATAC | | |
| | TACGCAGACTCCGTGAAGGGCCGTTTCACCATCTCCAGAGACAACGCC | | |
| | AACAACACGTGGTATCTGCAAATGAACAGCCTGAAACCTGAGGACACA | | |
| | GCCGTCTATGAATGTGCAGGAAAGGTCATTAGATGGTACTGGGGCCA | | |
| | GGGGACCCAGGTCACCGTTTCCTCAACTAGTGGCCCGGGAGGCCAACA | | |
| | CCATCACCACCATCATGGCGCAGAACAAAAACTCATCTCAGAAGAGGA | | |
| | TCTGTCTTAG | | |
| 62-S-sR2- | ATGAAATACCTATTGCCTACGGCGGCCGCTGGATTGTTATTACTCGCGG | 262 | 128 |
| 1A9 | CCCAGCCGGCCATGGCGCAGGTGCAGCTGCAGGAGTTTGGGGGAGGC | | |
| | TTGGTGCAGCCTGGGGGGTCTCTGAGACTCTCCTGTCAAGCCTCTACA | | |
| | AGTGTCTTCGGTAACACTGCCATGGCCTGGTACCGCCAGGCTCCTGGG | | |
| | AAGCAGCGCGAGTTGGTCGCACGAATTACTACCCTTGGTTTCACATACT | | |
| | ATGCAGACTCCGCGAAGGGCCGATTCACCATCTCTAGAGACAGCGCCA | | |
| | TGAACACGGTGTATCTGCAAATGAACAGCCTGAAACCTGAGGACACGG | | |
| | CCGTTTATTACTGTCGTGCACGTCGCGCTCTGCGAGAATCGCACTGGG | | |
| | GCCAGGGGACCCAGGTCACCGTCTCCTCAACTAGTGGCCCGGGAGGC | | |
| | CAACACCATCACCACCATCATGGCGCAGAACAAAAACTCATCTCAGAA | | |
| | GAGGATCTGTCTTAG | | |
| 63-S-sR2- | ATGAAATACCTATTGCCTACGGCGGCCGCTGGATTGTTATTACTCGCGG | 263 | 129 |
| 1G9 | CCCAGCCGGCCATGGCGCAGGTGCAGCTGCAGGAGTCTGGGGGAGGC | | |
| | TTCGTGCAGGCTGGGGGGTCTCTGAGACTCTCCTGTGCAGCCTCTGGA | | |
| | AGCATCTTCAGTATCAATTATGGTAACTGGTACCGCCAGGCTCCAGGG | | |
| | AAGCAGCGCGAATTGGTCGCAGGTATTAGTCGTGGAGGCCGCACAAA | | |
| | GTATGCAGACTCCGTGAAGGGCCGATTCACCATCTCCAGAGATAGCGC | | |
| | CAAGACACTGACGCTGCAGATGACTAGCTTGAAACCTGAGGACACGGC | | |
| | CATCTATTCTTGTAATGCTCGAAGTTTCGTTAGAACTTACTGGGGCCAG | | |
| | GGGACCCTGGTCACCGTCTCCTCAACTAGTGGCCCGGGAGGCCAACAC | | |
| | CATCACCACCATCATGGCGCAGAACAAAAACTCATCTCAGAAGAGGAT | | |
| | CTGTCTTAG | | |
| 64-S-sR2-2H2 | ATGAAATACCTATTGCCTACGGCGGCCGCTGGATTGTTATTACTCGCGG | 264 | 130 |
| | CCCAGCCGGCCATGGCGCAGGTGCAGCTGCAGGAGTCTGGGGGAGGC | | |
| | TTGGTGACGGCTGGAGGGTCTCTGAGACTCTCCTGTGCAGCCTCTAGA | | |
| | AACTTCTTCACTTTCAGAGCCATGGGCTGGTACCGCCAGGCTCCAGGG | | |
| | AAGCAGCGCGAAATGGTCGCATCTATTACTACCGGTGGTCGCACCGTC | | |
| | TATGCAGACTCCGTGAAGGGCCGATTCACCATCTCCAAATCCAACGCCA | | |
| | ATAACACAGTGTATCTCCAAATGAACAGCCTGGAAGCTGAGGACACGG | | |
| | CCGTCTATTACTGTAATGCACGACGCAGATTTCCGGTGCCGGGCCCGA | | |
| | CCGACTACTGGGGCCGGGGGACCCTGGTCACCGTCTCCTCAACTAGTG | | |
| | GCCCGGGAGGCCAACACCATCACCACCATCATGGCGCAGAACAAAAAC | | |
| | TCATCTCAGAAGAGGATCTGTCTTAG | | |
| 66-S-sR1- | ATGAAATACCTATTGCCTACGGCGGCCGCTGGATTGTTATTACTCGCGG | | |
| 2B10 | CCCAGCCGGCCATGGCGCAGGTGCAGCTGCAGGAGTCTGGGGGAGGC | 265 | 131 |
| | TTGGTGCAGGCTGGGGGGTCTCTGAGACTCTCCTGTGCAGCCTCTGGA | | |
| | ATCACCTTCAGGTTCAATGCCATGGGCTGGTACCGCCAGGCTCCAGGG | | |
| | AAAGAGCGCGAGTTGGTCGCAAGGGTTAGTAGTGGTGGTAGCACAAC | | |
| | CTATGCAGACTCCGTGAAGGCCCGATTCACCACCTTCAGAGACAACGT | | |
| | CAAGAACATAGGGTATCTGCAAATGACCAGCCTGAAACCTGAGGACAC | | |
| | GGCCGTCTATTACTGTAATGTGGGGAATTTCTGGGGCCAGGGGACCCA | | |
| | GGTCACCGTCTCCTCAACTAGTGGCCCGGGAGGCCAACACCATCACCA | | |
| | CCATCATGGCGCAGAACAAAAACTCATCTCAGAAGAGGATCTGTCTTA | | |
| | G | | |
| 70-S-sR2-1B3 | ATGAAATACCTATTGCCTACGGCGGCCGCTGGATTGTTATTACTCGCGG | 266 | 132 |
| | CCCAGCCGGCCATGGCGCAGGTGCAGCTGCAGGAGTCTGGGGGAGGC | | |
| | TTGGTGCGGACTGGGGAGTCTCTGGGACTCTCCTGTGCAGCCTCTGGA | | |
| | CGCAGCATCCTGATCAAAGGCATGGGCTGGTACCGCCAGGCTCCAGG | | |
| | GAAGGAGCGCGAAATGGTCGCGACTATTAGTATGGCCGGTGTCACTA | | |
| | ACTATTCAGACTCCGTGAAGGGCCGGTTCACCATCTCCAGAGATAACT | | |
| | ACAAGAAGACAGTGTCCCTGCAGATGAACAATTTGAGACCGGAGGAC | | |
| | ACGGCCGTCTATGTGTGTAATGCACAAACCCGACTCTGGAGCTACTGG | | |
| | GGCCAGGGGACCCAGGTCACCGTCTCCTCAACTAGTGGCCCGGGAGG | | |
| | CCAACACCATCACCACCATCATGGCGCAGAACAAAAACTCATCTCAGA | | |
| | AGAGGATCTGTCTTAG | | |
| 71-S-sR2-2A8 | ATGAAATACCTATTGCCTACGGCGGCCGCTGGATTGTTATTACTCGCGG | 267 | 133 |
| | CCCAGCCGGCCATGGCGCAGGTGCAGCTCGTGGAGTCTGGGGGAGGC | | |
| | CTGGTGCAGGCTGGAGGGTCTCTGAGACTCTCCTGTGCAGCCTCTGGA | | |
| | AGGATTTTCGGGCGCAATGCCATGGCCTGGTACCGCCAGGTTCCAGGG | | |
| | AAGGAGCGCGAGCTGGTTGCACGTATTACTAGGGATGGACGGACAAT | | |
| | GTATGTAGACTCCGCGAAGGGACGATTCACCATCTCCAGGGACAACGC | | |
| | CAAGAACACAGCGTATCTGCACATGGACAGCCTGAAACCTGAGGACAC | | |
| | GGCCGTCTATTACTGTAATGCACAAACCCGACTCTGGAGCTACTGGGG | | |
| | CCAGGGGACCCAGGTCACCGTCTCCTCAACTAGTGGCCCGGGAGGCCA | | |
| | ACACCATCACCACCATCATGGCGCAGAACAAAAACTCATCTCAGAAGA | | |
| | GGATCTGTCTTAG | | |
| 72-S-sR2- | ATGAAATACCTATTGCCTACGGCGGCCGCTGGATTGTTATTACTCGCGG | 268 | 134 |
| 2H3 | CCCAGCCGGCCATGGCGCAGNNNNCAGCTGCAGGAGTCTGGGGGAG | | |
| | GCTTGGTGCAGGCTGGGGGGTCTCTGAGACTCTCCTGTGCAGCCTCTA | | |
| | GAAGCACCTTCAGATTCAATGTCATGGGCTGGTACCGCCAGGCTCCAG | | |
| | GGAAGCAGCGCGAGTTGGTCGCAGCTATTAGTAGTCGTGGTGGTAGT | | |
| | ACAAACTATGCAGACTCCGTGCAGGGCCGATTCACCATCTCCAGAGAC | | |
| | AACGCCAAGAACACAGTGTCTCTGCAAATGAACAGCCTGAAACCTGAG | | |
| | GACACGGCCGTCTATTACTGTAATGTTCGAAGTTTCGTTAGAACCTACT | | |
| | GGGGCCAGGGGACCCAGGTCACCGTCTCCTCAACTAGTGGCCCGGGA | | |
| | GGCCAACACCATCACCACCATCATGGCGCAGAACAAAAACTCATCTCA | | |
| | GAAGAGGATCTGTCTTAG | | |
| 74-S-sR1- | ATGAAATACCTATTGCCTACGGCGGCCGCTGGATTGTTATTACTCGCGG | 269 | 135 |
| 2B12 | CCCAGCCGGCCATGGCGCAGGTGCAGCTGCAGGAGTTTGGGGGAGGC | | |
| | TTGGTGCAGGCTGGGGGGTCTCTGAGACTCTCCTGTGCAACCTCTGGA | | |
| | AGCATCTTCAGTATCAACGCCGTGGGCTGGTACCGCCAGGCTCCAGGG | | |
| | AATCAGCGCGAGTTGGTCGCAGCTATTAGTGGACGTGGTAGTACACAC | | |
| | TATGCAGACTCCGTGAAGGGCCGATTCACCATCTCCAGAGACACCGCC | | |
| | AAGAACACGGTGTATCTGCAAATGAACAGCCTGAAACCTGAGGACACA | | |
| | GCCGTCTATTACTGTGCATTAGATCAACATATGGAGGTTATTGTATCGC | | |
| | CGGGACGTATTGGTTCCTGGGGCCAGGGGACCCTGGTCACCGTCTCCT | | |
| | CAACTAGTGGCCCGGGAGGCCAACACCATCACCACCATCATGGCGCAG | | |
| | AACAAAAACTCATCTCAGAAGAGGATCTGTCTTAG | | |
| 78-S-sR2-1C9 | ATGAAATACCTATTGCCTACGGCGGCCGCTGGATTGTTATTACTCGCGG | 270 | 136 |
| | CCCAGCCGGCCATGGCGCAGGTGCAGTTGCAGGAGTCTGGGGGAGGC | | |
| | TTGGTGCAGGCTGGGGAGTCTCTGACACTCTCCTGTGCACTCTCAACAA | | |
| | CCATGTTCGGTTTCTGGCCCATGGCCTGGTTCCGCCAGACTCCAGGACA | | |
| | GCGGCGCGAATTGATTGCGACTATTGATAGTCGTGGTCGCACAAACAT | | |
| | CGCAGACTCCGTGAAGGGCCGATTTACCATCTCCAGAGACAACACCAA | | |
| | GAACACACTGTATCTGCGGATGAACAGCCTGAAACCTGAGGACACGGC | | |
| | CGTCTATTACTGTAATGCCCAGCGGAGGTGGCCTCTTCGTGACTATTGG | | |
| | GGCCAGGGGACCCAGGTCACCGTCTCCTCAACTAGTGGCCCGGGAGG | | |
| | CCAACACCATCACCACCATCATGGCGCAGAACAAAAACTCATCTCAGA | | |
| | AGAGGATCTGTCTTAG | | |
| 79-S-sR2- | ATGAAATACCTATTGCCTACGGCGGCCGCTGGATTGTTATTACTCGCGG | 271 | 137 |
| 2B3 | CCCAGCCGGCCATGGCGCAGGTGCAGCTGCAGGAGTTTGGGGGAGGC | | |
| | GTGGTGCAGTCTGGGGGGTCTCTGAGACTCTCCTGTGTAGCCTCTGGA | | |
| | AACATCTTCAGGATCAATGCCATGGGCTGGTACCGCCAGGCTCCAGGG | | |
| | AAGCAGCGCGAGTTGGTCGCCCGTATTAGTAGTGGTGGTAGCACAAA | | |
| | CTATGCAGACTCCGTGAAGGGCCGATTCACCATCTCCAGAGACAACGT | | |
| | CAAGAACACAGTGACTCTGCAAATGAACAGCCTGAAACCTGAGGACAC | | |
| | GGCCGTCTATTACTGTAATGCGCGGAGGCCATTGCGTTGGTATGAGTA | | |
| | CTGGGGCCAGGGGACCCTGGTCACCGTCTCCTCAACTAGTGGCCCGGG | | |
| | AGGCCAACACCATCACCACCATCATGGCGCAGAACAAAAACTCATCTC | | |
| | AGAAGAGGATCTGTCTTAG | | |
| 80-S-sR2- | ATGAAATACCTATTGCCTACGGCGGCCGCTGGATTGTTATTACTCGCGG | 272 | 138 |
| 2H5 | CCCAGCCGGCCATGGCGCAGGTGCAGCTGCAGGCGTCTGGGGGAGGC | | |
| | TTGGTGCAGCCTGGGGGATCTCTGAGACTCTCCTGTGCAGCCTCGGGA | | |
| | AGCATCTTCAGTACCAATGCCATGGGCTGGTACCGCCAGGCTCCAGGG | | |
| | AAGCAGCGCGAGGTGATCGCATCTATTACAAAATTTGGGAACACAGAC | | |
| | TATGCAGACTCTGTGAAGGGCCGATTCACCATCTCCAGAGACAACGCC | | |
| | AAGAACATAGTGTATCTGCAAATGAACAGCCTGAAACCTGAGGACACG | | |
| | GCCGTTTATTACTGTTATCAAAACAGTCGGGGGCGCTGGTATGATATTT | | |
| | TCAGGGACTACTGGGGCCAGGGGACCCTGGTCACCGTCTCCTCAACTA | | |
| | GTGGCCCGGGAGGCCAACACCATCACCACCATCATGGCGCAGAACAA | | |
| | AAACTCATCTCAGAAGAGGATCTGTCTTAG | | |
| 80-S-sR2- | >80-S-sR2-2H9 | 273 | 139 |
| 2H9 | ATGAAATACCTATTGCCTACGGCGGCCGCTGGATTGTTATTACTCGCGG | | |
| | CCCAGCCGGCCATGGCGCAGGTGCAGCTCGTGGAGTCTGGGGGTGGC | | |
| | TTCGTGCAGGCAGGGGGATCTCTAAGACTCTCCTGTGTAGCCTCGCGA | | |
| | AGCAGCTTCAGGATCACTACCATGAACTGGTACCGCCAGGCTCCAGGG | | |
| | AAGCAGCGCGAAATGGTCGCATCTATTACTACCGGTGGTCGCACCGTC | | |
| | TATGCAGACTCCGTGAAGGGCCGATTCACCATCTCCAAATCCAACGCCA | | |
| | ATAACACAGTGTATCTCCAAATGAACAGCCTGGAAGCTGAGGACACGG | | |
| | CCGTCTATTACTGTAATGCCCAGCGGAGGTGGCCTCTTCGTGACTATTG | | |
| | GGGCCAGGGGACCCTGGTCACCGTCTCCTCAACTAGTGGCCCGGGAG | | |
| | GCCAACACCATCACCACCATCATGGCGCAGAACAAAAACTCATCTCAG | | |
| | AAGAGGATCTGTCTTAG | | |
| 82-S-sR1- | ATGAAATACCTATTGCCTACGGCGGCCGCTGGATTGTTATTACTCGCGG | 274 | 140 |
| 2C11 | CCCAGCCGGCCATGGCGCAGGTGCAGTTGCAGGAGTCTGGGGGAGGC | | |
| | GTGGTGCAGTCTGGGGGGTCTCTGAGACTCTCCTGTGTAGCCTCTGGA | | |
| | AACATCTTCAGGATCAATGTCATGGGCTGGTACCGCCAGGCTCCAGGG | | |
| | AAGCAGCGCGAGTTGGTCGCGGTTGTAAAGAGTGGTGGTAGCACAAA | | |
| | CTATGTAGACTCCGCGAAGGGACGATTCACCATCTCCAGGGACAACGC | | |
| | CAAGAACACAGCGTATCTGCACATGGACAGCCTGAAACCTGAGGACAC | | |
| | GGCCGTCTATTACTGTAATGCACAAACCCGACTCTGGAGCTACTGGGG | | |
| | CCAGGGGACCCTGGTCACCGTCTCCTCAACTAGTGGCCCGGGAGGCCA | | |
| | ACACCATCACCACCATCATGGCGCAGAACAAAAACTCATCTCAGAAGA | | |
| | GGATCTGTCTTAG | | |
| 86-S-sR2-1D5 | ATGAAATACCTATTGCCTACGGCGGCCGCTGGATTGTTATTACTCGCGG | 275 | 141 |
| | CCCAGCCGGCCATGGCGCAGGTGCAGCTGCAGGAGTTTGGGGGAGGC | | |
| | GTGGTGCAGTCTGGGGGGTCTCTGAGACTCTCCTGTGTAGCCTCTGGA | | |
| | AACATCTTCAGGATCAATGCCATGGGCTGGTACCGCCAGGCTCCAGGG | | |
| | AAGCAGCGCGAGTTGGTCGCACATATTATTAGTGGTGGTAGCACAAAC | | |
| | TATGCAGACTCCGTGAAGGGCCGATTCACCATCTCCAGAGAATACGCC | | |
| | AAGAATATGGTGTATCTGCAAATGAACAGCCTGAAACCTGAGGACACG | | |
| | GCCGTCTATTACTGTAATGCCGAAAGGAGATTCGGGATGAGACAGGTC | | |
| | TGGGGCCAGGGGACCCAGGTCACCGTCTCCTCAACTAGTGGCCCGGG | | |
| | AGGCCAACACCATCACCACCATCATGGCGCAGAACAAAAACTCATCTC | | |
| | AGAAGAGGATCTGTCTTAG | | |
| 87-S-sR2-2D8 | ATGAAATACCTATTGCCTACGGCGGCCGCTGGATTGTTATTACTCGCGG | 276 | 142 |
| | CCCAGCCGGCCATGGCGCAGGTGCAGTTGCAGGAGTCTGGGGGAGGC | | |
| | TTGGTGCAGGCTGGGGGGTCTCTGAGACTCTCCTGTGCAGCCTCTGTG | | |
| | GTTCCCTTCAGATACTTTCCCATGGGCTGGTACCGCCAGGCTCCAGGGA | | |
| | GACAGCGCGAGTTGGTCGCGTCTATTACCAGCGGTGGTGGCGTAAACT | | |
| | ATGCAGATTTCGTAGAGGGCCGATTCACCATCTCCAGAGACAATGCCA | | |
| | AGAACACATTTTATCTACAAATGAGCAGCCTGAAACCTGAGGACACGG | | |
| | CCGTCTATTACTGTGCACGACTTCTCAGTCTGGGTAGTAGGTGGGGAT | | |
| | ACGGCATGTTCACCTGGGGCAAAGGGACCCTGGTCACCGTCTCCTCAA | | |
| | CTAGTGGCCCGGGAGGCCAACACCATCACCACCATCATGGCGCAGAAC | | |
| | AAAAACTCATCTCAGAAGAGGATCTGTCTTAG | | |
| 88-S-sR2- | ATGAAATACCTATTGCCTACGGCGGCCGCTGGATTGTTATTACTCGCGG | 277 | 143 |
| 2H8 | CCCAGCCGGCCATGGCGCAGGTGCAGNTNCAGGAGTCTGGGGGAGG | | |
| | CTTGGTGCAGCCTGGGGGGTCTCTGAGACTCTCCTGTGCAGCCTCTGG | | |
| | AAGCATCTTCAGTATCAAGACCATGGGCTGGTACCGCCAGGCTCCAGG | | |
| | GAAGCAGCGCGAGTTGGTCGCTGCTATAGCTAGTGGTGGTTTCACAAA | | |
| | CTATGCAGACTCCGTGAAGGGCCGATTCACCATCTCCAGAGACAACGC | | |
| | CAGGAACACGGTGTATCTGCAAATGAACAGCCTGAAACCTGAGGACAC | | |
| | GGCCGTCTATTACTGTAATGCCCAGCGGAGGTGGCCTCTTCGTGACTAT | | |
| | TGGGGCCAGGGGACCCTGGTCACCGTTTCCTCAACTAGTGGCCCGGGA | | |
| | GGCCAACACCATCACCACCATCATGGCGCAGAACAAAAACTCATCTCA | | |
| | GAAGAGGATCTGTCTTAG | | |
| 94-S-sR2- | ATGAAATACCTATTGCCTACGGCGGCCGCTGGATTGTTATTACTCGCGG | 278 | 144 |
| 1D10 | CCCAGCCGGCCATGGCGCAGGTGCAGNTGCAGNAGTCAGGGGGAGG | | |
| | CTTGGTACGGGACGGGGGGTCTCTGACACTCTCCTGTGCAGCCTCTGG | | |
| | AAGTGCCTTCAGGATGAATTCCATGGCCTGGTACCGCCAGGTTCCTGG | | |
| | GAAACAGCGCGAGTTAGTCGCAGCTATTAGCTTCCGTGGGAGCGCAA | | |
| | ATTATGCTAACTCCGTGAAGGGCCGATTCACCATCTCCAGAGACAACG | | |
| | GCAAGAACACGGTATATCTACAAATGAACAGCCTGAAACCTGAGGACA | | |
| | CAGCCGTCTATTACTGTGCAGCAGGCCGTCCATGGCAAAGGACTTTCT | | |
| | ACTGGGGCCAGGGGACCCAGGTCACCGTCTCCTCAACTAGTGGCCCGG | | |
| | GAGGCCAACACCATCACCACCATCATGGCGCAGAACAAAAACTCATCT | | |
| | CAGAAGAGGATCTGTCTTAG | | |
| 95-S-sR2- | ATGAAATACCTATTGCCTACGGCGGCCGCTGGATTGTTATTACTCGCGG | 279 | 145 |
| 2D10 | CCCAGCCGGCCATGGCGCAGGTGCAGTTGCAGGAGTCTGGGGGAGGC | | |
| | TTCGTGCAGGCTGGGGGGTCTCTGAGACTCTCCTGTGCAGCCTCTGGA | | |
| | AGCATCTTCAGTATCAATTATGGTAACTGGTACCGCCAGGCTCCAGGG | | |
| | AAGCAGCGCGAATTGGTCGCAGGTATTAGTCGTGGAGGCCGCACAAA | | |
| | GTATGCAGACTCCGTGAAGGGCCGATTCACCATCTCCAGAGATAGCGC | | |
| | CAAGACACTGACGCTGCAGATGACTAGCTTGAAACCTGAGGATTCGGG | | |
| | CGTCTACTACTGTGCTGCGACCCGCTGGAGTTGGGGTACTAAGAGTTA | | |
| | CTGGGGCCAGGGAACCCAGGTCACCGTCTCCTCAACTAGTGGCCCGGG | | |
| | AGGCCAACACCATCACCACCATCATGGCGCAGAACAAAAACTCATCTC | | |
| | AGAAGAGGATCTGTCTTAG | | |
| S-sR2-2H7 | ATGAAATACCTATTGCCTACGGCGGCCGCTGGATTGTTATTACTCGCGG | 280 | 221 |
| | CCCAGCCGGCCATGGCGCAGGTGCAGCTGCAGGAGTTTGGGGGAGGC | | |
| | GTGGTGCAGTCTGGGGGGTCTCTGAGACTCTCCTGTGTAGCCTCTGGA | | |
| | AACATCTTCAGGATCAATGCCATGGGCTGGTACCGCCAGGCTCCAGGC | | |
| | AAGTCACGCGTACTGGTCGCAAGCATTGATAGTGCCGGTAGGACAAAC | | |
| | TATGGTGACGCCGTAGAGGATCGATTCACCATCTCCAGAGACATCGCC | | |
| | AACAACATAGTGAATCTACAGATGAATAGCCTAAAACCTGAGGACACG | | |
| | GCCGTCTATTACTGTTCTACATGGCGACTAGGACGCAACTACTGGGGC | | |
| | CAGGGGACCCAGGTCACCGTCTCCTCAACTAGTGGCCCGGGAGGCCA | | |
| | ACACCATCACCACCATCATGGCGCAGAACAAAAACTCATCTCAGAAGA | | |
| | GGATCTGTCTTAG | | |
| 90-SsR1- | ATGAAATACCTATTGCCTACGGCGGCCGCTGGATTGTTATTACTCGCGG | | |
| 2D1 | CCCAGCCGGCCATGGCGCAGGTGCAGTTGCAGGAGTCTGGGGGAGGC | 281 | 343 |
| | TTGGTGCAGGCTGGGGGCTCTCTGAGACTCTCCTGTGCAGCCTCTGGA | | |
| | AGCATCTTCCGTATCAATGGCATGGGCTGGATCCGCCAGGCTCCAGGG | | |
| | AAGGAGCGTGAGGTTGTAGCAGCCGTTAACTGGAGTGGTGAACGCAC | | |
| | ATACTATGTTGACTCCGTGAAGGGCCGATTCACCATCTCCAGAGAAAA | | |
| | AGGCAACAGGATATATCTACAAATGAACGATTTGGAACCTGACGACAC | | |
| | GGCCGTTTATTACTGTGCAGCAGATACGGATTACCGTTTAGACGGTAG | | |
| | TACGTGGATTACCAACCTCTACTCTGGGTCCTTGGGCCAGGGGACCCA | | |
| | GGTCACCGTCTCCTCAACTAGTGGCCCGGGAGGCCAACACCATCACCA | | |
| | CCATCATGGCGCAGAACAAAAACTCATCTCAGAAGAGGATCTGTCTTA | | |
| | G | | |
| S-sR2-1F6 | CAGGTGCAGCTGCAGGAGTCTGGGGGAGGCTTGGTGCAGGCTGGGG | 282 | 225 |
| | GGTCTCTGAGACTCTCCTGTGCAGCCTCTGGAAGCACCTTCAGTAACAA | | |
| | TGCCATGGCCTGGTACCGCCAGGCTCCAGGGAAGCAGCGCGAGTTGG | | |
| | TCGCCTATATTAGTAGTGGTGGTTTCACAAATTATGGCGACTCCGTGAA | | |
| | GGGCCGATTCACCATCTCCGAAGACAACGCCAAGAGTACAGTGTATCT | | |
| | ACAAATGACCAGCCTGAAACCTGAGGACACGGCCGTCTATTATTGTAG | | |
| | CGCCGGGGGTACATACCGTAGTGGTAATGTCTACTTCTTTCCGCGTTCC | | |
| | TGGGGCCAGGGGACCCAGGTCACCGTCTCCTCAACTAGTGGCCCGGG | | |
| | AGGCCAA | | |
| S-sR2-1G4 | CAGGTGCAGTTGCAGCAGTCTGGGGGAGGGTTGGTGCAGCCTGGGG | 283 | 229 |
| | GGTCTCTGAGACTCTCCTGTGCAGCCTCTGGAAGCATCTTCAGTATCAA | | |
| | TTCCATGGCCTGGTACCGCCAGGCTCCAGGGAACCAGCGCGAGTTGGT | | |
| | CGCGACTATTAGTAGTCGTAGTACCACGTACTATGCGCCTTCCGTGAAG | | |
| | GGCCGGTTCACCATCTCCAGAGACAACGCCAAGAACATAGTGTACCTG | | |
| | CAAATGAACAGCCTCAAACCTGAGGACACGGCCGTGTATTACTGTAAG | | |
| | GCGGGTTCAGTGGGTCGCGTGTACTGGGGCCAGGGGACCCTGGTCAC | | |
| | CGTCTCCTCAACTAGTGGCCCGGGAGGCCAA | | |
| S-sR2-2C10 | CAGGTGCAGTTGCAGGAGTCTGGGGGAGGCTTGGTGCAGGCTGGGG | 284 | 233 |
| | GGTCTCTGAGACTCTCCTGTTTAGCCTCTATGACTACCCTCGGGTTCAA | | |
| | GACCATGGGCTGGTACCGCCAGGCTCCAGGGAAGCAGCGCGAGTTGG | | |
| | TCGCCGCTATTACGAGTGGTGGTACCGCAAACTATGCAGACTCCGTGA | | |
| | AGGGCCGATTCGCCATCTCCAGAGAGAACGCCAAGAACACGCTGTATC | | |
| | TGCAAATGAACAGCCTGAAACCTGAGGACACGGCCCTGTATTACTGTG | | |
| | CATCGACTACGGGTTGGACAGAGGTCGGCGGACGAAATGACTACTGG | | |
| | GGCCAGGGGACCCTGGTCACCGTTTCCTCAACTAGTGGCCCGGGAGGC | | |
| | CAA | | |
| S-sR2-2E9 | CAGGTGCAGCTGCAGGAGTCTGGGGGAGGATTGGTGCAGACTGGGG | 285 | 237 |
| | GGTCTCTTAGACTCTCCTGTGCGGCCTCTGGGCGCACCTTCAGAGTCAA | | |
| | TGCCATGGGCTGGTACCGCCAGGCTCCAGGGAAGCAGCGCGAGTTCG | | |
| | TCGCAGCTGTTACAAATGGTGGTAGTACAACCTATGCAGATTCCGTGA | | |
| | AGGGCCGATTCACCATCTCCAGGGACAACGCCAAGAATACAATATATC | | |
| | TGCAAATGAACAGACTGGAACCTGAAGACACGGCCCTCTATTATTGTA | | |
| | ATGCCGAAAGGAGATTCGGGATGAGACAGGTCTGGGGCCAGGGGAC | | |
| | CCTGGTCACCGTTTCCTCAACTAGTGGCCCGGGAGGCCAA | | |
| S-sR2-2G11 | CAGGTGCAGCTGCAGGAGTCTGGGGGAGGCTTGGTGCAGCCTGGGG | 286 | 241 |
| | GGTCTCTGAGACTCTCCTGTGCAGCCTCTGGAAGAGTCTTCAGTATCAA | | |
| | TACCATGGGCTGGTACCGCCAGGCTCCAGGGAAGCAGCGCGAGTTGG | | |
| | TCGCATCTATGACTAGAGGTGGTAGCGCAAATTATGCAGACTCCGTGA | | |
| | AGGGCCGATTCACCACATCCAGAGACAACGCCAAGAACATGGTGTATC | | |
| | TGCAAATGAATAGACTGAAAGCTGAGGACACGGCCGTCTATTACTGTA | | |
| | ATGCAGCTCGGGGTTGGAGGATCTACTGGGGCAAAGGGACCCTGGTC | | |
| | ACCGTTTCCTCAACTAGTGGCCCGGGAGGCCAA | | |

TABLE 4
Polynucleotide Sequences of Synuclein
Antibodies Enriched by Solution Phase Panning

| | | NA | AA |
|---|---|---|---|
| | | SEQ | SEQ |
| Antibody | | ID | ID |
| ID | Nucleotide Sequence | NO: | NO: |
| 02-S-bR1- | ATGAAATACCTATTGCCTACGGCGGCCGCTGGATTGTTATTACTCGCGGC | 287 | 191 |
| 2C7 | CCAGCCGGCCATGGCGCAGGTGCAGCTGCAGGAGTCTGGGGGAGGCTT | | |
| | GGTGCAGCCCGGGGACTCTCTGAAGCTCTCTTGTGCAGCCTCTGGCGGC | | |
| | ACCTTCGGTGCCGGTGTCGTGGCCTGGTACCGCCAGTCTCCAGGGAAAC | | |
| | AGCGTGAGATGGTCGGAAGTATGGGTAGTGATGGTTTCACGCAAATCGA | | |
| | AAACGGCATGAAGGGCCGATTCACTATCTCCGGGGCCGGCGACAAGAAA | | |
| | ACAGTGTTTTTACAGATGAACAATTTGAAGCCTGAGGACACGGCCGTCTA | | |
| | TTTCTGTCATTACGCCGATGGCCGGTTTGGCTCTTGGGGTCAGGGGACCC | | |
| | AGGTCACCGTCTCCTCAACTAGTGGCCCGGGAGGCCAACACCATCACCAC | | |
| | CATCATGGCGCAGAACAAAAACTCATCTCAGAAGAGGATCTGTCTTAG | | |
| 04-S-bR2- | ATGAAATACCTATTGCCTACGGCGGCCGCTGGATTGTTATTACTCGCGGC | 288 | 192 |
| 1C4 | CCAGCCGGCCATGGCGCAGGTGCAGCTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCAG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 20-S-bR2- | ATGAAATACCTATTGCCTACGGCGGCCGCTGGATTGTTATTACTCGCGGC | 289 | 192 |
| 1C9 | CCAGCCGGCCATGAAACAGGTGCAGCTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCAG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 27-S-bR2- | ATGAAATACCTATTGCCTACGGCGGCCGCTGGATTGTTATTACTCGCGGC | 290 | 192 |
| 1B1 | CCAGCCGGCCATGAAACAGGTGCAGCTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCAG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 28-S-bR2 | ATGAAATACCTATTGCCTACGGCGGCCGCTGGATTGTTATTACTCGCGGC | 291 | 192 |
| 1C12 | CCAGCCGGCCATGGCGCAGGTGCAGCTGCAGGAGTCTGGGGGAGGCTT | | |
| | GGTCCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACCACCAATTATCTA | | |
| | GCCTCCGTGAAGGGCCGATTCACTATCTCCAGAGACAACGCCAAGAACA | | |
| | CGGTGTCGCTACAAATGAACAGCCTGAAACCTGAGGACACGGCCGTCTA | | |
| | CACTTGTTATGCAGCCTGGCCGTTGAACACTTGGGGCCAGGGGACCCAG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 38-S-bR2- | ATGAAATACCTATTGCCTACGGCGGCCGCTGGATTGTTATTACTCGCGGC | 292 | 192 |
| 1H7 | CCAGCCGGCCATGNNNCAGGTGCAGCTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCAG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 44-S-bR2- | ATGAAATACCTATTGCCTACGGCGGCCGCTGGATTGTTATTACTCGCGGC | 293 | 192 |
| 1D2 | CCAGCCGGCCATGGCACAGGTGCAGCTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCAG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 60-S-bR2- | ATGAAATACCTATTGCCTACGGCGGCCGCTGGATTGTTATTACTCGCGGC | 294 | 192 |
| 1D12 | CCAGCCGGCCATGGCGCAGGTGCAGCTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCAG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 69-S-bR2- | ATGAAATACCTATTGCCTACGGCGGCCGCTGGATTGTTATTACTCGCGGC | 295 | 192 |
| 1F10 | CCAGCCGGCCATGGCGCAGGTGCAGCTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCAG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 75-S-bR2- | ATGAAATACCTATTGCCTACGGCGGCCGCTGGATTGTTATTACTCGCGGC | 296 | 192 |
| 1B11 | CCAGCCGGCCATGAAACAGGTGCAGCTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCAG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 91-S-bR2- | ATGAAATACCTATTGCCTACGGCGGCCGCTGGATTGTTATTACTCGCGGC | 297 | 192 |
| 1C3 | CCAGCCGGCCATGGCGCAGGTGCAGCTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCAG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 05-S-bR2- | ATGAAATACCTATTGCCTACGGCGGCCGCTGGATTGTTATTACTCGCGGC | 298 | 193 |
| 1E10 | CCAGCCGGCCATGGCGCAGGTGCAGCTGCAGGAGTCTGGGGGAGGCTT | | |
| | GGTGCAGGCTGGGGGGTCTCTGAGACTCTCCTGTGCAGCCTCTGGAAGC | | |
| | ACCTTCAGTATCACGTACATGGCCTGGTTCCGCCAGGCTCCAGAAAAGCA | | |
| | GCGCGAGTTGGTCGCAGAAATGAGTAGGCGTGGTAGTACATTCTATGCA | | |
| | GACTCCGTGAAGGGCCGATTCACCATCTCCAGAGACAACACCAAGAACA | | |
| | CAGTCTATCTGCAAATGAACAGCCTAGAACCTGAAGACACGGCCGTCTAT | | |
| | TATTGTAGTGTAGGCGCACGTCGCGACGAGGATGATTATGTCTACTGGG | | |
| | GCCAGGGGACCCAGGTCACCGTCTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 26-S-bR1- | ATGAAATACCTATTGCCTACGGCGGCCGCTGGATTGTTATTACTCGCGGC | 299 | 193 |
| 2H12 | CCAGCCGGCCATGGCGCAGGTGCAGCTGCAGNAGTCTGGGGGAGGCTT | | |
| | GGTGCAGGCTGGGGGGTCTCTGAGACTCTCCTGTGCAGCCTCTGGAAGC | | |
| | ACCTTCAGTATCACGTACATGGCCTGGTTCCGCCAGGCTCCAGAAAAGCA | | |
| | GCGCGAGTTGGTCGCAGAAATGAGTAGGCGTGGTAGTACATTCTATGCA | | |
| | GACTCCGTGAAGGGCCGATTCACCATCTCCAGAGACAACACCAAGAACA | | |
| | CAGTCTATCTGCAAATGAACAGCCTAGAACCTGAAGACACGGCCGTCTAT | | |
| | TATTGTAGTGTAGGCGCACGTCGCGACGAGGATGATTATGTCTACTGGG | | |
| | GCCAGGGGACCCAGGTCACCGTTTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 46-S-bR2- | ATGAAATACCTATTGCCTACGGCGGCCGCTGGATTGTTATTACTCGCGGC | 300 | 193 |
| 1H11 | CCAGCCGGCCATGGCGCAGGTGCAGCTGCAGGAGTCTGGGGGAGGCTT | | |
| | GGTGCAGGCTGGGGGGTCTCTGAGACTCTCCTGTGCAGCCTCTGGAAGC | | |
| | ACCTTCAGTATCACGTACATGGCCTGGTTCCGCCAGGCTCCAGAAAAGCA | | |
| | GCGCGAGTTGGTCGCAGAAATGAGTAGGCGTGGTAGTACATTCTATGCA | | |
| | GACTCCGTGAAGGGCCGATTCACCATCTCCAGAGACAACACCAAGAACA | | |
| | CAGTCTATCTGCAAATGAACAGCCTAGAACCTGAAGACACGGCCGTCTAT | | |
| | TATTGTAGTGTAGGCGCACGTCGCGACGAGGATGATTATGTCTACTGGG | | |
| | GCCAGGGGACCCAGGTCACCGTCTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 57-S-bR1- | ATGAAATACCTATTGCCTACGGCGGCCGCTGGATTGTTATTACTCGCGGC | 301 | 193 |
| 1H3 | CCAGCCGGCCATGGCGCAGGTGCAGCTGCAGGAGTCTGGGGGAGGCTT | | |
| | GGTGCAGGCTGGGGGGTCTCTGAGACTCTCCTGTGCAGCCTCTGGAAGC | | |
| | ACCTTCAGTATCACGTACATGGCCTGGTTCCGCCAGGCTCCAGAAAAGCA | | |
| | GCGCGAGTTGGTCGCAGAAATGAGTAGGCGTGGTAGTACATTCTATGCA | | |
| | GACTCCGTGAAGGGCCGATTCACCATCTCCAGAGACAACACCAAGAACA | | |
| | CAGTCTATCTGCAAATGAACAGCCTAGAACCTGAAGACACGGCCGTCTAT | | |
| | TATTGTAGTGTAGGCGCACGTCGCGACGAGGATGATTATGTCTACTGGG | | |
| | GCCAGGGGACCCAGGTCACCGTCTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 83-S-bR2- | ATGAAATACCTATTGCCTACGGCGGCCGCTGGATTGTTATTACTCGCGGC | 302 | 193 |
| 1C1 | CCAGCCGGCCATGGCGCAGGTGCAGTTGCAGGAGTCTGGGGGAGGCTT | | |
| | GGTGCAGGCTGGGGGGTCTCTGAGACTCTCCTGTGCAGCCTCTGGAAGC | | |
| | ACCTTCAGTATCACGTACATGGCCTGGTTCCGCCAGGCTCCAGAAAAGCA | | |
| | GCGCGAGTTGGTCGCAGAAATGAGTAGGCGTGGTAGTACATTCTATGCA | | |
| | GACTCCGTGAAGGGCCGATTCACCATCTCCAGAGACAACACCAAGAACA | | |
| | CAGTCTATCTGCAAATGAACAGCCTAGAACCTGAAGACACGGCCGTCTAT | | |
| | TATTGTAGTGTAGGCGCACGTCGCGACGAGGATGATTATGTCTACTGGG | | |
| | GCCAGGGGACCCAGGTCACCGTCTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 93-S-bR2- | ATGAAATACCTATTGCCTACGGCGGCCGCTGGATTGTTATTACTCGCGGC | 303 | 193 |
| 1G4 | CCAGCCGGCCATGGCGCAGGTGCAGCTGCAGGAGTCTGGGGGAGGCTT | | |
| | GGTGCAGGCTGGGGGGTCTCTGAGACTCTCCTGTGCAGCCTCTGGAAGC | | |
| | ACCTTCAGTATCACGTACATGGCCTGGTTCCGCCAGGCTCCAGAAAAGCA | | |
| | GCGCGAGTTGGTCGCAGAAATGAGTAGGCGTGGTAGTACATTCTATGCA | | |
| | GACTCCGTGAAGGGCCGATTCACCATCTCCAGAGACAACACCAAGAACA | | |
| | CAGTCTATCTGCAAATGAACAGCCTAGAACCTGAAGACACGGCCGTCTAT | | |
| | TATTGTAGTGTAGGCGCACGTCGCGACGAGGATGATTATGTCTACTGGG | | |
| | GCCAGGGGACCCAGGTCACCGTTTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 06-S-bR2- | ATGAAATACCTATTGCCTACGGCGGCCGCTGGATTGTTATTACTCGCGGC | 304 | 194 |
| 1G5 | CCAGCCGGCCATGGCGCAGGTGCAGTTGCAGCAGTCTGGGGGAGGCTT | | |
| | GGTGCAGGCTGGGGGGTCTCTGAGACTCTCCTGTGCAGCCTCTGGAAGC | | |
| | ACCTTCAGTATCACGTACATGGCCTGGTTCCGCCAGGCTCCAGAAAAGCA | | |
| | GCGCGAGTTGGTCGCAGAAATGAGTAGGCGTGGTAGTACATTCTATGCA | | |
| | GACTCCGTGAAGGGCCGATTCACCATCTCCAGAGACAACACCAAGAACA | | |
| | CAGTCTATCTGCAAATGAACAGCCTAGAACCTGAAGACACGGCCGTCTAT | | |
| | TATTGTAGTGTAGGCGCACGTCGCGACGAGGATGATTATGTCTACTGGG | | |
| | GCCAGGGGACCCAGGTCACCGTCTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 09-S-bR1- | ATGAAATACCTATTGCCTACGGCGGCCGCTGGATTGTTATTACTCGCGGC | 305 | 195 |
| 1B5 | CCAGCCGGCCATGGCGCAGGTGCAGTTGCAGGAGTCTGGGGGAGGCTC | | |
| | GGTGCAGGCTGGGGGGTCTCTGAGACTCTCCTGTGCAGACTCTAGAACC | | |
| | ATCTTCATTTTCAACGCCATGGCCTGGTACCGCCAGGCTCCAGGGAAGCA | | |
| | GCGCGAGTTGGTCGCAGCTATTTCTAGTGGTGGCAGTACAAAGTATGCA | | |
| | GACTCCGTGAAGGGCCGATTCACCATCTCCAGTAGCAACGCCAAGAACA | | |
| | CGAAGTATCTGCAGATGAACAGGCTGAAACCTGAGGACACAGCCGTCTA | | |
| | TTACTGTGCAGCCTCAAGGTCGGGTAGGTGGTTAGATGATGCACGAGAC | | |
| | TATGAGTACTGGGGCCCGGGGACCCAGGTCACCGTCTCCTCAACTAGTG | | |
| | GCCCGGGAGGCCAACACCATCACCACCATCATGGCGCAGAACAAAAACT | | |
| | CATCTCAGAAGAGGATCTGTCTTAG | | |
| 10-S-bR1- | ATGAAATACCTATTGCCTACGGCGGCCGCTGGATTGTTATTACTCGCGGC | 306 | 196 |
| 2D8 | CCAGCCGGCCATGGCGCAGGTGCAGCTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCTG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 13-S-bR2- | ATGAAATACCTATTGCCTACGGCGGCCGCTGGATTGTTATTACTCGCGGC | 307 | 196 |
| 1E12 | CCAGCCGGCCATGGCGCAGGTGCAGTTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGTTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCTG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 67-S-bR2- | ATGAAATACCTATTGCCTACGGCGGCCGCTGGATTGTTATTACTCGCGGC | 308 | 196 |
| 1B9 | CCAGCCGGCCATGGCGCAGGTGCAGCTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCTG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 81-S-bR1- | ATGAAATACCTATTGCCTACGGCGGCCGCTGGATTGTTATTACTCGCGGC | 309 | 196 |
| 2B7 | CCAGCCGGCCATGGCGCAGGTGCAGCTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCTG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 92-S-bR2- | ATGAAATACCTATTGCCTACGGCGGCCGCTGGATTGTTATTACTCGCGGC | 310 | 196 |
| 1E6 | CCAGCCGGCCATGGCGCAGGTGCAGCTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCTG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 18-S-bR1- | ATGAAATACCTATTGCCTACGGCGGCCGCTGGATTGTTATTACTCGCGGC | 311 | 196 |
| 2F1 | CCAGCCGGCCATGAAACAGGTGCAGCTGCAGGAGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGETTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCTG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 12-S-bR2- | ATGAAATACCTATTGCCTACGGCGGCCGCTGGATTGTTATTACTCGCGGC | 312 | 197 |
| 1C5 | CCAGCCGGCCATGGCGCAGGTGCAGNNNNANCAGTCTGGGGGAGGCTT | | |
| | GGTGCAGGCTGGGGGGTCTCTGAGACTCTCCTGTGCAGCCTCTGGAATT | | |
| | CCCTTCAGTATCATCTACATGGCCTGGTTCCGCCAGGCTCCAGAAAAGCA | | |
| | GCGCGAGTTGGTCGCAGAAATGAGTAGTCGTGGTAGTAAATTCTATGCA | | |
| | GACTCCGTGAAGGGCCGATTCACCATCTCTAGAGACAACGCCAAGAACA | | |
| | CACTCTATCTGCAAATGAACAGCCTAGAACCTGAAGATACGGCCGTCTAT | | |
| | TATTGCAGTGTAGGCGCACGTCGCGACGACAATGATTATGTGTATTGGG | | |
| | GCCAGGGGACCCAGGTCACCGTTTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 14-S-bR2- | ATGAAATACCTATTGCCTACGGCGGCCGCTGGATTGTTATTACTCGCGGC | 313 | 198 |
| 1G7 | CCAGCCGGCCATGGCGCAGGTGCAGTTGCAGGCGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCAG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 17-S-bR1- | ATGAAATACCTATTGCCTACGGCGGCCGCTGGATTGTTATTACTCGCGGC | 314 | 198 |
| 1C11 | CCAGCCGGCCATGGCGCAGGTGCAGTTGCAGGCGTCTGGGGGAGGCTT | | |
| | GGTGCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCAG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 21-S-bR2- | ATGAAATACCTATTGCCTACGGCGGCCGCTGGATTGTTATTACTCGCGGC | 315 | 199 |
| 1F1 | CCAGCCGGCCATGGCGCAGGTGCAGTTGCAGGAGTCTGGGGGAGGCTT | | |
| | GGTGCAGGCTGGGGGGTCTCTGAGACTCTCCTGTGCAGCCTCTGGAATT | | |
| | CCCTTCAGTATCATCTACATGGCCTGGTTCCGCCAGGCTCCAGAAAAGCA | | |
| | GCGCGAGTTGGTCGCAGAAATGAGTAGTCGTGGTAGTAAATTCTATGCA | | |
| | GACTCCGTGAAGGGCCGATTCACCATCTCTAGAGACAACGCCAAGAACA | | |
| | CACTCTATCTGCAAATGAACAGCCTAGAACCTGAAGATACGGCCGTCTAT | | |
| | TATTGCAGTGTAGGCGCACGTCGCGACGACAATGATTATGTGTATTGGG | | |
| | GCCAGGGGACCCAGGTCACCGTCTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 25-S-bR1- | ATGAAATACCTATTGCCTACGGCGGCCGCTGGATTGTTATTACTCGCGGC | 316 | 199 |
| 1D9 | CCAGCCGGCCATGGCGCAGGTGCAGCTGCAGGAGTCTGGGGGAGGCTT | | |
| | GGTGCAGGCTGGGGGGTCTCTGAGACTCTCCTGTGCAGCCTCTGGAATT | | |
| | CCCTTCAGTATCATCTACATGGCCTGGTTCCGCCAGGCTCCAGAAAAGCA | | |
| | GCGCGAGTTGGTCGCAGAAATGAGTAGTCGTGGTAGTAAATTCTATGCA | | |
| | GACTCCGTGAAGGGCCGATTCACCATCTCTAGAGACAACGCCAAGAACA | | |
| | CACTCTATCTGCAAATGAACAGCCTAGAACCTGAAGATACGGCCGTCTAT | | |
| | TATTGCAGTGTAGGCGCACGTCGCGACGACAATGATTATGTGTATTGGG | | |
| | GCCAGGGGACCCAGGTCACCGTCTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 35-S-bR2- | ATGAAATACCTATTGCCTACGGCGGCCGCTGGATTGTTATTACTCGCGGC | 317 | 199 |
| 1B2 | CCAGCCGGCCATGGCGCAGGTGCAGTTGNNNGAGTCTGGGGGAGGCTT | | |
| | GGTGCAGGCTGGGGGGTCTCTGAGACTCTCCTGTGCAGCCTCTGGAATT | | |
| | CCCTTCAGTATCATCTACATGGCCTGGTTCCGCCAGGCTCCAGAAAAGCA | | |
| | GCGCGAGTTGGTCGCAGAAATGAGTAGTCGTGGTAGTAAATTCTATGCA | | |
| | GACTCCGTGAAGGGCCGATTCACCATCTCTAGAGACAACGCCAAGAACA | | |
| | CACTCTATCTGCAAATGAACAGCCTAGAACCTGAAGATACGGCCGTCTAT | | |
| | TATTGCAGTGTAGGCGCACGTCGCGACGACAATGATTATGTGTATTGGG | | |
| | GCCAGGGGACCCAGGTCACCGTCTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 43-S-bR2- | ATGAAATACCTATTGCCTACGGCGGCCGCTGGATTGTTATTACTCGCGGC | 318 | 199 |
| 1B3 | CCAGCCGGCCATGGCGCAGGTGCAGNNNCAGGAGTCTGGGGGAGGCTT | | |
| | GGTGCAGGCTGGGGGGTCTCTGAGACTCTCCTGTGCAGCCTCTGGAATT | | |
| | CCCTTCAGTATCATCTACATGGCCTGGTTCCGCCAGGCTCCAGAAAAGCA | | |
| | GCGCGAGTTGGTCGCAGAAATGAGTAGTCGTGGTAGTAAATTCTATGCA | | |
| | GACTCCGTGAAGGGCCGATTCACCATCTCTAGAGACAACGCCAAGAACA | | |
| | CACTCTATCTGCAAATGAACAGCCTAGAACCTGAAGATACGGCCGTCTAT | | |
| | TATTGCAGTGTAGGCGCACGTCGCGACGACAATGATTATGTGTATTGGG | | |
| | GCCAGGGGACCCAGGTCACCGTCTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 22-S-bR2- | ATGAAATACCTATTGCCTACGGCGGCCGCTGGATTGTTATTACTCGCGGC | 319 | 200 |
| 1G10 | CCAGCCGGCCATGGCGCAGGTGCAGTTGCAGNNGTNATGGGGGAGGCG | | |
| | TGGTGCAGTCTGGGGGGTCTCTGAGACTCTCCTGTGTAGCCTCTGGAAAC | | |
| | ATCTTCGGGATCAATTCCATGGCCTGGTACCGCCAGGCTCCAGGGAAGC | | |
| | AGCGCGAATTGGTCGCTGACATTACACGTGGTAATAGAAAGTATGCAGA | | |
| | TTCCGTGAAGGGCCGATTCACCATCTCCCAAGACAACGCCAAGAACACG | | |
| | GTGTATCTGCAAATGAACAGGCTGAAACCAGAGGACACGGCCGTCTATT | | |
| | TCTGCAATGCAGAAATCGTCACGCAAATCCCTTTCCCGCCGCGTGAGTTC | | |
| | TGGGGCCGGGGGACCCTGGTCACCGTCTCCTCAACTAGTGGCCCGGGAG | | |
| | GCCAACACCATCACCACCATCATGGCGCAGAACAAAAACTCATCTCAGAA | | |
| | GAGGATCTGTCTTAG | | |
| 29-S-bR2- | ATGAAATACCTATTGCCTACGGCGGCCGCTGGATTGTTATTACTCGCGGC | 320 | 201 |
| 1F2 | CCAGCCGGCCATGGCGCAGGTGCAGTTGCAGGCGTCTGGGGGAGNNNN | | |
| | TGGTGCAGCCTGGGGGGTCTCTGAGACTCTCCTGTGCAGCCTCTGGATTC | | |
| | ACCTTTAGTAGCTACTGGATGTATTGGGTCCGTCAGGCTCCAGGGAAGG | | |
| | GGCTCGAGTGGGTCTCAACAATTAATACTGGTGGTTATACCACATACTAT | | |
| | TCAGACTCCGTGAAGGGCCGATTCACCATCTCCAGAGACAACGGCAAGA | | |
| | ACACGCTGTATCTCGAAATGAACAGTCTGAAATCTGAGGACACGGCCGT | | |
| | GTATTACTGTGCAAAGGCGTACGGTAGTATGTGGTCAGGGATCTGGGGC | | |
| | GGCATGGACTACTGGGGCAAAGGGACCCAGGTCACCGTCTCCTCAACTA | | |
| | GTGGCCCGGGAGGCCAACACCATCACCACCATCATGGCGCAGAACAAAA | | |
| | ACTCATCTCAGAAGAGGATCTGTCTTAG | | |
| 30-S-bR2- | ATGAAATACCTATTGCCTACGGCGGCCGCTGGATTGTTATTACTCGCGGC | 321 | 202 |
| 1H1 | CCAGCCGGCCATGGGGCAGGTCCAGTTGCAGCAGTCTGGGGGAGGCTT | | |
| | GGTCCAGGCTGGGGGGTCTCTGAGACTCTCCTGTGCAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACCACCAATTATCTA | | |
| | GCCTCCGTGAAGGGCCGATTCACTATCTCCAGAGACAACGCCAAGAACA | | |
| | CGGTGTCGCTACAAATGAACAGCCTGAAACCTGAGGACACGGCCGTCTA | | |
| | CACTTGTTATGCAGCCTGGCCGTTGAACACTTGGGGCCAGGGGACCCAG | | |
| | GTCACCCTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 33-S-bR1- | ATGAAATACCTATTGCCTACGGCGGCCGCTGGATTGTTATTACTCGCGGC | 322 | 203 |
| 1G3 | CCAGCCGGCCATGGCGCAGGTGCAGNTGCAGGAGTCTGGGGGAGGCTT | | |
| | GGTCCAGGCTGGGGGGTCTCTGAGACTCTCCTGNGCAGTCTCTGGAAGT | | |
| | ATCATCAGTCATAATGTGATGGCCTGGTACCGCCGCGCTCCAGGGAAGC | | |
| | AGCGCGACAAGGTTGCTTGTATCAGTGGTAGTGGTTTCACCAATTATATA | | |
| | GCCTCCGTGAAGGGCCGATTCACTATCTCCAGAGACAACGCCAAGAACA | | |
| | CGGTGTCTCTACAAATGAACAACCTGAAACCTGAGGACACGGCCGTCTAC | | |
| | TCTTGTTATACAGCCTGGCCGTAGAACACTTGGGGCCAGGGGACCCAGG | | |
| | TCACCGTCTCCTCAACTAGTGGCCCGGGAGGCCAACACCATCACCACCAT | | |
| | CATGGCGCAGAACAAAAACTCATCTCAGAAGAGGATCTGTCTTAG | | |
| 36-S-bR2- | ATGAAATACCTATTGCCTACGGCGGCCGCTGGATTGTTATTACTCGCGGC | 323 | 204 |
| 1D1 | CCAGCCGGCCATGGCGCAGGTGCAGTTGCAGGAGTCTGGGGGAGGCTT | | |
| | TGTGCACCCTGGAGGGTCTCTGACGCTCTCCTGTGCAGCCTCTGGCAGGA | | |
| | TCTTCAATATCGAGGACATGGGCTGGTATCGCCAGGGTCCAGGGGAACA | | |
| | GCGCGACTTGGTCGCAACGATCACCCGTACTGGTGCGCCAACCTATGCAA | | |
| | ACTCCGTGAAGGGCCGGTTCACCATCTCCAGAGACAACGCCAAGAACAC | | |
| | GGTTTATCTGCAGATGACCAGGCTGAAACCTGAGGACACGGCCGTCTATT | | |
| | ACTGTAATGCAAAAGACGTAACAGTCATACCTTTCCCCCCGAAAGACTAT | | |
| | TGGGGCCGGGGGACCCAGGTCACCGTCTCCTCAACTAGTGGCCCGGGAG | | |
| | GCCAACACCATCACCACCATCATGGCGCAGAACAAAAACTCATCTCAGAA | | |
| | GAGGATCTGTCTTAG | | |
| 41-S-bR1- | ATGAAATACCTATTGCCTACGGCGGCCGCTGGATTGTTATTACTCGCGGC | 324 | 205 |
| 1H1 | CCAGCCGGCCATGGCGCAGGTGCAGCTGCAGGAGTCTGGGGGAGGCTT | | |
| | GGTGCAGGCTGGGGGGTCTCTGAGACTCTCCTGTGAAGCCTCTGGAAGC | | |
| | GTTTCCGCGATCGAAACCATGGGCTGGTACCGCCAGGCTCCGGATGAAC | | |
| | AGCGCACATTTGTCGCGGTTATCAGTACGGGTGGAACCACAAAATACGC | | |
| | GCCCTCCGTGAAGGGCCGATTCACCATCTCCATAGACAACGCCAAGAGC | | |
| | ACGGTGACGCTTCAAATGAACAGCCTGAAACCTGAGGACACAGCCGTCT | | |
| | ACTACTGTGCAGCGGACTGGCGAACCATTTTGGGTTGGAAGACAAGGGA | | |
| | GCCCAACTACTTTGGCCAGGGGACCCTGGTCACCGTCTCCTCAACTAGTG | | |
| | GCCCGGGAGGCCAACACCATCACCACCATCATGGCGCAGAACAAAAACT | | |
| | CATCTCAGAAGAGGATCTGTCTTAG | | |
| 45-S-bR2- | ATGAAATACCTATTGCCTACGGCGGCCGCTGGATTGTTATTACTCGCGGC | 325 | 206 |
| 1F6 | CCAGCCGGCCATGGCGCAGGTGCAGTTGCAGGAGTCTGGGGGAGGCTT | | |
| | GGTGCAGGCTGGGGGGTCTCTGAGACTCTCCTGTGCAGCCTTTGGAAGC | | |
| | ACCTCCAGTATCACGTACATGGCCTGGTTCCGCCAGGCTCCAGAAAAGCA | | |
| | GCGCGAGTTGGTCGCAGAAATGAGTAGGCGTGGTAGCACATTCTATGCA | | |
| | GACTCCGTGAAGGGCCGATTCACCATTTATAGAGACAACACCAAGAACA | | |
| | CAGTCTATCTGCAAATGAACAGCCTAGAACCTGAAGACACGGCCGTCTAT | | |
| | TATTGTAGTGTAGGCGCACGTCGCGACGAGGATGATTATGTCTACTGGG | | |
| | GCCAGGGGACCCAGGTCACCGTCTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 61-S-bR2- | ATGAAATACCTATTGCCTACGGCGGCCGCTGGATTGTTATTACTCGCGGC | 326 | 206 |
| 1F8 | CCAGCCGGCCATGGCGCAGGTGCAGTTGCAGGAGTCTGGGGGAGGCTT | | |
| | GGTGCAGGCTGGGGGGTCTCTGAGACTCTCCTGTGCAGCCTTTGGAAGC | | |
| | ACCTCCAGTATCACGTACATGGCCTGGTTCCGCCAGGCTCCAGAAAAGCA | | |
| | GCGCGAGTTGGTCGCAGAAATGAGTAGGCGTGGTAGCACATTCTATGCA | | |
| | GACTCCGTGAAGGGCCGATTCACCATTTATAGAGACAACACCAAGAACA | | |
| | CAGTCTATCTGCAAATGAACAGCCTAGAACCTGAAGACACGGCCGTCTAT | | |
| | TATTGTAGTGTAGGCGCACGTCGCGACGAGGATGATTATGTCTACTGGG | | |
| | GCCAGGGGACCCAGGTCACCGTTTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 84-S-bR2- | ATGAAATACCTATTGCCTACGGCGGCCGCTGGATTGTTATTACTCGCGGC | 327 | 206 |
| 1E5 | CCAGCCGGCCATGGCGCAGGTGCAGNNNNNANNAGTNNGGGGGAGGC | | |
| | TTGGTGCAGGCTGGGGGGTCTCTGAGACTCTCCTGTGCAGCCTTTGGAA | | |
| | GCACCTCCAGTATCACGTACATGGCCTGGTTCCGCCAGGCTCCAGAAAAG | | |
| | CAGCGCGAGTTGGTCGCAGAAATGAGTAGGCGTGGTAGCACATTCTATG | | |
| | CAGACTCCGTGAAGGGCCGATTCACCATTTATAGAGACAACACCAAGAA | | |
| | CACAGTCTATCTGCAAATGAACAGCCTAGAACCTGAAGACACGGCCGTCT | | |
| | ATTATTGTAGTGTAGGCGCACGTCGCGACGAGGATGATTATGTCTACTGG | | |
| | GGCCAGGGGACCCAGGTCACCGTCTCCTCAACTAGTGGCCCGGGAGGCC | | |
| | AACACCATCACCACCATCATGGCGCAGAACAAAAACTCATCTCAGAAGAG | | |
| | GATCTGTCTTAG | | |
| 49-S-bR1- | ATGAAATACCTATTGCCTACGGCGGCCGCTGGATTGTTATTACTCGCGGC | 328 | 207 |
| 1H2 | CCAGCCGGCCATGGCGCAGGTGCAGTTGCAGGAGTCTGGGGGAGGCTT | | |
| | GGTGCAGGCTGGGGGGTCTCTGAGACTCTCCTGTGCAGCCTCTGGAGGA | | |
| | CCTGTCAGTGACAATGTCATGGCCTGGTTCCGCCAGGCTCCAGGGAGCC | | |
| | AGCGCGAATTGGTCGCACAGATTACAAGTGGTGGGGCCACAAGCTACGC | | |
| | GGACTCCGTGAAGGGCCGATTCACCATCTCCAGAGACAACGCCAGGAGC | | |
| | ACAGTGGACCTGCAAATGAACAGCCTGAAACCTGAGGACACGGCCGTCT | | |
| | ATTACTGTAACGTCGCCTTACGTTACTGGGGCCGGGGGACCCAGGTCACC | | |
| | GTCTCCTCAACTAGTGGCCCGGGAGGCCAACACCATCACCACCATCATGG | | |
| | CGCAGAACAAAAACTCATCTCAGAAGAGGATCTGTCTTAG | | |
| 51-S-bR2- | ATGAAATACCTATTGCCTACGGCGGCCGCTGGATTGTTATTACTCGCGGC | 329 | 208 |
| 1B7 | CCAGCCGGCCATGGCGCAGGTGCAGNNNNAANCAGTNTGGGGGAGGC | | |
| | TTGGTGCAGGCTGGGGGGTCTCTGAGACTCTCCTGTGCAGCCTCTGGAA | | |
| | GCACCTTCAGTATCACCTACATGGCCTGGTTCCGCCAGGCTCCAGGGAAA | | |
| | CAGCGCGAATTGGTCGCAGAAATAAGTAGCCGTGGTAGTGTGTTCTATG | | |
| | CAGACTCCGTGAAGGGCCGATTCACCATCTCCAGAGACAACGCCAAGAA | | |
| | GACAGTGTATCTGCAAATGAACAGCCTGGAAACTGAAGACACGGCCGCC | | |
| | TATTATTGTAGTGTAGGCGCACGTCGCGACGAAGATGACTATGTCTACTG | | |
| | GGGCCAGGGGACCCAGGTCACCGTTTCCTCAACTAGTGGCCCGGGAGGC | | |
| | CAACACCATCACCANCNNCATGGCGCAGAACAAAAACTCATCTCAGAAG | | |
| | AGGATCTGTCTTAG | | |
| 52-S-bR2- | ATGAAATACCTATTGCCTACGGCGGCCGCTGGATTGTTATTACTCGCGGC | 330 | 209 |
| 1D6 | CCAGCCGGCCATGGCGCAGGTGCAGTTGCAGGAGTCTGGGGGAGGCTT | | |
| | GGTGCAGGCTGGGGGGTCTCTGAGACTCTCCTGTGCAGCCTCTGGAAGC | | |
| | ACCTTCAGTATCACGTACATGGCCTGGTTCCGCCAGGCTCCAGAAAAGCA | | |
| | GCGCGAGTTGGTCGCAGAAATGAGTAGGCGTGGTAGTACATTCTATGCA | | |
| | GACTCCGTGAAGGGCCGATTCACCATCTCCAGAGACAACACCAAGAACA | | |
| | CAGTCTATCTGCAAATGAACAGCCTAGAACCTGAAGACACGGCCGTCTAT | | |
| | TATTGTAGTGTAGGCGCACGTCGCGACGAGGATGATTATGTCTACTGGG | | |
| | GCCAGGGGACCCTGGTCACCGTTTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 54-S-bR2- | ATGAAATACCTATTGCCTACGGCGGCCGCTGGATTGTTATTACTCGCGGC | 331 | 209 |
| 1H12 | CCAGCCGGCCATGGCGCAGGTGCAGTTGCAGGAGTCTGGGGGAGGCTT | | |
| | GGTGCAGGCTGGGGGGTCTCTGAGACTCTCCTGTGCAGCCTCTGGAAGC | | |
| | ACCTTCAGTATCACGTACATGGCCTGGTTCCGCCAGGCTCCAGAAAAGCA | | |
| | GCGCGAGTTGGTCGCAGAAATGAGTAGGCGTGGTAGTACATTCTATGCA | | |
| | GACTCCGTGAAGGGCCGATTCACCATCTCCAGAGACAACACCAAGAACA | | |
| | CAGTCTATCTGCAAATGAACAGCCTAGAACCTGAAGACACGGCCGTCTAT | | |
| | TATTGTAGTGTAGGCGCACGTCGCGACGAGGATGATTATGTCTACTGGG | | |
| | GCCAGGGGACCCTGGTCACCGTCTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 53-S-bR2- | ATGAAATACCTATTGCCTACGGCGGCCGCTGGATTGTTATTACTCGCGGC | 332 | 210 |
| 1F7 | CCAGCCGGCCATGGCGCAGGTGCAGTTGCAGGAGTCTGGGGGAGGCTT | | |
| | GGTGCAGGCTGGGGGGTCTCTGAGACTCTCCTGTGCAGCCTCTGGAATT | | |
| | CCCTTCAGTATCATCTACATGGCCTGGTTCCGCCAGGCTCCAGAAAAGCA | | |
| | GCGCGAGTTGGTCGCAGAAATGAGTAGTCGTGGTAGTAAATTCTATGCA | | |
| | GACTCCGTGAAGGGCCGATTCACCATCTCTAGAGACAACGCCAAGAACA | | |
| | CACTCTATCTGCAAATGAACAGCCTAGAACCTGAAGATACGGCCGTCTAT | | |
| | TATTGCAGTGTAGGCGCACGTCGCGACGACAATGATTATGTGTATTGGG | | |
| | GCCAGGGGACCCTGGTCACCGTCTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 59-S-bR2- | ATGAAATACCTATTGCCTACGGCGGCCGCTGGATTGTTATTACTCGCGGC | 333 | 211 |
| 1B8 | CCAGCCGGCCATGGCGCAGGTGCAGCTGCAGGAGTTTGGGGGAGGCTT | | |
| | GGTGCAGGCTGGGGGGTCTCTGAGACTCTCCTGTGCAGCCTCTGGAAGC | | |
| | ACCTTCAGTATCACGTACATGGCCTGGTTCCGCCAGGCTCCAGAAAAGCA | | |
| | GCGCGAGTTGGTCGCAGAAATGAGTAGGCGTGGTAGTACATTCTATGCA | | |
| | GACTCCGTGAAGGGCCGATTCACCATCTCCAGAGACAACACCAAGAACA | | |
| | CAGTCTATCTGCAAATGAACAGCCTAGAACCTGAAGACACGGCCGTCTAT | | |
| | TATTGTAGTGTAGGCGCACGTCGCGACGAGGATGATTATGTCTACTGGG | | |
| | GCCAGGGGACCCAGGTCACCGTCTCCTCAACTAGTGGCCCGGGAGGCCA | | |
| | ACACCATCACCACCATCATGGCGCAGAACAAAAACTCATCTCAGAAGAG | | |
| | GATCTGTCTTAG | 334 | 212 |
| 68-S-bR2- | ATGAAATACCTATTGCCTACGGCGGCCGCTGGATTGTTATTACTCGCGGC | | |
| 1E2 | CCAGCCGGCCATGGCGCAGGTGCAGCTGCAGGAGTTTGGGGGAGGCTT | | |
| | GGTGCAGGCAGGGGAATCTCTAAGACTCTCCTGTGTAGCCTCTGGAAGT | | |
| | AACATCAGTACTAATGTGATGGCCTGGTACCGCCGCGCTCCAGGGAACC | | |
| | AGCGCGACATGGTTGCTTCTATCAGTACTAGTGGTACTACCAATTATCTA | | |
| | GCCTCCGTGAAGGGCCGATTCACTATCTCCAGAGACAACGCCAAGAACA | | |
| | CGGTGTCGCTGCAAATGAACAGCCTGAAACCTGAGGACACGGCCGTCTA | | |
| | CACTTGTTATGCAGCCTGGCCGTTGAACACTTGGGGCCAGGGGACCCTG | | |
| | GTCACCGTCTCCTCAACTAGTGGCCCGGGAGGCCAACACCATCACCACCA | | |
| | TCATGGCGCAGAACAAAAACTCATCTCAGAAGAGGATCTGTCTTAG | | |
| 73-S-bR1- | ATGAAATACCTATTGCCTACGGCGGCCGCTGGATTGTTATTACTCGCGGC | 335 | 213 |
| 2B2 | CCAGCCGGCCATGGCGCAGGTGCAGTTGCAGGAGTCTGGGGGAGGCTT | | |
| | GGTGCAGGCCGGGGGGTCTCTGAGACTCTCCTGTGTAGGCGCTGGAAGC | | |
| | GCCTTCGGTTGGAATGCCGTGCACTGGTACCGCCAGGCTCCAGGTCAGC | | |
| | AGCGCGAATGGCTCGCCACTATTGAGAGTGGTGGCTGGGCAGACTATTC | | |
| | AGTGTCCGTGAAGGGCCGATTCATCGTCTCCAGAGACAACGCCAGGAAC | | |
| | ACAGCGTATTTGCAAATGAACAACCTAAAACTTGAAGACACGGCCGTCTA | | |
| | TTACTGTAATCAACTTACTTACTGGGGCCAGGGGACCCAGGTCACCGTCT | | |
| | CCTCAACTAGTGGCCCGGGAGGCCAACACCATCACCACCATCATGGCGC | | |
| | AGAACAAAAACTCATCTCAGAAGAGGATCTGTCTTAG | | |
| 76-S-bR2- | ATGAAATACCTATTGCCTACGGCGGCCGCTGGATTGTTATTACTCGCGGC | 336 | 214 |
| 1E3 | CCAGCCGGCCATGGCGCAGGTGCANCTGCAGGAGTNTGGGGGAGGCTT | | |
| | GGTGCAGGCTGGGGGGTCTCTGAGACTCTCCTGTGCAGCCTCTGGAAGC | | |
| | ACCTTCAGTATCACGTACATGACCTGGTTCCGCCAGGCTCCAGAAAAGCA | | |
| | GCGCGAGTTGGTCGCAGAAATGAGTAGGCGTGGTAGTACATTCTATGCA | | |
| | GACTCCGTGAAGGCCCGATTCACCATCTCCAGAGACAACACCAAGAACAC | | |
| | AGTCTATCTGCAAATGAACAGCCTAGAACCTGAAGACACGGCCGTCTATT | | |
| | ATTGTAGTGTAGGCGCACGTCGCGACGAGGATGATTATGTCTACTGGGG | | |
| | CCAGGGGACCCAGGTCACCGTCTCCTCAACTAGTGGCCCGGGAGGCCAA | | |
| | CACCATCACCACCATCATGGCGCAGAACAAAAACTCATCTCAGAAGAGG | | |
| | ATCTGTCTTAG | | |
| 77S-bR2- | ATGAAATACCTATTGCCTACGGCGGCCGCTGGATTGTTATTACTCGCGGC | 337 | 215 |
| 1G1 | CCAGCCGGCCATGGCGCAGGTGCAGTTGCAGGAGTTTGGGGGAGGCTT | | |
| | GGTGCAGGCTGGGGGGTCTCTGAGACTCTCCTGTGCAGCCTCTGGAAGC | | |
| | ACCTTCAGTATCACGTACATGGCCTGGTTCCGCCAGGCTCCAGAAAAGCA | | |
| | GCGCGAGTTGGTCGCAGAAATGAGTAGGCGTGGTAGTACATTCTATGCA | | |
| | GACTCCGTGAAGGGCCGATTCACCATCTCCAGAGACAACACCAAGAACA | | |
| | CAGTCTATCTGCAAATGAACAGCCTAGAACCTGAAGACACGGCCGTCTAT | | |
| | TATTGTAGTGTAGGCGCACGTCGCGACGAGGATGATTATGTCTACTGGG | | |
| | GCCAGGGGACCCTGGTCACCGTTTCCTCAACTAGTGGCCCGGGAGGCCA | | |
| | ACACCATCACCACCATCATGGCGCAGAACAAAAACTCATCTCAGAAGAG | | |
| | GATCTGTCTTAG | | |
| 85-S-bR2- | ATGAAATACCTATTGCCTACGGCGGCCGCTGGATTGTTATTACTCGCGGC | 338 | 216 |
| 1G2 | CCAGCCGGCCATGGCGCAGGTGCAGTTGCAGCAGTCTGGGGGAGGCTT | | |
| | GGTGCAGGCTGGGGGGTCTCTGAGACTCTCCTGTGCAGCCTCTGGAAGC | | |
| | ACCTTCAGTATCACCTACATGGCCTGGTTCCGCCAGGCTCCAGGGAAACA | | |
| | GCGCGAATTGGTCGCAGAAATAAGTAGCCGTGGTAGTGTGTTCTATGCA | | |
| | GACTCCGTGAAGGGCCGATTCACCATCTCCAGAGACAACGCCAAGAAGA | | |
| | CAGTGTATCTGCAAATGAACAGCCTGGAAACTGAAGACACGGCCGCCTA | | |
| | TTATTGTAGTGTAGGCGCACGTCGCGACGAAGATGACTATGTCTACTGG | | |
| | GGCCAGGGGACCCTGGTCACCGTCTCCTCAACTAGTGGCCCGGGAGGCC | | |
| | AACACCATCACCACCATCATGGCGCAGAACAAAAACTCATCTCAGAAGAG | | |
| | GATCTGTCTTAG | | |
| 89-S-bR1- | ATGAAATACCTATTGCCTACGGCGGCCGCTGGATTGTTATTACTCGCGGC | 339 | 217 |
| 2C6 | CCAGCCGGCCATGGCGCAGGTGCAGCTGCAGGAGTCTGGGGGAGGCTT | | |
| | GGTGCAGCCTGGGGGGTCTCTGAGACTCTCCTGCACACCCTCTGGATCTA | | |
| | TCTTCAGTTTCGATGTCATGGCCTGGTATCGCCAGGCCCCAGGGAAGCGG | | |
| | CGCGAGTTGGTCGCACAGCATCGTACTCCGGGTGCTATAGATTATGCCGA | | |
| | TCCTGTCCGGGGCCGATTCACTATTAGCAGAGACGCTGGGGACGTACTG | | |
| | CTGCAAATGGACAGCCTGAAACCCGAAGACACGGCCGTCTACTTCTG | | |
| | TAATCTCCGAAGGTGGTCTTACGACTACTGGGGCCAGGGGACCCTGGTC | | |
| | ACCGTCTCCTCAACTAGTGGCCCGGGAGGCCAACACCATCACCACCATCA | | |
| | TGGCGCAGAACAAAAACTCATCTCAGAAGAGGATCTGTCTTAG | | |

### EXAMPLES

Examples are provided herein to illustrate the present invention. These examples are not meant to constrain the present invention to any particular application or theory of operation.

### Example 1—Production of Llama Single Domain Antibodies Against Human α-Synuclein

Immunizations: A llama was immunized with alpha-synuclein according to the following schedule. In general, immunizations occurred once per month, with serum and/or PBMCs taken 1 week after boosts.

α-synuclein Immunization Schedule

| | | |
|---|---|---|
| Pre-bleed | | Day 0 |
| Immunization 1 | 200 ug CFA, SQ | Day 0 |
| Immunization 2 | 100 ug IFA, SQ | Day 21 |
| Bleed 1 | | Day 28 |
| Immunization 3 | 100 ug IFA 100 ug | Day 49 |
| | Adjuplex, SQ | |
| Bleed 2 | | Day 56 |
| Immunization 4 | 100 ug IFA 100 ug | Day 77 |
| | Adjuplex, SQ | |
| Bleed 3 | | Day 84 |
| Immunization 5 | 100 ug IFA 100 ug | Day 105 |
| | Adjuplex, SQ | |
| Bleed 4 | | Day 112 ~1 × 10⁸ PBMCs |
| Immunization 6 | 200 ug Adjuplex | Day 144 |
| | split IM/SQ | |
| Bleed 5 | | Day 151 ~1 × 10⁸ PBMCs |
| Immunization 7 | 200 ug Adjuplex | Day 188 |
| | split IM/SQ | |
| Bleed 6 | | Day 196 |

Complete Freund's Adjuvant (CFA), Incomplete Freund's Adjuvant (IFA), Adjuplex Adjuvant(Sigma), Subcutaneous(SQ), Intramusclular(IM).

Serum titer levels were determined by indirect ELISA using the following protocol:
1) Antigen was coated in 96 well Corning 9018 high-binding polystyrene assay plates at 2 ug/mL in PBS, 4 degrees ON.
2) Wash plate 4 times with PBS containing 0.05% Tween20 (PBST).
3) Block with PBST/5% non-fat dried milk (PBST/milk) for 2 hr at room temperature.
4) Wash 4 times with PBST.
5) Dilutions of sera were made in PBST/milk and incubated in plate for 1 hr at room temperature.
6) Wash 4 times with PBST.
7) Incubate with Rabbit anti-Llama IgG (H+L) (Life Technologies), 1:10,000 dilution in PBST/milk, 1 hr room temperature.
8) Wash 4 times with PBST.
9) Bound antibody was detected with Gt anti-Rb IgG (H+L)-HRP (Jackson), 1:10,000 in PBST/milk, 45 min room temperature.

Library Construction: Approximately 100 mL of llama blood was collected in EDTA-coated tubes (Becton Dickenson) and peripheral blood mononuclear cells (PBMCs) were isolated using Hisotpaque-1077 (Sigma). Approximately 1×10⁸ PBMCs were isolated from the specific bleeds listed above and RNA prepared using RNeasy (Qiagen). cDNA was synthesized from a total of 50 ug of RNA (25 ug from each bleed) using random hexanucleotide primers and SuperScript reverse transcriptase (Life Technologies). Single domain antibodies (VHH) were cloned in a two step PCR process using the cDNA as template. The first PCR amplified the variable domains of all immunoglobulin heavy chains, both single chain (VHH) and conventional antibodies (VH) using CaL1/CaL2 primers:

(SEQ ID NO: 344)
5′-GTCCTGGCTGCTCTTCTACAAGG-3′
(SEQ ID NO: 345)
5′-GGTACGTGCTGTTGAACTGTTCC-3′

The second nested PCR specifically amplified llama single domain antibodies (ProSci proprietary primers). PCR products were gel purified and the DNA fragments encoding the VHH domains were cloned into phage display vector pADL-23c (Antibody Design Labs) and electroporated into E. coli TG1 cells, yielding a library of approximately 1×10⁹ in size.

### Example 2—Selection of Single Domain Antibodies Against α-Synuclein

Phage displaying VHH antibodies were rescued from individual libraries with helper phage M13K07 and subjected to biopanning in two different ways: solid-phase and solution-phase panning. For solid-phase panning, α-synuclein was adsorbed on 2 um diameter polystyrene beads (Polysciences, Inc), blocked with PBST containing 5% non-fat dried milk, and incubated with 2×10¹¹ phage in 1 mL PBST/milk for 1 hr at room temperature. For solution-phase panning, proteins were biotinylated using Lightning-Link Rapid Biotin (Innova Biosciences) and 1 ug of biotinylated α-synuclein incubated with 2×10¹¹ phage in 1 mL PBST/milk for 1 hr at room temperature. Polystyrene beads were captured by centrifugation and washed extensively with PBST. Biotinylated proteins bound to phage were captured with streptavidin-coated magnetic Dynabeads (Life Technologies) and extensively washed with PBST. In both cases, bound phage were eluted with both high pH (100 mM triethylamine, pH˜10) and low pH (100 mM glycine, pH˜2.5) for 5 min and neutralized with 1M Tris pH 7.5. E. coli strains TG1 and SS320 were then infected with eluted phage and used for a subsequent round of panning (TG1) or to express antibodies (SS320).

### Example 3—Expression of Single Domain Antibodies for Screening

Individual SS320 clones were grown in 96 well plate format in 100 μL of 2YT/1% glucose/100 μg/mL ampicillin overnight at 37° C. 10 μL of the overnight cultures were inoculated into 1 mL each of 2YT/0.1% glucose/100 μg/mL ampicillin in deep 96 well blocks and grown at 37° C., 200 rpm for 3 hrs until cells were in log phase. Single domain antibody expression was then induced with IPTG (1 mM final) and the temperature reduced to 30° C. overnight. The next day, bacterial cultures were centrifuged at 3200 g for 10 min and supernatant removed. The remaining bacterial pellets were frozen at −70° C. Bacterial pellets were then thawed, resuspended in 300 μL of PBS, and incubated for 30 min at room temperature. Cellular debris was removed by centrifugation at 3200 g for 10 min and the antibody-containing supernatants transferred to a fresh 96 well plate for storage at −70° C. until used.

ELISA screening of the supernatants was performed using the following protocol:
1) coat antigen at 2 ug/mL in PBS, 4 degrees ON (use 96 well Corning 9018 high-binding polystyrene assay plates).
2) wash 4 times with PBST
3) 2 hr block with PBST/5% milk, room temp
4) wash 4 times PBST
5) add dilutions of sdAb supes (1:1) in PBST/5% milk, 1 hr room temp
6) wash 4 times PBST
7) add ProSci's anti-c-myc-tag antibody (Cat. No. PM-7669), 1:1,000 in PBST/5% milk, 1 hr room temp
8) wash 4 times PBST
9) detect with goat anti-mouse IgG-HRP (Jackson Cat. No. 115-035-164), 1:5,000 in PBST/5% milk, incubate 30 to 45 min room temperature
10) wash 4 times PBST
11) develop 10 min to 1 hr depending on signal

### Example 4—Single Domain Antibody Purification

Single Domain Antibodies were purified from 50 mL cultures as follows. Inoculated 3 mL of 2YT/1% glucose/100 ug/mL Amp with specific clone in SS320 cells and grew overnight 37 degrees, 200 rpm. Next day, 500 uL of overnight culture was added to 50 mL of 2YT/0.1% glucose/100 ug/mL ampicillin and grown at 37 degrees, 200 rpm. After OD₆₀₀ reached 0.7, induced the culture with IPTG (1 mM final) and grown overnight at 30 degrees.

The next day, bacteria was collected by centrifugation for 15 min, 3500 g at room temperature. The cell pellet was resuspended in 2.5 mL of ice-cold TES (20 mM Tris, 0.5 mM EDTA, 17% sucrose), and incubated for 1 hr on ice. 5 mL of TES/4 (TES diluted 1 to 4 in water) was added, and incubation on ice continued for 45 min mixing occasionally. The suspension was centrifuged at 10,000 g for 30 min, 4 degrees and the supernatant was collected. The His-tagged single domain antibodies were purified by using IMAC (Immobilized Metal Affinity Chromatography) according to the manufacturer's instructions (Qiagen).

### Example 5—Characterization of Alpha-Synuclein Single Domain Antibodies

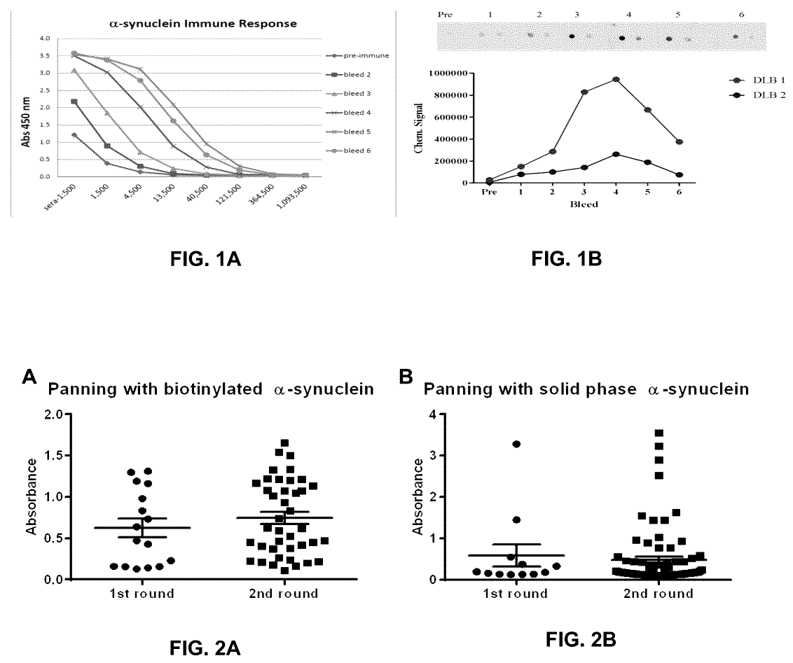

Single domain antibody (sdAb) titer following administration of α-synuclein as described in Example 1 was measured by ELISA. ELISA plates were coated with 2 μg/ml of recombinant (rec) α-synuclein (140 aa) and reacted with various bleeds obtained after several immunizations with rec α-synuclein. The hydrogen peroxidase (HRP)-linked secondary antibody recognizes single-domain llama antibodies. Some auto-sdAbs against α-synuclein were detected in the pre-bleed but a strong sdAb response against α-synuclein was detected in bleed 2 and onwards. FIG. 1 (left graph) shows that llamas immunized with the α-synuclein produced a strong sdAb titer against the immunogen after the sixth immunization.

All of the collected bleeds (1:1000 dilution) were reacted on a dot blot with brain homogenate from two different human brains. These brain samples were selected based on their strong reactivity with commercial α-synuclein antibodies suggesting extensive Lewy bodies. The secondary antibody detects single domain llama antibodies. All blots were developed together for direct comparison of binding. For both cases, signal was strongest in bleeds 3-5. As shown in FIG. 1 (right graph), the single domain antibodies from an α-synuclein immunized llama are strongly immunoreactive with brain homogenates from cases with suspected Dementia with Lewy Bodies (DLB).

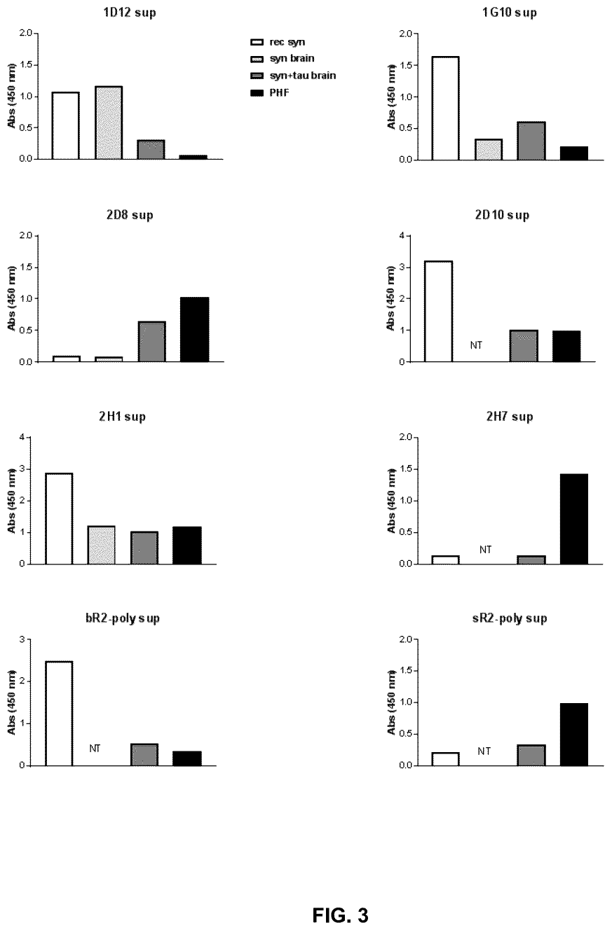

The α-synuclein sdAbs were next selected for by binding to α-synuclein. Phage display library panning against biotinylated-α-synuclein (solution phase) resulted in enrichment of positive clones (Abs>0.1) with 9% (1^{st} round: two 96 well plates) and 48% (2^{nd} round-one plate) positivity (FIG. 2A). Panning against solid phase α-synuclein resulted in 6% (1^{st} round) and 43% (2^{nd} round, 2 plates) positivity (FIG. 2B).

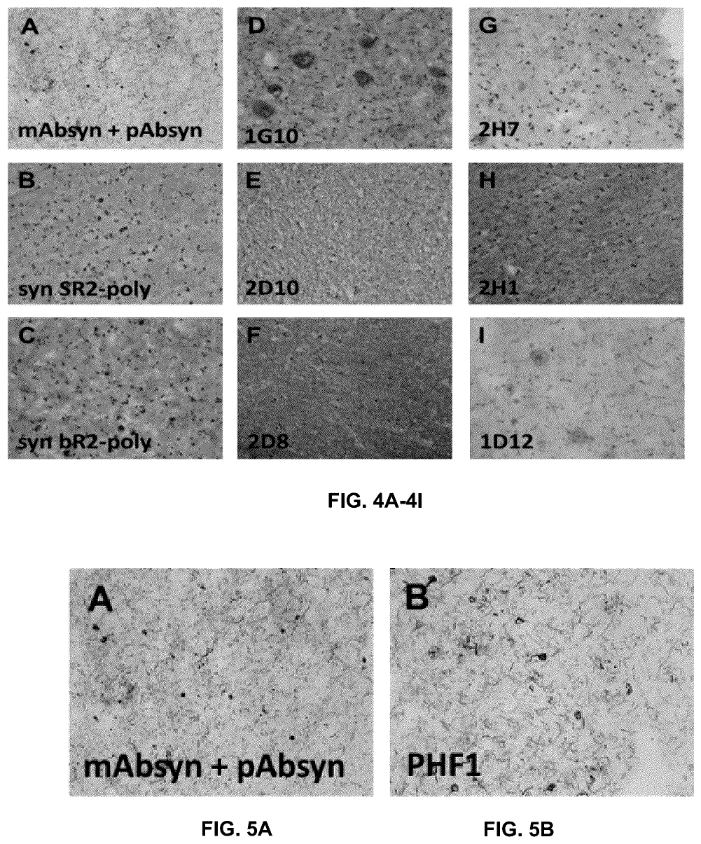

To test the binding profiles of the selected sdAbs, cell culture supernatant from sdAb clones was incubated with ELISA plates that were coated with: 1) recombinant α-synuclein protein (rec syn); 2) supernatant from a human brain with a strong α-synuclein signal (syn brain) on western blots as detected by a commercial α-synuclein antibody; 3) supernatant from a DLB brain, positive for tau and α-synuclein on western (syn+tau brain), and; 4) PHF-tau-enriched brain fraction from Alzheimer's brain. As shown in FIG. 3, the α-synuclein sdAb clones have diverse binding profiles to the different brain/protein fractions. Note that the concentration of sdAb in each supernatant is unknown. Hence, it is not appropriate to compare binding strength of individual clones, only their profile of binding to the different proteins/brains.

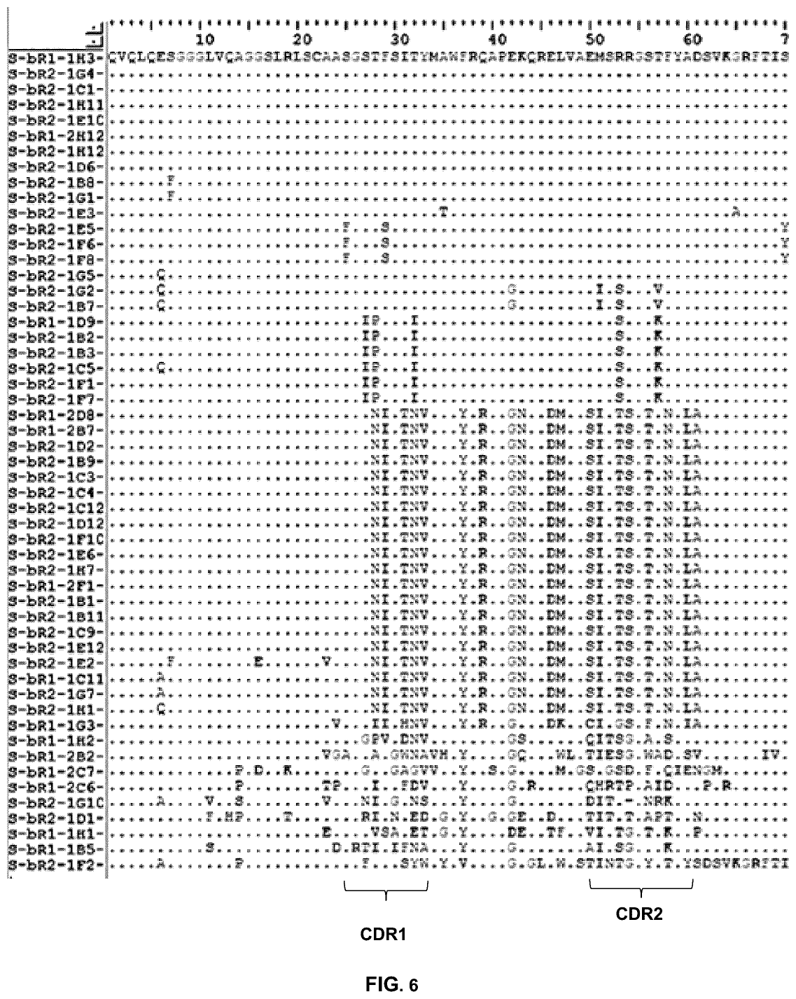

To examine sdAb binding in human brain tissue, purified sdAbs were incubated with tissue sections of human brain with Lewy Body Dementia (LBD). A mixture of two commercial α-synuclein antibodies, i.e., mAb 211 and pAb C20 (Santa Cruz), known to stain Lewy bodies (LB) and Lewy neurites (LN) that sometimes circulate plaques were used as positive controls (FIG. 4A). As shown in FIGS. 4B and 4C, the polyclonal sdAbs panned against solid phase α-synuclein (sR2) (FIG. 4B) and biotinylated soluble phase α-synuclein (bR2) (FIG. 4C) recognize LB like structures and some plaques, sdAb 1G10 binds to various structures including LB, LN and plaques (FIG. 4D), as well as vasculature and axons, sdAb 2D10 and 2D8 binds to LB (FIGS. 4E, 4F), sdAb 2H7 binds to diverse structures including LB, plaques (FIG. 4G) and axons (not shown), sdAb 2H1 binds to LB (FIG. 4H), sdAb 1D12 is the least reactive and binds to plaques, LB and vasculature (FIG. 4I). The plaque-like structures that some of the sdAbs bind to are peculiar but α-synuclein antibodies have previously been shown to stain plaques, because α-synuclein can accumulate in dystrophic neurites (Yang F et al, Brain Res 853:381-383 (2000), which is hereby incorporated by reference in its entirety.

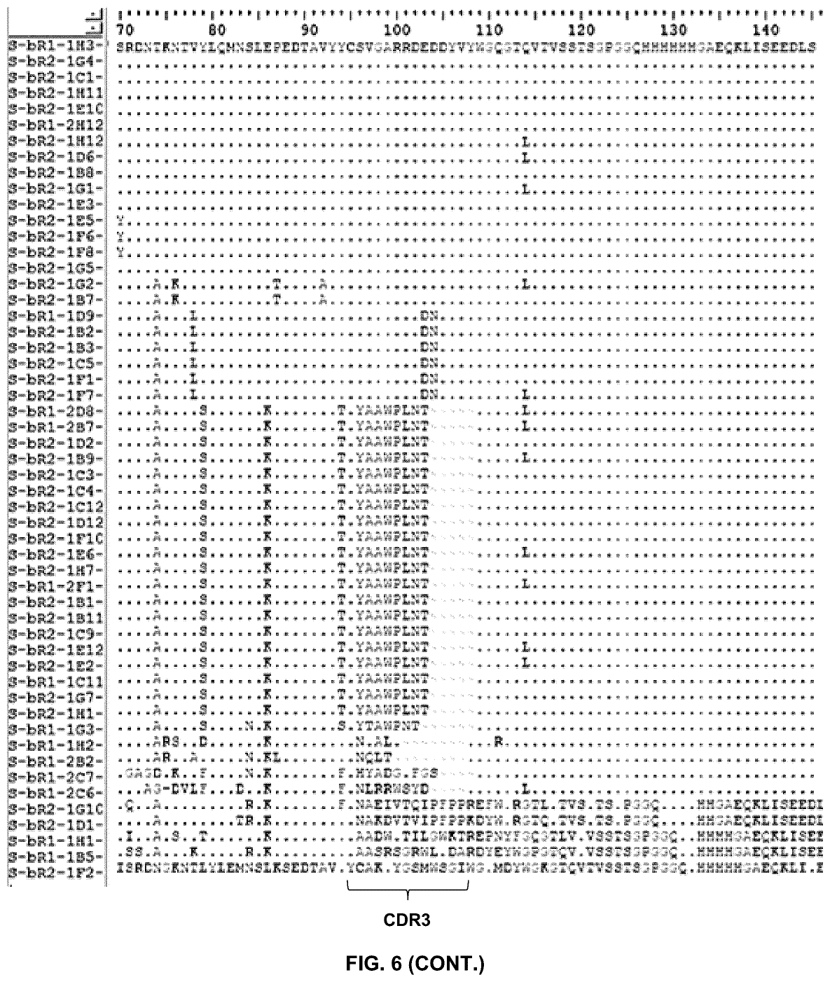

As shown in FIGS. 4A-4I, extensive Lewy bodies and Lewy neurites are seen in the DLB brain. The DLB brain also contains extensive tau pathology neurofibrillary tangles, pretangles and dystrophic neurites as shown by positive immunoreactivity with the PHF1 antibody (FIG. 5B). Although some of the sdAbs bind to tau fractions on ELISA, they do not appear to bind to pathological tau on brain sections as shown in FIGS. 4A-4I.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. An anti-alpha-synuclein single-domain antibody or binding fragment thereof, said antibody or binding fragment thereof comprising a heavy chain variable region that comprises:
a complementarity-determining region 1 (H-CDR1) comprising an amino acid sequence of any one of SEQ ID NOs: 33, 147, 151, or 218;
a complementarity-determining region 2 (H-CDR2) comprising an amino acid sequence of any one of SEQ ID NOs: 69, 162, 166, or 219; and
a complementarity-determining region 3 (H-CDR3) comprising an amino acid sequence of any one of SEQ ID NOs: 105, 177, 181, or 220.

2.-3. (canceled)

4. The antibody or binding fragment thereof of claim 1, wherein said antibody or binding fragment thereof is a camelid antibody or binding fragment thereof.

5. (canceled)

6. The antibody or binding fragment thereof of claim 1, wherein said heavy chain variable region comprises:
the H-CDR1 comprising the amino acid sequence of ASGSNISTNV (SEQ ID NO: 147);
the H-CDR2 comprising the amino acid sequence of SISTSGTTNYLA (SEQ ID NO: 162); and
the H-CDR3 comprising the amino acid sequence of YAAWPLNT (SEQ ID NO: 177.

7. The antibody or binding fragment thereof of claim 1, wherein said heavy chain variable region comprises:
the H-CDR1 comprising the amino acid sequence of ASVVPFRYF (SEQ ID NO: 33;
the H-CDR2 comprising the amino acid sequence of ASITSGGGVN (SEQ ID NO: 69); and
the H-CDR3 comprising the amino acid sequence of ARLLSLGSRWGYGMFT (SEQ ID NO: 105).

8. The antibody or binding fragment thereof of claim 1, wherein said heavy chain variable region comprises:
the H-CDR1 comprising the amino acid sequence of ASGNIFGINS (SEQ ID NO: 151);
the H-CDR2 comprising the amino acid sequence of DITRGNRKYAD (SEQ ID NO: 166); and
the H-CDR3 comprising the amino acid sequence of NAEIVTQIPFPPR (SEQ ID NO: 181).

9. The antibody or binding fragment thereof of claim 1, wherein said heavy chain variable region comprises:
the H-CDR1 comprising the amino acid sequence of ASGNIFRINA (SEQ ID NO: 218);
the H-CDR2 comprising the amino acid sequence of ASIDSAGRTNYG (SEQ ID NO: 219); and
the H-CDR3 comprising the amino acid sequence of CSTWRLGRNY (SEQ ID NO: 220).

10. The antibody or binding fragment thereof of claim 1, wherein said heavy chain variable region further comprises human or humanized immunoglobulin heavy chain framework regions.

11.-12. (canceled)

13. The antibody or binding fragment thereof of claim 1, wherein the heavy chain variable region comprises the amino acid sequence selected from the group consisting of SEQ ID NOs: 110, 142, 200, and 221.

14.-26. (canceled)

27. An antibody composition comprising two or more antibodies or binding fragments thereof of claim 1.

28. An isolated polynucleotide encoding the antibody or binding fragment thereof of claim 1.

29. A vector comprising the isolated polynucleotide of claim 28.

30. The vector of claim 29, wherein said vector is an adeno-associated viral vector.

31. A host cell comprising the vector of claim 29.

32. A pharmaceutical composition comprising:
the antibody or binding fragment thereof of claim 1; and
a pharmaceutical carrier.

33. A method of inhibiting onset of one or more symptoms of an α-synucleinopathy in a subject, said method comprising:
administering to the subject the pharmaceutical composition of claim 32, wherein said composition is administered in an amount effective to inhibit onset of one or more symptoms of the α-synucleinopathy in the subject.

34. A method of treating an α-synucleinopathy in a subject, said method comprising:
administering to the subject the pharmaceutical composition of claim 32, wherein said composition is administered in an amount effective to treat the α-synucleinopathy in the subject.

35.-36. (canceled)

37. A method of diagnosing an α-synucleinopathy in a subject, said method comprising:
detecting, in the subject, the presence of accumulated α-synuclein protein or peptide using the antibody or binding fragment thereof of claim 1, and
diagnosing the α-synucleinopathy in the subject based on said detecting.

38.-40. (canceled)

41. A method of monitoring the progression of an α-synucleinopathy in a subject, said method comprising:
detecting, in the subject, the presence of α-synuclein protein or peptide using the antibody or binding fragment thereof of claim 1;
repeating said detecting periodically; and
monitoring the progression of the α-synucleinopathy in the subject based on said repeated detecting.

42.-45. (canceled)

46. A diagnostic kit comprising:
the antibody or binding fragment thereof of claim 1 and
a detectable label.

47. A fusion polypeptide comprising two or more of the heavy chain variable regions of the anti-alpha-synuclein single-domain antibody according to claim 1.
